SEQUENCE LISTING (1 sequence)

SEQ ID NO: 1 - protein, 37 aa, synthetic construct
  SITE 11: note = Alpha-aminoisobutyric acid
  SITE 18: note = (1S,28S)-1-amino-7,16,25,30-tetraoxo-9,12,18,21-tet raoxa-6,15,24,29-tetraazaoctatetracontane-1,28,48-tricarbo xylic acid
  SITE 37: note = Amidated residue
EGTFTSDYSIXLDKIAQKAFVQWLIAGGPSSGAPPPS

(19) United States
(12) Patent Application Publication
(10) Pub. No.: US 2026/0137758 A1
(43) Pub. Date: May 21, 2026

(54) TREATMENT OF STEATOTIC LIVER DISEASE ASSOCIATED WITH METABOLIC DYSFUNCTION

(71) Applicants: Carmot Therapeutics Inc., South San Francisco, CA (US); Genentech, Inc., South San Francisco, CA (US); Hoffmann-La Roche Inc., Little Falls, NJ (US)

(72) Inventors: Muhamed Barakovic, Basel (CH); Manu Chakravarthy, Newton, MA (US); Brandon David Kayser, South San Francisco, CA (US); Alexandra Vyacheslavovna Shteynberg, Alameda, CA (US); Jiawen Zhu, South San Francisco, CA (US)

(73) Assignees: Carmot Therapeutics Inc., South San Francisco, CA (US); Genentech, Inc., South San Francisco, CA (US); Hoffmann-La Roche Inc., Little Falls, NJ (US)

(21) Appl. No.: 19/396,003

(22) Filed: Nov. 20, 2025

Related U.S. Application Data
(60) Provisional application No. 63/723,083, filed on Nov. 20, 2024.
(60) Provisional application No. 63/784,857, filed on Apr. 7, 2025.
(60) Provisional application No. 63/819,443, filed on Jun. 6, 2025.
(60) Provisional application No. 63/829,596, filed on Jun. 24, 2025.
(60) Provisional application No. 63/900,015, filed on Oct. 15, 2025.

(51) Int. Cl. A61K 38/26; A61P 1/16

(52) U.S. Cl. CPC A61K 38/26; A61P 1/16

(57) ABSTRACT

The present disclosure provides methods of treating a steatotic liver disease associated with metabolic dysfunction, such as metabolic dysfunction-associated steatohepatitis (MASH), metabolic dysfunction-associated fatty livers disease (MAFLD), or metabolic dysfunction-associated steatotic liver disease (MASLD). The treatment comprises administration of a dual GLP-1 receptor and GIP receptor agonist.

### CROSS-REFERENCE TO RELATED APPLICATIONS

The present application claims priority from U.S. Applications 63/723,083, filed Nov. 20, 2024; 63/784,857, filed Apr. 7, 2025; 63/819,443, filed Jun. 6, 2025; 63/829,596, filed Jun. 24, 2025; and 63/900,015, filed Oct. 15, 2025. The contents of the priority applications are incorporated by reference herein in their entirety.

### SEQUENCE LISTING

The instant application contains a Sequence Listing that has been submitted electronically in XML format and is hereby incorporated by reference in its entirety. Said XML copy, created on Nov. 19, 2025, is named 124921_US011_SL.xml and is 2,987 bytes in size.

### BACKGROUND OF THE INVENTION

Steatotic liver disease, also known as fatty liver disease, is characterized by excess accumulation of fat in the liver. A normal human liver contains a small amount of fat, typically less than 5% of its weight. A fatty liver or hepatic steatosis is diagnosed when more than 5% (e.g., more than 5% to 10%) of the liver's weight is fat. Hepatic steatosis may be diagnosed by imaging (e.g., ultrasound, computerized tomography (CT) scan, magnetic resonance imaging (MRI)) or biopsy, and may be supported by blood tests.

Steatotic liver disease starts with accumulation of lipids as intrahepatic vesicles without affecting essential liver functions. The disease can progress to steatohepatitis, characterized by hepatocyte ballooning, apoptosis, accumulation of Mallory-Denk bodies, and inflammation within the liver parenchyma and/or portal vein. Persistent disease condition may lead to tissue scarring (fibrosis), and if left untreated, cirrhosis and liver failure.

Fatty liver disease not caused by excess alcohol consumption was known as nonalcoholic fatty liver disease (NAFLD). To more accurately capture the disease condition and enable better treatment, the medical community has now adopted the term “metabolic dysfunction-associated steatotic liver disease” (MASLD) (Rinella et al., Hepatology (2023) 78:1966-86) or “metabolic dysfunction-associated fatty liver disease” (MAFLD) (see, e.g., Eslam et al., J Hepatology. (2020) 73:202-9 and Tacke et al., J Hepatology. (2024) 81(3):492-542). MASLD and MAFLD are diagnosed with different criteria but cover largely overlapping patient populations. Both diseases are characterized by steatosis in the liver and are associated with metabolic dysfunction.

An advanced stage of MASLD or MAFLD is metabolic dysfunction-associated steatohepatitis (MASH), formerly known as nonalcoholic steatohepatitis (NASH). MASH is characterized by inflammation and liver cell damage and potentially leads to cirrhosis, liver failure, or even liver cancer.

In the United States, approximately 25% of adults have MASLD, about 20-25% of which have progressed to MASH. Given the prevalence of steatotic liver disease, there is an acute need for developing safe, effective, and well-tolerated therapies for its treatment.

### SUMMARY OF THE INVENTION

The present disclosure provides a method of treating steatotic liver disease associated with metabolic dysfunction, e.g., metabolic dysfunction-associated steatohepatitis (MASH), metabolic dysfunction-associated fatty liver disease (MAFLD), or metabolic dysfunction-associated steatotic liver disease (MASLD), in an adult patient in need thereof. The method comprises administering by subcutaneous injection to the patient a compound of Formula I, or a pharmaceutically acceptable salt or ester thereof:

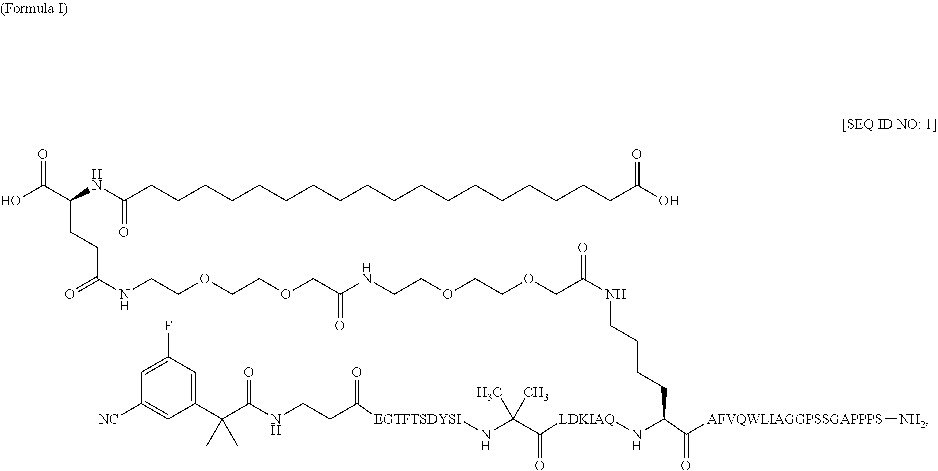

wherein the compound is administered at a body weight-independent dose of about 2.0 mg to about 25 mg. In some embodiments, the method is for treating MAFLD or MASLD, and the patient optionally has a magnetic resonance imaging-proton density fat fraction (MRI-PDFF) of ≥5%. In some embodiments, the method is for treating MASH, and the patient optionally has an MRI-PDFF of ≥10%.

In another aspect, the present disclosure provides a method of decreasing liver fat as compared to baseline (optionally by 30% or more), decreasing liver fat to <5% (e.g., to a MRI-PDFF value of <5%), or decreasing fibro-inflammation evaluated via iron corrected T1 (cT1) as compared to baseline (optionally by 40 ms or more), decreasing Pro-C3 as compared to baseline (optionally by 15 ng/mL or more), or treating liver fibrosis (optionally by improving fibrosis by at least one stage) in an adult patient in need thereof, comprising administering by subcutaneous injection to the patient a compound of Formula I, or a pharmaceutically acceptable salt or ester thereof:

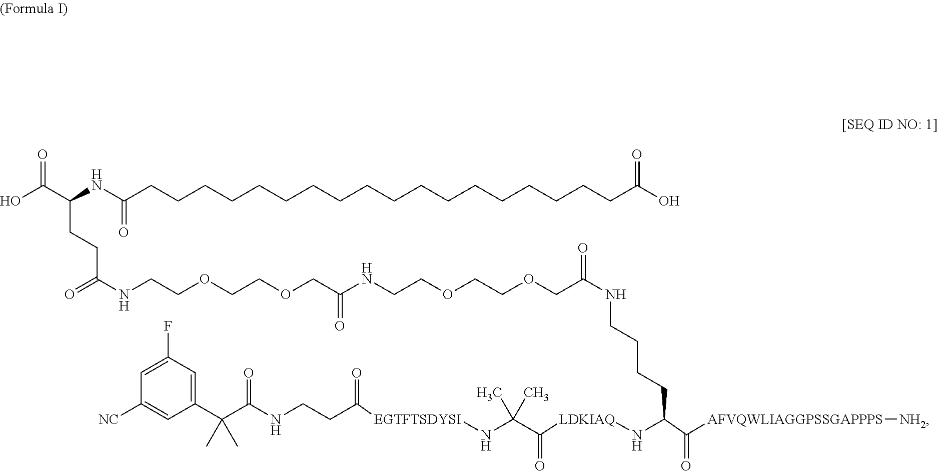

wherein the compound is administered at a body weight-independent dose of about 2.0 mg to about 25 mg.

In some embodiments, the patient is an overweight or obese adult patient. In some embodiments, the patient has a BMI of ≥25 kg/m², ≥27 kg/m², ≥30 kg/m², ≥35 kg/m², or ≥40 kg/m². The patient may be obese and may optionally have a BMI of ≥30.0 kg/m². In some embodiments, the patient has liver fibrosis. The liver fibrosis may be mild, moderate, or advanced or may be stage F1, F2, or F3 fibrosis. In some embodiments, the patient receives additional therapy such as diet therapy or exercise therapy.

In some embodiments, the patient does not have type 2 diabetes mellitus or is prediabetic. In other embodiments, the patient has type 2 diabetes mellitus. In some embodiments, the patient has one or more weight-related comorbidities, optionally prediabetes, hypertension, dyslipidemia, obstructive sleep apnea, or previously diagnosed cardiovascular disease.

The administering step may be repeated once every week, once every two weeks, once every four weeks, or once every month. The dose may be about 2.0, about 4.0, about 5.0, about 8.0, about 12.0, about 16.0, about 17.0, about 20.0, about 22.0, or about 24.0 mg, and optionally may be up-titrated over a period of 57 days. In some embodiments, the dose is up-titrated once a month or longer (e.g., once every two months). In some embodiments, the starting dose for the up-titration is about 5 mg. In some embodiments, the maximum dose for the up-titration is about 22 mg.

Also provided is a compound of Formula I, or a pharmaceutically acceptable salt or ester thereof:

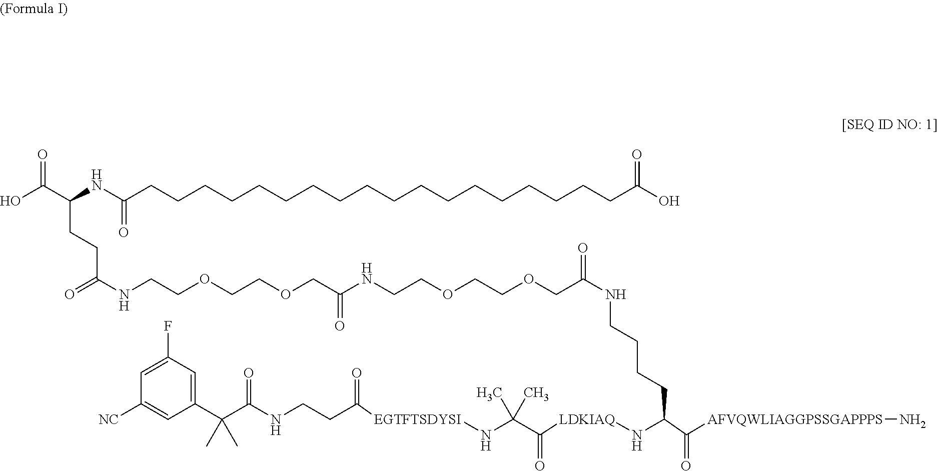

for use in a method described herein.

The present disclosure also provides use of a compound of Formula I, or a pharmaceutically acceptable salt or ester thereof:

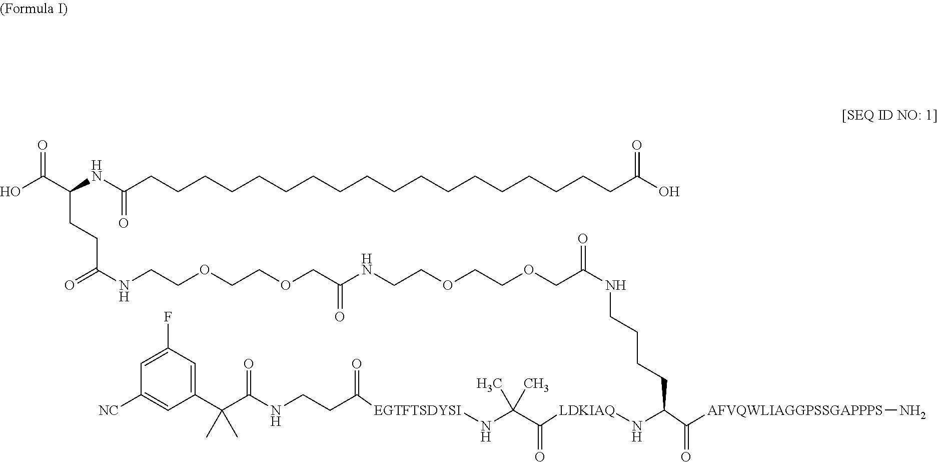

in the manufacture of a medicament for use in a method disclosed herein.

Also provided is an article of manufacture or kit for use in a method described herein, wherein the article of manufacture or kit comprises one or more units of said dose. The article of manufacture or kit may comprise a syringe or an injector, optionally a single-use syringe or injector.

Other features, objectives, and advantages of the invention are apparent in the detailed description that follows. It should be understood, however, that the detailed description, while indicating embodiments and aspects of the invention, is given by way of illustration only, not limitation. Various changes and modifications within the scope of the invention will become apparent to those skilled in the art from the detailed description.

### BRIEF DESCRIPTION OF THE FIGURES

FIG. 1 is a diagram showing the cohort 12 subject dispositions. *Adjustments included skip doses or dose down titrations; **Food poisoning; ***Drug holiday at W23 (bi-weekly dosing) due to BMI<23 kg/m². The MRI-PDFF analysis population includes participants who had baseline, week 12 and EOT (Week 24) MRI-PDFF data.

FIG. 2 is a diagram showing the weight loss (%) thresholds associated with certain health outcomes in patients.

FIG. 3A is a box and whisker plot showing observed percent change from baseline in body weight over time in patients in cohorts 11 and 12 of the clinical trial described in Example 1. The squares represent the means; lines within the squares represent medians; and the whiskers represent the 5th and 95th percentiles.

FIG. 3B is a plot showing the % change in body weight from baseline with multiple dosing over 24 weeks. For placebo, n=7, and for participants treated with CT-388 (22 mg), n=24.

FIG. 4A is a box and whisker plot showing liver fat at baseline, week 12, and week 24 based on the overall population with MRI-PDFF (LFC (%)) data. FIG. 4B is a box and whisker plot showing the percent change in liver fat at weeks 12 and 24 based on the overall population.

FIGS. 5A-5C are a series of bar graphs showing the effect of CT-388 on liver MRI-PDFF (liver fat content (LFC)) in the overall population of participants with obesity, represented as percent of patients (%) (proportion reaching target (%)), including liver fat reduction by target at week 12 (FIG. 5A) and week 24 (FIG. 5B) and percentage of participants reaching liver MRI-PDFF<5% (normal) at baseline, week 12 and week 24 (FIG. 5C).

FIG. 6A is a scatter plot showing relative liver fat reduction as compared to percent change in body weight. FIG. 6B is a scatter plot showing relative liver fat reduction as compared to change in waist circumference.

FIG. 7A is a box and whisker plot showing liver fat at baseline, week 12, and week 24 in participants with an MRI-PDFF baseline of ≥5%. FIG. 7B is a box and whisker plot showing percent change in liver fat at weeks 12 and 24 in participants with an MRI-PDFF baseline of ≥5%.

FIGS. 8A-8C are a series of bar graphs showing liver fat reduction by target in participants with an MRI-PDFF baseline of ≥5%. FIG. 8A and FIG. 8B show the relative reduction in liver fat at weeks 12 and 24, respectively. FIG. 8C shows the proportion of participants (proportion reaching target) with baseline MRI-PDFF≥5% with liver fat <5% (normal) at baseline and weeks 12 and 24.

FIG. 9A is a box and whisker plot showing liver fat at baseline, week 12, and week 24 in participants with an MRI-PDFF baseline of ≥10%. FIG. 9B is a box and whisker plot showing observed percent change in liver fat at weeks 12 and 24 in participants with an MRI-PDFF baseline of ≥10%.

FIGS. 10A-10C are a series of bar graphs showing liver fat reduction by target in participants with an MRI-PDFF baseline of ≥10%. FIG. 10A and FIG. 10B show the relative reduction in liver fat at weeks 12 and 24, respectively. FIG. 10C shows the proportion of participants (proportion reaching target) with baseline MRI-PDFF≥10% with liver fat <5% (normal) at baseline and weeks 12 and 24.

FIG. 11 is a chart showing the LS mean of relative liver fat reduction at weeks 12 and 24 in participants with an MRI-PDFF baseline of ≥10%. This data was generated using an MMRM model to adjust for within-subject variation.

FIG. 12 is a chart showing the placebo-adjusted LS mean of relative liver fat reduction at weeks 12 and 24 in participants with an MRI-PDFF baseline of ≥10%. This data was generated using an MMRM model to adjust for within-subject variation.

FIG. 13A is a line plot showing individual cT1 measurements at baseline and week 24. FIG. 13B shows cT1 in participants at baseline, week 12, and week 24.

FIGS. 14A and 14B are bar graphs showing cT1 reductions (percent of patients or proportion of participants reaching target) of >40 or >80 ms in participants in the MRI-PDFF analysis population of Cohort 12 at week 12 (FIG. 14A) and week 24 (FIG. 14B).

FIGS. 15A and 15B are bar graphs showing cT1 reductions (percent of patients or proportion of participants reaching target) of >40 or >80 ms in participants with obesity and MASLD with MRI-PDFF baseline ≥5% at weeks 12 (FIG. 15A) and 24 (FIG. 15B). FIG. 15C is a line graph showing cT1 reduction in each participant.

FIGS. 16A and 16B are bar graphs showing cT1 reduction from baseline at weeks 12 (FIG. 16A) and 24 (FIG. 16B) in participants with an MRI-PDFF baseline ≥10%. FIG. 16C is a line graph showing cT1 reduction in each participant with an MRI-PDFF baseline ≥10%.

FIG. 17A is a box and whisker plot showing cT1 levels at baseline and weeks 12 and 24 in participants with an MRI-PDFF baseline ≥10%. FIG. 17B is a box and whisker plot showing change in baseline at weeks 12 and 24 of cT1 levels in participants with an MRI-PDFF baseline ≥10%.

FIG. 18A shows percent change in median score (cT1) vs. percent change in weight at week 12 and week 24 in cohort 12 (obese without T2DM). FIG. 18B shows change in median score (cT1) vs. percent change in waist circumference at week 12 and week 24 in cohort 12 (obese without T2DM).

FIG. 19A shows mean % change in ALT and AST in patients. FIG. 19B shows mean % change in GGT and ALP in patients.

FIG. 20A is a box plot showing the observed ALT (U/L) in participants. FIG. 20B is a box plot showing the change from baseline in ALT (U/L) in participants.

FIG. 21A is a box plot showing the observed AST (U/L) in participants. FIG. 21B is a box plot showing the Change from Baseline (Bottom) in AST (U/L)

FIG. 22A is a box plot showing the observed total bilirubin (mg/dL) in participants. FIG. 22B is a box plot showing the change from baseline in total bilirubin (mg/dL) in participants.

FIG. 23 is a box plot showing change in liver fat content (LFC) in participants with obesity (with or without T2D) with LFC≥5% at baseline.

FIG. 24A shows liver MRI-PDFF images from a 24-year-old female participant with MASLD at baseline (left) and after 24 weeks of treatment (right).

FIG. 24B shows liver cT1 images from a 24-year-old female participant with MASLD at baseline (left) and after 24-weeks of treatment (right).

FIGS. 25A and 25B are schematics showing the clinical study design for CT-388. Abbreviations are as follows: BMI, body mass index; C, cohort; D, day; EOT, end of treatment; MAD, multiple ascending dose; QW, once a week; SAD, single ascending dose; SC, subcutaneous.

FIGS. 26A-26F are graphs showing the in vitro activity of native GLP-1, GIP, and CT-388 on the human GLP-1 and GIP receptors. FIGS. 26A and 26D show representative concentration response curves for mean (SD) cAMP accumulation. FIGS. 26B and 26E show mean (SD) β-arrestin-2 recruitment. FIGS. 26C and 26F show mean (SE) receptor internalization in vitro following treatment with native GLP-1 or CT-388.

FIGS. 27A-27D are graphs showing the in vitro activity of native GLP-1, GIP, and CT-388 on the mouse GLP-1 and GIP receptors. Representative concentration response curves for cAMP accumulation (FIGS. 27A and 27C) and β-arrestin-2 recruitment (FIGS. 27B and 27D) are provided.

FIG. 28 is a graph showing glucose-stimulated insulin secretion in primary human beta cells treated with CT-388.

FIGS. 29A-29D are graphs showing the effects of CT-388 on glucose homeostasis during an intraperitoneal glucose tolerance test in mice or an intravenous glucose tolerance test (ivGTT) in cynomolgus monkeys. FIG. 29A shows blood glucose levels during an ipGTT assay in lean male C57BL/6J mice. FIGS. 29B-D, respectively, show insulin, C-peptide, and blood glucose levels during an ivGTT in lean cynomolgus monkeys (n=4 per group). *P<0.05, **P<0.01, ***P<0.001 as compared to vehicle control. Abbreviations are as follows: AUC, area under the concentration-time curve; ipGTT, intraperitoneal glucose tolerance test; ivGTT, intravenous glucose tolerance test.

FIGS. 30A-30J show the effects of CT-388 on body weight, food consumption, and glucose control after acute and sub-chronic administration in mice. FIGS. 30A and 30B show, respectively, body weight and food intake in lean C57BL/6J mice treated with 6 nmol/kg of CT-388 or vehicle. FIGS. 30C and 30D show, respectively, body weight and liver weight in DIO mice treated with 6 nmol/kg of CT-388 or vehicle. FIGS. 30E-30J show body weight (FIG. 30E), food intake (FIG. 30F), FBG levels (FIG. 30G), fasting insulin levels (FIG. 30H), iWAT weight (FIG. 30I), and liver weight (FIG. 30J) in MC4RKO mice treated with 30 nmol/kg of CT-388 or vehicle. Abbreviations are as follows: DIO, diet-induced obesity; FBG, fasting blood glucose; iWAT, inguinal white adipose tissue; MC4RKO, melanocortin receptor 4 knock-out. *P<0.05, **P<0.01, ***P<0.001 vs vehicle control.

FIGS. 31A-31J show the body weight (FIG. 31A), iWAT weight (FIG. 31B), liver weight (FIG. 31C), FBG levels (FIG. 31D), fasting insulin levels (FIG. 31E), AST levels (FIG. 31F), ALT levels (FIG. 31G), lipid area (FIG. 31H), ballooning hepatocyte density (FIG. 31I), and NAS score (FIG. 31J) in GAN-DIO mice treated with 6 nmol/kg of CT-388 or vehicle. Abbreviations are as follows: ALT, alanine aminotransferase; AST, aspartate aminotransferase; GAN-DIG, Gubra-Amylin diet-induced obese; FBG, fasting blood glucose; iWAT, inguinal white adipose tissue; NAS, nonalcoholic fatty liver disease activity score. **P<0.01, ***P<0.001, ****P<0.0001 vs vehicle control.

FIGS. 32A and 32B show participant disposition in the SAD portion of the study (FIG. 32A) and the MAD portion of the study (FIG. 32B). The SAD portion of the study began before the MAD portion of the study. Abbreviations are as follows: MAD, multiple ascending dose; SAD, single ascending dose. ^{a}The randomization was performed within each study cohort. ^{b}One participant who received a single dose of 6 mg of CT-388 discontinued from the study (due to withdrawal by participant).

FIGS. 33A and 33B show CT-388 plasma concentrations after administration in the SAD (FIG. 33A) and MAD (FIG. 33B) portions of the study. Values for the MAD portion of the study were measured after the CT-388 dose was administered on day 8.

FIG. 34 is a graph showing weight loss after treatment with CT-388 in the SAD portion of the study.

FIGS. 35A-35F are graphs showing mean (SD) percent change from baseline to day 43 in body weight (FIG. 35A) and mean (SD) absolute change from baseline to day 29 in hip circumference (FIG. 35B), waist circumference (FIG. 35C), fasting glucose levels (FIG. 35D), fasting insulin levels (FIG. 35E), and HOMA-IR (FIG. 35F) in patients. Abbreviations are as follows: C, CT-388 cohort; EOT, end of treatment; HOMA-IR, Homeostatic Model Assessment for Insulin Resistance; MAD, multiple ascending dose.

FIGS. 36A-36H are graphs showing glucose, insulin, and HOMA-IR responses during an oral glucose tolerance test in the MAD portion of the clinical study: mean (SEM/SD) difference between day −1 and day 23 during an oral glucose tolerance test for glucose levels (FIG. 36A), AUC₀₋₁₂₀ for difference in glucose levels (FIG. 36B), insulin levels (FIG. 36C), AUC₀₋₁₂₀ for difference in insulin levels (FIG. 36D), HOMA-IR (FIG. 36E), HOMA-IR at t0 (FIG. 36F), C-peptide levels (FIG. 36G), and AUC₀₋₁₂₀ for difference in C-peptide levels (FIG. 3611). Abbreviations are as follows: AUC, area under the concentration-time curve; AUC₀₋₁₂₀, area under the concentration-time curve from time 0 to 120 minutes; HOMA-IR, Homeostatic Model Assessment for Insulin Resistance; MAD, multiple ascending dose; SEM, standard error of the mean; to, time 0 of the oral glucose tolerance test.

FIGS. 37A and 37B are graphs showing effects of CT-388 on satiety and appetite in the MAD portion of the study: mean (SD) change from baseline to day 29 for satiety total score (FIG. 37A) and appetite VAS score (FIG. 37B). Abbreviations are as follows: C, CT-388 cohort; MAD, multiple ascending dose; VAS, visual analog scale appetite after treatment with CT-388 in the MAD portion of the study.

FIGS. 38A-38C are diagrams showing the Phase 1B clinical trial design for CT-388 in adults with obesity, with and without T2D (FIG. 38A), as well as diagrams showing Cohort 12 (FIG. 38B) and Cohort 13 (FIG. 38C) subject dispositions.

FIG. 39A is a box and whisker plot showing liver fat at baseline and week 12 based on the overall population with MRI-PDFF (LFC (%)) data of Cohorts 12 and 13. FIG. 39B is a box and whisker plot showing liver fat change from baseline based on the overall population with MRI-PDFF (LFC (%)) data of Cohorts 12 and 13. FIG. 39C is a box and whisker plot showing liver fat at baseline and week 12 in participants with an MRI-PDFF baseline of ≥5% in Cohorts 12 and 13. FIG. 39D is a box and whisker plot showing liver fat change from baseline based on the overall population with and MRI-PDFF baseline of ≥5% in Cohorts 12 and 13. FIG. 39E is a box and whisker plot showing liver fat at baseline and week 12 in participants with an MRI-PDFF baseline of ≥10% in Cohorts 12 and 13. FIG. 39F is a box and whisker plot showing liver fat change from baseline based on the overall population with and MRI-PDFF baseline of ≥10% in Cohorts 12 and 13.

FIGS. 40A-40D are a series of bar graphs showing the proportion (%) of participants reaching target reduction of liver fat by week 12 in the MRI analysis population of all participants without T2D in Cohort 12 (FIG. 40A) and participants without T2D in Cohort 12 with baseline LFC≥5% (FIG. 40B), as well as of all participants with T2D in Cohort 13 (FIG. 40C) and participants with T2D in Cohort 13 with baseline LFC≥5% (FIG. 40D).

FIGS. 41A and 41B are scatter plots showing the correlation between percent (%) change from baseline to week 12 in body weight and LFC for participants with baseline LFC≥5% in Cohort 12 (FIG. 41A) and Cohort 13 (FIG. 41B).

FIGS. 42A and 42B are scatter plots showing the correlation between percent (%) change from baseline to week 12 in body weight (kg) (FIG. 42A) and relative change in weight (%) (FIG. 42B) for participants in Cohort 12 (blue and dark gray datapoints) and Cohort 13 (green and light grey datapoints).

FIG. 43A shows cT1 in participants at baseline and week 12, as well as cT1 change from baseline (ms) for Cohorts 12 and 13. FIG. 43B shows cT1 in participants with baseline LFC≥5% at baseline and week 12, as well as cT1 change from baseline (ms) for Cohorts 12 and 13. FIG. 43C shows cT1 overtime in participants with baseline LFC≥5% from baseline to week 12. FIG. 43D shows cT1 in participants with baseline LFC 10% at baseline and week 12, as well as cT1 change from baseline (ms) for Cohorts 12 and 13. FIG. 43E shows cT1 overtime in participants with baseline LFC≥10% from baseline to week 12.

FIG. 44A shows mean % change in visceral adipose tissue (VAT) fat in patients. FIG. 44B shows mean % change in subcutaneous adipose tissue (SAT) fat in patients.

FIGS. 45A and 45B are a set of box and whisker plots showing body weight (kg) of all participants and participants with baseline LFC≥5%, at baseline and week 12, in participants without T2D (FIG. 45A) and with T2D (FIG. 45B).

FIGS. 46A-46D are a set of box and whisker plots showing ProC3 (ng/mL) (FIGS. 46A and 46B) and ALT (FIGS. 46C and 46D) of all participants and participants with baseline LFC≥5%, at baseline and week 12, in participants without T2D (FIGS. 46A and 46C) and with T2D (FIGS. 46B and 46D).

FIG. 47 shows a phase 1b trial design for CT-388 in adults with obesity with or without T2D. Dashed lines indicate delayed titration, which was allowed on a weekly basis during weeks 3-12 (low-dose non-T2D cohorts) or weeks 4-12 (high-dose non-T2D and T2D cohorts) if participants experienced moderate-to-severe tolerability issues. ^{a}T2D diagnosis according to the 2022 American Diabetes Association Standards of Medical Care in Diabetes. ^{b}T2D managed with diet and/or exercise alone or treated with a stable dose of metformin monotherapy for ≥3 months before screening. BMI=body mass index, EOT=end of treatment, MD=multiple dose, OGTT=oral glucose tolerance test, QW=once weekly, SC=subcutaneous, t=time, T2D=type 2 diabetes, y=years.

FIG. 48 shows participant disposition in the study. Patients who were ‘eligible but not enrolled’ were not enrolled because they did not attend the check-in visit or because the trial enrolment had already been completed. Planned regimen 1=5 mg×3 wks/8 mg×9 wks (low-dose non-T2D cohort). Planned regimen 2=5 mg×2 wks/8 mg×2 wks/12 mg×2 wks/17 mg×2 wks/22 mg×16 wks (high-dose non-T2D cohort). Planned regimen 3=5 mg×2 wks/8 mg×2 wks/12 mg×2 wks/17 mg×2 wks/22 mg×4 wks (high-dose T2D cohort). All placebo=low- and high-dose non-T2D cohorts were pooled. T2D=type 2 diabetes.

FIG. 49 shows the weekly incidence of vomiting over 24 weeks in the high-dose CT-388 non-T2D cohort. Doses were up-titrated at the indicated time points. Weekly TEAE incidence vs week curves were smoothed using spline methods. EOT=end of treatment, T2D=type 2 diabetes, TEAE=treatment-emergent adverse event.

FIG. 50 shows the weight loss change from baseline (panels (a) and (b)) and achievement of weight loss targets (panels (c) and (d)) in non-T2D and T2D cohorts, respectively. Percent change from baseline in body weight was analyzed using mixed model repeated measures. Results include all participants who received treatment, regardless of the final dose. ^{a}Placebo groups for low- and high-dose non-T2D cohorts are pooled for this analysis. ^{b}No participants in the T2D cohort met weight loss targets of ≥15. LS=least squares, SE=standard error, T2D=type 2 diabetes, WL=weight loss.

FIG. 51 shows the HbA1c change from baseline (panel (a)), achievement of HbA1c targets (panel (b)), and 7-point SMBG (panel (c)) in the high-dose T2D cohort. ^{a}HbA1c≤6.5% is the target of glycemic control per AACE Consensus, and HbA1c lowering from 7.0% to 6.0% is associated with reduction in microvascular complications. In panel (c), the optimal SMBG range of 70-100 mg/dL is shaded. BL=baseline, CI=confidence interval, h=hour, HbA1c=glycated hemoglobin, LS=least squares, min=minimum, max=maximum, pbo=placebo; pts=participants, SE=standard error; SMBG=self-monitoring of blood glucose, T2D=type 2 diabetes.

FIG. 52 shows the fasting glucose change from baseline in the high-dose T2D cohort. LS=least squares, SE=standard error, T2D=type 2 diabetes.

FIG. 53 shows the glycemic status at week 24 in the high-dose non-T2D cohort. Results include all participants who received treatment, regardless of the final dose. ^{a}Normoglycemia defined according to three ADA criteria: HbA1c<5.7%, fasting glucose <100 mg/dL and 2-hour glucose during OGTT<140 mg/dL. ^{b}Two participants had diabetes at baseline; at week 24, one became normoglycemic and one had missing data. ADA=American Diabetes Association, HbA1c=glycated hemoglobin, OGTT=oral glucose tolerance test, T2D=type 2 diabetes.

FIG. 54 shows observed means of glucose, insulin and C-peptide during OGTT. OGTT curves are observed mean±SEM in the intent-to-treat population. ^{a}Placebo groups for low- and high-dose non-T2D cohorts are pooled for this analysis. OGTT=oral glucose tolerance test, SEM=standard error of the mean, T2D=type 2 diabetes.

FIG. 55 shows the percent change from baseline (CFB) in AUC for glucose, insulin and C-peptide during OGTT. A mixed-effect model for repeated measures analysis of percent CFB in AUC (calculated using the trapezoidal rule) was used in the intent-to-treat population. Adjusted mean % CFB of AUC are shown above/below bars. ^{a}Placebo groups for low- and high-dose non-T2D cohorts are pooled for this analysis. adj=adjusted, AUC=area under the curve, OGTT=oral glucose tolerance test, SEM=standard error of the mean, T2D=type 2 diabetes.

FIG. 56 shows the Matsuda Index in patients during OGTT. A mixed effect model for repeated measures was used for analysis of CFB in the intent-to-treat population. Adjusted mean change from baseline are shown above/below bars. ^{a}Placebo groups for low- and high-dose non-T2D cohorts are pooled for this analysis. adj=adjusted, CFB=change from baseline, OGTT=oral glucose tolerance test, SEM=standard error of the mean, T2D=type 2 diabetes.

FIG. 57 shows liver fat content (LFC) at Baseline and Week 12. The median is indicated by the line and number value, IQR is indicated by the box, range is indicated by the whiskers, and outliers are indicated by the dots. A healthy LFC is <5%; LFC≥5% indicates MASLD; LFC≥10% may indicate severe MASLD. IQR=interquartile range, LFC=liver fat content, pts=participants, T2D=type 2 diabetes.

FIG. 58 is a series of bar graphs showing the participants achieving target liver fat content (LFC) reductions by Week 12. pts=participants, T2D=type 2 diabetes.

FIG. 59 is a series of plots showing LFC in Participants with MASLD (LFC≥5%) at baseline and at week 24. In the left-hand plot, median is indicated by the line and number value, IQR is indicated by the box, range is indicated by the whiskers, and outliers are indicated by the dots. CI=confidence interval, IQR=interquartile range, LFC=liver fat content, LS=least squares, MASLD=metabolic dysfunction-associated steatotic liver disease, PBO=placebo.

FIG. 60 shows cT1 by week 24 in participants with obesity ±MASLD (LFC≥5%) at baseline. CI=confidence interval, cT1=iron-corrected T1, LS=least squares, MASLD=metabolic dysfunction-associated steatotic liver disease, PBO=placebo, SD=standard deviation.

### DETAILED DESCRIPTION OF THE INVENTION

The present disclosure provides a therapy (e.g., monotherapy or adjunctive therapy) for treatment of a steatotic liver disease, such as MASH, MAFLD, MetALD, or MASLD. The therapy comprises administration of a unimolecular agonist for both glucagon-like peptide-1 (GLP-1) receptor (GLP-1R) and glucose-dependent insulinotropic polypeptide (GIP) receptor (GIPR).

MASLD closely associates with obesity and type 2 diabetes (T2D). GLP-1R and dual GLP-1R/GIPR agonists may have promise for treating MASLD and obesity through weight loss, glycemic control, and potential direct effects on the liver, including reductions in steatosis, fibrosis, and inflammation (see, e.g., Zafer et al., Aliment Pharmacol Ther. (2025) 61:1872-88). Reducing liver fat in people with obesity is clinically important because it may mitigate progression of MASLD and improve metabolic health (Younossi et al., Clin Mol Hepatol. (2025) 31:S32-50).

### I. Dual GLP-1R/GIPR Agonist

GLP-1 and GIP are primary incretins released from the gastrointestinal tract in response to food intake. Both hormones modulate glucose-dependent insulin secretion. GLP-1 also decreases secretion of glucagon, slows gastric emptying, promotes satiety, and reduces food intake. Several GLP-1 mimetics have been approved for the treatment of type 2 diabetes mellitus (T2DM), but their efficacy is limited by gastrointestinal side effects such as nausea, vomiting, and diarrhea.

The primary action of GIP is the stimulation of glucose-dependent insulin secretion. It also modulates the secretion of glucagon in an inverse glucose-dependent manner to protect against hypoglycemia and does not delay gastric emptying. GIP also stimulates glucose uptake in adipocytes and promotes bone strength.

The dual GLP-1R/GIPR agonist used in the present therapy, referred to as “CT-388,” is in some embodiments a once-weekly, subcutaneous injection for obesity, T2D, and other weight-related comorbidities (see, e.g., Chakravarthy et al., Diabetes (2023) 72:75). CT-388 may also be referred to as CT-388. CT-388 potently activates production of cyclic adenosine monophosphate (cAMP), but has no or minimal activity on the β-arrestin signaling pathways on either GLP-1R or GIPR. That is, the agonist is fully biased towards cAMP activation, as opposed to being partially biased (i.e., with some β-arrestin activity) or unbiased (i.e., with full β-arrestin activity), on both GLP-1R and GIPR. β-arrestin activates kinase signaling pathways, but also causes the GLP-1R and GIPR to be turned off and internalized (Hsia et al., Curr Opin Endocrinol Diabetes Obes. (2017) 24(1):73-9). The present agonist causes minimal or no internalization and consequently, desensitization of either GLP-1R or GIPR, and thus has enhanced signaling efficacy (see, e.g., Chakravarthy et al., Diabetes (2023) 72:75; Rodriguez et al., Cell Rep Med. (2025) 6:102156).

CT-388 has the following structural formula:

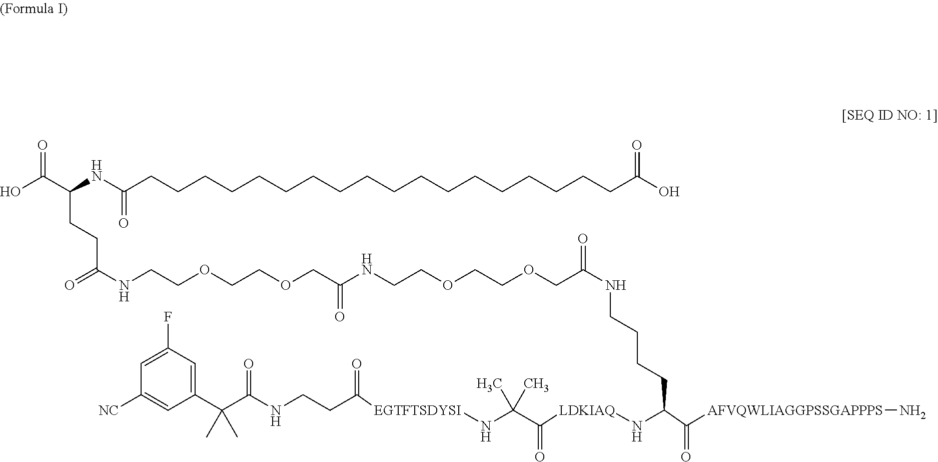

CT-388's structure can also be depicted as follows:

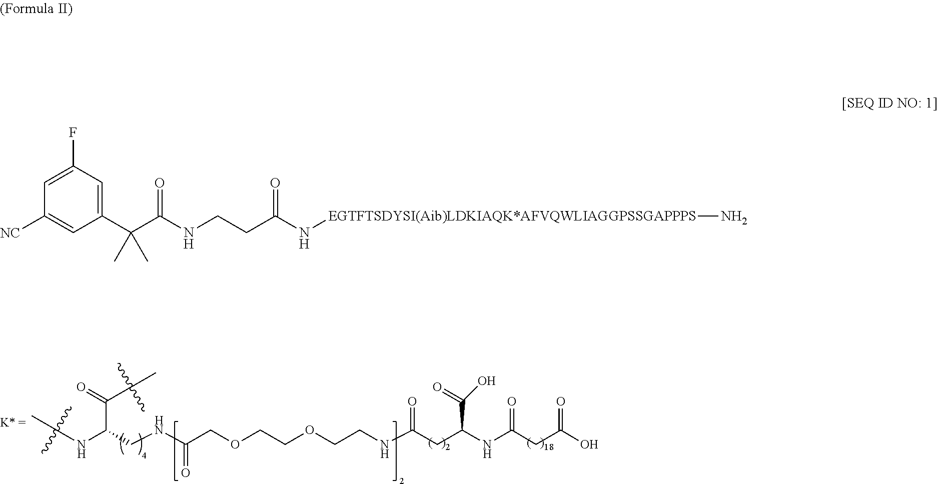

Formula I and Formula II both refer to the same compound.

A pharmaceutically acceptable salt (e.g., sodium or phosphate salt) of the above-depicted compound is also within the meaning of “CT-388” herein. Pharmaceutically acceptable esters of the above-illustrated compound may also be used in the present therapy.

The present disclosure provides pharmaceutical compositions comprising CT-388 and a pharmaceutically acceptable excipient. In some embodiments, the only active pharmaceutical ingredient (API) of the pharmaceutical compositions is the compound of Formula I or II.

In some embodiments, CT-388 is provided as a sterile, lyophilized powder that can be reconstituted as a solution suitable for subcutaneous injection. In some embodiments, CT-388 is provided in a sterile aqueous solution suitable for subcutaneous injection. For example, CT-388 may be provided at a concentration of 20 mg/mL in sodium phosphate buffer comprising mannitol, pH 7.0.

CT-388 may be well tolerated and cause fewer adverse effects than other incretin mimetics. For example, chronic toxicology data show that CT-388 was well tolerated in repeat dosing over 26-weeks and 39-weeks in rats and monkeys, respectively, which exceeds the pharmacologically active dose by 100-fold, and resulted in weight loss up to 10%. Thus, CT-388 can be a potent drug that induces significant weight loss and helps achieve optimal glycemic control, with acceptable tolerability, in overweight or obese adults with or without T2DM or other weight-related comorbidities. CT-388 can potentially be used as a non-invasive alternative to bariatric surgery for weight management.

### II. Patient Populations

In some embodiments, the patient is at least 18 (i.e., is 18 years of age or more). In some embodiments, the patient is up to 75 years of age. In some embodiments, the patient is at least 75 (i.e., is 75 years of age or older). As used herein, an adult is a human aged 18 or older. In some embodiments, the present therapy treats an adult who is between 18 and 65 years of age, inclusive. In some embodiments, the patient is 77 years of age or less (for example, 65 years of age or less, 66 years of age or less, 67 years of age or less, 68 years of age or less, 69 years of age or less, 70 years of age or less, 71 years of age or less, 72 years of age or less, 73 years of age or less, 74 years of age or less, 75 years of age or less, or 76 years of age or less). In some embodiments, the patient is between 21-61 years of age (e.g., between 21-51, between 35-49, between 21-50, between 30-61, about 29, or about 43 years of age.

In some embodiments, the patient is female. In some embodiments, the patient is male.

### Liver Conditions

In some embodiments, the patient to be treated by the present methods has a steatotic liver disease associated with metabolic dysfunction. The term “steatotic” or “steatosis” refers to excessive accumulation of fat within a cell or tissue (e.g., the liver). Accumulation of fat may lead to subsequent liver damage and inflammation. Hepatic steatosis may be characterized by extensive liver fat content (LFC) via imaging techniques, e.g., a magnetic resonance imaging-proton density fat fraction (MRI-PDFF) value of ≥5% in the liver. Exemplary steatotic liver diseases include metabolic dysfunction-associated steatotic liver disease (MASLD), metabolic dysfunction-associated fatty liver disease (MAFLD), and metabolic dysfunction-associated steatohepatitis (MASH).

MASLD may be defined as the presence of hepatic steatosis in conjunction with at least one cardiometabolic risk factor and no other discernible cause (see, e.g., Tacke et al., J Hepatology. (2024) 81(3):492-542).

In some embodiments, the patient has metabolic dysfunction-associated steatotic liver disease (MASLD). Besides hepatic steatosis, an adult MASLD patient may also meet at least one of the following five cardiometabolic criteria: (1) BMI≥25 kg/m² [BMI≥23 kg/m² Asia] or waist circumference >94 cm (M) and 80 cm (F) or ethnicity-adjusted equivalent; (2) fasting serum glucose ≥5.6 mmol/L [100 mg/dL] or 2-hour post-load glucose levels ≥7.8 mmol/L [140 mg/dL] or HbA1c≥5.7% [39 mmol/L] or type 2 diabetes or treatment for type 2 diabetes; (3) blood pressure ≥130/85 mmHg or specific antihypertensive drug treatment; (4) plasma triglycerides ≥1.70 mmol/L [150 mg/dL] or lipid lowering treatment; or (5) plasmid HDL-cholesterol ≤1.0 mmol [40 mg/dL](M) and ≤1.3 mmol/L [50 mg/dL](F) or lipid lowering treatment. See, e.g., Rinella, supra.

In some embodiments, the patient has metabolic dysfunction-associated fatty liver disease (MAFLD), formerly referred to as non-alcoholic fatty liver disease (NAFLD). Besides hepatic steatosis, an MAFLD patient may also meet at least one of the following criteria: (1) overweight or obesity (BMI≥25 kg/m² [BMI≥23 kg/m² Asia]), (2) type 2 diabetes, or (3) evidence of metabolic dysregulation, which refers to having at least two of the following metabolic risk abnormalities: (a) waist circumference ≥102/88 cm [M/F](≥90/80 cm Asia M/F), (b) blood pressure ≥130/85 mmHg or specific drug treatment, (c) plasma high-density lipoprotein cholesterol (HDL-C)<40 mg/dL (<1.0 mmol/L) for males and <50 mg/dL (<1.3 mmol/L) for females or specific drug treatment, (d) plasma triglyceride ≥150 mg/dL (≥1.70 mmol/L) or specific drug treatment, (e) pre-diabetes (HbA1c of 5.7-6.4% (39-47 mmol/L), or fasting plasma glucose of 100 to 125 mg/dL (5.6-6.9 mmol/L), or 2-hour post-load glucose levels of 140-199 mg/dL (7.8-11.0 mmol/L), (f) homeostasis model assessment of insulin resistance (HOMA-IR) score ≥2.5, and (g) plasma hypersensitive C-reactive protein level >2 mg/L. See, e.g., Eslam, supra.

In some embodiments, the patient has metabolic dysfunction-associated steatohepatitis (MASH), formerly referred to as nonalcoholic steatohepatitis (NASH) or noncirrhotic nonalcoholic steatohepatitis. MASH may also be referred to as significant liver steatosis. Steatohepatitis refers to hepatic steatosis accompanied by inflammation and/or liver damage (e.g., necrosis). Steatohepatitis may also present with fibrosis. See, e.g., Burt, Current Diagnostic Pathology. (2001) 7(2):141-7. MASH may be diagnosed by any means known in the art (see, e.g., EASL-EASD-EASO Clinical Practice Guidelines on the management of metabolic dysfunction-associated steatotic liver disease (MASLD), Clin Prac Guide. (2024) 81(3):492-542).

In some embodiments, MASH is characterized by an MRI-PDFF of ≥10% in the patient. MASH may be characterized by liver biopsy histologic evidence, including the presence of key histological features of MASH according to NASH Clinical Research Network (CRN) criteria (see, e.g., Younossi et al., Hepatology. (2018) 68(1):349-60). The patient may also have histologic evidence of fibrosis stage 2 or stage 3 as defined by the NASH (now referred to as MASH) CRN scoring of fibrosis, or histologic evidence of fibrosis stage 1a or stage 1b, optionally accompanied by one or more of the following risk factors: obesity (BMI≥30 kg/m2), type 2 diabetes diagnosed per 2013 American Diabetes Association criteria, or alanine aminotransferasse (ALT)>1.5× upper limit of normal (ULN).

MASH may also be diagnosed based on levels of liver enzymes (e.g., ALT and ALT/AST ratio) or fibrosis biomarkers (e.g., pro-C3), or via ultrasound, transient elastography, ultrasound elastography, or magnetic resonance elastography. In some embodiments, MASH may be diagnosed based on an ALT of about 70 IU/L (e.g., about 50-90, about 60-80, or about 65-75 IU/L) and/or an AST of about 50 IU L (e.g., about 30-70, about 40-60, or about 45-55 IU/L) In some embodiments, MASH is diagnosed via ultrasound. In some embodiments, the patient does not have harmful alcohol intake.

In some embodiments, the patient has metabolic dysfunction-associated alcohol-related liver disease (MetALD). MetALD may be defined as MASLD with consumption of 20-50 g alcohol/day for females or 30-60 g alcohol/day for males) (see, e.g., Tacke et al., J Hepatology. (2024) 81(3):492-542).

In some embodiments, the present methods are for treating steatohepatitis and may result in resolution of steatohepatitis. In some embodiments, the steatohepatitis in the patient is not worsened (i.e., does not progress).

Improvements in liver conditions, e.g., MAFLD, MASLD, or MASH, may be measured by the nonalcoholic fatty liver disease activity score (NAS). NAS may be determined based on assessment of steatosis, inflammation, and ballooning hepatocytes in liver biopsy. The score ranges from zero to eight, with higher scores indicating more serious disease. In some embodiments, the present methods improve the NAS of treated patients, by one, two, three, four, five, six, seven, or eight points.

In some embodiments, the present methods improve the liver condition of a patient as indicated by histomorphometric analysis. The methods may reduce lipid accumulation in the liver of a patient with steatosis. In some embodiments, the present methods reduce ballooning hepatocytes in a patient. Ballooning hepatocytes may be identified by, e.g., cell diameter and/or the presence of disrupted cytoskeletal structures.

In some embodiments, the hepatic steatosis patient does not have fibrosis (i.e., F0). In other embodiments, the steatosis patient has liver fibrosis. The fibrosis may be stage 1 (i.e., F1), characterized by portal fibrosis without septa; stage 2 (i.e., F2), characterized by portal fibrosis with few septa; stage 3 (i.e., F3), characterized as numerous septa without cirrhosis; or stage 4 (i.e., F4), characterized as cirrhosis. In some embodiments, stage 2 fibrosis is characterized by a cT1 score of ≥875 ms. Stage 2 and 3 fibrosis may be referred to, respectively, as moderate and advanced fibrosis.

In some embodiments, the patient to be treated by the present method may have an MRI-PDFF value of about 5.0 kPa to about 15.0 kPa (e.g., about 7.5 kPa to about 12.5 kPa, about 10.0 kPa or about 10.3 kPa) at baseline (see, e.g., Caussy et al., Hepatology. (2018) 68(2):763-72 and Qadri et al., JHEP Reports. (2024) 6(1):100928).

In some embodiments, the patient has a baseline MRI-PDFF liver fat content (LFC) between 1.7-30.7% (e.g., between 2.3-20.5%, between 4.9-15.5%, between 3.4-13.3%, about 8.0%, about 8.5%, or about 9.5%.

In some embodiments, the patient has a mean cT1 of about 700 to 950 ms (e.g., about 718 to about 942 ms, about 725 to about 875 ms, about 750 to about 950 ms, about 756 to about 985 ms, about 848 to about 898 ms, about 825 to about 875 ms, about 804 ms, about 834 ms, about 850 ms, about 851 ms, about 853 ms, or about 858 ms) at baseline. In some embodiments, the patient has high-risk MASH, characterized by a cT1 score of 825 ms or more.

### Obesity

In some embodiments, the patient is not obese or overweight (e.g., BMI<25 kg/m²).

In some embodiments, the patient is overweight or obese. Body mass index (BMI) is an index of weight-for-height that is commonly used to classify overweight and obesity in adults. It is defined as a person's weight in kilograms divided by the square of his height in meters (kg/m²). For adults, WHO defines overweight as having a BMI of 25 to 29.9 kg/m², and obesity as having BMI≥30 kg/m². Obesity is frequently subdivided into the following categories:
Obesity class I—BMI 30 to 34.9 kg/m²
Obesity class II—BMI 35 to 39.9 kg/m²; and
Obesity class III—BMI≥40 kg/m² (also referred to as severe, extreme, or massive obesity)

In some embodiments, the definitions of overweight and obesity in Asian and South Asian populations may be as follows:
Overweight—BMI 23 to 24.9 kg/m²
Obesity—BMI≥25 kg/m²

In some embodiments, the patient has a BMI of 30-90 kg/m² (e.g., 30-52 kg/m², 31-39 kg/m², 31-40 kg/m², about 33 kg/m², about 36 kg/m², about 38 kg/m², or about 39 kg/m²).

In some embodiments, the patient to be treated herein is overweight. In other embodiments, the patient to be treated herein is obese, with class I, II, or III obesity.

In some embodiments, the patient is an obese or overweight otherwise healthy adult. An overweight or obese adult who is otherwise healthy lacks clinically significant history or active clinical manifestation(s) of any significant metabolic, proliferative, allergic, dermatological, hepatic, renal, hematological, pulmonary, cardiovascular, gastrointestinal, neurological, or psychiatric disorder.

### Type 2 Diabetes Mellitus and Prediabetes

In some embodiments, the patient does not have type 2 diabetes mellitus. In some embodiments, the patient has type 2 diabetes mellitus or prediabetes. T2DM and prediabetes may be defined by the 2022 ADA standards of Medical Care in Diabetes (American Diabetes Association's (ADA), “Standards of Medical Care in Diabetes,” Clin Diabetes (2022) 40(1):10-38). The ADA definitions are shown in Table 1 below.

TABLE 1
Glycemic Status Definitions

| | Definitions* | | |
|---|---|---|---|
| Diagnostic Tests | Normoglycemia | Prediabetes | Diabetes |
| (FPG obtained separately or at | <100 mg/dL | 100-125 mg/dL | ≥126 mg/dL |
| time = 0 minutes during an OGTT | (<5.6 mmol/L) | (5.6-6.9 mmol/L) | (≥7.0 mmol/L) |
| 2-hour glucose obtained at | <140 mg/dL | 140-199 mg/dL | ≥200 mg/dL |
| time = 120 minutes during an OGTT | (<7.8 mmol/L) | (7.8-11.0 mmol/L) | (≥11.1 mmol/L) |
| HbA1c | <5.7% | 5.7%-6.4% | ≥6.5% |
| | (<39 mmol/mol) | (39-47 mmol/mol) | (≥48 mmol/mol) |

*FPG: fasting plasma glucose. OGTT: oral glucose tolerance test. HbA1c: hemoglobin 1C.

To make the diagnosis of prediabetes or diabetes, at least one of the above tests (i.e., FPG, 2-hour post-challenge plasma glucose, or HbA1c) needs to be abnormal (i.e., outside/above the normoglycemic range/values for each corresponding test).

In some embodiments, the patient has type 2 diabetes mellitus (T2DM). In some embodiments, the T2DM patient has a BMI of ≥25 kg/m², ≥27 kg/m², ≥30 kg/m², ≥35 kg/m², ≥40 kg/m², or ≥45 kg/m². In some embodiments, the T2DM patient is not overweight or obese.

In some embodiments, T2D in the patient has been managed with diet and/or exercise alone or treated with a stable dose of metformin monotherapy for ≥3 months prior to treatment.

In some embodiments, the patient receives additional therapy for glycemic control. Such glycemic control therapy may be an oral glucose lowering therapy (e.g., metformin).

### Comorbidities

In some embodiments, the adult patients have one or more weight-related (treated or untreated) comorbidities (e.g., prediabetes, T2DM, hypertension, dyslipidemia, obstructive sleep apnea, or previously diagnosed cardiovascular disease. In some embodiments, the previously diagnosed cardiovascular disease is ischemic cardiovascular disease or Class I-II heart failure as classified by the New York Heart Association (NYHA) Functional Classification. In some embodiments, the patient has normoglycemia or type 2 diabetes mellitus.

In some embodiments, the patient is prediabetic and the therapy improves glycemic control in the patient.

In some embodiments, the patient has one or more T2DM-related (treated or untreated) comorbidities (e.g., hypertension and dyslipidemia).

In some embodiments, the patient treated herein has one or more comorbidities, such as prediabetes, hypertension, and dyslipidemia. Hypertension can be defined as current use of blood pressure lowering agents initiated for hypertension, or with systolic blood pressure (SBP)≥130 mmHg or diastolic blood pressure (DBP)≥80 mmHg. Dyslipidemia can be defined as current use of lipid-lowering agents initiated for dyslipidemia, or with low-density lipoprotein (LDL)≥160 mg/dL (4.1 mmol/L) or triglycerides ≥150 mg/dL (1.7 mmol/L), or high-density lipoprotein (HDL)<40 mg/dL (1.0 mmol/L) for men or HDL<50 mg/dL (1.3 mmol/L) for women.

### Other Patient Characteristics

In some embodiments, the patient herein is not pregnant or breastfeeding.

In some embodiments, the patient herein does not have a history of bariatric surgery or gastric banding (including LAP-BAND), history of any gastrointestinal surgery that may induce malabsorption (e.g., bowel resection), or any GI motility disorders, gastroparesis, delayed gastric emptying, inflammatory bowel disease, pancreatitis, malabsorption syndromes, chronic diarrhea, chronic constipation, and clinically significant irritable bowel syndrome. In some embodiments, the patient has not undergone a LAD-BAND, intragastric balloon, gastric sleeve, duodenal mucosal resurfacing, liposuction, or abdominoplasty procedure. In some embodiments, patients with history of devices such as LAP-BAND® or intragastric balloon are treated with the present methods if devices were removed >one year prior to treatment. Patients with a history of liposuction or abdominoplasty performed >one year prior to treatment may also be treated with the present methods.

In some embodiments, the patient does not have semi-supine blood pressure systolic >160 mm Hg and/or diastolic >95 mm Hg. In some embodiments, the patient does not have hepatocellular carcinoma (HCC).

In some embodiments, the patient herein does not have an active or untreated malignancy or patients who have been in remission from a clinically significant malignancy (other than basal or squamous cell skin cancer, in situ carcinomas of the cervix, or in situ prostate cancer) for <5 years.

In some embodiments, the patient herein does not have a personal or family history (first-degree relative) of medullary thyroid carcinoma (MTC), or a genetic condition that predisposes to MTC (i.e., thyroid C-cell hyperplasia, or multiple endocrine neoplasia type 2A or type 2B).

In some embodiments, the patient herein does not have a history of atopy (severe or multiple allergic manifestations) or clinically significant multiple or severe drug allergies, or intolerance to topical corticosteroids, or severe posttreatment hypersensitivity reactions; known allergy to any component of the pharmaceutical composition, GLP-1 analogues, or related compounds.

In some embodiments, the patient herein has not undergone hormone replacement therapy.

In some embodiments, the patient herein does not concurrently receive other pharmacotherapy for obesity including, but not limited to: Saxenda® (liraglutide), Wegovy® (semaglutide), Mounjaro® (tirzepatide), Xenical® or Alli® (orlistat), Meridia® (sibutramine), Acutrim® (phenylpropanolamine), Sanorex® (mazindol), Apidex® (phentermine), BELVIQ® (lorcaserin), Qsymia® (phentermine/topiramate combination), Contrave® (naltrexone/bupropion), Asenlix® (Clobenzorex), Solucaps® (Mazindol), Redotex® (aloine, atropine, diazepam, norpseudoephedrine, triyodotironine), berberine, resveratrol, other sympathomimetic agents, thyroid hormones (for nonthyroid indications), growth hormone, or any non-herbal supplements/alternative remedies with unknown/unspecified content.

In some embodiments, the patient to be treated herein does not concurrently receive other drugs that target GLP-1R and/or GIPR.

In some embodiments, the patient is 18 years of age and up to 75 years of age (inclusive). In some embodiments, the patient is obese as defined as having a BMI of ≥30.0 kg/m². In some embodiments, the patient is overweight as defined as having a BMI≥27.0 and <30.0 kg/m². The patient may also have a BMI of ≥25.0 kg/m² and <30.0 kg/m². In some embodiments, the patient has normal ECG findings or normal variants, within QTcF range <450 msec for males and <470 msec for females. In some embodiments, the patient has a resting heart rate in the range of 45-100 bpm.

In some embodiments, the patient has been previously diagnosed with at least one of the following weight-related comorbidities:
a. prediabetes, as defined by the 2024 ADA Standards of Medical Care in Diabetes;
b. hypertension, defined as current use of blood pressure lowering agents initiated for hypertension, or with systolic blood pressure ≥130 mm Hg or diastolic blood pressure ≥80 mm Hg;
c. dyslipidemia, defined as current use of lipid-lowering agents initiated for dyslipidemia, or with low-density lipoprotein (LDL)≥160 mg/dL (4.1 mmol/L) or triglycerides ≥150 mg/dL (1.7 mmol/L), or high-density lipoprotein (HDL)<40 mg/dL (1.0 mmol/L) for men or HDL<50 mg/dL (1.3 mmol/L) for women;
d. obstructive sleep apnea; or
   cardiovascular disease (for example, ischemic cardiovascular disease, New York Heart Association (NYHA) Functional Class I-II heart failure) In some embodiments, the patient has a history of ≥1 self-reported unsuccessful diet/exercise effort to lose body weight. In some embodiments, the patient has not participated in unbalanced/extreme diets such as very low calorie, low carbohydrate, very high protein, ketogenic, or intermittent diets.

In some embodiments, the patient does not have a prior history or diagnosis of any type of diabetes mellitus (e.g., type 1, type 2, gestational), or does not have a history of ketoacidosis or hyperosmolar state. The patient may not have diabetes mellitus as determined based on fasting glucose or HbA1c.

Alternatively, the patient may have type 2 diabetes mellitus (according to, e.g., the World Health Organization classification). The patient's HbA1c level may be ≥7% and ≤10.5%. In some embodiments, a patient has managed their T2DM with diet and exercise alone or diet and exercise and treatment with metformin or a SGLT-2 inhibitor as monotherapy or in combination. In some embodiments, the T2DM patient does not have type 1 diabetes mellitus. In some embodiments, the T2DM patient does not have proliferative diabetic retinopathy, diabetic macular edema, non-proliferative diabetic retinopathy (e.g., that requires acute treatment based on a dilated fundoscopic examination), or active neuropathy (including resting tachycardia, orthostatic hypotension, or diabetic diarrhea).

In some embodiments, the patient has a diagnosis of prediabetes (determined by fasting glucose or HbA1c levels). Such patients may not be on any type of glucose-lowering treatments (pharmacologic or dietary supplements), for example metformin or berberine.

In some embodiments, the patient has not had a prior surgical treatment of any type for obesity. In some embodiments, the patient has not undergone LAP-BAND©, intragastric balloon, duodenal sleeve, resurfacing, liposuction, or abdominoplasty procedures. In some embodiments, the patient may have undergone liposuction or abdominoplasty one or more years prior to treatment.

In some embodiments, the patient does not have a known clinically significant or active gastric emptying abnormality (e.g., severe gastroparesis or gastric outlet obstruction, intestinal obstruction, or any gastrointestinal (GI) motility disorders); malabsorption, including chronic constipation/diarrhea, celiac disease, inflammatory bowel disease, or bowel resection; or has chronically take drugs that directly affect GI motility (e.g., anticholinergics, 5-hydroxytryptamine [serotonin] antagonists, or opiates).

The patient may not have chronic or acute pancreatitis. In some embodiments, the patient does not have a history of complications from gall stones and/or acute cholecystitis or uncontrolled hypertension (systolic blood pressure ≥160 mm Hg and/or diastolic blood pressure ≥100 mm Hg). In some embodiments, the patient does not have a resting pulse rate of ≥100 bpm.

In some embodiments, the patient does not have chronic liver disease of any etiology, history of hepatic cirrhosis, or acute viral hepatitis. The patient may have metabolic dysfunction-associated fatty liver disease or metabolic dysfunction-associated steatohepatitis.

In some embodiments, the patient does not have congestive heart failure (for example, NYHA Functional Classification III or IV congestive heart failure), myocardial infarction, cerebrovascular accident (stroke), transient ischemic attack, unstable angina, coronary artery bypass graft, or percutaneous coronary intervention. In some embodiments, the patient does not have a personal or family history of long QT syndrome, family history of sudden death in a first-degree relative (parents, sibling, or children) before the age of 40 years, or a personal history of unexplained syncope.

In some embodiments, the patient does not have uncontrolled thyroid disease, defined as active symptoms (e.g., palpitations, lethargy, weight gain/loss) and/or having a thyroid-stimulating hormone (TSH) level outside the normal reference range.

In patients with obesity, the obesity may not be induced by other endocrinologic disorders (e.g., Cushing syndrome, acromegaly, inadequately treated hypothyroidism) or diagnosed monogenetic or syndromic forms of obesity (e.g., Melanocortin 4 Receptor deficiency or Prader-Willi syndrome). In some embodiments, the patient does not have a family or personal history of medullary thyroid carcinoma or a genetic condition that predisposes to medullary thyroid carcinoma (i.e., thyroid C-cell hyperplasia, or multiple endocrine neoplasia Type 2A or Type 2B).

In some embodiments, the patient does not have a history or diagnosis of major depressive disorder or any history/diagnosis of other severe psychiatric conditions (e.g., schizophrenia; bipolar disorder; eating disorder; or other serious mood, anxiety, or hyperactivity disorder). Participants with depression or anxiety whose disease state is considered stable and expected to remain stable throughout the course of the study may be treated only if their conditions are adequately controlled by stable doses of escitalopram (Lexapro®) or other selective serotonin reuptake inhibitor expected to continue at the same dose for the entire study duration. In some embodiment, the patient has a PHQ-9 score of ≥15. In some embodiments, the patient does not have a lifetime history of a suicide attempt, or suicidal behavior within 30 days prior to treatment, or suicidal ideation on the C-SSRS assessed at the Screening Visit or Day 1 and determined as a) A “yes” to either Question 4 (Active Suicidal Ideation with Some Intent to Act, Without Specific Plan) OR, b) A “yes” answer to Question 5 (Active Suicidal Ideation with Specific Plan and Intent) on the “Suicidal Ideation” portion of C-SSRS, OR c) A “yes” answer to any of the suicidal-related behaviors (Actual Attempt, Interrupted Attempt, Aborted Attempt, Preparatory Act or Behavior) on the “Suicidal Behavior” portion of the C-SSRS, AND d) The ideation or behavior occurred within the past month.

In some embodiments, the patient does not have ECG indicative of active cardiac disease or clinically significant variants/abnormalities, corrected QT interval ≥450 msec for male participants and >47 msec for female participants, or resting heart rate <45 bpm or >100 bpm.

In some embodiments, the patient does not have:
a. alanine aminotransferase (ALT), aspartate aminotransferase (AST), or gamma glutamyl transferase (GGT)>3.0× or >5.0× the upper limit of normal (ULN) for the reference range;
b. alkaline phosphatase (ALP)>1.5× or >3.0×ULN for the reference range;
c. total bilirubin >1× or >2×ULN for the reference range;
d. amylase or lipase >2× or >3.0×ULN for the reference range;
e. calcitonin ≥20 ng/L; optionally ≥20 ng/L if eGFR is ≥60 mL/min/1.73 m² or ≥35 ng/L if eGFR is <60 mL/min/1.73 m²;
f. estimated glomerular filtration rate (eGFR)<45 or <60 mL/min/1.73 m² by the 2021 CKD-EPI creatinine equation;
g. fasting plasma triglycerides ≥500 mg/dL;
h. acute or chronic viral hepatitis (determined based on hepatitis B surface antigen [HbsAg] or presence of HCV RNA);
i. positive HIV antibody screen;
j. hemoglobin value <11 g/dL (men) or <10 g/dL (women);
k. white blood cells (WBC)<the lower limit of normal (LLN) for the reference range; or
l. platelets <LLN for the reference range.

In some embodiments, the patient does not have:
a. alanine aminotransferase (ALT), aspartate aminotransferase (AST), or gamma glutamyl transferase (GGT)>3.0× the upper limit of normal (ULN) for the reference range;
b. alkaline phosphatase (ALP)>1.5×ULN for the reference range;
c. total bilirubin >1×ULN for the reference range;
d. amylase or lipase >2×ULN for the reference range;
e. calcitonin ≥20 ng/L; optionally ≥20 ng/L if eGFR is ≥60 mL/min/1.73 m² or ≥35 ng/L if eGFR is <60 mL/min/1.73 m²;
f. estimated glomerular filtration rate (eGFR)<60 mL/min/1.73 by the 2021 CKD-EPI creatinine equation; optionally decreased to non-eGFR limitations (or to 15-30 mL/min/1.73 m²);
g. fasting plasma triglycerides ≥500 mg/dL;
h. acute or chronic viral hepatitis (determined based on hepatitis B surface antigen [HbsAg] or presence of HCV RNA); or
i. positive HIV antibody screen.

### III. CT-388 Therapy

The pharmaceutical composition comprising CT-388 may be injected subcutaneously at an interval deemed appropriate by a physician, for example, every three days, every four days, every five days, every six days, every week, every two weeks, every three weeks, every four weeks, every six weeks, every eight weeks, every month, or every two months. In some embodiments, CT-388 is injected subcutaneously at an interval of every three days to every two months. In some embodiments, CT-388 is injected subcutaneously at an interval according to the judgement of the patient or administrator. For example, CT-388 may be injected every 10 days, or may be administered only during certain times of the year (e.g., fall or winter). The pharmaceutical composition comprising CT-388 may be injected subcutaneously by the patient (i.e., by self-administration).

In some embodiments, the pharmaceutical composition containing CT-388 is administered by injection at a body site (e.g., abdomen, upper arm, thighs, or hips) subcutaneously once weekly (QW), every two weeks (i.e., biweekly or Q2W), or monthly (QM). In some embodiments, the pharmaceutical composition comprising CT-388 is administered by subcutaneous injection at a certain interval (e.g., as described above) over a period of 4 to 28 weeks, or longer (e.g., 6, 12, 18, or 24 months, or longer). In some embodiments, the pharmaceutical composition is administered irrespective of meals. In some embodiments, the pharmaceutical composition is administered following a fast from food (e.g., an overnight fast from food or a ≥10 hour fast from food).

In some embodiments, CT-388 is administered subcutaneously to an adult patient as described in the present disclosure at a flat (i.e., weight-independent) dose of about 0.5 mg to about 30 mg, for example, about 1 mg to about 28 mg, about 2 mg to about 27 mg, about 3 mg to about 26 mg, about 4 mg to about 25 mg, about 5 mg to about 24 mg, about 5 mg to about 22 mg, about 8 mg to about 22 mg, about 8 mg to about 24 mg, about 5 mg to about 17 mg, about 2 mg to about 16 mg, about 4 mg to about 16 mg, about 2 mg to about 20 mg, about 4 mg to about 20 mg, about 2 mg to about 24 mg, or about 4 mg to about 24 mg. In some embodiments, treatment with CT-388 is initiated on an up-titration schedule, i.e., starting treatment at a low dosing amount, and gradually increasing the dosing amount over a period of time to the higher or highest, tolerated dosing amount. In some embodiments, the dosing amount increases by an increment of 2 to 10 mg each time, e.g., by an increment of 2 mg, 3 mg, 4 mg, 5 mg, 6 mg, 7 mg, 8 mg, 9 mg, or 10 mg.

In some embodiments, a pharmaceutical composition comprising CT-388 is injected subcutaneously to a patient at a QW, Q2W, or QM dose of about 0.5 mg.

In some embodiments, a pharmaceutical composition comprising CT-388 is injected subcutaneously to a patient at a QW, Q2W, or QM dose of about 2 mg.

In some embodiments, a pharmaceutical composition comprising CT-388 is injected subcutaneously to a patient at a QW, Q2W, or QM dose of about 4 mg.

In some embodiments, a pharmaceutical composition comprising CT-388 is injected subcutaneously to a patient at a QW, Q2W, or QM dose of about 5 mg.

In some embodiments, a pharmaceutical composition comprising CT-388 is injected subcutaneously to a patient at a QW, Q2W, or QM dose of about 6 mg.

In some embodiments, a pharmaceutical composition comprising CT-388 is injected subcutaneously to a patient at a QW, Q2W, or QM dose of about 7.5 mg.

In some embodiments, a pharmaceutical composition comprising CT-388 is injected subcutaneously to a patient at a QW, Q2W, or QM dose of about 8 mg.

In some embodiments, a pharmaceutical composition comprising CT-388 is injected subcutaneously to a patient at a QW, Q2W, or QM dose of about 12 mg.

In some embodiments, a pharmaceutical composition comprising CT-388 is injected subcutaneously to a patient at a QW, Q2W, or QM dose of about 16 mg.

In some embodiments, a pharmaceutical composition comprising CT-388 is injected subcutaneously to a patient at a QW, Q2W, or QM dose of about 17 mg.

In some embodiments, a pharmaceutical composition comprising CT-388 is injected subcutaneously to a patient at a QW, Q2W, or QM dose of about 20 mg.

In some embodiments, a pharmaceutical composition comprising CT-388 is injected subcutaneously to a patient at a QW, Q2W, or QM dose of about 22 mg.

In some embodiments, a pharmaceutical composition comprising CT-388 is injected subcutaneously to a patient at a QW, Q2W, or QM dose of about 24 mg.

In some embodiments, treatment with CT-388 is initiated on an up-titration schedule, i.e., starting treatment at a low dosing amount, and gradually increasing the dosing amount over a period of time to the higher or highest, tolerated dosing amount. In some embodiments, the titration occurs over a period of about 3, 4, 8, 12, 16, 20, 24, 28, or 32 weeks, or longer; in further embodiments, during the titration period, the pharmaceutical composition containing CT-388 is injected QW. In some embodiments, the titration starts with a dosing amount (starting dose) of 5 mg, and the dosing amount increases by an increment of 2 to 10 mg each time, e.g., by an increment of 2 mg, 3 mg, 4 mg, 5 mg, 6 mg, 7 mg, 8 mg, 9 mg, or 10 mg, finally reaching a dosing amount (maintenance or maximum dose) that is between 7.5 mg and 24 mg, for example, 22 mg or 24 mg. In some embodiments during the titration period, the patient may stay on a new dose for 1 to 10 weeks (e.g., 1, 2, 3, 4, 5, 6, 7, 8, 9, or 10 weeks) for acclimatization, before progressing to the next higher dose. In some embodiments, the titration occurs at any time during treatment (i.e., does not follow a preset titration regimen).

In some embodiments, the titration occurs over a period of 3 or more (e.g., 4) weeks according to the following regimen:
5 mg in week 1;
5 mg in week 2;
5 mg in week 3; and
7.5 mg in week 4.

In some embodiments, the titration occurs over a period of 3 or more (e.g., 4) weeks according to the following regimen:
5 mg in week 1;
5 mg in week 2;
8 mg in week 3; and
12 mg in week 4.

In some embodiments, the titration occurs over a period of 3 or more (e.g., 4) weeks according to the following regimen:
5 mg in week 1;
8 mg in week 2;
12 mg in week 3; and
12 mg in week 4.

In some embodiments, the titration occurs over a period of 3 or more (e.g., 4) weeks according to the following regimen:
8 mg in week 1;
8 mg in week 2;
12 mg in week 3; and
16 mg in week 4.

In some embodiments, the titration occurs over a period of 3 or more (e.g., 4) weeks according to the following regimen:
8 mg in week 1;
12 mg in week 2;
16 mg in week 3; and
20 mg in week 4.

In some embodiments, the titration occurs over a period of 3 or more (e.g., 4) weeks according to the following regimen:
5 mg in week 1;
8 mg in week 2;
12 mg in week 3; and
16 mg in week 4.

In some embodiments, the titration occurs over a period of 3 or more (e.g., 4) weeks according to the following regimen:
5 mg in week 1;
5 mg in week 2;
5 mg in week 3; and
8 mg in week 4.

In some embodiments, the titration occurs over a period of 8 or more (e.g., 9) weeks according to the following regimen:
5 mg in week 1;
5 mg in week 2;
8 mg in week 3;
8 mg in week 4;
12 mg in week 5;
12 mg in week 6;
17 mg in week 7;
17 mg in week 8; and
22 mg in week 9.

In some embodiments, the titration occurs over a period of 8 or more (e.g., 9) weeks according to the following regimen:
8 mg in week 1;
8 mg in week 2;
8 mg in week 3;
8 mg in week 4;
16 mg in week 5;
16 mg in week 6;
16 mg in week 7;
16 mg in week 8; and
24 mg in week 9.

In some embodiments, the titration occurs over a period of 8 or more (e.g., 9) weeks according to the following regimen:
8 mg in week 1;
8 mg in week 2;
12 mg in week 3;
12 mg in week 4;
16 mg in week 5;
16 mg in week 6;
20 mg in week 7;
20 mg in week 8; and
24 mg in week 9.

In some embodiments, the titration occurs over a period of 11 or more (e.g., 12) weeks according to the following regimen:
5 mg weekly in weeks 1-3; and
8 mg weekly in weeks 4-9.

In some embodiments, the titration occurs over a period of 23 or more (e.g., 25) weeks according to the following regimen:
2 mg in weeks 1-4;
4 mg in weeks 5-8,
8 mg in weeks 9-12,
12 mg in weeks 13-16,
16 mg in weeks 17-20,
20 mg in weeks 21-24, and
24 mg in week 25.

In some embodiments, the titration occurs over a period of 16 or more (e.g., 17) weeks according to the following regimen:
2 mg in weeks 1-4;
4 mg in weeks 5-8,
8 mg in weeks 9-12,
12 mg in weeks 13-16, and
16 mg in week 17.

In some embodiments, the titration occurs over a period of 12 or more (e.g., 13) weeks according to the following regimen:
2 mg in weeks 1-4;
4 mg in weeks 5-8,
8 mg in weeks 9-12, and
12 mg in weeks 13.

In some embodiments, the titration occurs over a period of four or more (e.g., five) weeks according to the following regimen:
4 mg in weeks 1-4, and
8 mg in week 5.

In some embodiments, the titration occurs over a period of 23 or more (e.g., 25) weeks according to the following regimen:
2 mg in weeks 1-4;
4 mg in weeks 5-8,
8 mg in weeks 9-12,
12 mg in weeks 13-16,
16 mg in weeks 17-20,
20 mg in weeks 21-24, and
24 mg in week 25.

In some embodiments, the titration occurs over a period of 16 or more (e.g., 17) weeks according to the following regimen:
2 mg in weeks 1-4;
4 mg in weeks 5-8,
8 mg in weeks 9-12,
12 mg in weeks 13-16, and
16 mg in week 17.

In some embodiments, the titration occurs over a period of four or more (e.g., five) weeks according to the following regimen:
4 mg in weeks 1-4, and
8 mg in week 5.

In some embodiments, the titration occurs according to the following regimen:
2 mg in weeks 1-12, and
4 mg in week 13.

In some embodiments, the titration occurs according to the following regimen:
4 mg in weeks 1-12, and
8 mg in week 13.

In some embodiments, the titration occurs according to the following regimen:
4 mg in weeks 1-4,
8 mg in weeks 5-8,
12 mg in weeks 9-12, and
16 mg in week 13.

In some embodiments, the titration occurs according to the following regimen:
2 mg in weeks 1-4,
4 mg in weeks 5-8,
8 mg in weeks 9-12,
12 mg in weeks 13-16,
16 mg in weeks 17-20,
20 mg in weeks 21-24, and
24 mg in week 25.

In some embodiments, the titration occurs over a period of 20 or more (e.g., 21) weeks according to the following regimen:
4 mg in weeks 1-4;
8 mg in weeks 5-8,
12 mg in weeks 9-12,
16 mg in weeks 13-16,
20 mg in weeks 17-20, and
24 mg in week 21.

When titrating dose upwards, if a dose is not tolerated at any stage, the dosing may be reverted to the previous tolerated dose for the next dose (i.e., down-titration is allowed). In some embodiments, the patient may then attempt up-titration at the subsequent scheduled dosing time.

In some embodiments, a pharmaceutical composition comprising CT-388 is administered to a patient as an adjunct to diet and/or exercise therapy (i.e., physical activity). In some embodiments, the diet therapy comprises a hypocaloric diet with, e.g., a recommended macronutrient composition of the following: maximum 25% of energy from fat, approximately 25% of energy from protein, and approximately 50% of energy from carbohydrates. In some embodiments, the diet therapy comprises an energy deficit of approximately 500 kcal/day compared with the participant's estimated total energy expenditure.

In some embodiments, the exercise therapy (i.e., physical activity) comprises regular physical activity (e.g., 150 minutes of moderate intensity physical activity per week). The moderate intensity physical activity may be, e.g., brisk walking.

In some embodiments, a pharmaceutical composition comprising CT-388 is administered to a patient as an adjunct to an anti-diabetic treatment, e.g., treatment with metformin. In some embodiments, CT-388 is administered in combination with a therapeutic agent for glycemic control. The therapeutic agent for glycemic control may be an oral glucose lowering therapy (e.g., metformin).

In some embodiments, a pharmaceutical composition comprising CT-388 is administered to a patient in lieu of bariatric surgery.

In some embodiments, CT-388 may be administered in combination with one or more of additional therapeutic agents. Representative additional therapeutic agents include, but are not limited to, anti-obesity agents, therapeutic agents for diabetes, therapeutic agents for diabetic complications, therapeutic agents for hyperlipidemia, antihypertensive agents, diuretics, chemotherapeutics, immunotherapeutics, anti-inflammatory drugs, antithrombotic agents, anti-oxidants, therapeutic agents for osteoporosis, vitamins, antidementia drugs, erectile dysfunction drugs, therapeutic drugs for urinary frequency or urinary incontinence, other therapeutic agents for MAFLD or MASH (e.g., resmetirom), and therapeutic agents for dysuria. In some embodiments, CT-388 is administered in combination with a therapeutic agent that preserves muscle mass.

In some embodiments, CT-388 is administered to the patient in combination with a therapeutic agent for glycemic control. The therapeutic agent for glycemic control may be an oral glucose lowering therapy (e.g., metformin).

In some embodiments, CT-388 is administered to the patient in combination with a therapeutic agent used to manage or treat type 2 diabetes mellitus (e.g., a thiazolidinedione such as pioglitazone or rosiglitazone).

### IV. Treatment Outcomes

In some embodiments, patients treated with CT-388 experience weight loss. For example, patients may experience 3 or more (e.g., 3.7 or more, 4 or more, 5 or more, 6.0 or more, 7.0 or more, or 7.4 or more) kg of placebo-adjusted weight loss. In some embodiments, obese patients experience 7.0 kg or more (e.g., 7.7 kg or more) of weight loss. Patients may also experience placebo-adjusted weight loss of 5% or more (e.g., 9.3% or more, 9.6% or more, 10% or more, 10.2% or more, 10.4% or more, 10.9% or more, 11.5% or more, 11.8% or more, 12.2% or more, 12.4% or more, 12.6% or more, 13% or more, 13.10% or more, 15% or more, 16.4% or more, 18.3% or more, 18.8% or more, 18.9% or more, 20% or more, 20.7 or more, or 25% or more) of their initial body weight. In some embodiments, body weight is measured ten hours after fasting (including no more than two glasses of fluids), after voiding, and while wearing a gown with undergarments only. In some embodiments, the weight loss is clinically significant. For short-term duration of treatment (one dose), a 1.0% decrease in weight within one week of treatment is considered clinically significant. In general, for longer treatment durations, ≥5% body weight reduction is considered to be clinically meaningful, and ≥10% weight loss is considered to represent a disease-modifying effect. In some embodiments, the patient does not experience a weight loss plateau. Weight loss may be maintained beyond the cessation of treatment (e.g., for two or more or two or fewer weeks). Treatment with CT-388 may also result in clinically meaningful decreases in hip and waist circumference. In some embodiments, patients treated with CT-388 have reduced food intake.

The treatment methods may result in a decrease in HbA1c, fasting glucose, or fating insulin in a patient compared to baseline. In some embodiments, a patient treated with CT-388 experiences a decrease in HbA1c of 0.10% or more (e.g., 0.20% or more, 0.30% or more, 0.33% or more, 0.38% or more, 0.39% or more, 0.44% or more, 0.43% or more, 0.44% or more, or 0.50% or more) compared to baseline. In some embodiments, fasting glucose in a patient is decreased by 1 or more mg/dL (e.g., 2.0 or more, 2.8 or more, 3.0 or more, 4.0 or more, 4.3 or more, 5.0 or more, 6.0 or more, 7.0 or more, 7.7 or more, 7.8 or more, 8.0 or more, 9.0 or more, 9.2 or more, 10.0 or more, 10.3 or more, or 11.0 or more mg/dL) compared to baseline. CT-388 treatment may result in a decrease in fasting insulin levels of 1.0 or more μIU/mL (e.g., 1.4 or more, 2.0 or more, 2.5 or more, 2.8 or more, 3.0 or more, 4.0 or more, 4.1 or more, 4.2 or more, 4.5 or more, 5.0 or more, 5.5 or more, or 6.0 or more μIU/mL) compared to baseline.

In some embodiments, patients treated with CT-388 experience a decrease in appetite. Appetite may be assessed using a visual analog scale with eight items scored using a 5-point scale. Possible scores range from 8-40, with lower scores indicating worse appetite. In some embodiments, patient appetite decreases by 5 or more units (e.g., 6 or more, 7 or more, 8 or more, 9 or more, or 10 or more units).

In some embodiments, patients treated with CT-388 experience an increase in satiety. Satiety may be assessed using a visual analog scale with four questions using a 100-millimeter scale. Patients may be asked, based on the previous seven days, to rate their general satiety/satisfaction (100=completely satisfied, 0=not at all satisfied); fullness (100=totally full, 0=not at all full), hunger (100=never been more hungry, 0=not hungry at all), and prospective food consumption (100=a lot, 0=nothing at all). Overall satiety scores may be calculated as the average of the four individual scores (satiety+fullness+[100-prospective food consumption]+[100-hunger]/4), with a higher overall score indicating more satiety. In some embodiments, satiety is increased by 10 or more units (e.g., 15 or more, 20 or more, 25 or more, 30 or more, 35 or more, or 40 or more units).

In some embodiments, the present methods decrease liver fat in a patient. Liver fat may be decreased relative from baseline by 20% or more, 21.4% or more, 30% or more, 40% or more, 50% or more, 58.5% or more, 59% or more, 60% or more, or 70% or more in a patient. In some embodiments, liver fat in a patient is decreased to an absolute value of 5% or less (e.g., 2.5% or less). In patients with baseline MRI-PDFF of ≥5%, liver fat may be reduced by 60% or more (e.g., 70% or more, 78.1% or more, or 80% or more) compared to baseline, or treatment may result in an absolute MRI-PDFF of 7% or less (e.g., 6% or less, 5% or less, 4% or less, or 3% or less). In patients with baseline MRI-PDFF of ≥10%, liver fat may be reduced by 30% or more (e.g., 46% or more, 50% or more, 54% or more, 60% or more, 70% or more, 80% or more, 86.5% or more, or 90% or more) compared to baseline, or treatment may result in an absolute MRI-PDFF of 4% or less (e.g., 3.5% or less). In some embodiments, treatment results in normalization of liver fat in a patient.

Liver fat may be measured by any technique known in the art. An exemplary technique is ultrasound. Another exemplary technique is magnetic resonance imagining protein density fat fraction (MRI-PDFF), also known as liver fat content (LFC), a reliable and accurate measure of liver fat. See, e.g., Alkhouri et al., J Hepatology. (2024) 1-8.

In some embodiments, the present methods reduce inguinal white adipose tissue weight in a patient compared to baseline.

In some embodiments, the present methods reduce fasting blood glucose (i.e., fasting glucose levels) in a patient compared to baseline. Fasting blood glucose may decrease by 2 or more mg/dL (e.g., 3 or more mg/dL, 4 or more mg/dL, 5 or more mg/dL, 5.2 or more mg/dL, 6 or more mg/dL, 7 or more mg/dL, or 7.8 or more mg/dL compared to baseline. In some embodiments, improvement in fasting blood glucose is greater and quicker in patients with higher baseline fasting glucose levels (e.g., 90 or more, 91 or more, 92 or more, 93 or more, 94 or more, 95 or more, 96 or more, or 96.5 or more mg/dL) as compared to patients with lower baseline levels (e.g., 88.8 mg/dL). In some embodiments, fasting insulin levels are not significantly affected by the present methods.

In some embodiments, patients treated with CT-388 have improved glucose tolerance. In some embodiments, improvement in glucose tolerance is greater and quicker in patients with obesity who had elevated fasting glucose levels (e.g., 90 or more, 91 or more, 92 or more, 93 or more, 94 or more, 95 or more, 96 or more, or 96.5 or more mg/dL) and HOMA-IR (4.0 or more, 4.5 or more, 4.9 or more, or 5.0 or more) at baseline. CT-388 treatment may reduce mean glucose levels during an oral glucose tolerance test (OGTT) by 10 or more units (e.g., 15 or more, 20 or more, 25 or more, 30 or more, 35 or more, 37 or more, 39 or more, or 40 or more units) compared to baseline. AUC_{0-120 min} glucose from baseline, insulin, HOMA-IR, and C-peptide may also be decreased during OGTT in patients treated with CT-388. Insulin sensitivity may be improved. The present treatment methods may also decrease cT1 in a patient. cT1 may be decreased by 40 ms or more, 44.0 ms or more, 50 ms or more, 51.7 ms or more, 60 or more, 70 or more, or 73.1 ms or more, or 80 ms or more compared to baseline. In patients with baseline MRI-PDFF≥5%, cT1 may be decreased by 40 ms or more (e.g., 44.0 ms or more). In patients with baseline MRI-PDFF≥10%, cT1 may be decreased by 40 ms or more (e.g., 50 ms or more, 60 ms or more, 70 ms or more, 73.1 ms or more, or 73.5 ms or more) compared to baseline. In patients with baseline MRI-PDFF of ≥5%, cT may be reduced by 30% or more (e.g., 40% or more, 44% or more, 50% or more, 60% or more, 70% or more, or 80% or more) compared to baseline. In patients with baseline MRI-PDFF of ≥10%, cT1 may be reduced by 40% or more (e.g., 50% or more, 60% or more, 70% or more, 73.1% or more, or 80% or more) compared to baseline. In some embodiments, patients experience an 80 ms reduction in cT1 compared to baseline, suggestive of an improvement in fibrosis by one stage.

The present treatment methods may improve or prevent a fibrotic condition in a patient. In some embodiments, the methods decrease fibrosis formation. In some embodiments, the methods prevent or delay onset of fibrosis, or prevent or slow down progression of fibrosis in a patient. In some embodiments, liver fibrosis in a patient is not worsened. The present treatment methods may improve fibrosis (i.e., may result in improvement in fibrosis in a patient). Fibrosis may be improved by one or more stages (e.g., one or two stages). Outcomes in patients with MASH may be driven by improvements in fibrosis (e.g., reversal of fibrosis or prevention of the progression of fibrosis) (see, e.g., Newsome et al., J Hepatology. (2023) 79:1557-65).

The methods may also decrease ALT, AST, Fib-4, GGT, or ALP in a patient. In some embodiments, ALT is decreased by 10% or more (e.g., 15% or more, 16.7% or more, 17% or more, 20% or more, 25% or more, 30% or more, 34% or more, 35% or more, 37% or more, or 39.0% or more) compared to baseline. In some embodiments, AST is decreased by 5% or more (e.g., 8.0% or more or 10.0% or more) compared to baseline. In some embodiments, Fib-4 is decreased by 1% or more (e.g., 1.85% or more) compared to baseline.

The methods may also decrease N-terminal type III collagen propeptide (Pro-C3) in a patient. A healthy reference range for Pro-C3 is 6.1-14.7 ng/mL, while a Pro-C3 value over 20 ng/mL is predictive of fibrosis progression and a worse metabolic state (see, e.g., Nielsen et al., J Hepatol. (2021) 75:1292-300). In some embodiments, Pro-C3 is decreased by 10 or more, 15 or more, 18 or more, 19 or more, 20 or more, or 25 or more ng/mL, or 10% or more, 20% or more, 25% or more, 30% or more, 33% or more, 34% or more, 35% or more, or 40% or more, compared to baseline. In some embodiments, Pro-C3 in a treated patient is between about 20 and 40 ng/mL, between about 25 and about 35 ng/mL, about 31 ng/mL, 32 ng/mL, about 33 ng/mL, about 34 ng/mL, about 35 ng/mL, or about 36 ng/mL. In some embodiments, patients experience a reduction of Pro-C3 levels compared to baseline, suggestive of an improvement in fibrosis by one stage (see, e.g., Hartman et al., Diabetes Care. (2020) 43:1352-55).

In some embodiments, the present treatment methods improve liver fibrosis or prevent progression of fibrosis in a patient. In some embodiments, fibrosis is evaluated via imaging (e.g., FibroScan®), pathophysiology (e.g., liver biopsy), or blood biomarkers (e.g., ProC3, alanine aminotransferase (ALT), or aspartate aminotransferase (AST)). Fibrosis may be measured by any technique known in the art including, e.g., FibroScan (including FAST, Agile3+, Agile4), MR Elastography, MRI cT1, NASH FibroSure Plus (BioPredictive), ELF (FDA approved prognostic biomarker, HA+PIIINP+TIMP-1), Nordic panels (including PRO-C3, ADAPT, fibrolysis markers), FIB-4 (age, ALT, AST, platelet count), SomaLogic NASH, NIS2+, or NIS4.

In some embodiments, the present treatment methods improve inflammation and hepatocellular injury (MASH activity) in a patient. Inflammation and hepatocellular injury (MASH activity) may be measured by any technique known in the art including, e.g., transaminase levels, CK-18, NASH2, SomaLogic NASH, NASH FibroSure Plus, NIS2+, NIS4, or MRI cT1.

In some embodiments, the present treatment methods improve steatosis in a patient. Steatosis may be measured by any technique known in the art including, e.g., SteatoTest, MRI-PDFF, FibroScan, SomaLogic NASH, or NASH FibroSure Plus.

In some embodiments, the present treatment methods improve live function in a patient. Liver function may be measured by any technique known in the art including, e.g., HepQuant DuO.

In some embodiments, the present methods may improve the nonalcoholic fatty liver disease activity score (NAS) in a patient compared to baseline. NAS may be determined based on assessment of steatosis, inflammation, and ballooning hepatocytes in liver biopsy. The score ranges from zero to eight, with higher scores indicating more serious disease. In some embodiments, the present methods improve the NAS of treated patients, by one, two, three, four, five, six, seven, or eight points.

### V. Articles of Manufacture and Formulations

The present disclosure provides an article of manufacture (e.g., a kit) for use in the present therapy. The article of manufacture may contain one or more doses of CT-388. The CT-388 dose(s) may be housed in a pre-filled syringe or injector, optionally a single-use syringe or injector (e.g. an autoinjector). The article of manufacture may also include use instructions. In some embodiments, CT-388 is provided in a pharmaceutical composition. The pharmaceutical composition may comprise CT-388 at a concentration of 20 mg/mL, and may also comprise a sodium phosphate buffer or mannitol, optionally at pH 7.0. In some embodiments, the pharmaceutical composition comprises CT-388 at a concentration of 20 mg/mL, sodium phosphate buffer, and mannitol at pH 7.0.

The pharmaceutical composition may comprise the components set out in Table 2.

TABLE 2
CT-388 Hydrochloride Salt Drug Product Composition

| | Amount per Vial | | Quality |
|---|---|---|---|
| Component | (mg/mL) | Function | Standard |
| CT-388 | 20 | Active Ingredient | In-house |
| Sodium monophosphate | 1.38 | Buffer | USP/NF, BP, |
| monobasic monohydrate | | | Ph. Eur., JP |
| Trehalose dihydrate | 25 | Tonicity modifier/ | USP/NF, |
| | | Bulking agent | Ph. Eur., JP |
| Mannitol | 40 | Tonicity modifier/ | USP/NF, BP, |
| | | Bulking agent | Ph. Eur. |
| 10% Polysorbate 20 solution | 1 | Surfactant | USP/NF, |
| | | | Ph. Eur., JP |
| 1N NaOH | q.s. to pH 6.5 ± 0.2 @ | pH adjustment | USP/NF, BP, |
| | 25 ± 1° C. | | Ph. Eur., JP |
| Water for Injection | q.s. | Solvent | USP/NF |

Abbreviations:
BP: British Pharmacopeia;
NF: National Formulary;
Ph. Eur.: European Pharmacopeia;
JP: Japanese Pharmacopeia:
q,s: Quantum satis, water added in the amount needed to fill 1.0 mL;
USP: United States Pharmacopeia

In some embodiments, the pharmaceutical composition is provided in a vial (e.g., a 5 mL glass vial). The vial may be a 20 mm glass vial with a single vent lyo stopper closed with a 20 mm flip-off TruEdge matte white seal. In some embodiments, each vial comprises 1.0 mL of the pharmaceutical composition. In some embodiments, each vial comprises 20 mg of CT-388. In some embodiments, the pharmaceutical composition is provided in an autoinjector.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety, as if each individual reference were specifically and individually indicated to be incorporated by reference in its entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context. All ranges should be interpreted as inclusive (e.g., a range of 1-3, or between 1-3, includes the numbers 1, 2, and 3).

According to the present disclosure, back-references in the dependent claims are meant as short-hand writing for a direct and unambiguous disclosure of each and every combination of claims that is indicated by the back-reference. Any compound disclosed herein can be used in any of the treatment method here, wherein the individual to be treated is as defined anywhere herein. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: A Phase 1 Clinical Trial Protocol

This Example outlines the protocol for a Phase 1 randomized, double-blind, placebo-controlled study to evaluate the safety, tolerability, pharmacokinetics, and pharmacodynamics of CT-388 in otherwise healthy overweight and obese adult participants and in obese patients with type 2 diabetes mellitus (NCT04838405).

For once weekly dosing, CT-388 is supplied as a 1-mL or 2-mL lyophilized powder or sterile solution at a concentration of 20 mg/mL, or placebo, as sterile solutions for subcutaneous (SC) injection.

### A. Objectives

### Single-Ascending Dose (SAD) Once Weekly (QW)

The primary objective of this study is to determine the safety and tolerability of CT-388 in otherwise healthy overweight and obese adult participants when administered as a single dose of 0.5, 2.0, 5.0, 6.0, or 7.5 mg.

The secondary objectives are:
To evaluate the pharmacokinetics of single-ascending doses of CT-388 in otherwise healthy overweight and obese adult participants; and
To evaluate the pharmacodynamic (PD) effect of a single dose of CT-388 on body weight and glucose homeostasis in both the fasted state and in the setting of a mixed meal tolerance test (MMTT) challenge in otherwise healthy overweight and obese adult participants.

### Multiple-Ascending Dose (MAD) QW

The primary objective of this study is to determine the safety and tolerability of CT-388 in otherwise healthy overweight and obese adult participants when administered once weekly over 4 consecutive weeks with titrated dosing.

The secondary objectives of this study are:
To evaluate the pharmacokinetics of CT-388 when titrated across 4 consecutive weekly doses; and
To evaluate the PD effect of 4 consecutive weekly titrated doses of CT-388 on body weight and glucose homeostasis in both the fasted state and in the setting of an oral glucose tolerance test (OGTT) in otherwise healthy overweight and obese participants.

### Multiple Dose (MD) QW

The primary objective of this study is to determine the safety and tolerability of CT-388 in participants with and without type 2 diabetes mellitus (T2DM) when administered once weekly over 12 consecutive weeks for Cohorts 11 and 14 with titrated dosing (and additionally, up to 24 weeks for Cohorts 12 and 13).

The secondary objectives of this study are:
To evaluate the pharmacokinetics of CT-388 across 12 consecutive weekly doses for Cohorts 11 and 14 (and additionally, up to 24 weeks for Cohorts 12 and 13); and
To evaluate the PD effect of CT-388 across 12 consecutive weekly doses for Cohorts 11 and 14 (and additionally up to 24 weeks for Cohorts 12 and 13) on body weight and glucose homeostasis in both the fasted state and in the setting of an OGTT in participants with or without T2DM.

The exploratory objectives of this study are:
To evaluate the PD effect of CT-388 when titrated across 12 consecutive weekly doses and additionally up to 24 weeks in participants without T2DM (Cohort 12) or with T2DM (Cohort 13) on MRI-PDFF (proton density fat fraction) and adiposity; and
To evaluate the PD effect of CT-388 when titrated across 12 consecutive weekly doses in Cohorts 11-14 on biomarkers (e.g., lipid profile, adiponectin, alanine aminotransferase (ALT), Pro-C3, CK18, FIB-4), and additionally up to 24 weeks in participants without T2DM (Cohort 12) or with T2DM (Cohort 13).

### B. Endpoints

Safety and tolerability will be assessed by monitoring adverse events (AE) including local injection-site reactions, measuring vital signs and ECGs, and performing clinical laboratory blood and urine analyses.

### C. Pharmacodynamics

The PD evaluation includes changes in body weight, fasting glucose, fasting insulin, HOMA-IR, waist and hip circumference, fructosamine, HbA1c, fasting lipid profile, fasting free fatty acids, ketone bodies, adiponectin, MRI-PDFF, biomarkers (ALT, PRO-C3, CK18, and FIB-4), as well as AUC, baseline-adjusted AUC, Cmax, and Tmax from baseline to a pre-determined day.

### D. Main Criteria for Inclusion

Cohorts 1-6 will include overweight or obese otherwise healthy adult male or female participants aged 18-65 years (inclusive) with a body mass index (BMI)≥25.0 kg/m² (inclusive, at Screening).

Cohorts 7-12 and 14 will include only obese otherwise healthy adult male or female participants aged 18-65 years (inclusive) with a BMI≥30.0 kg/m² (inclusive, at Screening). Abnormalities in blood pressure (i.e., treated hypertension), serum lipids (i.e., treated hyperlipidemia), and serum glucose values consistent with a diagnosis of prediabetes are acceptable.

Cohort 13 will include adult male or female participants aged 18-65 years (inclusive) with a BMI≥30.0 kg/m² (inclusive, at Screening) and have a diagnosis of T2DM for ≥6 months that is managed with diet and exercise alone, or treated with a stable dose of metformin monotherapy for at least 3 months prior to Screening Visit and with an HbA1c between 7.0% and 10.0%. Other abnormalities of serum glucose, serum lipids, urinary glucose and urinary protein consistent with a diagnosis of T2DM are acceptable.

### E. Inclusion Criteria

Inclusion criteria for participants of all cohorts include the following:
1) Males or females, between 18 and 65 years of age, inclusive, at time of consent.
2) Female participants must not be pregnant, or breast feeding.
3) BMI≥25.0 kg/m², inclusive at Screening and Admission (Day −2/Day −1) for Cohorts 1-6, and BMI≥30.0 kg/m², inclusive at Screening and Admission (Day 2/Day 1) for Cohorts 7-14.
4) Stable body weight (defined as ≤4.0 kg gain or loss) in the 2 months prior to Screening as per verbal report by participant and for the period between Screening and Admission (Day −2/Day −1) as per measured weight.
5) No clinically significant symptoms, signs, or active manifestations of stable conditions in the opinion of the Principal Investigator (PI) or delegate from medical history, physical exam, vital signs, and laboratory findings at Screening and Admission (Day −2/Day −1).

### F. Exclusion Criteria

Exclusionary criteria for participants of all cohorts include the following:
1) Clinically significant history or active clinical manifestation(s) of any significant metabolic, allergic, dermatological, hepatic, renal, hematological, pulmonary, cardiovascular, gastrointestinal, neurological, or psychiatric disorder (as determined by the PI or delegate). However, the following exceptions may be considered:
   a) Participants with non-severe gastroesophageal reflux if stable on therapy with a proton pump inhibitor for >3 months prior to Screening may be allowed.
   b) Participants with stable asthma (i.e., no exacerbations or use of asthma relievers within 3 months prior to Screening) and anticipated to be stably managed for the duration of the study may be allowed.
   c) Participants with stable depression being treated with stable doses and regimen of selective serotonin reuptake inhibitors (SSRIs) or serotonin norepinephrine reuptake inhibitors (SNRIs) for more than 12 months prior to screening may be allowed; however, a history of depression requiring inpatient admission/ECT/past treatment with antipsychotics and/or requiring ongoing psychotherapy and/or on treatment with any central nervous system stimulants (e.g., Ritalin-SR® for ADHD) is exclusionary.
   d) Participants may be on medications for the control of hypertension (e.g., ACE-inhibitors) or hyperlipidemia (e.g., statins/fibrates) provided that their blood pressure and lipid parameters are stably controlled, and must be on a dose and regimen that has been stable for more than 3 months prior to the Screening Visit, and that the medication/dose/regimen is not anticipated to change during the course of the study.
2) Current diagnosis of any type of diabetes (as defined by the 2022 American Diabetes Association Standards of Medical Care in Diabetes (Clin Diabetes (2022) 40(1):10-38), except for Cohort 13 where diagnosis of T2DM is required)
   a) Participants with prediabetes (as defined by the 2022 American Diabetes Association Standards of Medical Care in Diabetes (Clin Diabetes (2022) 40(1):10-38) may be allowed, but only if they are controlled and maintained on diet/exercise alone (i.e., no pharmacotherapy of any type, including no metformin, nor any other type of over-the-counter/herbal/nonprescription/supplement treatments are allowed).
3) History of bariatric surgery (of any type) or gastric banding (including LAP-BAND), history of any gastrointestinal surgery that may induce malabsorption (e.g., bowel resection), or any GI motility disorders, gastroparesis, delayed gastric emptying, inflammatory bowel disease, pancreatitis, malabsorption syndromes, chronic diarrhea, chronic constipation, clinically significant irritable bowel syndrome.
4) Semi-supine blood pressure systolic >160 mm Hg and/or diastolic >95 mm Hg at Screening, Admission (Day 2/Day 1), and Day 1 (predose).
5) An active or untreated malignancy or have been in remission from a clinically significant malignancy (other than basal or squamous cell skin cancer, in situ carcinomas of the cervix, or in situ prostate cancer) for <5 years prior to screening. Personal or family history (first-degree relative) of medullary thyroid carcinoma (MTC), or a genetic condition that predisposes to MTC (i.e., thyroid C-cell hyperplasia, or multiple endocrine neoplasia type 2A or type 2B)
6) History of atopy (severe or multiple allergic manifestations) or clinically significant multiple or severe drug allergies, or intolerance to topical corticosteroids, or severe posttreatment hypersensitivity reactions; known allergy to any component of the study drug, GLP-1 analogues, or related compounds
7) Any of the following laboratory abnormalities at Screening Visit (some exceptions to this criterion would apply to Cohort 13 where diagnosis of T2DM is required; see below):
   a) Aspartate transaminase [AST], alanine aminotransferase [ALT], alkaline phosphatase [ALP], gamma glutamyl transpeptidase [GGT]>2× the upper limit of normal (ULN) or
   b) Total bilirubin >ULN
   c) Amylase and Lipase >ULN
   d) Fasting triglycerides ≥250 mg/dL (2.83 mmol/L)
   e) Estimated glomerular filtration rate (eGFR)<60 mL/min/1.73 m² as calculated by the central laboratory using the Chronic Kidney Disease Epidemiology Collaboration (CKD-EPI) formula at Screening.
   f) HbA1c level ≥6.5%
   g) Fasting glucose ≥126 mg/dL (6.9 mmol/L)
   h) Hemoglobin <11 g/dL (males) or <10 g/dL (females)
8) Current use or use within 3 months prior to Screening of any prescribed or non-prescribed drugs that are known to interfere with gut motility including but not limited to chronic opioids, anticholinergics, and antispasmodics.
9) Use of hormone replacement therapy (other than oral contraceptive products/IUDs/IUSs) 60 days prior to Admission (Day −2/Day −1).

For eligibility to enroll into Cohort 13 (multiple doses for 24 weeks in obese participants with T2DM), all the above inclusion and exclusion criteria should be met, including meeting the following additional criteria:
1) Diagnosis of T2DM as defined by the 2022 American Diabetes Association Standards of Medical Care in Diabetes (Clin Diabetes (2022) 40(1):10-38) for at least 6 months prior to Screening Visit,
2) T2DM should be managed with diet and/or exercise alone or treated with a stable dose of metformin monotherapy (either immediate release or extended release ≥500 mg/day and not more than the locally approved dose) for at least 3 months prior to the Screening Visit.
3) BMI≥30.0 kg/m²,
4) HbA1c between 7.0% and 10.0%, inclusive,
5) Alanine transaminase, aspartate transaminase, alkaline phosphatase, gamma glutamyl transpeptidase) up to 3× the upper limits of the normal (ULN) range and total bilirubin up to 1.5×ULN at Screening may be allowed; other abnormalities in serum (e.g., glucose, lipids) and urine (e.g., glucose, protein) consistent with a diagnosis of T2DM are acceptable,
6) Fasting serum triglyceride levels up to 500 mg/dL (5.6 mmol/L) at Screening may be allowed,
7) eGFR should be >45 mL/min/1.73 m² as calculated by the central laboratory using the CKD-EPI formula at Screening,
8) Fasting blood glucose should be <270 mg/dL (14.8 mmol/L) at Screening.

### G. Test Product(s), Dose, and Mode of Administration

Participants will receive one of the following treatments:

### SAD QW—Cohorts 1-5

Cohort 1: One dose containing 0.5 mg of CT-388 or matched placebo administered subcutaneously in the abdomen
Cohort 2: One dose containing 2.0 mg of CT-388 or matched placebo administered subcutaneously in the abdomen
Cohort 3: One dose containing 5.0 mg of CT-388 or matched placebo administered subcutaneously in the abdomen
Cohort 4: One dose containing 7.5 mg of CT-388 or matched placebo administered subcutaneously in the abdomen
Cohort 5: One dose containing 6.0 mg of CT-388 or matched placebo administered subcutaneously in the abdomen

### MAD QW—Cohorts 6-10

Cohort 6: One weekly dose titrated across 4 consecutive weeks with CT-388 doses of . . .
   5.0 mg at Day 1
   5.0 mg at Day 8
   5.0 mg at Day 15
   7.5 mg at Day 22
      . . . or matched placebo administered subcutaneously in the abdomen.
Cohort 7: One weekly dose titrated across 4 consecutive weeks with CT-388 doses of . . .
   5.0 mg at Day 1
   5.0 mg at Day 8
   8.0 mg at Day 15
   12.0 mg at Day 22
      . . . or matched placebo administered subcutaneously in the abdomen.
Cohort 8: One weekly dose titrated across 4 consecutive weeks with CT-388 doses of . . .
   5.0 mg at Day 1
   8.0 mg at Day 8
   12.0 mg at Day 15
   12.0 mg at Day 22
      . . . or matched placebo administered subcutaneously in the abdomen (the third dose in Cohort 8 can be lowered to 8.0 mg based on tolerability data from Cohort 7)
Cohort 9: One weekly dose titrated across 4 consecutive weeks with CT-388 doses of . . .
   8.0 mg at Day 1
   8.0 mg at Day 8
   12.0 mg at Day 15
   16.0 mg at Day 22
      . . . or matched placebo administered subcutaneously in the abdomen.
Cohort 10: the following two dosing options are proposed in Table 3 below:

TABLE 3
Cohort 10 Options

| Option 1 | Option 2 |
|---|---|
| 8.0 mg at Day 1 | 5.0 mg at Day 1 |
| 12.0 mg at Day 8 | 8.0 mg at Day 8 |
| 16.0 mg at Day 15 | 12.0 mg at Day 15 |
| 20.0 mg at Day 22 | 16.0 mg at Day 22 |

Note:
The 4^{th} dose could be lowered to 16.0 mg based on tolerability data from Cohort 9

### MD QW—Cohorts 11-14

The primary aim for these cohorts is to enable a longer titration regimen to better delineate the tolerability profile to allow each participant to get to their individual maximal tolerated dose. In Cohorts 12 and 13, participants would also have an opportunity to further continue on such a dose for an additional 12 weeks (i.e., 24 weeks of dosing in total) to better assess safety and tolerability of the study product over an extended period.

In general, when titrating dose upwards, if a dose is not tolerated at any stage, revert to the previous tolerated dose for the next dose (i.e., down-titration is allowed), then attempt up-titration at the subsequent scheduled dosing time/week.

For all MD QW cohorts, the following should be performed to enable smooth up-titration of doses:
Flexible and an individual-based titration schedule tailored to the participant's specific tolerability level,
Temporarily withholding the study drug, and/or
Managing relevant AEs by reinforcing conservative measures (i.e., eating smaller-sized meal portions, less frequent/skipping meals), as well as with appropriate concomitant medications as clinically indicated.

The cohorts in the MD QW study are as follows.
Cohort 11: Once weekly dose administered SC in the abdomen across 12 consecutive weeks with CT-388 or matched placebo doses of 5.0 mg administered for the first 3 weeks and then titrated to 8.0 mg for the duration of the treatment period. If a participant(s) experiences moderate to severe tolerability issues after the third dose of 5 mg, the same 5 mg dose may be continued for up to an additional 2 weeks, i.e., up-titration to 8 mg may be attempted after the fourth or fifth 5 mg dose. If participant(s) still unable to tolerate 8 mg by Day 36, then that participant(s) may continue at 5 mg for the remaining duration of the treatment period.
Cohorts 12 and 13: Once weekly dose administered SC in the abdomen titrated across 12 consecutive weeks with CT-388 or matched placebo doses of:
   5.0 mg at Day 1 and Day 8
   8.0 mg at Day 15 and Day 22
   12.0 mg (or 8.0 mg based on tolerability) at Day 29 and Day 36
   17.0 mg (or 12.0 mg based on tolerability) at Day 43 and Day 50
   22.0 mg (or 12.0 mg or 17.0 mg based on tolerability) at Day 57.
      If at 22 mg on Day 57, continue that dose for the duration of the treatment period.
      If at 12 mg or 17 mg on Day 57, titration to the corresponding next higher dose level may be attempted on a weekly basis based on the individual's tolerability (e.g., if on Day 57, 12 mg dose was administered and tolerated, on Day 64, dose of 17 mg may be attempted).
      If not able to tolerate, continue at the previously tolerated dose level until the following week (e.g., to Day 71), at which point another attempt can be made to the next higher dose level. Attempts to the next higher dose level can continue to be made until Day 78.
      The dose level achieved at Day 78 should be maintained for an additional 12 weeks of weekly dosing in the extension period, resulting in a cumulative of 24 weeks of total doses for participants in Cohorts 12 and 13. At any time during the extension period, if the maintenance dose is not tolerated, then that dose may be either lowered by one corresponding dose level (e.g., if at 22 mg, decrease to 17 mg; if at 17 mg decrease to 12 mg), and/or the dosing may be withheld for up to 2 weeks. Following this temporary dose adjustment/withholding period, attempt to titrate back up to the previous (higher) dose level should be made. If that still results in poor toleration or if the Investigator believes participant may discontinue study due to poor toleration, the previously tolerated dose (as long as it is at least 12 mg) may be continued for the remainder of the extension period.
Dosing in Cohort 14 may follow one of these two dosing options shown in Table 4 below.

TABLE 4
Cohort 14 Options

| OPTION 1 | OPTION 2 |
|---|---|
| Minimum dose to tolerate is 16 mg | Minimum dose to tolerate is 12 mg |
| Once weekly dose administered SC in the | Once weekly dose administered SC |
| abdomen titrated across 12 consecutive | in the abdomen titrated across 12 |
| weeks with CT-388 or matched placebo | consecutive weeks with CT-388 or |
| doses of: | matched placebo doses of: |
| 8.0 mg at Day 1, Day 8, Day 15, and Day 22 | 8.0 mg at Day 1 and Day 8 |
| 16.0 mg at Day 29, Day 36, Day 43, and | 12.0 mg at Day 15 and Day 22 |
| Day 50 | 16.0 mg (or 12.0 mg based on |
| 12.0 mg may be provided on Day 36 and | tolerability) at Day 29 and Day 36 |
| Day 43 if 16 mg is not tolerated on Day 29 | 20.0 mg (or 12.0 mg or 16 mg |
| Attempts must be made to get to 16 mg | based on tolerability) at Day 43 |
| by Day 50; however, if an individual | and Day 50 |
| participant is experiencing tolerability | 24.0 mg (or 12.0 mg or 16.0 mg |
| issues, continued attempts to get them to | or20 mg based on tolerability) at |
| the 16 mg dose can be made up to Day 78 | Day 57. |
| 24.0 mg at Day 57, Day 64, Day 71, and | If at 24 mg on Day 57, continue that |
| Day 78 | dose for the duration of the treatment |
| 20.0 mg may be provided on Day 64 | period. |
| and Day 71 if 24 mg is not tolerated by | If at 12 mg or 16 mg or 20 mg on |
| an individual on Day 57 | Day 57, titration to the |
| Attempts must be made to get to 24 mg | corresponding next higher dose level |
| by Day 78 | may be attempted on a weekly basis |
| If tolerability remains a concern for a | based on the individual's tolerability |
| given individual to reach a dose up to24 | (e.g., if on Day 57, 16 mg dose was |
| mg, they can continue at the 20 mg dose; | administered and tolerated, on Day |
| continued attempts toget them to the 24 | 64, dose of 20 mg may be attempted). |
| mg dose can bemade up to Day 78 | If not able to tolerate, continue at the |
| If an individual participant is not able to tolerate | previously tolerated dose level until |
| the above titration regimen to 24 mg with the | the following week (e.g., to Day 71), |
| recommended flexibility and adjustments, a | at which point another attempt can |
| minimum dose of 16 mg is required to | be made to the next higher dose level. |
| continue in this cohort. The dose level | Attempts to the next higher dose level can |
| achieved at Day 78 should be maintained | continue to be made until Day 78. The dose |
| until EOT (Day 85). | level achieved at Day 78 should be |
| | maintained until EOT (Day 85). |

### H. Study Assessments

In Cohorts 12 and 13, participant evaluation will include magnetic resonance imaging (MRIs) at Screening (between Day −14 and Day −1, after all other inclusion/exclusion criteria have been met), Day 85 (week 12), and Day 169 (week 24). During the MIRI, proton density fat fraction (PDFF) will be assessed as a measure of liver fat, in addition to a body composition (lean and fat mass) analysis (skeletal muscle, visceral fat, and SC fat at the L3 vertebral body level). No contrast is needed for these MRI assessments. Participants will undergo a single imaging assessment at each indicated time point during which all the above MRI data will be captured.

### Example 2: Effect of CT-388 on Liver-Related Parameters in Participants with Obesity Without T2D

This Example describes analyses performed on the magnetic resonance imaging protein density fat fraction (MRI-PDFF) analysis population, which included participants with baseline, week 12, and end of trial (EOT; week 24) MRI-PDFF. A 4:1 randomization ratio was used (4 participants treated with 22 mg CT-388 (via up-titration) per 1 participant treated with placebo). Percentage changes in MRI-PDFF from baseline and changes in fibro-inflammation evaluated via iron corrected T1 (cT1) from baseline were analyzed using a mixed model for repeated measures.

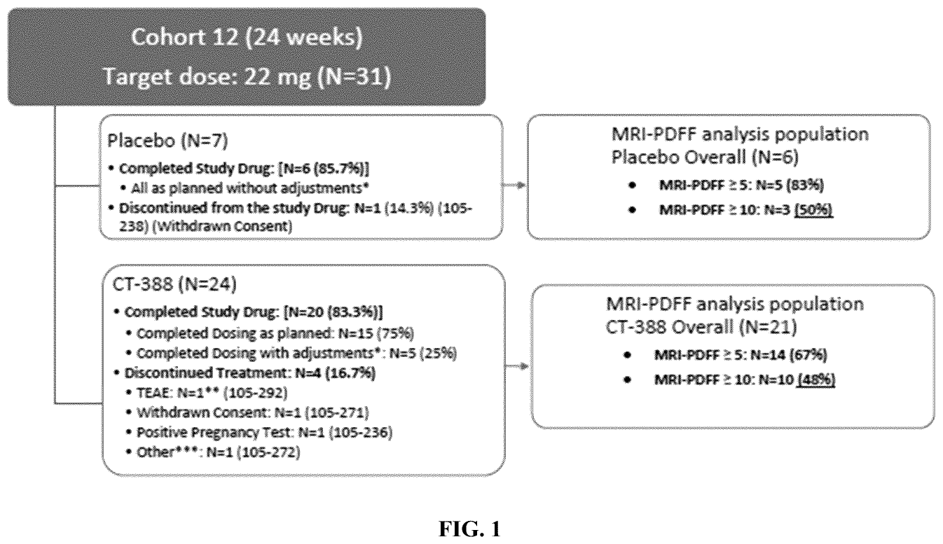

Cohort 12 included 31 otherwise healthy adults with obesity (BMI≥30.0 kg/m²). MRI was performed at baseline (BL) and weeks 12 and 24 to evaluate liver fat content (LFC) via MRI-PDFF and inflammation via cT1. 27 patients with complete MRI data (i.e., MRI measures taken at all three timepoints; 21 of which were treated with CT-388 and six of which were treated with placebo) were analyzed for percent change in liver fat content (LFC) and absolute reduction in cT1 from baseline using a mixed model for repeated measures. FIG. 1 shows the subject dispositions for the Cohort 12 MRI-PDFF analysis population. 75% of participants were dosed per planned path and completed treatment on 22 mg. 25% had dose down-titration and completed treatment on 12 mg. There were no participants who completed treatment on 17 mg.

The demographic and baseline characteristics of the MRI analysis population in the CT-388-101 MD (Cohort 12) are shown in Table 5. Participants treated with CT-388 were mostly females (61.9%), with a mean age of 31.4 yrs [SD: 9.05], BMI of 38.5 kg/m² [SD: 5.79], baseline MRI-PDFF Liver Fat Content (LFC) of 10.3% [SD: 7.87%], and cT1 858.2 ms [SD: 77.56]. Placebo participants were males (100%), with a mean age of 32.3 years [SD: 11.96], BMI of 34.9 kg/m² [SD: 3.30], MRI-PDFF LFC 8.9% [SD: 3.82%] and cT1 of 799.5 ms [SD: 67.35]. The majority of CT-388 participants had Obesity Class 2-3 (BMI≥35 kg/m²). Approximately 48% of the overall population (CT-388 and placebo) had liver steatosis ≥10%, illustrating significant liver steatosis and main eligibility criteria in non-invasive phase 2 MASH studies. Among these patients, CT-388 patients (n=10) had baseline LFC of 17% (SD 7%) vs 12% (SD 1%) in placebo patients (n=3).

Mean cT1, an imaging marker of fibroinflammatory conditions, in the CT-388 arm was 858, supportive of high-risk participants with possible liver-related clinical events and possible outcomes (cut off of 825 ms optimal rule-out threshold for high-risk NASH; see, e.g., Andersson et al., Clinical Gastroenterology and Hepatology. (2022) 20(11):2451-61.e3).

TABLE 5
Demographic and Baseline Characteristics

| | | | Planned Target CT - | |
|---|---|---|---|---|
| | Pooled Placebo | | 388 Dose (22 mg)^{b} | |
| | (N = 6) | | (N = 21) | |
| Age (years), Mean (SD) | 32.3 | (11.96) | 31.4 | (9.05) |
| Age (years), Median | 29 | (21-50) | 28 | (21-51) |
| (range) | | | | |
| Sex, females n (%) | 0 | (0) | 13 | (61.9) |
| Ethnicity, n (%) | | | | |
| Hispanic or Latino | 6 | (100) | 21 | (100) |
| Body Weight (kg), Mean | 102.0 | (11.32) | 108.1 | (20.04) |
| (SD) | | | | |
| Body weight (kg), Median | 100.1 | (86.8-115.8) | 105.8 | (77.4-143.2) |
| (range) | | | | |
| BMI (kg/m²), Mean (SD) | 34.9 | (3.30) | 38.5 | (5.79) |
| BMI (kg/m²), Median | 35.6 | (30.5-39.9) | 38.6 | (30.3-52.0) |
| (range) | | | | |
| Waist Circumference, | 112.3 | (4.37) | 116.9 | (14.04) |
| Mean (SD) | | | | |
| HbA1c, Mean (SD) | 5.5 | (0.36) | 5.4 | (0.32) |
| HbA1c, Median (range) | 5.5 | (5.0-5.9) | 5.4 | (4.6-5.9) |
| HOMA-IR, Mean (SD) | 5.2 | (2.18) | 4.3 | (1.94) |
| MRI-PDFF, Mean (SD) | 8.9 | (3.82) | 10.3 | (7.87) |
| MRI-PDFF ≥5%, Mean | 10.0 (3.03) | | 13.8 (7.46) | |
| (SD) | n = 5 | | n = 14 | |
| MRI-PDFF ≥10%, Mean | 11.9 (1.40) | | 16.5 (7.19) | |
| (SD) | n = 3 | | n = 10 | |
| LFC, Median (range) | 9.5 | (3.4-13.3) | 8.5 | (1.7-30.7) |
| cT1, Median (range) | 804 | (725-875) | 851 | (756-985) |
| ALT, Median (range)^{c} | 31 | (23-58) | 24 | (10-59) |
| AST, Median (range)^{d} | 25 | (19-36) | 22 | (14-40) |
| Pro-C3, Median (range)^{e} | 48 | (28-97) | 52 | (27-88) |
| cT1, Mean (SD) | 799.5 | (67.35) | 858.2 | (77.56) |
| ALT, Mean (SD) | 36.8 | (15.91) | 26.95 | (13.00) |
| AST, Mean (SD) | 26.7 | (7.63) | 21.4 | (6.03) |

^{b}Cohort 12 titration path: 5 mg, 8 mg, 12 mg and 17 mg, 2 weeks each in this order, then 22 mg from Week 8 to Week 24.
^{c}Healthy range for ALT is: ~29-33 U/L (males), ~19-25 U/L (females).
^{d}Healthy upper reference limit for AST is ~18 U/L.
^{e}Healthy reference range for Pro-C3 is 6.1-14.7 ng/mL (Erhardtsen et al., JHEP Rep. (2021) 3: 100317).
ALT = alanine aminotransferase, AST = aspartate aminotransferase, BMI = body mass index, cT1 = iron corrected T1, LFC = liver fat content, MRI = magnetic resonance imaging, Pro-C3 = N-terminal type III collagen propeptide.

### Weight Loss and Change in Waist Circumference

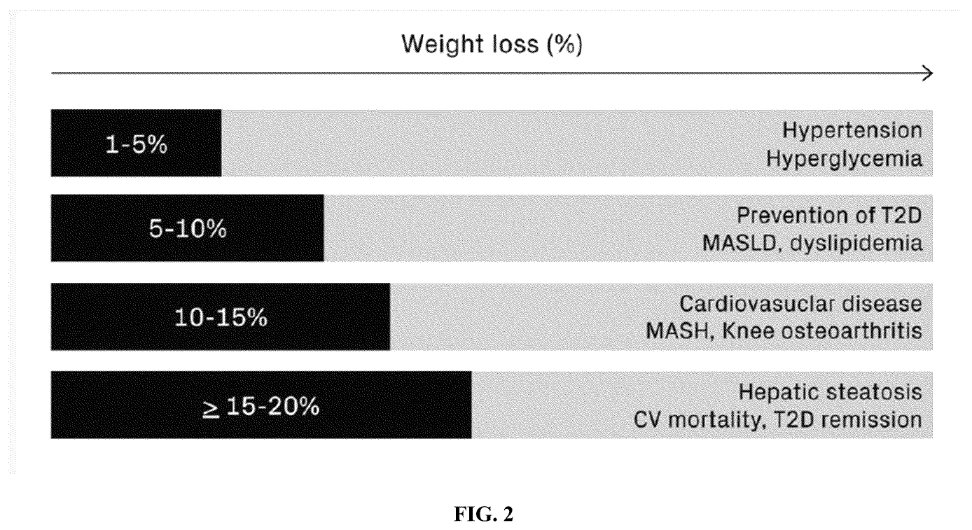

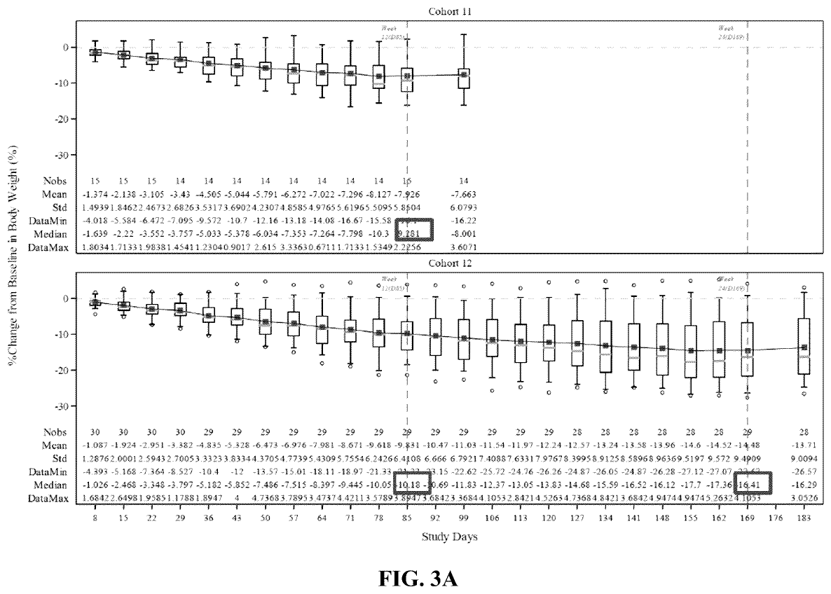

Differing amounts of weight loss have been shown to achieve certain health benefits in participants (FIG. 2).

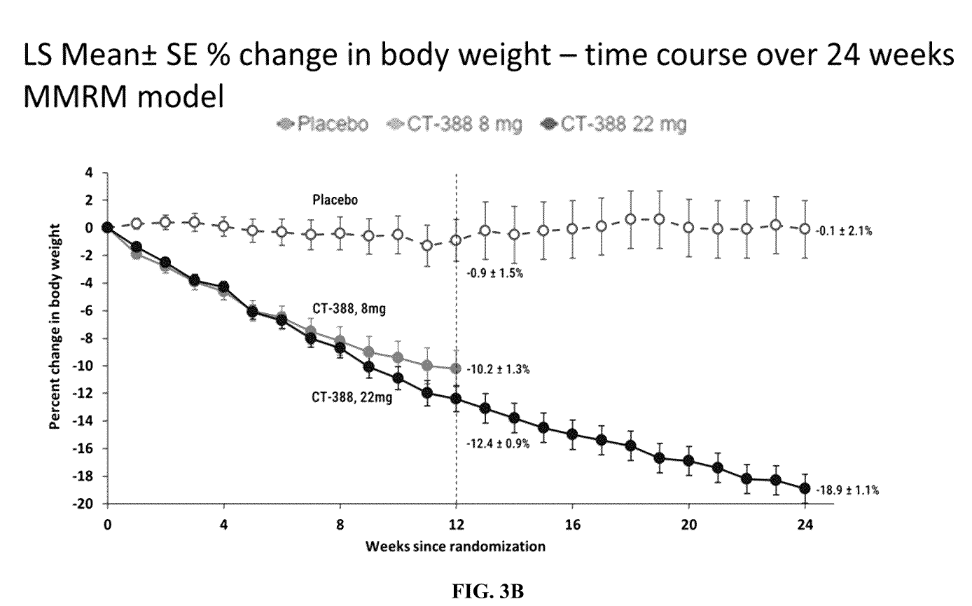

Cohorts 11 and 12 of the clinical trial described in Example 1 experienced weight loss while on treatment with CT-388 (FIGS. 3A and 3B). Evaluation of weight loss was performed by pooling placebo and CT-388 together using summary statistics (no inferential stats methods were used to evaluate drug effect based on baseline characteristics or placebo) and all observed values (including weight measured more than 8 days of the last dose of the study drug). At week 12, the median weight loss in cohort 11 (CT-388/placebo; 8 mg; n=15) was −9.3% and the median weight loss in cohort 12 (CT-388/placebo; 22 mg; n=29) was −10.2%. At week 24, treatment resulted in clinically meaningful weight loss (18.9% (95% CI 14.0%, 23.6%) placebo-adjusted).

### Liver-Related Imaging Parameters

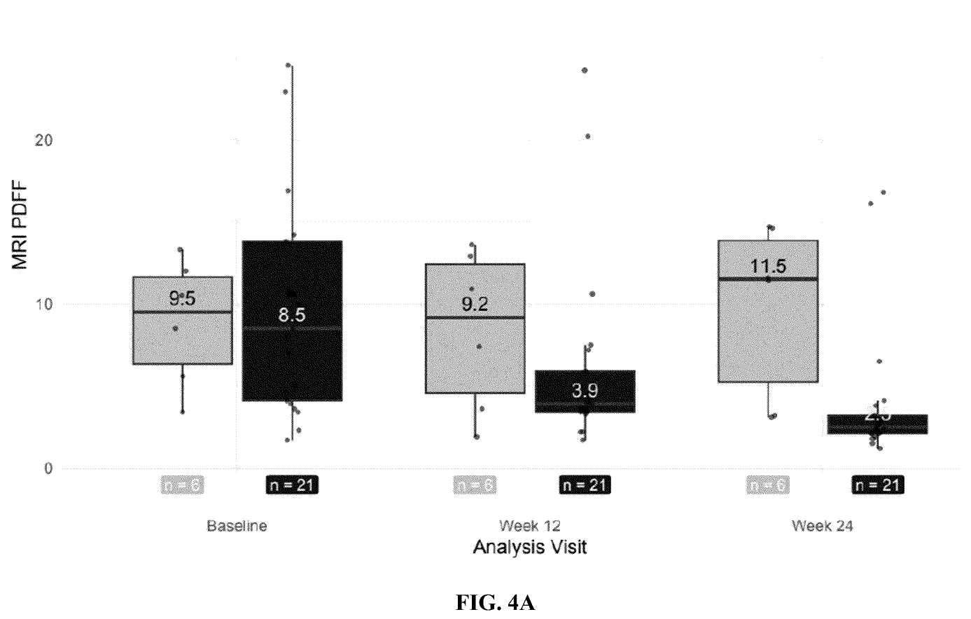

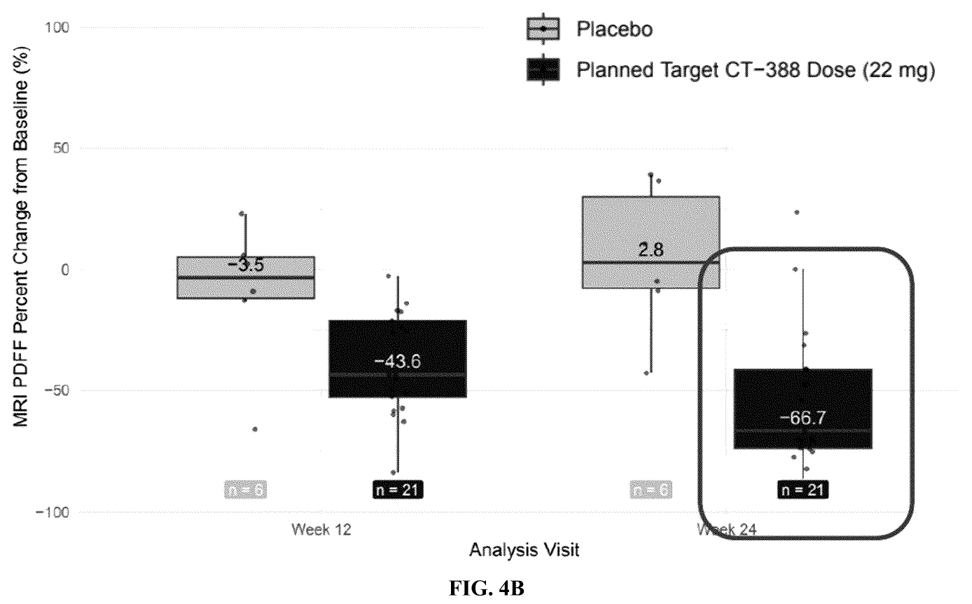

CT-388 resulted in significant reduction in liver steatosis in participants with obesity. Reduction was noted after 12 weeks of treatment and improved with longer duration of treatment. After 24 weeks, liver fat content was reduced by 58.5% (95% CI: 85.9%-31.1%, placebo-adjusted, percentage change, LS mean) (FIGS. 4A and 4B; Table 6). Median MRI-PDFF (LFC (%)) achieved 2.5% at Week 24, compared to 8.5% at baseline. Robust liver fat content (LFC) reduction was observed in all CT-388-treated participants.

TABLE 6
Analysis of Percent Change from Baseline in Median Liver PDFF in Cohort 12 Participants

| | | Least | Difference in | | | 95% CI |
|---|---|---|---|---|---|---|
| | | Squres | least squares | | | Estimate |
| | | Mean | mean from | | Standard | for |
| Visit | Treatment | Estimate | placebo estimate | P value | Error | Difference |
| Week | Pooled | −10.373 | | | | |
| 12 | Placebo | | | | | |
| | CT-388 5-8- | −30.639 | −20.266 | 0.238 | 16.776 | (−54.717, 14.184) |
| | 12-17-22 mg | | | | | |
| Week | Pooled | 4.061 | | | | |
| 24 | Placebo | | | | | |
| | CT-388 5-6- | −53.344 | −57.405 | <0.001*** | 13.624 | (−85.365, −29.446) |
| | 12-17-22 mg | | | | | |

The linear mixed model is specified as follows. The response variable is the PD change from baseline (or % CFB) and the model includes treatment, visit and treatment*visit as a fixed effects, and baseline value as a covariate and subject as a blocking effect within the residual covariance matrix (general pattern, autoregressive(1), or compound symmetry).
The model-adjusted means at each treatment are calculated using the average value of the covariate (baseline).
MD QW = multiple dose, once weekly;
CHG = change from baseline;
PCHG = percent
change from baseline

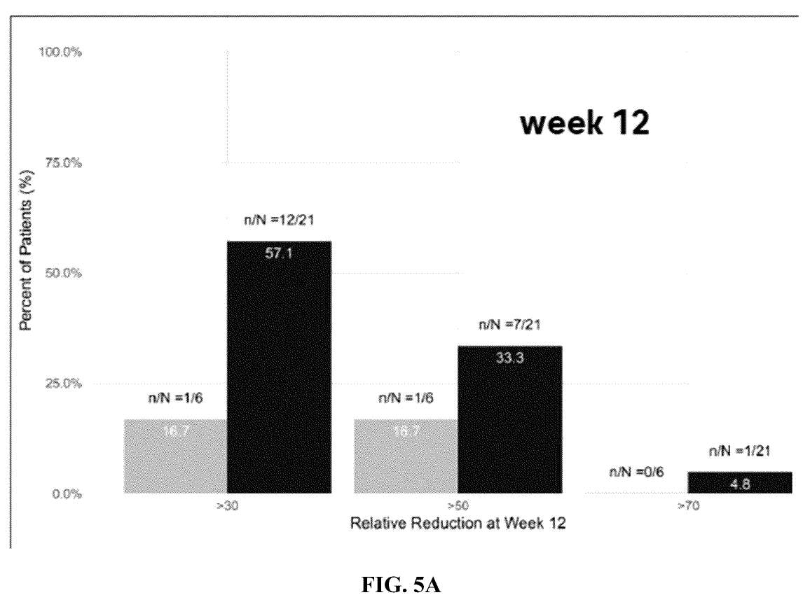

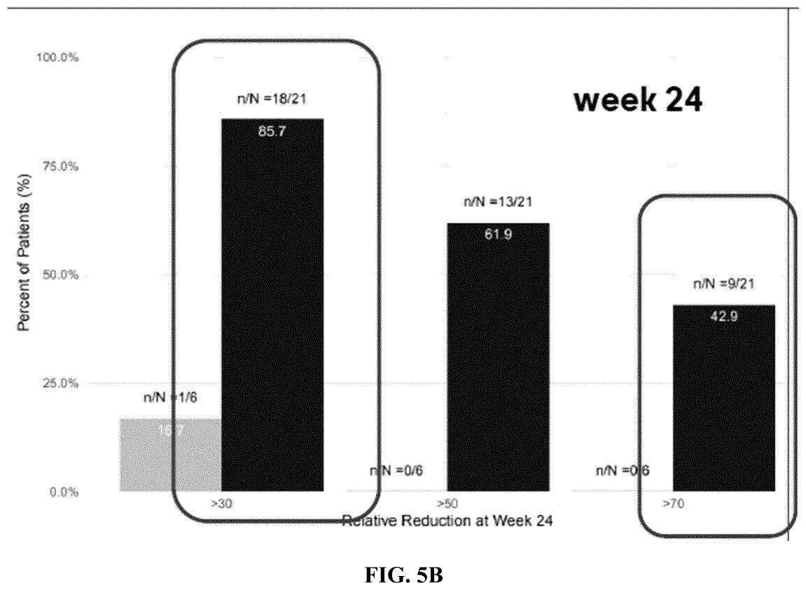

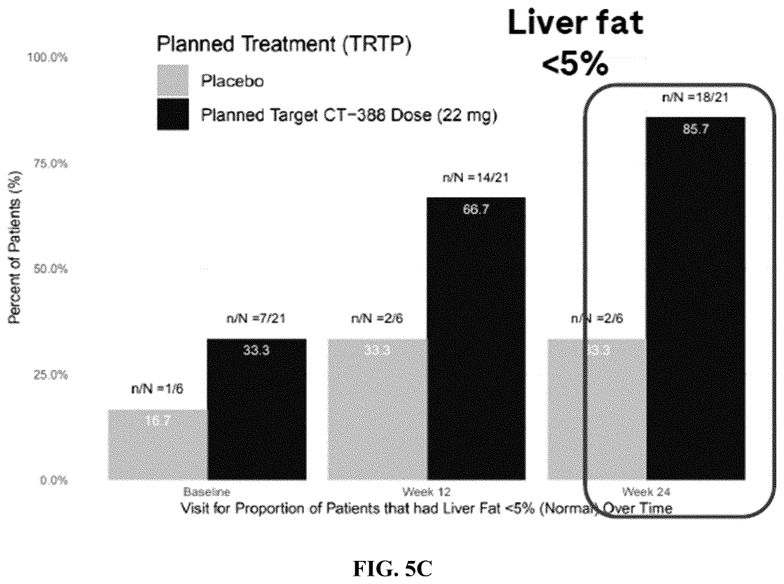

24 weeks of treatment with CT-388 resulted in significant reduction in liver steatosis in participants with obesity. Liver fat was reduced by ≥30% in 85.7% (18/21) of participants and by ≥70% in 42.9% (9/21) of participants (FIGS. 5A-5C). Robust liver fat content reduction was observed in all CT-388-treated participants.

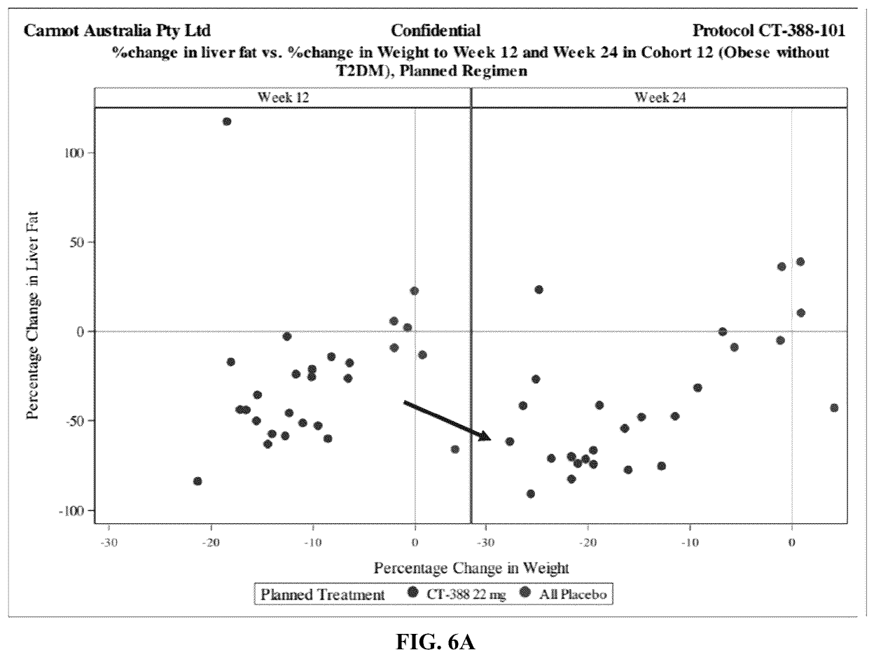

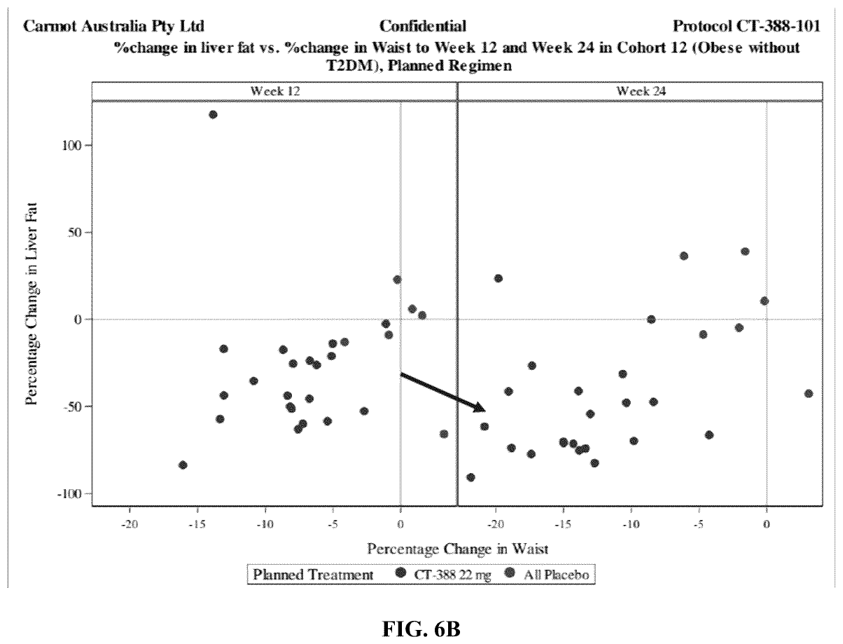

Only 16.7% of placebo participants had an MRI-PDFF decrease of ≥30%. An MRI-PDFF reduction by at least 30% is an independent factor of fibrosis regression and associated with higher odds of a histologic improvement in fibrosis (see, e.g., Tamaki et al., Gut. (2022) 71(5):983-990; Stine, 2020). Liver fat decreased to <5% in majority of participants (85.7%, 18/21), compared to 33.3% at baseline. CT-388 treatment also showed trends between change in liver steatosis and weight loss (FIG. 6A) and between change in liver steatosis and waist circumference (FIG. 6B).

Baseline liver MRI-PDFF levels in the overall participant population, participants with MRI-PDFF≥5%, and participants with MRI-PDFF≥10% are shown in Table 7.

TABLE 7
Baseline Liver MRI-PDFF Levels

| | | Planned Target CT - |
|---|---|---|
| | Pooled Placebo | 388 Dose (22 mg)^{b} |
| | (N = 6) | (N = 21) |
| MRI-PDFF, | 8.9 (3.82) | 10.3 (7.87) |
| Mean (SD) | | |
| MRI-PDFF ≥5%, | 10.0 (3.03) | 13.8 (7.46) |
| Mean (SD) | n = 5 | n = 14 |
| MRI-PDFF ≥10%, | 11.9 (1.40) | 16.5 (7.19) |
| Mean (SD) | n = 3 | n = 10 |

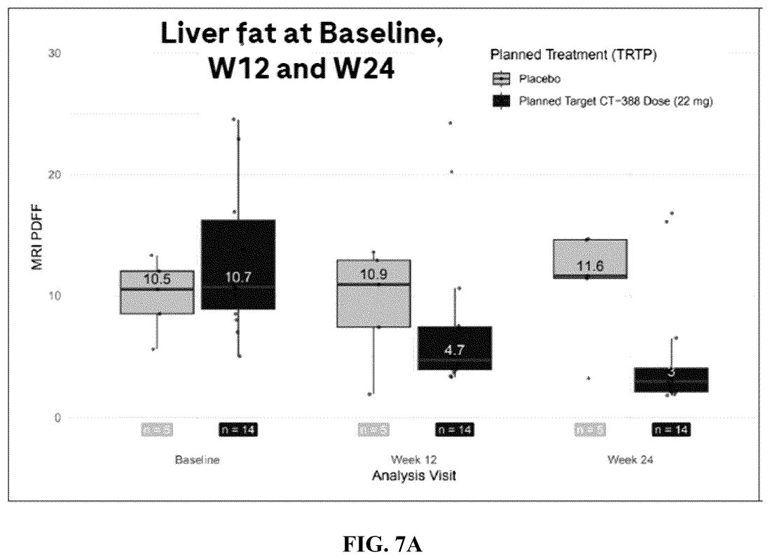

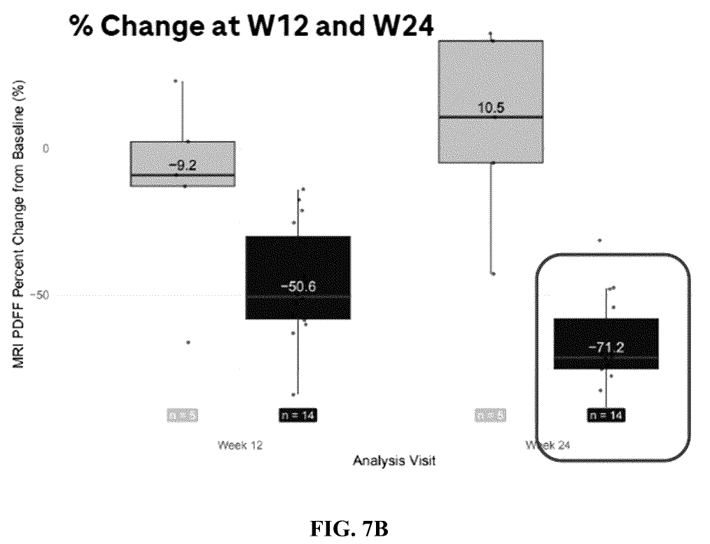

The effect of CT-388 on liver MRI-PDFF in participants with a baseline MRI-PDFF of ≥5% was tested. In participants with liver steatosis ≥5% at baseline, a decrease in liver fat was noted after 12 weeks of treatment and improved with longer duration of treatment. The effect of 24 weeks of treatment with CT-388 was more prominent than the effect of CT-388 after 12 weeks (FIG. 7A). After 24 weeks, liver fat content was reduced by 78.1% (95% CI: 102.5%-53.8%, placebo-adjusted, percentage change) (FIG. 7B, Table 8), and treatment achieved a median MRI-PDFF of 3%.

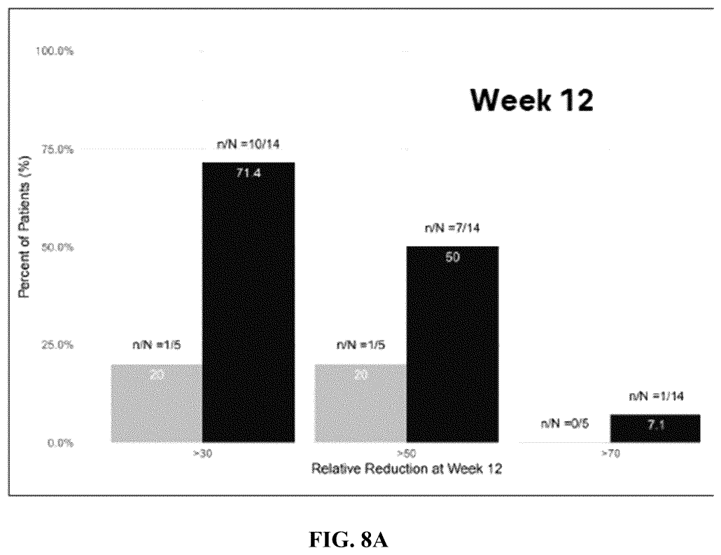

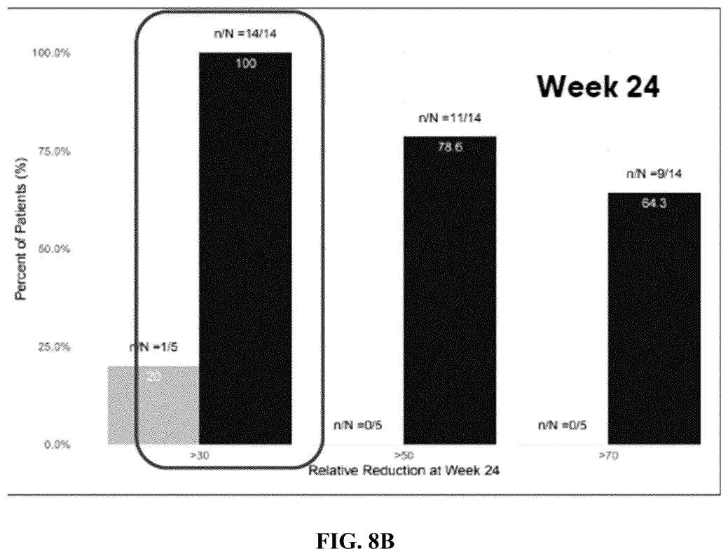

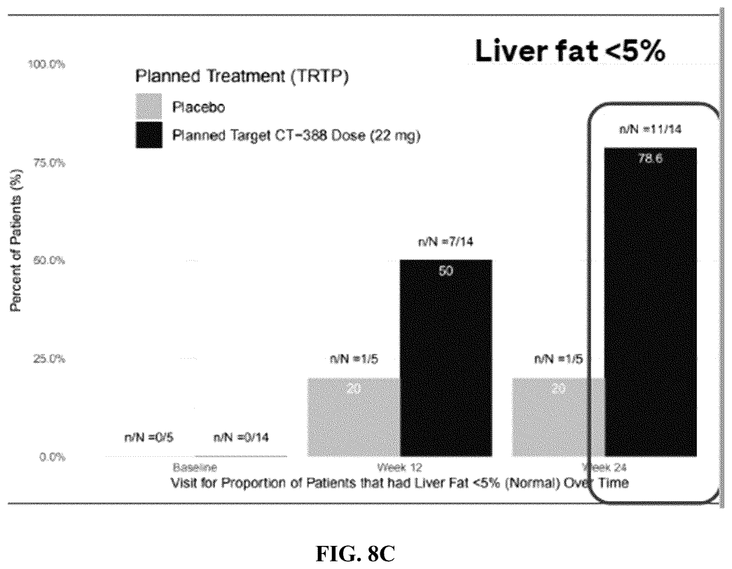

A reduction in MRI-PDFF (LFC) of ≥30% was noted in 100% (14/14) of participants. Greater liver fat content reduction was observed in participants with MASLD (LFC≥5%) at baseline. 64.3% (9/14) of participants experienced a reduction in liver fat by ≥70%. The trend is consistent across week 12 and week 24. 78.6% (11/14) participants had a liver MRI-PDFF<5% (normal) at week 24 (FIGS. 8A-8C).

TABLE 8
Effect of CT-388 on Liver MRI-PDFF in Participants
with Baseline MRI-PDFF of ≥5%

| | LSmean PCHG CT-388 |
|---|---|
| | 22 mg vs. PBO (%, 95% CI) |
| Week 12** | −36.9 (−63.7, −10.1) |
| Week 24*** | −78.1 (−102.5, −53.8) |

***p < 0.001
**p < 0.01;
* p < 0.05

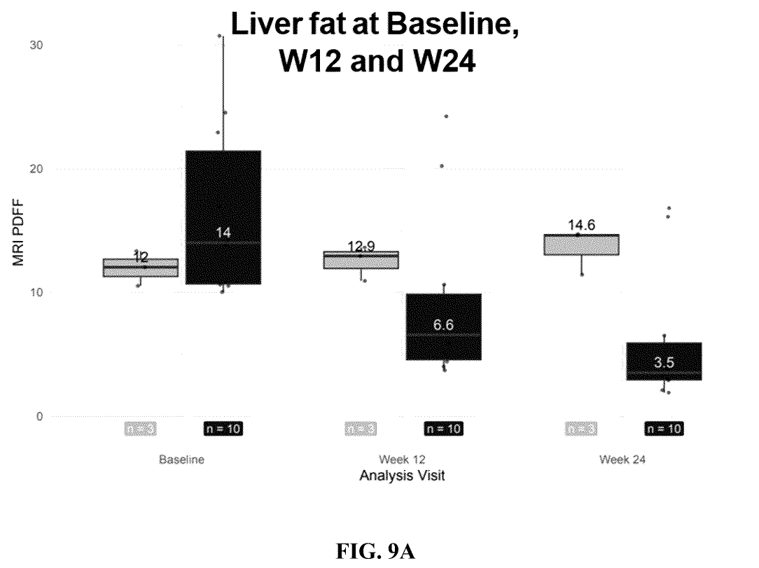

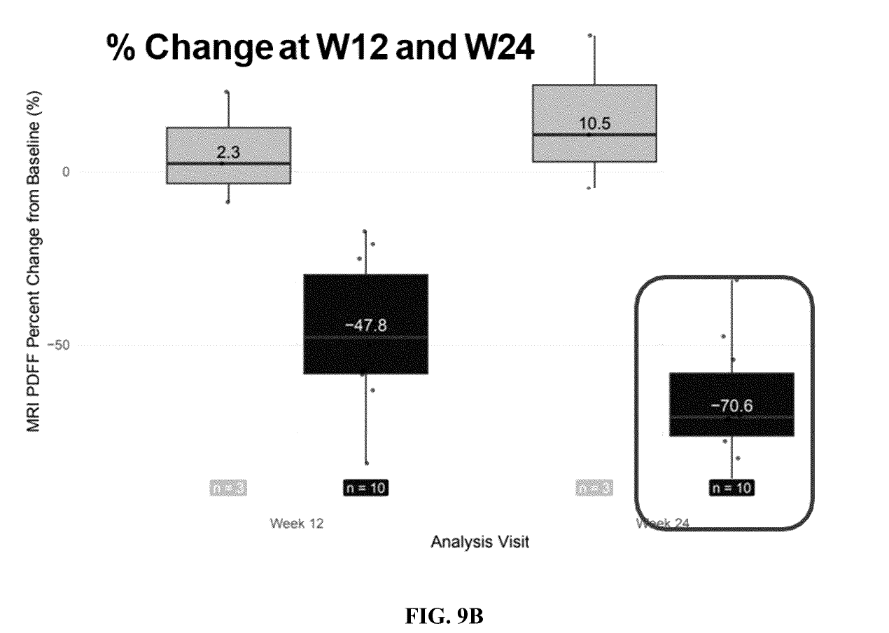

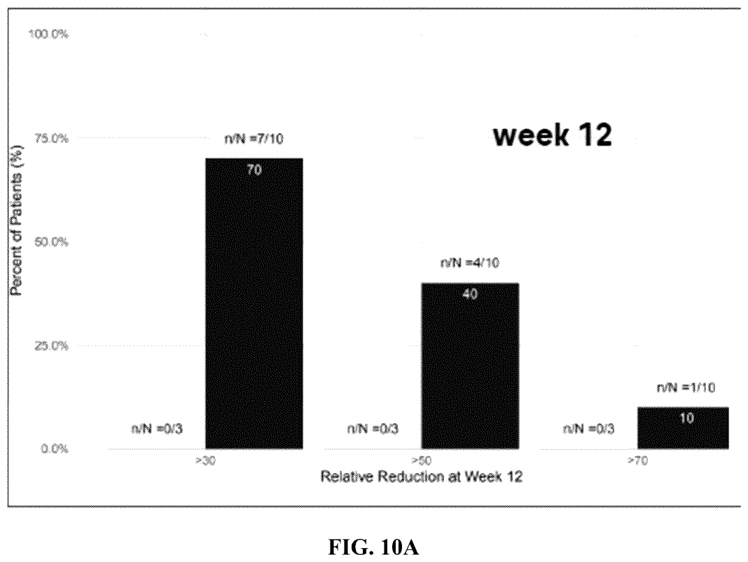

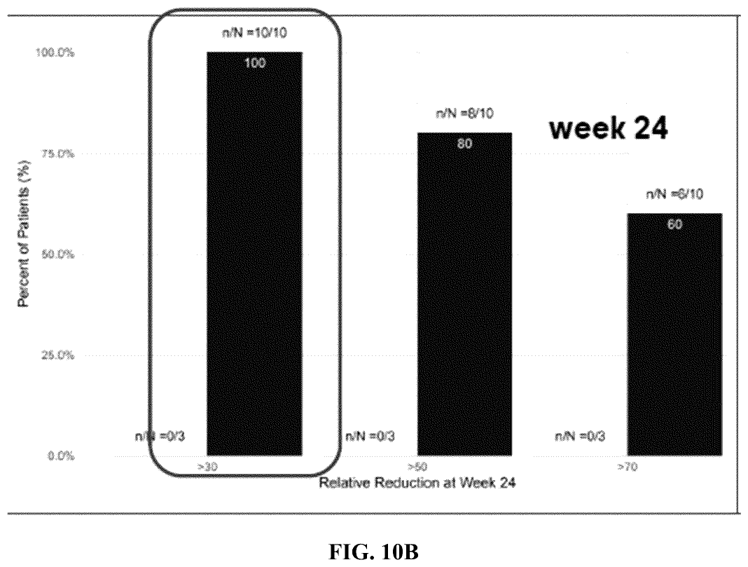

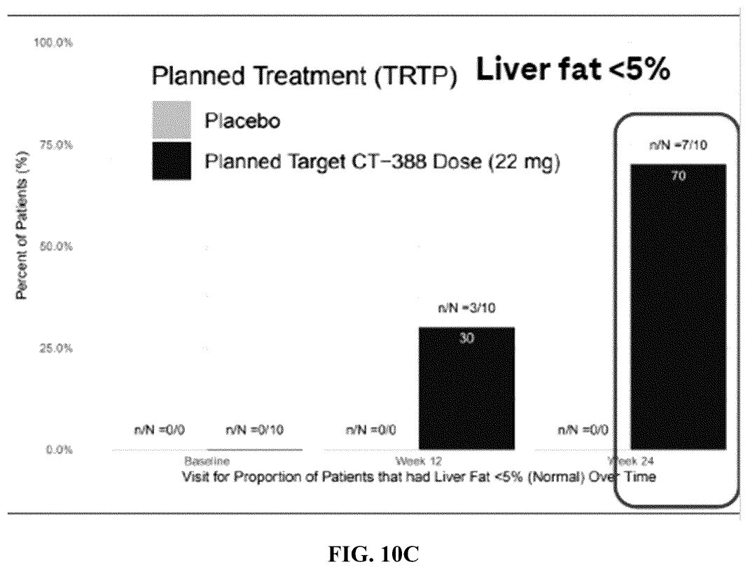

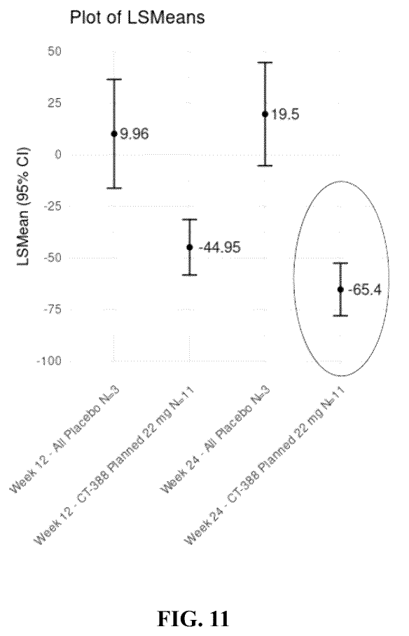

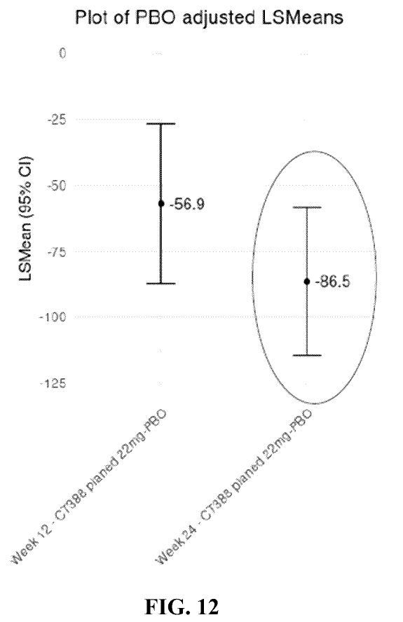

The effect of CT-388 on liver MRI-PDFF in participants with a baseline MRI-PDFF of ≥10% was also tested. Those on CT-388 (n=10) had a mean baseline MRI-PDFF of 16.5% [SD: 7.19%], while placebo participants (n=3) had a mean baseline MRI-PDFF of 11.9%[SD: 1.40%]. The median MRI-PDFF achieved 3.5% at week 24 compared to 14% at baseline (FIG. 9A). Liver MRI-PDFF (LFC) decreased by 86.5% (95% CI: 114.4%-58.5% placebo-adjusted, percentage change) at week 24 (FIGS. 9B, 11, and 12; Table 9). The largest liver fat content reductions were observed in participants with severe MASLD (LFC≥10%) at baseline. Liver MRI-PDFF (LFC) 30% reduction was noted in 100% (10/10) of participants, and 6 out of 10 participants who had MRI-PDFF data at week 24 reached 70% reductions in liver fat. 70% (7/10) were able to decrease their liver steatosis below <5% (normal) (FIGS. 10A-10C). 0% of placebo patients reached LFC<5%.

TABLE 9
Effect of CT-388 on Liver MRI-PDFF in Participants
with Baseline MRI-PDFF of ≥10%

| | LSmean PCHG CT-388 |
|---|---|
| | 22 mg vs. PBO (%, 95% CI) |
| Week 12** | −56.9 (−87.2, −26.6) |
| Week 24*** | −86.5 (−114.4, −58.5) |

***p < 0.001
**p < 0.01;
* p < 0.05

At baseline, 67.7% pts had MAFLD defined as MRI-PDFF≥5 and 50% pts had MRI-PDFF≥10% suggestive of MASH. CT-388 resulted in normalization of liver fat in the vast majority of participants: MRI-PDFF reached <5% in 85.7% of participants after 24 weeks of treatment as compared to 17% in the placebo cohort.

24 weeks of treatment with CT-388 resulted in a significant reduction in liver steatosis in participants with obesity. A 30% reduction in liver MRI-PDFF was noted in 70.8% of all participants; a 70% reduction was noted in 33.3% of all participations. In addition, 70% of participants decreased their liver steatosis to <5%.

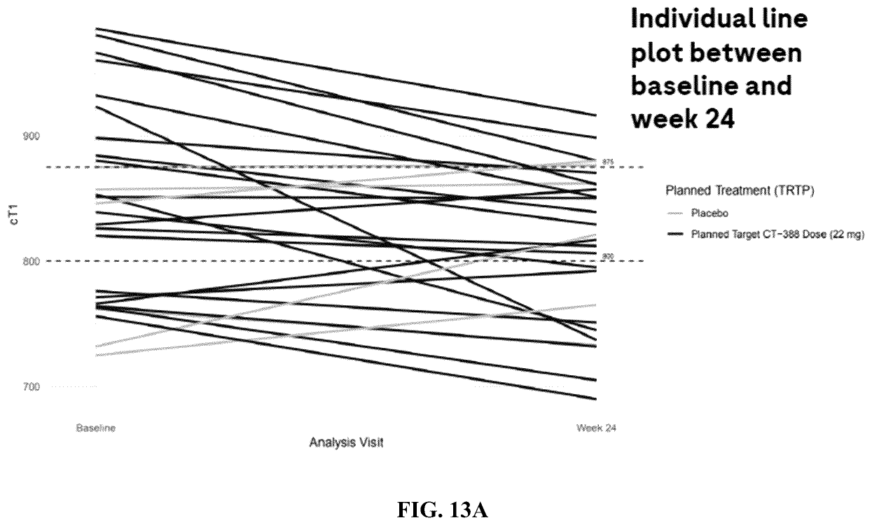

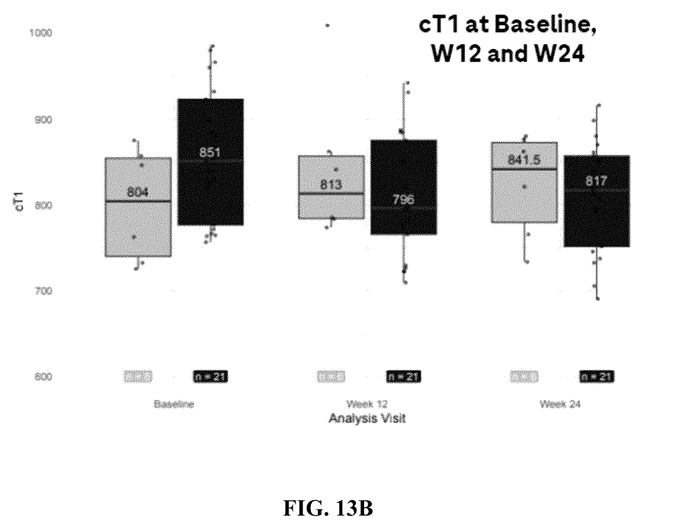

The effect of CT-388 on cT1 was also assessed in the MRI-PDFF analysis population. cT1 was reduced over 24 weeks in participants treated with CT-388 (FIGS. 13A and 13B). A clinical meaningful reduction of cT1 was observed (Table 10). Most of the reduction in cT1 occurred in the first 12 weeks of treatment. Due to the variation of the placebo arm, placebo-adjusted cT1 reduction from baseline is numerically larger at week 12 than week 24, while the unadjusted cT1 reduction was similar cross week 12 and week 24. In CT-388 participants, cT1 decreased by an average of 51.7 ms (95% CI: 8.1-95.2, placebo-adjusted, LS mean) at week 24. There were zero participants in the placebo arm with at least 40 ms decrease.

TABLE 10
Effect of CT-388 on cT1

| | LSmean CHG CT-388 | |
|---|---|---|
| | 22 mg vs. PBO (95% CI) | |
| Week 12 | −73.5** | (−121.3, −25.8) |
| Week 24 | −51.7* | (−95.2, −8.1) |

***p < 0.001
**p < 0.01;
*p < 0.05

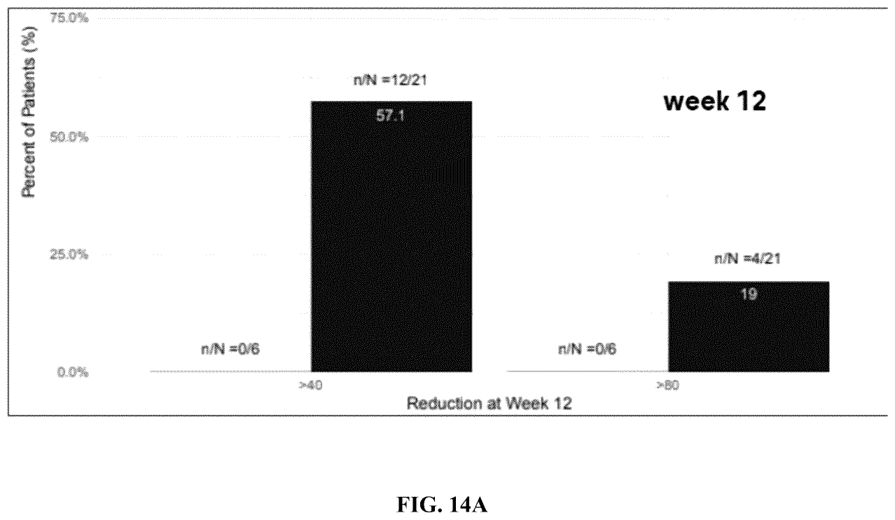

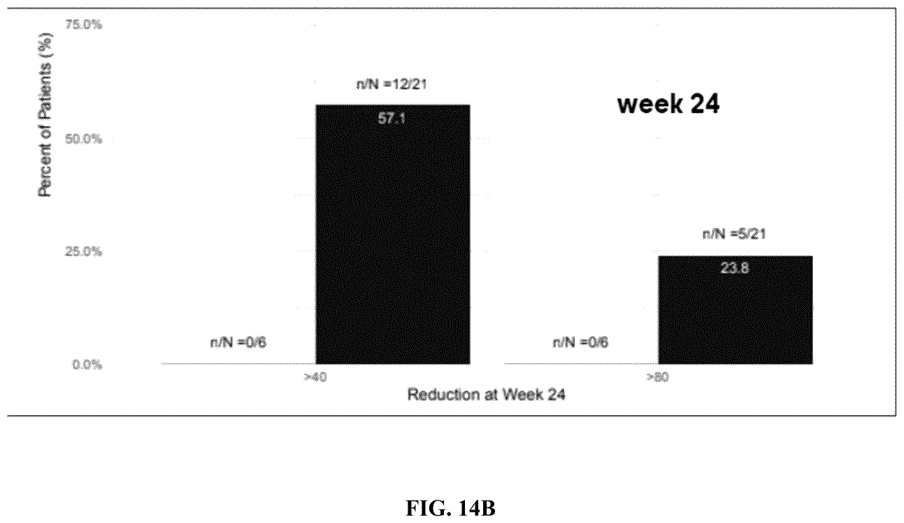

cT1 reduction was noted in 57.1% (12/21) of participants at week 24, and 23.8% (5/21) participants achieved an 80 ms reduction (FIGS. 14A and 14B). Clinically meaningful reductions in cT1 were noted within 24 weeks. Reductions of ≥40 ms in cT1 are clinically meaningful (see, e.g., Beyer et al., J Magn Reson Imaging (2024) 61:1947-55), and reductions of ≥80 ms may predict resolution of steatohepatitis (see, e.g., Alkhouri et al., J Hepatol (2025) 82:438-45).

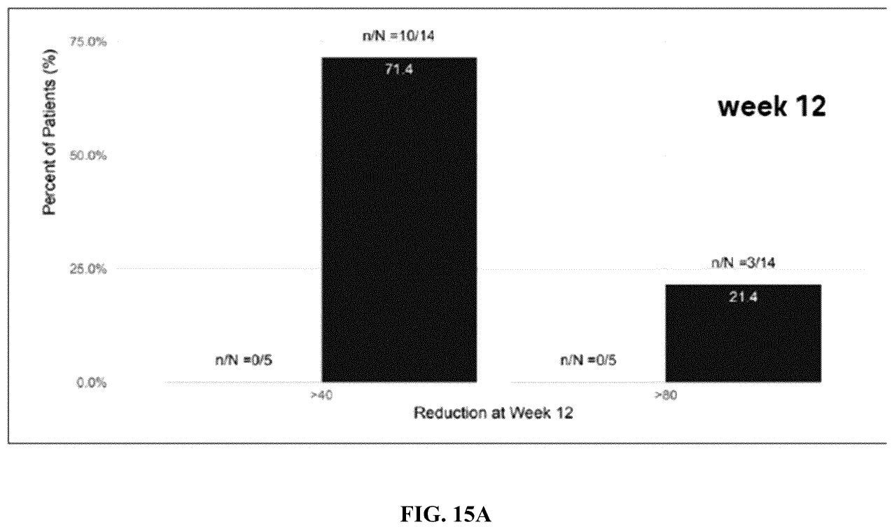

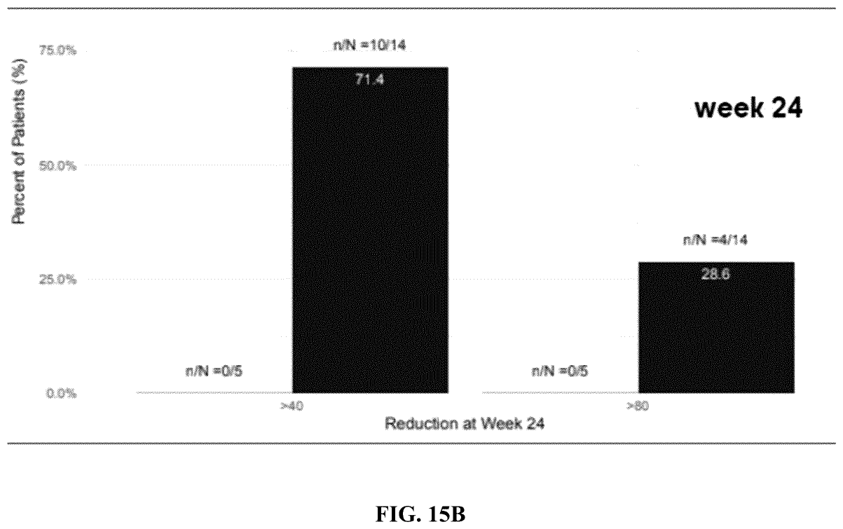

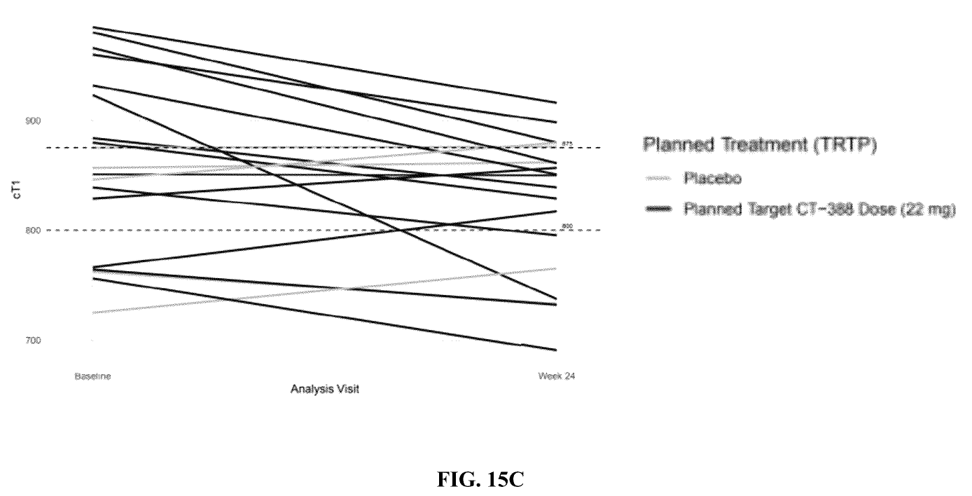

In participants with MRI-PDFF baseline ≥5%, cT1 was decreased by an average of 44 ms (95% CI: −10.7-98.7, placebo-adjusted) at week 24 (Table 11). cT1 reduction by 40 ms was noted in 71.4% (10/14) of participants and by 80 ms in 28.6% (4/14) participants. There were zero participants in placebo arm with at least 40 ms decrease (FIGS. 15A-15C).

TABLE 11
Effect of CT-388 on cT1 in Participants
with MRI-PDFF Baseline ≥5%

| | LSmean CHG CT-388 | |
|---|---|---|
| | 22 mg vs. PBO (95% CI) | |
| Week 12** | −82.7* | (−146.2, −19.2) |
| Week 24* | −44.0 | (−98.7, 10.7) |

***p < 0.001;
**p < 0.01;
*p < 0.05

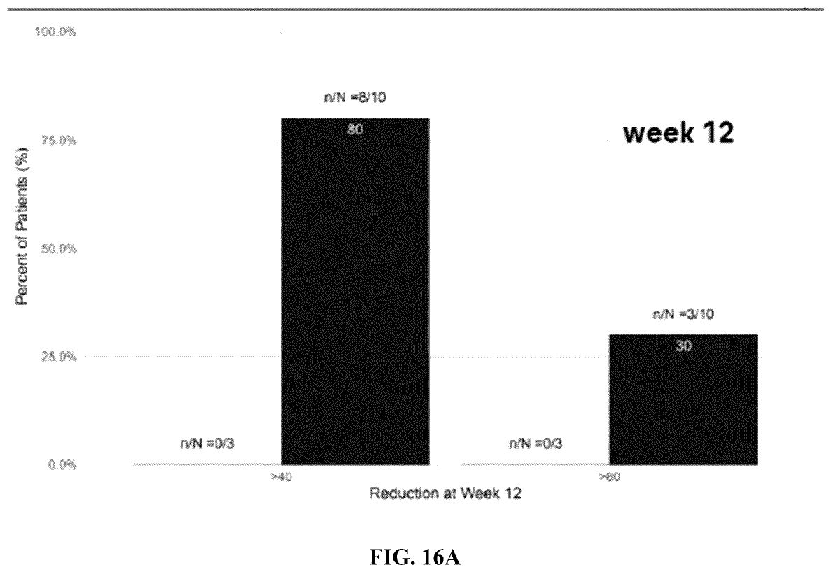

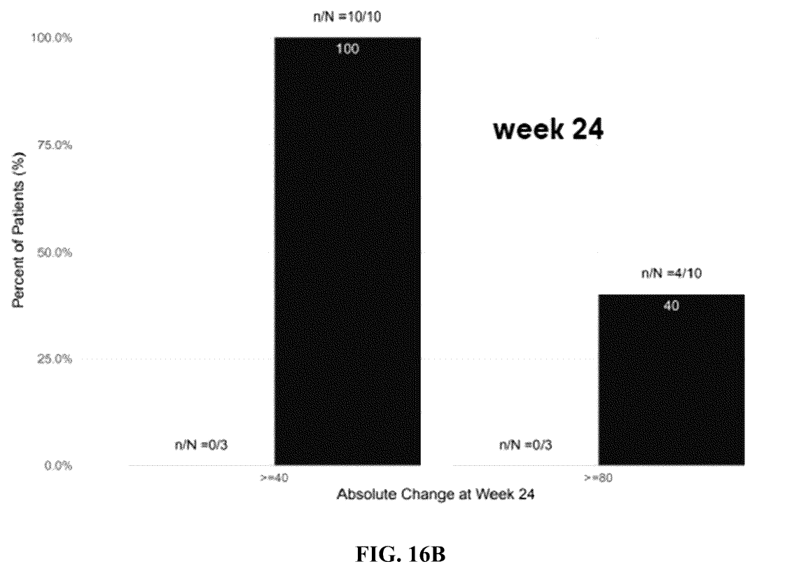

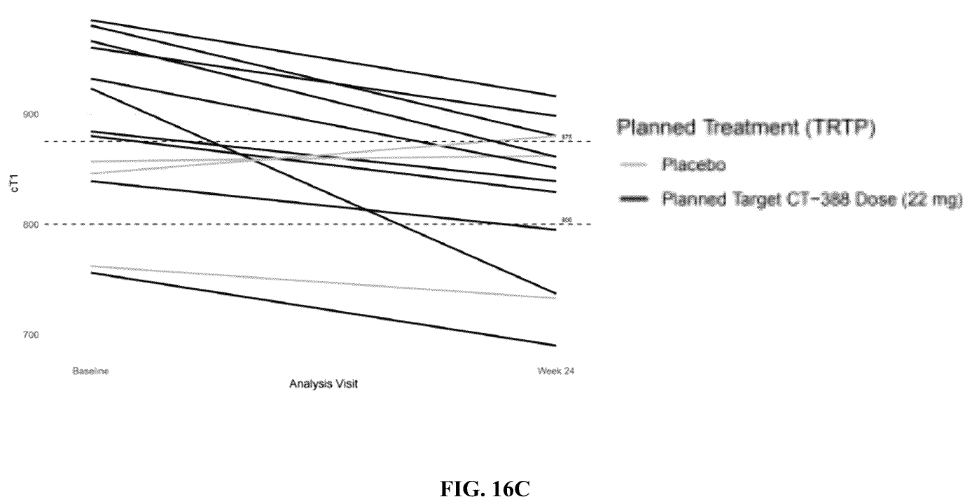

In participants with MRI-PDFF baseline ≥10% (significant liver steatosis), 8/10 CT-388 participants entered the study with combo of MRI-PDFF and cT1≥875 ms, suggestive of fibrosis stage 2 (F2). In CT-388 pts, cT1 decreased by average 73.1 ms (95% CI: 0.2-146, placebo-adjusted) at week 24 (Table 12). A reduction in liver cT1 of 40 units (e.g., by >40 ms) was noted in 100% (10/10) participants at 24 weeks (FIGS. 16A-16C). 40% (4/10) of CT-388 participants achieved an 80 ms reduction, suggestive of improvement in fibrosis by one stage (see, e.g., Dennis et al., Front Endocrinol (2021) 11:575843). No placebo participants met the 40 or 80 ms thresholds at week 12 or 24.

TABLE 12
Effect of CT-388 on cT1 in Participants
with Baseline MRI-PDFF of ≥10%

| | LSmean CHG CT-388 | |
|---|---|---|
| | 22 mg vs. PBO (95% CI) | |
| Week 12** | −122.8 | (−217.4, −28.3) |
| Week 24* | −73.1 | (−146.0, −0.2) |

***p < 0.001
**p < 0.01;
*p < 0.05

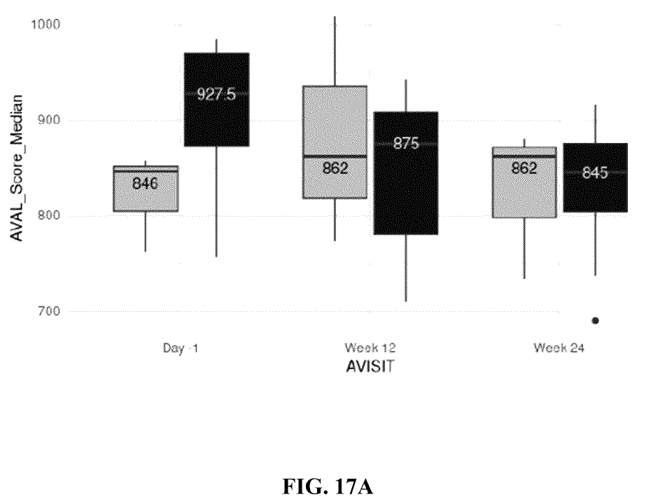

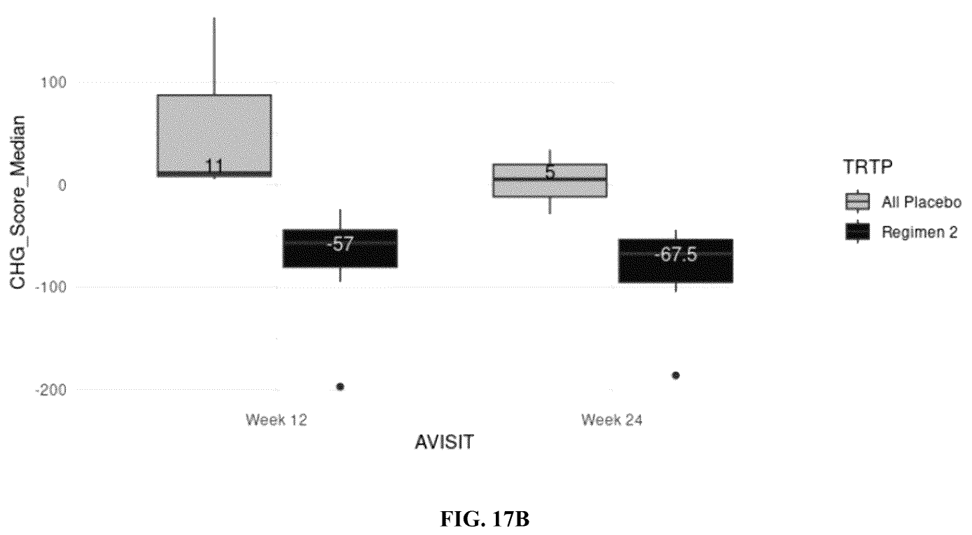

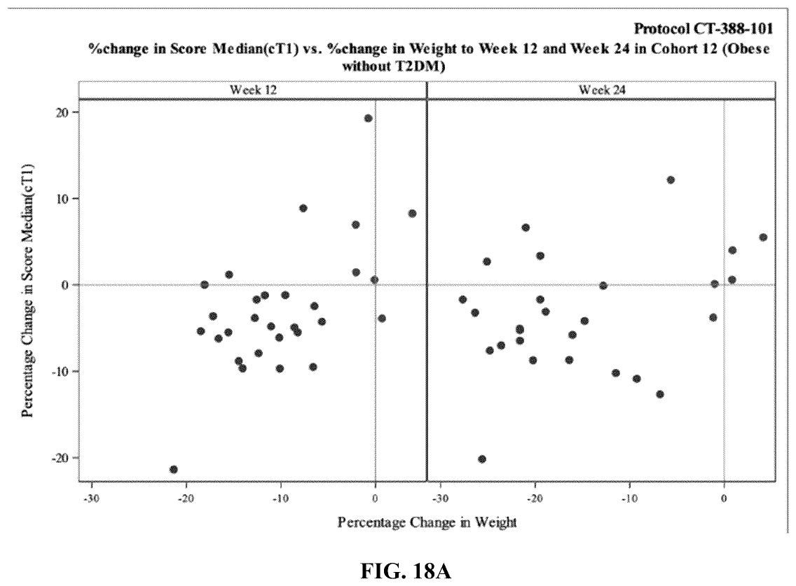

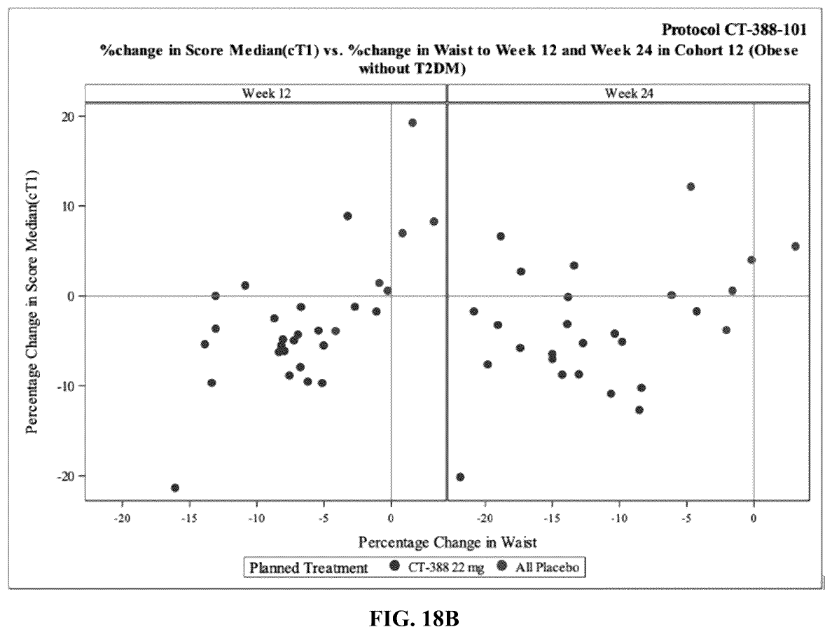

In participants with MRI-PDFF baseline ≥10%, CT-388 administered for 24 weeks resulted in clinically significant and a clinically meaningful reduction of liver fat and cT1, suggesting that the drug might have a beneficial effect in participants with MAFLD and and MASH (FIGS. 17A and 17B). Percent change in cT1 score was also found to correlate with percent change in weight (FIG. 18A) and percent change in waist circumference (FIG. 18B).

### Liver-Related Biomarkers

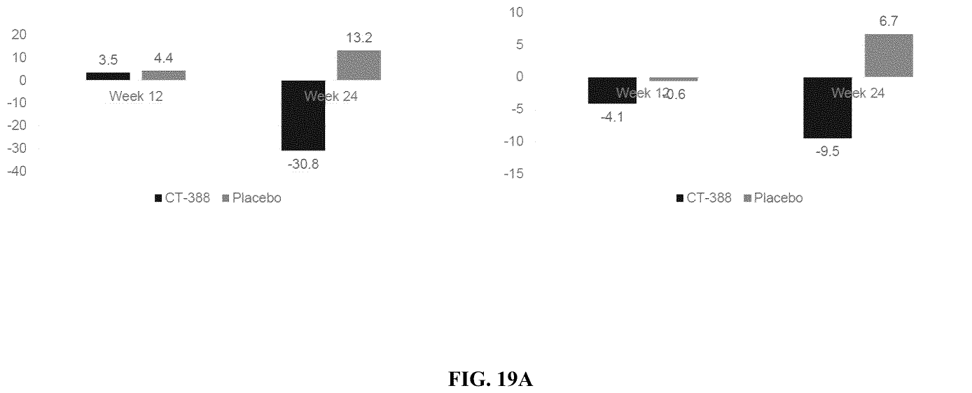

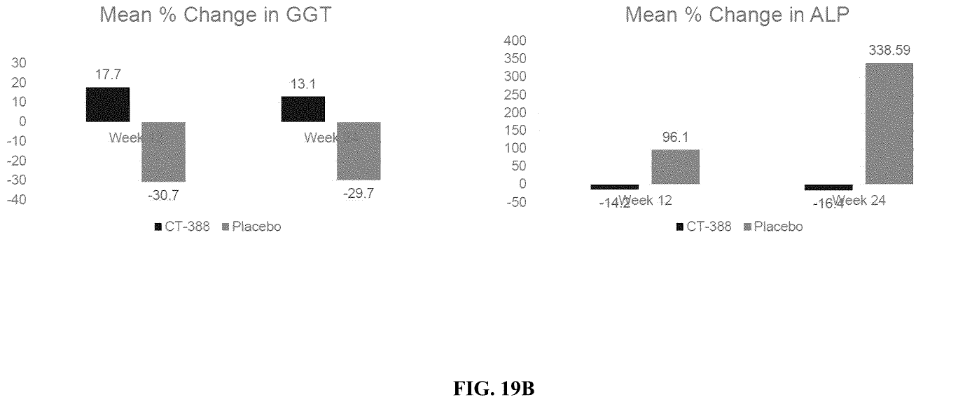

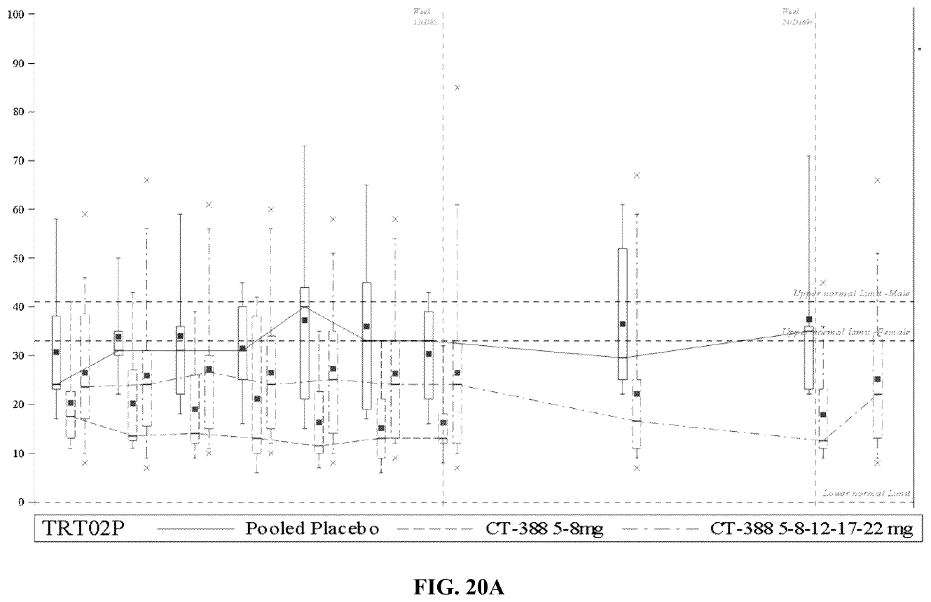

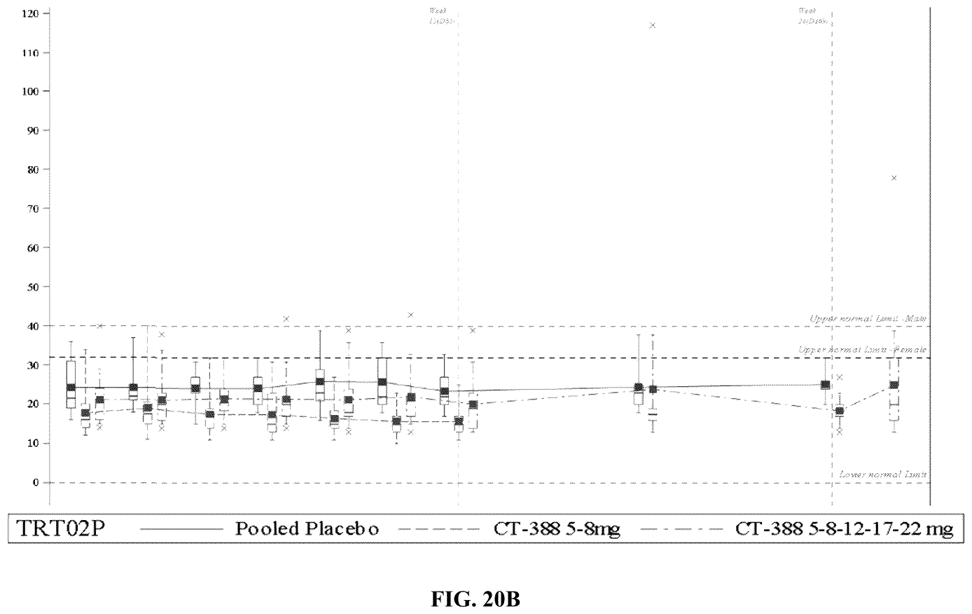

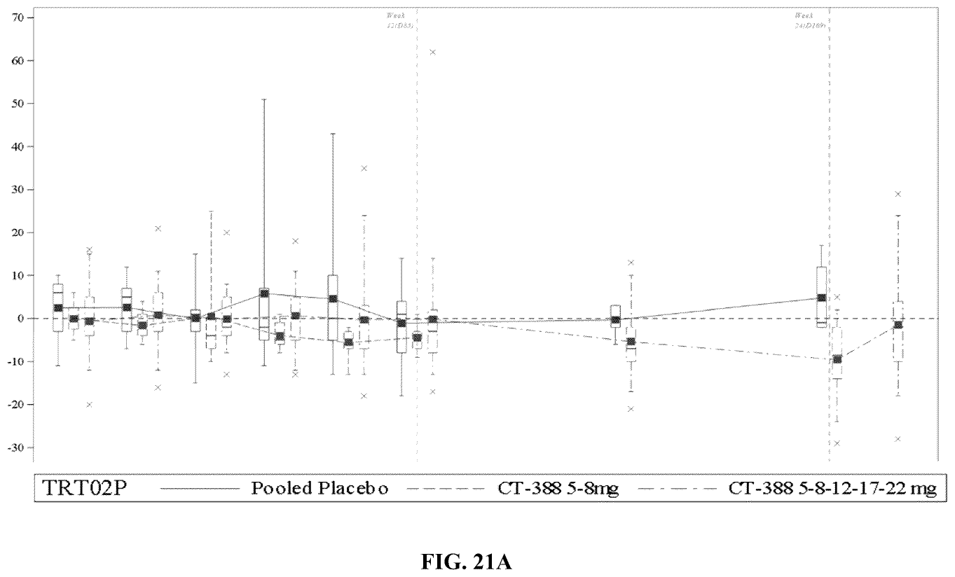

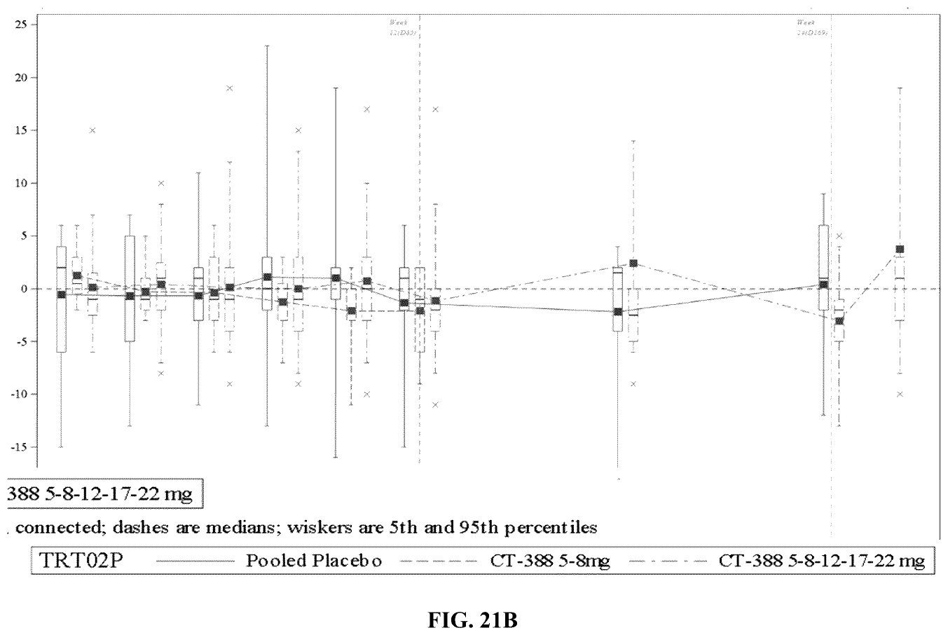

ALT, AST and Fib-4 were within normal range at baseline and during the study. ALT had a tendency to decrease (FIGS. 19A, 20A, and 20B). The median change in ALT at week 12 was −16.7% in participants treated with CT-388 compared to −8.9% in placebo participants. At week 24, the median change in ALT was −39.0% in CT-388 participants compared to −4.2% in placebo participants (Table 13). The median change in AST at week 12 was −8.0% in CT-388 participants, and −5% in placebo participants. At week 24, the median change in AST was −10.0% in CT-388 participants, compared to 5.3% in placebo participants (FIGS. 19B, 21A, and 21B, Table 14). The median change in Fib-4 at week 12 was −1.85% in CT-388 participants, compared to −0.135% in placebo participants. At week 24, median change in Fib-4 was 15.0% in CT-388 participants, as compared to −9.2% in placebo participants (Table 15).

TABLE 13
ALT: Observed Values

| | Baseline | Week 12 | Week 24 |
|---|---|---|---|
| CT-388 | N = 21 | N = 21 | N = 19 |
| Mean (SD) | 27.0 (13.00) | 27.4 (18.71) | 16.8 (9.82) |
| Placebo | N = 6 | N = 6 | N = 5 |
| Mean (SD) | 36.8 (15.91) | 32.8 (10.23) | 37.4 (19.88)) |

TABLE 14
AST: Observed Values

| | Baseline | Week 12 | Week 24 |
|---|---|---|---|
| CT-388 | N = 21 | N = 21 | N = 19 |
| Mean (SD) | 21.4 (6.03) | 20.5 (6.80) | 18.3 (3.06) |
| Placebo | N = 6 | N = 6 | N = 5 |
| Mean (SD) | 26.7 (7.63) | 23.2 (4.54) | 25.2 (4.38) |

TABLE 15
Fib-4 Observed Values

| | Baseline | Week 12 | Week 24 |
|---|---|---|---|
| CT-388 | N = 21 | N = 21 | N = 19 |
| Mean (SD) | 0.46 (0.173) | 0.45 (0.172) | 0.52 (0.243) |
| Placebo | N = 6 | N = 6 | N = 5 |
| Mean (SD) | 0.68 (0.586) | 0.53 (0.228) | 0.61 (0.270) |

GGT and ALP values were also measured. Median change in observed GGT values at week 12 was −35.0% in CT-388 participants compared to −16.7% in placebo participants; at week 24, the median change in observed GGT values was −34.5% in CT-388 participants compared to 0% in placebo participants (Table 16). Median change in observed ALP at week 12 was −14.3% in CT-388 participants compared to 1.2% in placebo participants. At week 24, median change in observed ALP was −16.4% in CT-388 participants compared to −3.8% in placebo participants (Table 17).

TABLE 16
GGT Observed Values

| | Baseline | Week 12 | Week 24 |
|---|---|---|---|
| CT-388 | N = 24 | N = 23 | N = 20 |
| Mean (SD) | 28.8 (16.91) | 21.7 (18.15) | 23.2 (22.0) |
| Placebo | N = 10 | N = 9 | N = 5 |
| Mean (SD) | 28.7 (9.72) | 31.6 (16.02) | 32.0 (5.87) |

TABLE 17
ALP Observed Values

| | Baseline | Week 12 | Week 24 |
|---|---|---|---|
| CT-388 | N = 24 | N = 23 | N = 20 |
| Mean (SD) | 82.3 (14.94) | 70.6 (14.76) | 71.6 (13.1) |
| Placebo | N = 10 | N = 9 | N = 5 |
| Mean (SD) | 82.2 (31.21) | 88.1 (14.5) | 92.8 (21.5) |

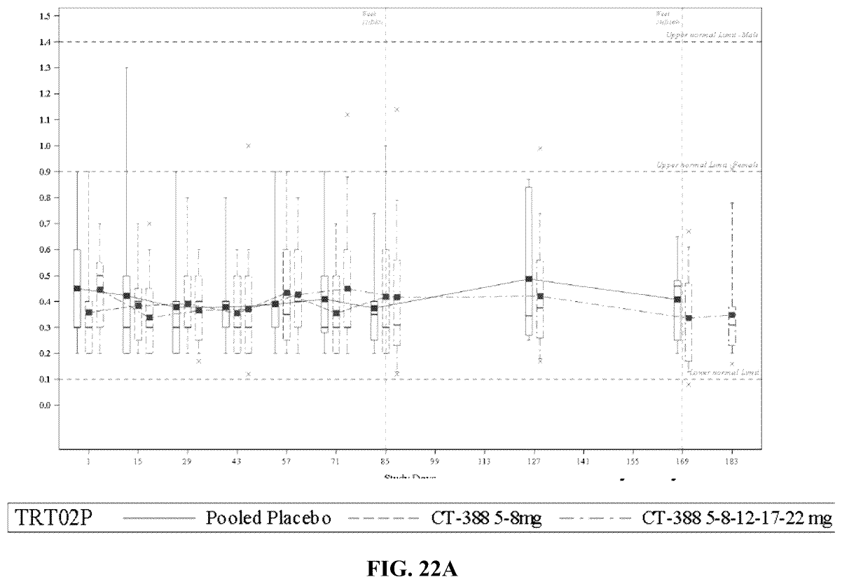

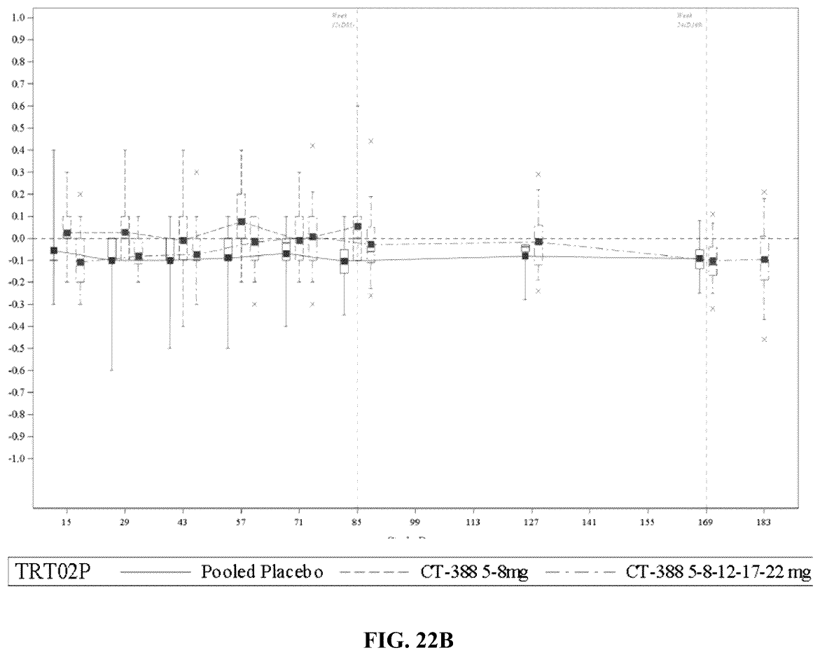

Total bilirubin over 24 weeks was also measured (FIGS. 22A and 22B). Total bilirubin was WNL at baseline and was relatively stable during the study. Adiponectin, beta-hydroxybutyrate, and FIB-4 were also measured (Table 18).

TABLE 18
Adiponectin, Beta-Hydroxybutyrate, and FIB-4

| | | | |
|---|---|---|---|
| | CT-388 8 mg | CT-388 22 mg | Placebo |
| Parameter Mean (SD) | N = 12/0 | N = 24/20 | N = 9/5 |

Adiponectin (μg/mL)

| Baseline | 4.4 | 3.4 | 3.4 | |
|---|---|---|---|---|
| % CFB at W 12 | 40.5 (41.6) | 28.4 (26.4) | 13.9 | (34.7) |
| % CFB at W 24 | NA | 15.4 (34.5) | 23.2 | (25.3) |

Beta-Hydroxybutyrate (μmol/L)

| | | | | |
|---|---|---|---|---|
| Baseline | 4.4 (2.4) | 3.1 (2.2) | 4.3 | (2.0) |
| % CFB at W 12 | −37.9 (36.0) | 92.8 | 23.1 | (78.9) |
| % CFB at W 24 | NA | 88.2 (59.9) | 121.5 | (146.4) |

FIB-4

| | | | | |
|---|---|---|---|---|
| Baseline | 13.5 (13.9) | 11.8 (5.7) | 20.6 | (12.3) |
| | 0.6 (0.35) | 0.5 (0.20) | 0.7 | (0.54) |
| % CFB at W 12 | −16.0 (28.8) | 0.02 (48.4) | −1.5 | (30.3) |
| % CFB at W 24 | | | 9.6 | (33) |

### SUMMARY

Obese population without T2D have significant amount of liver steatosis at baseline. After 24 weeks of treatment, CT-388 resulted in significant decrease in liver fat, with all participants with liver steatosis at baseline reduced MRI-PDFF by at least 30%. Decrease in cT1 is suggestive of potential beneficial effect of histological response in participants with obesity. In participants with significant liver steatosis, effect of CT-388 on liver fat and cT1 was more prominent and consistent with incretins tested in MASH-focused studies. These data support the beneficial effect of CT-388 on liver steatosis and grants further evaluation of the effect of CT-388 on MAFLD/MASH.

Obese participants enrolled into the CT-388 trial had a significant amount of liver steatosis. 70% of participants (19/27) had an MRI-PDFF of ≥5%, suggestive of MAFLD, and 48% of pts (13/27) had MRI-PDFF of ≥10%, suggestive of MASH. After 24 weeks of treatment, CT-388 resulted in significant decrease in liver fat. MRI-PDFF decreased by 58.5% (95% CI: 31.1%-85.9%) adjusting for placebo, and liver fat decreased by ≥30% in 85.7% pts (18/21). Liver fat decreased to <5% (normal) in 85.7% pts (18/21), compared to 33.3% (7/21) of participants at baseline.

In participants with baseline MRI-PDFF≥5% and MRI-PDFF≥10%, MRI-PDFF decreased by 78.1% (95% CI: 53.8%-102.5%) and 86.5% (95% CI 58.5%-114.4%) respectively. A decrease in liver fat by ≥30% was noted in 14/14 (100%) of participants and 10/10 (100%) of participants, respectively. Reduction in liver fat tend to associate with weight loss and decrease in waist circumference. cT1 also decreased at 24 weeks. cT1 was decreased by 51.7 ms (95% CI: 8.1-95.2) in overall trial population. In participants with baseline MRI-PDFF≥5%, cT1 decreased by 44.0 ms (95% CI: −10.7-98.7), and in pts with baseline MRI-PDFF≥10% cT1 decreased by 73.1 ms (95% CI: 0.2-146.0).

ALT, AST and Fib-4 were within normal range at baseline and during the study. ALT levels tended to decrease during the study. CT-388 was generally safe and tolerable, and no liver-related adverse events were observed. Treatment with CT-388 potentially results in clinically meaningful improvements in liver steatosis and inflammation in participants with obesity, with a mean liver steatosis decrease of 59% overall and 87% in participants with significant liver steatosis. These data support the beneficial effect of CT-388 on liver-related parameters.

### Example 3: Additional Non-Invasive Biomarkers to Provide Exploratory Data on the Impact of CT-388 on Liver Fibrosis

Several other non-invasive tests and biomarkers are contemplated for measuring the efficacy of CT-388 in treating MASH (Table 19). Histological changes to measure liver fibrosis may also be used to measure MASH in participants. Several non-invasive MASH biomarkers are used to diagnose severity of steatosis, MASH activity, and fibrosis. For some fibrosis biomarkers, changes are associated with clinical outcomes in MASH drugs. Measuring consistent changes for several orthogonal biomarkers may be an approach to increase confidence in biomarker findings. Participants with mild MASH may not have significant fibrosis, and therefore may have a lower likelihood to observe changes. For assessments of changes in fibrosis, 24 weeks may be an early time point.

TABLE 19
Non-Invasive Tests and Biomarkers for MASH Traits

| | |
|---|---|
| MASH Trait | Non-Invasive Test or Biomarker |
| Fibrosis | FibroScan (including FAST, Agile3+, Agile4) |
| | MR Elastography, MRI cT1 |
| | NASH FibroSure Plus (BioPredictive) |
| | ELF (FDA approved prognostic biomarker, HA + |
| | PIIINP + TIMP-1) |
| | Nordic panels (including PRO-C3, ADAPT, |
| | fibrolysis markers) |
| | FIB-4 (age, ALT, AST, platelet count) |
| | SomaLogic NASH |
| | NIS2+, NIS4 |
| Inflammation & | Transaminases |
| Hepatocellular Injury | CK-18 |
| (MASH activity) | NASH2 |
| | SomaLogic NASH |
| | NASH FibroSure Plus |
| | NIS2+, NIS4 |
| | MRI cT1 |
| Steatosis | SteatoTest |
| | MRI-PDFF |
| | FibroScan |
| | SomaLogic NASH |
| | NASH FibroSure Plus |
| Liver Function | HepQuant DuO |
| (overlaps all traits) | |

Biomarkers that are associated with fibrosis—including enhanced liver fibrosis (ELF) and Pro-C3—are measured at baseline, week 12, and week 24. For ELF, treatment is expected to show results at the 24 week time point, and testing requires 1 mL of blood from participants. For Pro-C3, changes are observed at the week 16 or later time point.

Participants are tested using the FIB-NIT panel combination of Pro-C3/ProC6/CTX-III. CTX-III is a biomarker of fibrolysis, which is necessary for resolution of fibrosis. The Pro-C3/CTX-III ratio tells about balance of collagen deposition and fibrolysis, and could differentiate progressive vs regressive fibrosis.

Pro-C6 is a marker of fibroblast activity and prognostic for adverse outcomes in HTFpEF.

Additional exploratory data is collected using the SomaLogic MASH panel, which gives outputs of proteomic based assessments for fibrosis, steatosis, inflammation and ballooning.

### Example 4: Effect of CT-388 on Liver Fat in Adults with Obesity, With or Without Type 2 Diabetes

This Example describes a 12-week phase 1b trial in participants (pts) with obesity (BMI≥30 kg/m²) with or without type 2 diabetes (T2D). Patients (n=50) were randomized 4:1 to receive 22 mg of CT-388 or placebo. MRI-proton density fat fraction was used to evaluate liver fat content (LFC). Patients with baseline (BL) and week 12 MRI data were included in the analysis (n=42; 29 without T2D, 13 with T2D).

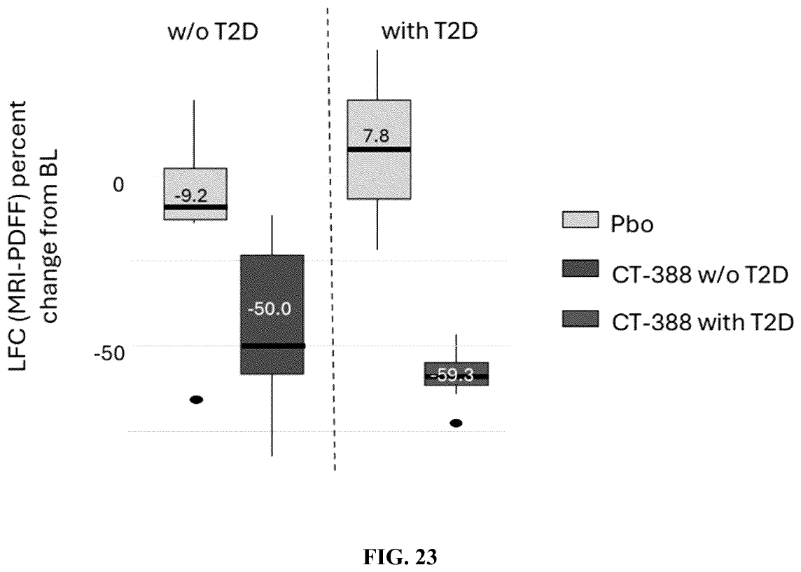

The baseline characteristics for the MRI population are summarized in Table 20 below. Overall, 71% (30/42) of patients had baseline LFC≥5% and 45% (19/42) had baseline LFC≥10%. In patients with baseline LFC≥5%, CT-388 treatment was associated with reductions in LFC of 50% in patients without T2D and reductions in LFC of 59% in patients with T2D at Week 12 as compared to baseline (FIG. 23). Overall, 78% (18/23) of patients on CT-388 achieved LFC reduction ≥30% (67% of patients without T2D and 100% of patients with T2D; compared to 14% of pooled placebo patients), and 53% (12/23) reached normal (<5%) LFC levels (47% of patients without T2D and 63% of patients with T2D; as compared to 14% of pooled placebo patients). In patients with significant liver steatosis (baseline LFC≥10%), similar results were observed with CT-388 (LFC reductions of 46% in patients without T2D and 54% in patients with T2D; 73% [11/15] of patients had an LFC reduction of >30% and 27% [4/15] reached normal LFC levels). No liver-related adverse events were reported with CT-388 treatment. This trial resulted in clinically meaningful reductions in body weight of ˜9% (patients with T2D) to ˜12% (patients without T2D); in patients receiving 22 mg of CT-388, those with T2D had a placebo-adjusted least square mean change from baseline of −7.4% [95% CI: −9.8, −4.9] and −11.5% [95% CI: −15.1, −7.9%] for CT-388 22 mg in pts without T2D (Steinberg et al., ADA 85th Scientific Sessions (2025) Poster 763-P and Chakravarthy et al. European Association for the Study ofDiabetes Annual Meeting (2024) Presentation LBA 65).

TABLE 20
Baseline Characteristics and Change in Liver Fat
Content (LFC) in Participants with Obesity (With
or Without T2D) With LFC ≥5% at Baseline

| Characteristic, | Without T2D | | With T2D | |
|---|---|---|---|---|
| Mean (SD) | Pbo | CT-388 | Pbo | CT-388 |
| Unless Stated | (n = 6) | (n = 23) | (n = 3) | (n = 10) |
| Age (yrs) | 32 (12) | 32 (9) | 42 (7) | 47 (10) |
| Sex, female, n | 0 (0) | 15 (65) | 3 (100) | 5 (50) |
| (%) | | | | |
| BMI (kg/m²) | 35 (3) | 39 (6) | 36 (4) | 33 (3) |
| LFC (%), Median | 10 (5) | 9 (10) | 8 (5) | 9 (6) |
| (IQR) | | | | |
| LRC ≥5%, | 11 (4) | 11 (9) | 12 (4) | 10 (5) |
| Median (IQR) | n = 5 | n = 15 | n = 2 | n = 8 |

BL, baseline; IQR, interquartile range; LFC, liver fat content; MRI-PDFF, MRI-proton density fat fraction; Type 2 diabetes, T2D.

In conclusion, CT-388 treatment was associated with clinically meaningful reductions in LFC in patients with obesity, with or without T2D. After 12 weeks of CT-388 treatment, patients with LFC≥5% at BL—which is indicative of metabolic dysfunction-associated steatotic liver disease (MASLD)—experienced median LFC reductions of 50-60%, and most of these patients achieved target LFC reductions of ≥30%. Together with previously reported weight loss and glycemic control improvements for CT-388 treatment, these data suggest enhancements in metabolic health.

### Example 5: Evaluation of CT-388 on MRI Biomarkers MRI-PDFF and cT1 in Participants with MASLD or MASH

This Example describes the effects of CT-388 on liver-related MRI biomarkers MRI-PDFF and cT1 in MASLD and MASH patients. MRI-PDFF was used to accurately quantify liver fat content (LFC) within liver tissue (see, e.g., Tang et al., Radiology. (2015) 274:416-25). An LFC<5% is considered healthy, while LFC≥5% is indicative of MASLD. Severe liver steatosis is characterized by LFC≥10% (see, e.g., Andersson et al., Clin Gastroenterol Hepatol. (2022) 20:2451-61). A ≥30% reduction in LFC leads to a ≥4 times higher likelihood to resolve steatohepatitis (see, e.g., Alkhouri et al., J Hepatol. (2025) 82:438-45). A liver MRI-PDFF image from a patient with MASLD may show elevated LFC in multiple regions.

The MRI biomarker cT1 was used to evaluate liver fibrosis and inflammation (see, e.g., Alkhouri et al., J Hepatol. (2025) 82:438-45 and Andersson et al., Clin Gastroenterol Hepatol. (2022) 20:2451-61). A healthy cT1 reference range is approximately 600-800 ms. A cT1≥800 ms is predictive of MASH, and a cT1 875 ms indicates advanced MASH (see, e.g., Andersson et al., Clin Gastroenterol Hepatol. (2022) 20:2451-61 and Mojtahed et al., Abdom Radiol (NY) (2019) 44:72-84). A reduction in cT1 of ≥40 ms is considered the minimum clinically meaningful change (see, e.g., Beyer et al., J Magn Reson Imaging. (2025) 61:1947-55). A cT1 reduction of ≥80 ms has been associated with a greater than or equal to five times higher likelihood to resolve steatohepatitis (see, e.g., Alkhouri et al., J Hepatol. (2025) 82:438-45). A liver cT1 image in a patient with MASLD may show elevated cT1 values in multiple regions.

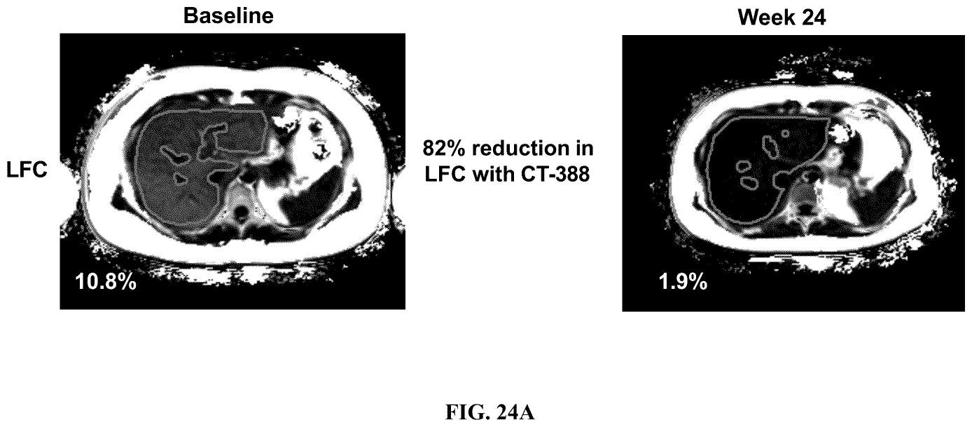

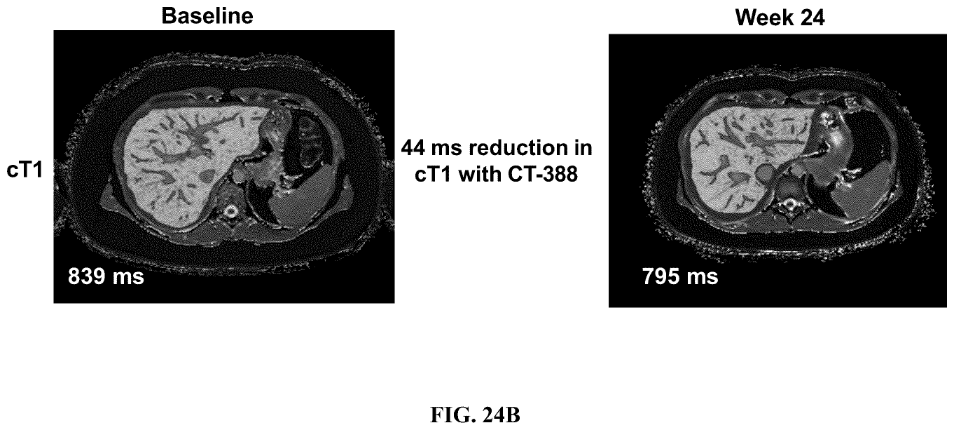

FIGS. 24A and 24B illustrate the impact of CT-388 on a representative 24-year-old female participant with a baseline BMI of 33.2 kg/m², liver MRI-PDFF of 10.8%, and T1 of 839 ms. The participant was treated with up-titration of CT-388 over 24 weeks. At end of treatment, LFC in the patient had decreased to 1.9%, corresponding to an 82% reduction from baseline, and cT1 had decreased to 795 ms (a 44 ms reduction). The participant lost 21% of her body weight (82.3 kg at baseline compared to 64.5 kg at end of treatment), a decrease in ALT from 19 U/L to 8 U/L (both within normal limits), and a decrease in Pro-C3 from 52.6 ng/mL to 32.6 ng/mL. At baseline, the MRI-PDFF image showed elevated LFC in multiple regions as indicated by the circles (FIG. 24A, left image). A second MRI-PDFF following treatment showed resolved steatohepatitis and a reduction in LFC as indicated by the circled region (FIG. 24A, right image). At baseline, liver cT1 images at showed elevated cT1 values in multiple regions (FIG. 24B, left image). Resolved steatohepatitis and a reduction in cT1 values were observed following treatment (FIG. 24B, right image).

### Example 6: Evaluation of CT-388 on Blood Biomarkers Related to Liver Health in Participants with Obesity, With or Without MASLD

This Example describes changes in blood biomarkers following 24 weeks of treatment with CT-388 in participants with obesity, with or without MASLD (Table 21). Following treatment, blood biomarkers related to liver health trend towards improvement and are in line with results of the imaging biomarkers as described in Example 5. Biomarker analyses included serum alanine aminotransferase (ALT) and N-terminal type III collagen propeptide (Pro-C3).

TABLE 21
Changes in Blood Biomarkers Following 24 Weeks of Treatment with
CT-388 in Participants with Obesity, With or Without MASLD

| | | Patients with | |
|---|---|---|---|
| Patients with Obesity | | Obesity and MASLD | |
| Placebo | CT-388 22 mg | Placebo | CT-388 22 mg |
| (n = 6) | (n = 21) | (n = 5) | (n = 14) |

ALT

| | | | | |
|---|---|---|---|---|
| Baseline (U/L), | 37 (16) | 27 (13) | 39 (16) | 31 (14) |
| mean (SD) | | | | |
| Week 24 (U/L), | 42 (21) | 17 (10) | 46 (21) | 19 (11) |
| mean (SD) | | | | |
| Mean change from | +5 | −10 | +6 | −12 |
| baseline, U/L | | | | |

Pro-C3^{a}

| | | | | |
|---|---|---|---|---|
| Baseline (ng/mL), | 52 (24) | 51 (14) | 53 (26) | 53 (14) |
| mean (SD) | | | | |
| Week 24 (ng/mL), | 48 (14) | 32 (8) | 49 (16) | 34 (8) |
| mean (SD) | | | | |

^{a}A Pro-C3 value over 20 ng/mL is predictive of fibrosis progression and a worse metabolic state (Nielsen M J et al., J Hepatol. (2021) 75: 1292-300).
ALT = alanine aminotransferase, AST = aspartate aminotransferase, ELFS = enhanced liver fibrosis score, FIB-4 = fibrosis-4, MASLD = metabolic dysfunction-associated steatotic liver disease, Pro-C3 = fibrogenesis serological marker; eflects formation of N-terminal type III collagen propeptide, and SD = standard deviation.

### Example 7: Effects of CT-388, a Once-Weekly Signaling-Biased Dual GLP-1/GIP Receptor Agonist, on Weight Loss and Glycemic Control in Preclinical Models and Participants with Obesity

This Examples describes the pharmacological activity and metabolic effects of CT-388 in preclinical in vitro and in vivo models and evaluates the safety, tolerability, pharmacokinetics (PK), and initial efficacy of CT-388 in a first-in-human phase 1 study. These assessments demonstrate robust body weight-lowering effects with improvements in glucose homeostasis after CT-388 treatment, translating across various mouse models of obesity, cynomolgus monkeys, and human volunteers with overweight or obesity without type 2 diabetes (T2D). These data support the use of CT-388 as a once-weekly therapy in humans.

### A. Methods

CT-388 was formulated at 20 mg/mL in 1.38 mg/mL sodium monophosphate monobasic monohydrate, 25 mg/mL trehalose dihydrate, 40 mg/mL mannitol, 1.0 mg/mL 10% polysorbate 20 solution, and adjusted to pH 6.5 at 25° C. using 1N sodium hydroxide. CT-388 was supplied as a sterile, lyophilized powder in glass vials and reconstituted in sterile water before injection. Placebo was supplied as a sterile solution in glass vials (1.38 mg/mL sodium monophosphate monobasic monohydrate, 25 mg/mL trehalose dihydrate, 40 mg/mL mannitol, 1.0 mg/mL 10% polysorbate 20 solution, and adjusted to pH 6.5 at 25° C. using 1N sodium hydroxide). Each dose was administered as a subcutaneous injection in the abdomen. Doses were preceded by an overnight fast from food (≥10 hours).

### In Vitro Characterization

cAMP production was measured using the HitHunter® cAMP Assay for Small Molecules kit in mammalian cell lines overexpressing either GLP-1R or GIPR. CHO-K1 cells stably expressing SNAP-human glucagon-like peptide-1 receptor (GLP-1R) or SNAP-human glucose-dependent insulinotropic polypeptide receptor (GIPR) were made in-house and used for human receptor cyclic adenosine monophosphate (cAMP) assays. Cells were detached with cell dissociation media, counted, spun down, and resuspended at a 1:2 ratio of anti-cAMP antibody: 1×HBSS/10 mM HEPES/625 μM IBMX. A total of 10,000 cells in 5 μL of media were added to each well of a 384-well, low-volume assay plate. For mouse receptor cAMP assays, either U2OS cells stably expressing mouse GLP-1R (DiscoverX 95-0179C3) or CHO-K1 cells stably expressing mouse GIPR (DiscoverX 95-0154C2) were used. Twenty-four hours before the assay, 5000-10,000 cells per well were plated in 384-well, low-volume tissue culture-treated plates in Assay Complete Cell Plating reagent 2 (murine GIPR cells) or 5 (murine GLP-1R cells). Before starting the assay, the media was replaced with 5 μL of a 1:2 ratio of anti-cAMP antibody: 1×HBSS/10 mM HEPES/625 M IBMX. Compounds were diluted 1:1 in DMSO and 5 nL was transferred to the tissue-culture plate wells using an ECHO Acoustic Liquid Handler (Beckman Coulter). After 30 minutes of incubation, cAMP detection reagents were added according to the manufacturer's specifications; after the suggested incubation times, luminescence was measured using an EnVision multimode plate reader (PerkinElmer; Waltham, MA).

β-arrestin recruitment was measured using Promega's NanoBiT technology and Nano-Glo® Live Cell Substrate in HEK293 cells. β-arrestin-2 recruitment was measured using Promega's NanoBiT technology and Nano-Glo® Live Cell Substrate (catalog number N2012), which uses the NanoLuc luciferase split into two subunits called LgBiT and SmBiT. HEK293 cells were either stably or transiently transfected with equal amounts of DNA plasmids expressing the fusion proteins human GLP-1R-LgBiT or GIPR-LgBiT and SmBiT-(human)-β-arrestin-2; similar methods were used for mouse GLP-1R, GIPR, and β-arrestin-2. Cells were lifted using TrypLE express enzyme (ThermoFisher Scientific) 24 hours before the assay and plated at 10,000 cells per well in tissue culture-treated 384-well low-volume plates. The following day, the media was removed and replaced with 10 μL of a 1:100 dilution of Nano-Glo® Live Cell Substrate with NanoBiT buffer and OptiMem and equilibrated to room temperature for ten minutes covered. Background luminescence was measured before adding 100 nL of compound dilutions (made in a 1:1 ratio in DMSO). Compounds were transferred using an ECHO Acoustic Liquid Handler (Beckman Coulter) and luminescence was measured at 1.5-minute intervals for 15 minutes using an EnVision Multimode Plate Reader (PerkinElmer; Waltham, MA).

Receptor internalization for human GLP-1R and GIPR was measured with Promega's Nano-Glo® HiBit extracellular detection system in HEK293 cells. Receptor internalization for human GLP-1R and GIPR was measured using the Promega Nano-Glo© HiBit extracellular detection system (Promega Corporation). HEK293 cells were transiently transfected with 1000:1 ratio of carrier plasmid (pcDNA3.1) and plasmids containing HiBiT-tagged human GLP-1R or human GIPR sequences (Promega Corporation). Cells were dissociated the following day using TrypLE express enzyme (ThermoFisher Scientific) and plated in 96-well plates at 80,000-100,000 cells per well. Forty-eight hours post-transfection, media was replaced with equal parts CO2 independent media (Gibco) and Nano-Glo extracellular buffer containing LgBiT protein (diluted 1:100), and Nano-Glo HiBiT extracellular substrate (diluted 1:50). After a 15-minute equilibration and background luminescence reading, test compounds were added and the luminescence was measured for 120 minutes at two-minute intervals using an EnVision Multimode Plate Reader (PerkinElmer).

Static glucose-stimulated insulin secretion (GSIS) was measured using EndoC-bH5® cells, a primary human beta cell line. EndoC-OH5 cells were seeded onto βCoat®-coated 96-well plates (10⁵ cells/well). Six days later, medium was replaced with Ulti-ST® starvation medium for 24 hours. Medium was then replaced with βKrebs® GSIS buffer supplemented with 0.1% fraction V fatty acid free BSA for 60 minutes. Cells were then incubated with βKrebs® supplemented with 0.1% fraction V fatty acid free BSA and 11 mM glucose with or without CT-388 (1-1000 nM; batch AQ5) for 40 minutes. Incubation medium was collected and insulin levels were quantified using the U-PLEX human insulin assay (Meso Scale Discovery [MSD]; catalog number K1516HK-24) and recorded with an MSD plate reader (MSD; Rockville, Maryland).

Insulin levels were quantified in media supernatants using the Meso Scale Discovery U-PLEX mouse insulin assay.

For cAMP activity and β-arrestin recruitment, data were normalized to either GLP-1 or GIP and vehicle control before non-linear regression fitting. Each dilution series was normalized to the adjacent high (100%) and low (0%) wells on the plate. Dose-response data were fitted to a curve using nonlinear regression analysis using the “log(agonist) vs response-variable slope” setting, where:

Y=Bottom+(Top−Bottom)/(1+10\hat{ }((Log EC₅₀−X)*HillSlope))

and the Hillslope was constrained to 1. Compound potency and efficacy were extracted from this regression analysis. When maximal activity was less than 10%, a curve was not fitted. For NanoBiT assays, a single time point during the 30-minute activity measurements was selected for maximum luminescence response. Then, each well at this time point was normalized to the background signal and to the low (vehicle) and high (1 M GLP-1/GIP) controls in every row. For HiBiT assays, each well was first normalized to the background signal and then to the vehicle control at each time point.

### In Vivo Pharmacodynamic Studies in Mice and Cynomolgus Monkeys

Lean C57BL/6J, diet-induced obese (DIO) C57BL/6J, and melanocortin receptor 4 knock-out (MC4RKO; B6. 129S4-Mc4^{tm1Low1}/J) male mice were obtained from The Jackson Laboratory. Male C57BL/6NTac mice (NASH-B6-M) were obtained from Taconic and maintained on a Gubra-Amylin diet (GAN-DIO; Research Diets, D09100310) starting at six weeks of age. C57BL/6J mice received regular chow (Teklad rodent diet 2920x, Inotiv), DIO C57BL/6J mice received high-fat diet (HFD; rodent diet 60% kcal from fat; Research Diets #D12492), and MC4RKO mice were placed on a breeder diet (breeder diet, 5K20-LabDiet) for five weeks to accelerate their weight gain and then switched to regular chow diet (Teklad rodent diet 2920x, Inotiv). Mice had ad libitum access to water and food.

Mice were treated daily with the indicated dose of CT-388 administered subcutaneously or vehicle, based on body weight at the time of administration. Assessments included percent change from baseline in body weight, food consumption, fasting blood glucose and insulin levels, weight of inguinal white adipose tissue and liver, and changes in liver parameters in GAN-DIO mice.

Mice were singly housed under standard environmental conditions (22° C., 12 h:12 h light:dark cycle). Lean mice were tested between 8-10 weeks of age; diet-induced obese (DIO) mice were maintained on a high-fat diet for at least 18 weeks before experimentation and used at approximately 23 weeks of age. DIO and melanocortin receptor 4 knock-out (MC4RKO) mice were acclimated to daily weighing and handling for approximately one week until weight had stabilized. GAN-DIO mice were maintained on a Gubra-Amylin diet (Research Diets, D09100310) starting at six weeks of age.

Mouse injections were performed six hours before the start of the dark cycle. Percent change from baseline in body weight was calculated by dividing the daily body weight by the body weight taken before the first dose of CT-388 or vehicle, multiplied by 100%. Food consumption was monitored in individually housed mice provided with a premeasured amount of food; consumption was determined by weighing the remaining food 24 hours after administration of CT-388 or vehicle. After euthanization, whole trunk blood was collected in K2-EDTA microvettes (Sarstedt) and kept on ice until centrifugation at 5000 rcf for 10 minutes at 4° C. Fasting blood glucose levels were measured at the time of study termination using an AlphaTRAK2 blood glucose meter. Insulin levels were quantified using a mouse/rat insulin kit (catalog #K152BZC, Mesoscale Discovery, Rockville, MD) according to the manufacturer's instructions. Inguinal white adipose tissue and the liver were removed after euthanization and weighed.

At 32 weeks, GAN-DIO mice underwent liver biopsies as previously described (Heymsfield et al., N Engl J Med. (2017) 376:254-66). Three weeks post-surgery, mice were randomized based on the non-alcoholic fatty liver disease activity score from the biopsy histological analysis. GAN-DIO mice were treated daily with CT-388 by subcutaneous injection for 8 weeks before terminal sacrifice for analysis of changes in liver parameters. For histological analyses, liver sections were fixed in 4% paraformaldehyde at 4° C. for 36 hours, after which they were transferred to 70% alcohol and stored at 4° C. Livers were paraffin-embedded and sectioned at 4 m. Reveal Biosciences performed the histological analyses in both baseline (biopsy) and terminal liver samples using their proprietary deep-learning platform, ImageDx™.

For analyses of liver steatosis in GAN-DIO mice, the lipid regions (white) within the hematoxylin and eosin-stained liver sections were identified and quantified as a percentage of the total area analyzed in the image. Ballooning hepatocytes were identified based on cell diameter and the presence of disrupted cytoskeletal structures, and were quantified as the number of ballooning cells within the total analysis area of hematoxylin and eosin-stained liver sections. Nonalcoholic fatty liver disease activity scores were calculated as the sum of the scores for steatosis, inflammation, and ballooning hepatocytes. For biochemical and hormone analyses in GAN-DIO mice, alanine aminotransferase and aspartate aminotransferase levels were measured using colorimetric assay kits (catalog #13803 and #13801, AAT Bioquest Sunnyvale, CA) per the manufacturer's instructions.

Intraperitoneal glucose tolerance tests (ipGTT) were performed using male lean C57BL/6J mice dosed with CT-388 or vehicle via SC administration at 1, 2, or 4 nmol/kg (5 mice per group) 24 hours before the ipGTT assay. Mice were fasted for 5 hours before dosing of a 20% glucose solution at 2 g/kg (2 mL/kg) via intraperitoneal administration. Blood glucose was measured via tail vein sampling at 0 (pre-glucose), 10, 20, 40, 60, 90, and 120 minutes post-glucose administration using an AlphaTRAK 2 Blood Glucose Meter.

Intravenous glucose tolerance tests (ivGTT) were performed in lean cynomolgus monkeys (n=4 per group) who received a single dose of CT-388 at 0.15 or 0.5 mg/kg or vehicle administered subcutaneously 24 hours before the ivGTT. All monkeys were fasted overnight and anaesthetized before the ivGTT, then intravenously administered a bolus of 50% glucose at a dose of 0.5 g/kg (1 mL/kg). Whole blood samples were collected just before glucose dosing and at 1, 3, 5, 10, 20, 40, 60, and 90 minutes after glucose administration; serum samples were stored at −80° C. until analysis of blood glucose, insulin, and C-peptide concentrations.

For intravenous glucose tolerance test (ivGTT) analyses in cynomolgus monkeys, eight 3-year-old to 5-year-old male monkeys were assigned to two groups (n=4). CT-388 was dissolved in phosphate-buffered saline at 5 mg/mL and diluted in phosphate-buffered saline to the desired dose concentration. On day 1 of the study, all animals were dosed with vehicle (1 mL/kg) via subcutaneous administration 24 hours before the baseline ivGTT. On day 8, the animals were dosed with CT-388 (1 mL/kg) at 0.15 or 0.50 mg/kg (31.1 and 104 nmol/kg, respectively) via subcutaneous administration 24 hours before the ivGTT. The ivGTT was conducted on days 2 and 9 of the study. All animals were fasted overnight and were anaesthetized (Zoletil 50, administered intramuscularly, 5 mg/kg initial dose, and then maintenance dose at 2.5-5 mg/kg as needed). The animals were intravenously administered a bolus of 50% glucose at a dose of 0.5 g/kg (1 mL/kg). Whole blood samples (2 mL) were collected into coagulation-promoting containers just before glucose dosing and at 1, 3, 5, 10, 20, 40, 60, and 90-minutes after glucose administration. The blood samples were centrifuged at 3500 rpm at 4° C. for 10 minutes. The collected serum samples (0.4 mL each) were stored at −80° C. until analysis for blood glucose, insulin, and C-peptide concentrations.

### Phase 1 Single Ascending Dose and Multiple Ascending Dose Clinical Studies

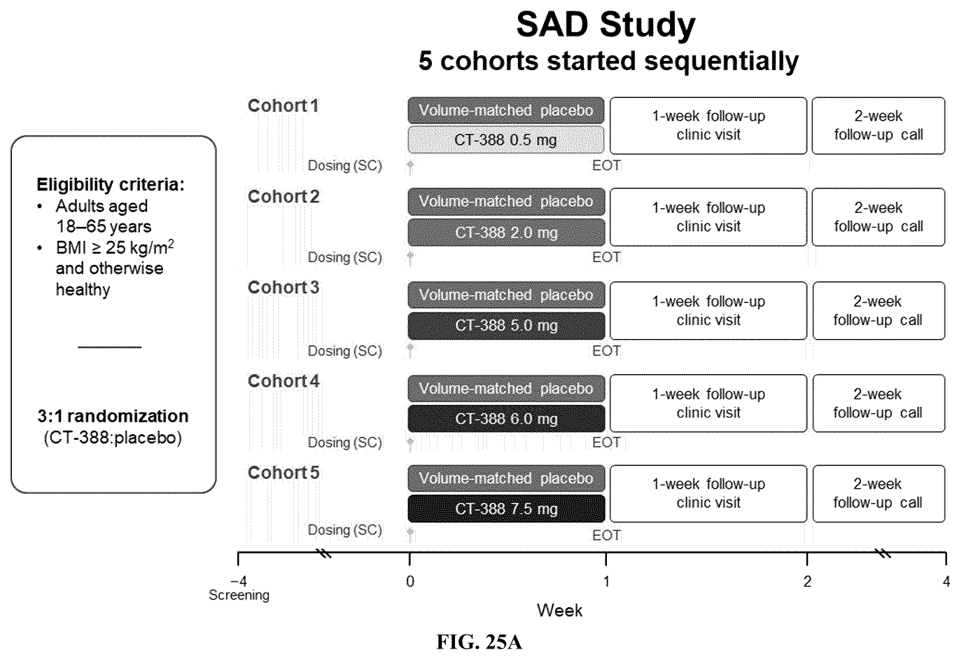

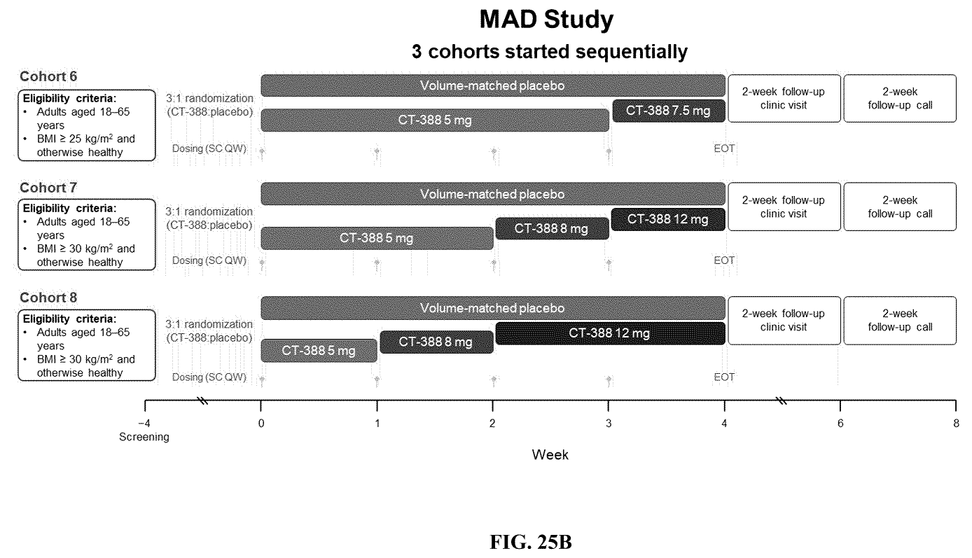

Study design, participants, and treatment: The safety, tolerability, PK, and PD of CT-388 were evaluated in a first-in-human, double-blind, placebo-controlled, randomized phase 1 study (NCT04838405; FIGS. 25A-B). Eight participants were planned per cohort. The single ascending dose (SAD) portion of the study was conducted at a single study site in Australia, while the multiple ascending dose (MAD) portion was conducted at a single study site in Mexico. Both the SAD and MAD study arms enrolled participants aged 18-65 years. Participants in the SAD portion of the study were overweight (BMI≥25 kg/m²) at screening (cohorts 1-5), while the MAD portion included both overweight (BMI≥25 kg/m²; cohorts 6) and obese participants (≥30 kg/m²; cohorts 7-8). Participants were excluded if they had a current diagnosis of diabetes. Participants with prediabetes were allowed (if their condition was controlled and maintained using diet and exercise alone); diabetes and prediabetes were defined using the 2022 American Diabetes Association Standards of Medical Care in Diabetes guidelines (Standards of Medical Care in Diabetes: 2022 Abridged for Primary Care Providers, Clin Diabetes. (2022) 40:10-38).

In each of the SAD and MAD cohorts, eight participants were planned and randomized 3:1 to receive CT-388 (n=6) or placebo (n=2); each cohort was enrolled sequentially after a blinded review of the data from the previous cohorts. Participants in the SAD portion allocated to cohorts 1-5 received one dose of CT-388 (0.5, 2.0, 5.0, 6.0, or 7.5 mg, respectively) administered subcutaneously or volume-matched placebo. Participants in the SAD cohorts were admitted to the clinical site on day −1, dosed on day 1, and discharged on day 3. In the MAD portion of the study, different up-titration schemes were utilized in three sequential cohorts: participants received once-weekly subcutaneous CT-388 5.0 mg on days 1, 8, and 15 followed by 7.5 mg on day 22 (cohort 6); 5.0 mg on days 1 and 8, 8.0 mg on day 15, and 12.0 mg on day 22 (cohort 7); or 5.0 mg on day 1, 8.0 mg on day 8, and 12.0 mg on days 15 and 22 (cohort 8); or placebo (cohorts 6-8; volume-matched to the study drug at each dose level). Participants in the MAD cohorts were admitted to the clinical study site on days −2, 7, 14, and 21 and discharged on days 1, 10, 17, and 24.

Assessments: The primary objective of this phase 1 study was to investigate the safety and tolerability of CT-388 after single and multiple doses in participants with overweight or obesity. Safety assessments included monitoring of treatment-emergent adverse events (TEAEs), serious adverse events (SAEs), TEAEs leading to study drug discontinuation, and safety laboratory parameters. TEAEs were defined as adverse events that began on or after the time of first study drug administration and were coded using the Medical Dictionary for Regulatory Activities (version 25.1) system organ class and preferred terms. Secondary and exploratory objectives were to characterize the PK and PD of CT-388. PK parameters included area under the concentration-time curve from time zero to infinity (AUC_{0-inf}), maximum observed concentration (Cₘₐₓ), elimination half-life (t_{1/2}), and time to maximum concentration (tₘₐₓ), which were calculated using non-compartmental methods. PD assessments included changes from baseline in body weight; hip and waist circumference; glycated hemoglobin (HbA1c), fasting glucose, and fasting insulin levels; HOMA-IR; appetite and satiety by visual analog scale; and glucose and insulin responses after an oral glucose tolerance test (OGTT) performed at baseline (one day before the first CT-388 dose) and day 23 (at the maximal concentration [Cₘₐₓ] of CT-388).

For PK evaluations, serum concentrations of CT-388 were measured pre-dosing and at 8, 24, 48, 72, 96, 120, 168, and 336 hours post-dosing in the SAD portion of the study, and pre-dosing and at 8, 24, 48, 72, and 168 hours post-dosing on days 8, 15, and 22 of the MAD portion of the study.

Body weight was measured ten hours after fasting (including no more than two glasses of fluids), after voiding, and while wearing a gown with undergarments only; body weight was averaged from three measurements using calibrated scales.

Appetite was assessed using a visual analog scale with eight items scored using a 5-point scale; possible scores ranged from 8-40, with lower scores indicating worse appetite. Satiety was assessed using a visual analog scale with four questions using a 100-millimeter scale. Based on the previous seven days, participants were asked to rate their general satiety/satisfaction (100=completely satisfied, 0=not at all satisfied); fullness (100=totally full, 0=not at all full), hunger (100=never been more hungry, 0=not hungry at all), and prospective food consumption (100=a lot, 0=nothing at all). Overall satiety scores were calculated as the average of the four individual scores (satiety+fullness+[100-prospective food consumption]+[100-hunger]/4), with a higher overall score indicating more satiety.

An oral glucose tolerance test (OGTT) was conducted during the MAD portion of the study at baseline (1 day before the first CT-388 dose) and day 23. Participants were fasted for ten hours before ingesting a 75 g glucose solution. Blood was drawn 15 minutes pre-drink and at 0, 30, 60, 90, and 120 minutes post-drink. Assessments during the OGTT included mean (SD) plasma levels and area under the concentration-time curve (AUC) from baseline to day 29 for glucose, insulin, glucagon, C-peptide and HOMA-IR.

### Statistical Analysis

Results for in vitro, mouse, and cynomolgus monkey analyses are presented as means±standard error unless otherwise indicated. For in vitro dose response data, potency (half-maximal effective concentration, EC₅₀) and efficacy (maximal efficacy, Eₘₐₓ) were extracted from a non-linear regression analysis with the hill slope constrained to 1. For detecting differences between groups in glucose homeostasis, a one-way analysis of variance (ANOVA) with Tukey's test for multiple comparisons was used. For all other statistical comparisons, a two-way ANOVA with group and time as between-subject factors was used. When significant differences were observed, multiple comparisons were performed using a Tukey correction. Statistical significance was set at P<0.05. GraphPad Prism (GraphPad Software, Boston, MA) was used to normalize data, perform statistical analyses, and generate non-linear regression curves and graphs.

All human data were summarized by treatment group (placebo groups were pooled for cohorts 1-5 and 6-8, respectively) using descriptive statistics; mixed model repeated measures and ANOVA models were used to analyze the change from baseline for PD endpoints. PK parameters were also summarized using descriptive statistics (number of participants, mean, geometric mean, standard deviation, coefficient of variation, median, minimum, and maximum). In the SAD portion, the power model was used to assess dose proportionality.

### B. Results

cAMP Production, β-Arrestin Recruitment, Receptor Internalization, and GSIS

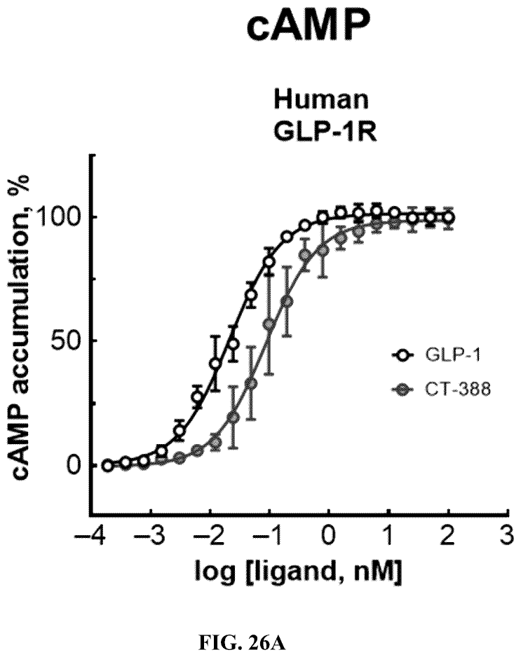

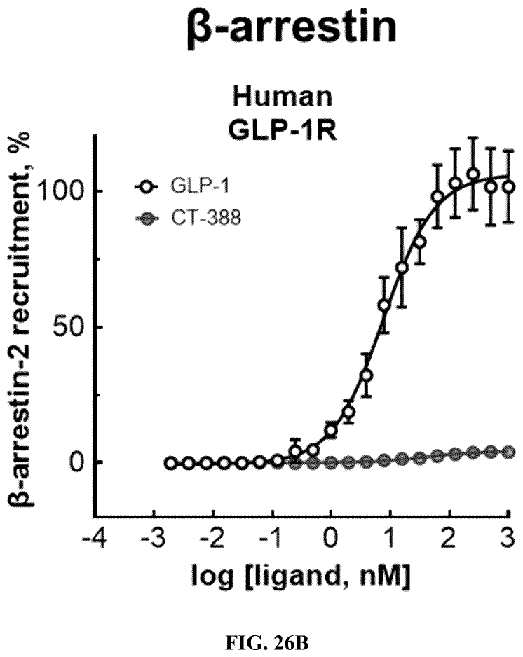

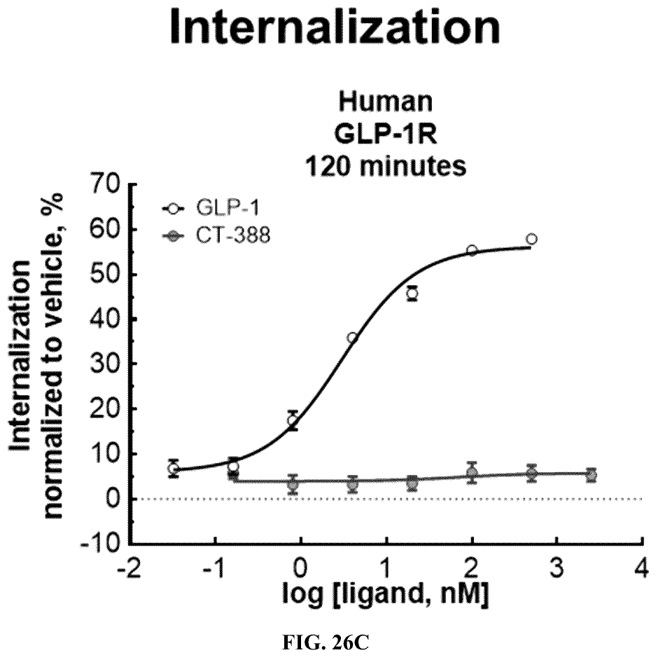

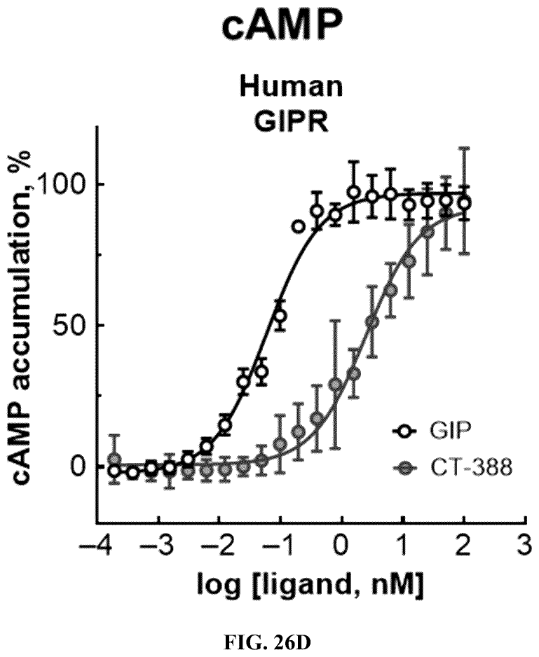

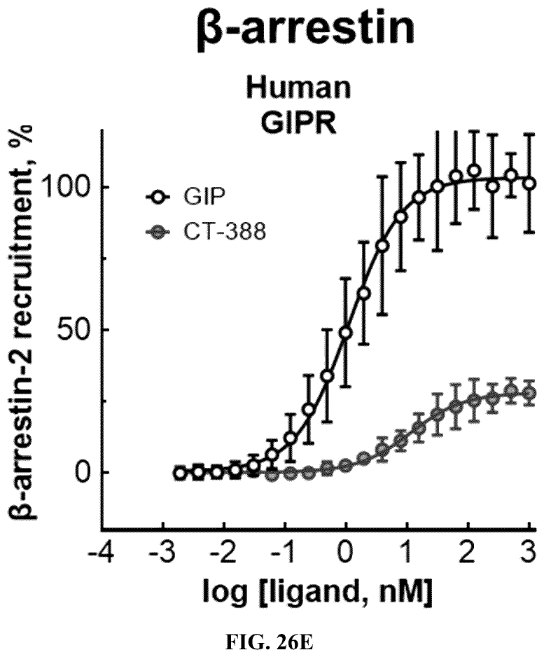

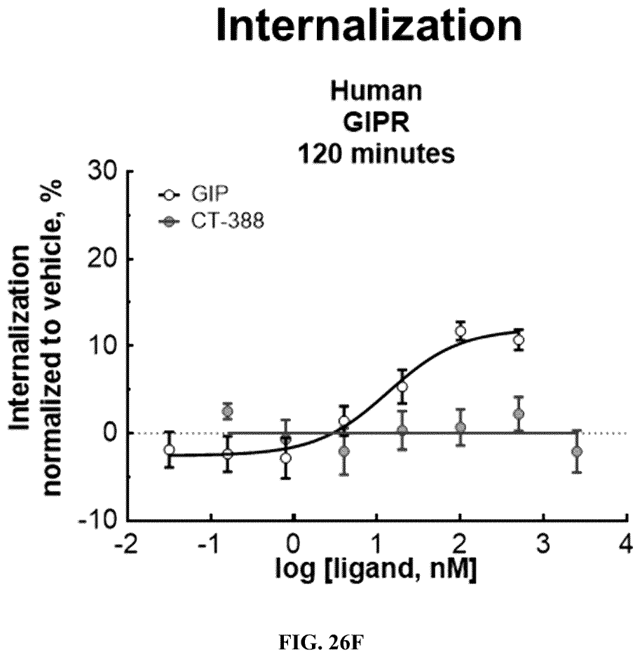

CT-388 was pharmacologically active in vitro on the human and mouse GLP-1 and GIP receptors (Tables 22 and 23). Using an in vitro cAMP production assay, CT-388 demonstrated potent full agonism at the human GLP-1 receptor; the half-maximal effective concentration (EC₅₀) for CT-388 was 0.087 nM with 99% maximal efficacy (Eₘₐₓ, FIG. 26A). Unlike native GLP-1, CT-388 did not recruit 0-arrestin-2 to the human GLP-1 receptor (Eₘₐₓ=4%; FIG. 26B). In addition to the lack of 0-arrestin-2 engagement, human GLP-1 receptor internalization with CT-388 was substantially decreased (Eₘₐₓ=6%), particularly when compared with the response to treatment with native human GLP-1 (FIG. 26C). CT-388 also demonstrated cAMP-biased signaling at the human GIP receptor (cAMP EC₅₀=2.47 nM; Eₘₐₓ=92%; FIG. 26D); 0-arrestin-2 recruitment at the human GIP receptor was minimal (0-arrestin-2 EC₅₀=11 nM; Eₘₐₓ=28%; FIG. 26E). Human GIP receptor internalization was very low with CT-388 treatment, with EC₅₀ and Eₘₐₓ below the level of detection (FIG. 26F).

TABLE 22
Compound Potency (EC₅₀) and Efficacy
(% Eₘₐₓ) Values for Human GIPR

Compound
Human GLP-1R EC₅₀, nM (% Eₘₐₓ, N)

| Modality | GLP-1 | | CT-388 | |
|---|---|---|---|---|
| cAMP | 0.021 | (101, 4) | 0.087 | (99, 4) |
| β-arrestin-2 | 7.6 | (106, 4) | 39 | (4, 4) |
| Internalization | 3.0 | (56, 8) | 67 | (6, 8) |

TABLE 23
Compound potency (EC₅₀) and Efficacy
(% Eₘₐₓ) Values for Human GLP-1R

| | Compound | | |
|---|---|---|---|
| | Human GIPR EC₅₀, nM (% Eₘₐₓ, N) | | |
| Modality | | GIP | CT-388 |

| | | | | |
|---|---|---|---|---|
| cAMP | 0.059 | (97, 4) | 2.47 | (92, 4) |
| β-arrestin-2 | 1.1 | (104) | 11 | (28, 4) |
| Internalization | 14 | (12, 12) | No fit | (—, 12) |

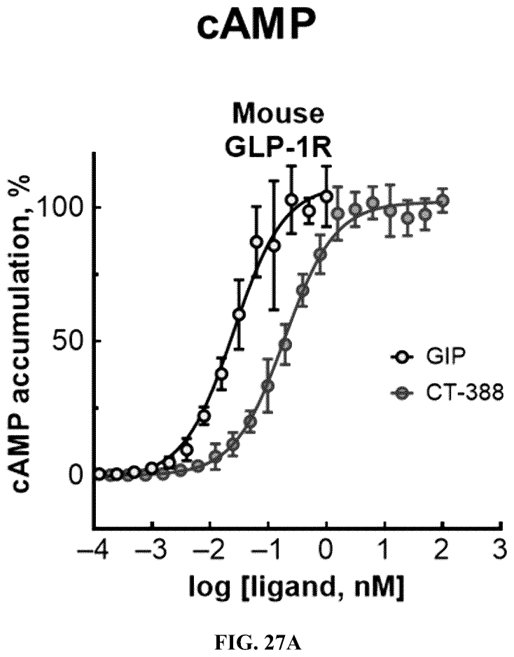

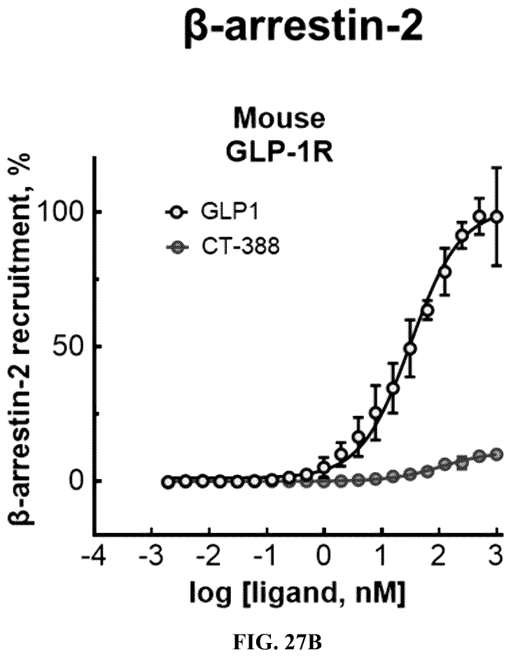

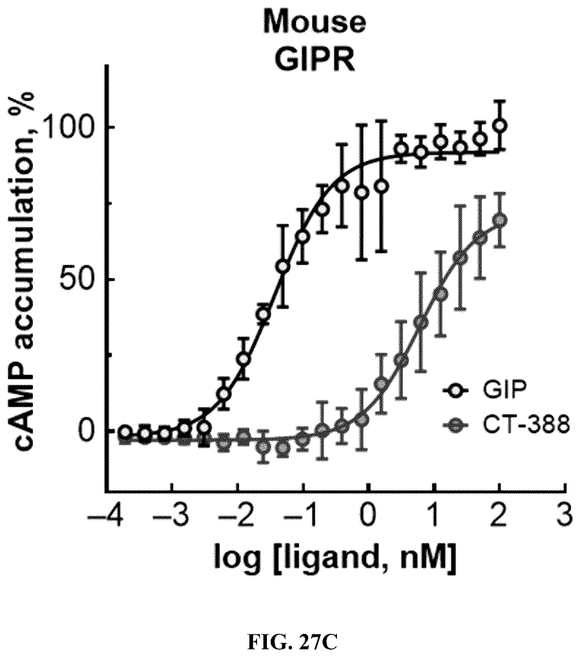

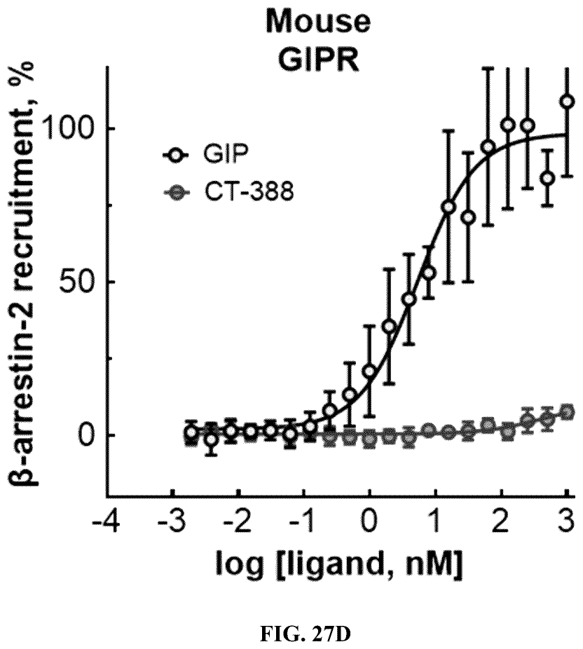

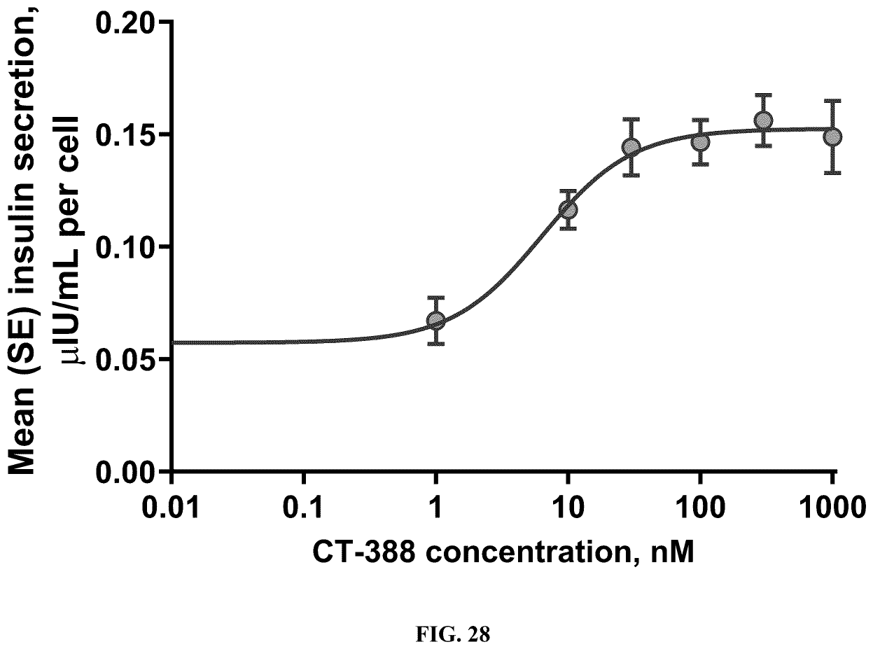

With signaling bias and decreased internalization maintained on both receptors, potency of CT-388 is skewed toward the GLP-1 receptor, as it was four-fold less potent as the native ligand on the GLP-1 receptor but 42-fold less potent on the GIP receptor. CT-388 did not produce any off-target effects at orthologous incretin receptors, as it induced no cAMP accumulation at glucagon or GLP-2 receptors. Similar results for cAMP activity and β-arrestin recruitment were observed for the mouse GLP-1 and GIP receptors with CT-388 treatment in vitro (FIGS. 27A-D; Tables 24 and 25); results for β-arrestin-1 on the human and mouse GLP-1 receptor were similar to those observed for β-arrestin-2 (data not shown). To assess the direct physiological response after signaling activation by CT-388 through the GLP-1 and GIP receptors, the effects of CT-388 on GSIS were tested in primary human beta cells. CT-388 dose-dependently enhanced GSIS in primary human beta cells with a EC₅₀ of 6.356 nM (FIG. 28). Cells were treated with or without increasing concentrations of CT-388 in the presence of 11 mM glucose. Cells were treated for 40 minutes with stimuli, and secreted human insulin was collected and measured thereafter. The EC₅₀ of CT-388-mediated GSIS was calculated to be 6.365 nM (n=5-6).

TABLE 24
EC₅₀ and Eₘₐₓ Values for Mouse GLP-1R

| | Compound | | |
|---|---|---|---|
| | Mouse GLP-1R EC₅₀, nM (% Eₘₐₓ, N) | | |
| Modality | | GLP-1 | CT-388 |

| | | | | |
|---|---|---|---|---|
| sAMP | 0.025 | (108, 4) | 0.19 | (102, 4) |
| β-arrestin-2 | 32 | (101, 4) | 109 | (11, 4) |

TABLE 25
EC₅₀ and Eₘₐₓ Values for Mouse GIPR

| | Compound | | |
|---|---|---|---|
| | Mouse GIPR EC₅₀, nM (% Eₘₐₓ, N) | | |
| Modality | | GIP | CT-388 |

| | | | | |
|---|---|---|---|---|
| cAMP | 0.037 | (92, 4) | 6.0 | (72, 4) |
| β-arrestin-2 | 5.3 | (99, 4) | 381 | (10, 4) |

ipGTT and ivGTT Evaluation

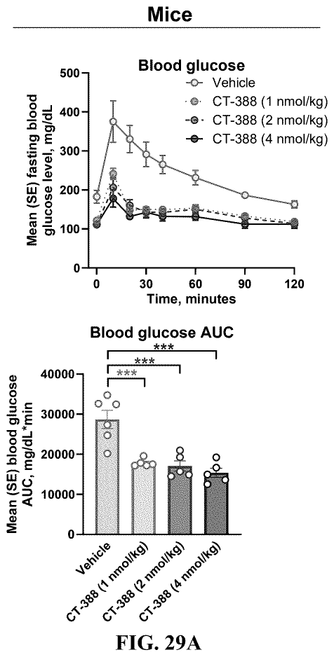

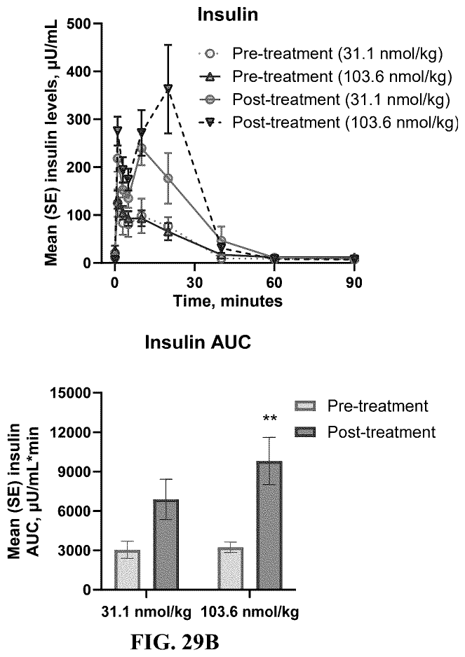

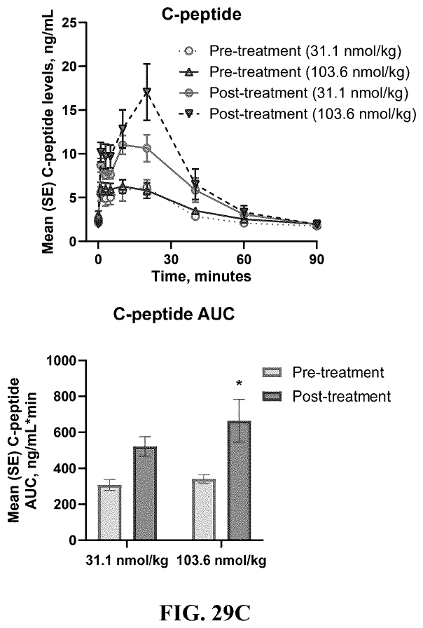

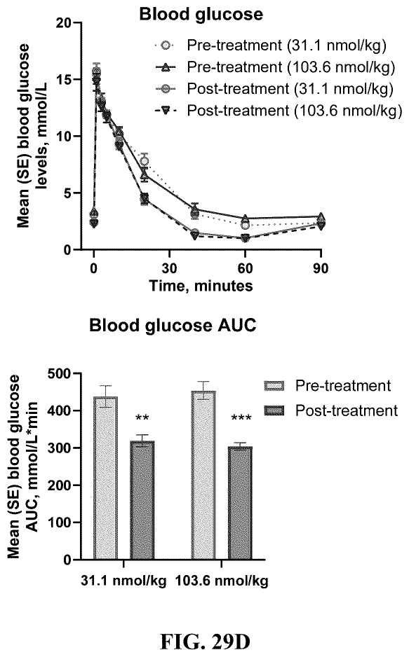

In lean mice, a single dose of CT-388 (administered at doses of 1-4 nmol/kg) was found to substantially lower post-prandial blood glucose levels and blood glucose AUC during an ipGTT relative to vehicle controls (P<0.001; FIG. 29A). In cynomolgus monkeys, a single dose of CT-388 was found to dose-dependently increase insulin and C-peptide concentrations compared with vehicle control during an ivGTT (FIGS. 29B-C). The increases in insulin and C-peptide levels were biphasic, a characteristic feature of GSIS during an ivGTT. In association with the increased insulin concentrations, both doses of CT-388 (31.1 nmol/kg and 103.6 nmol/kg) were found to enhance glucose elimination as demonstrated by significantly lower blood glucose levels compared with vehicle controls (P<0.01 and P<0.001, respectively; FIG. 29D). These data demonstrate that a biased dual GLP-1/GIP receptor agonist can significantly increase insulin secretion and lower blood glucose in lean and otherwise healthy animals.

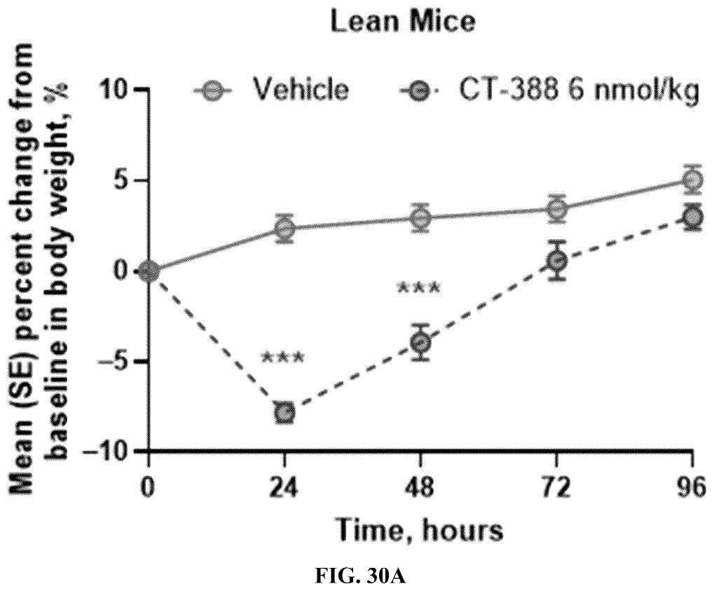

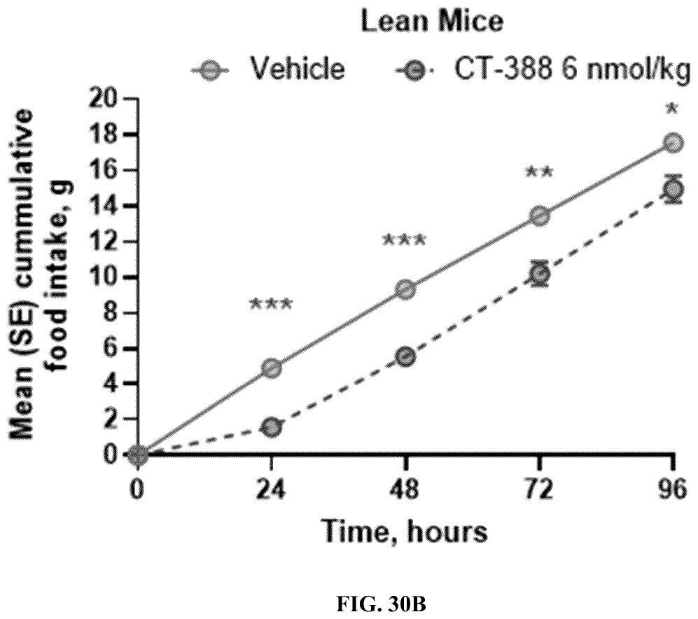

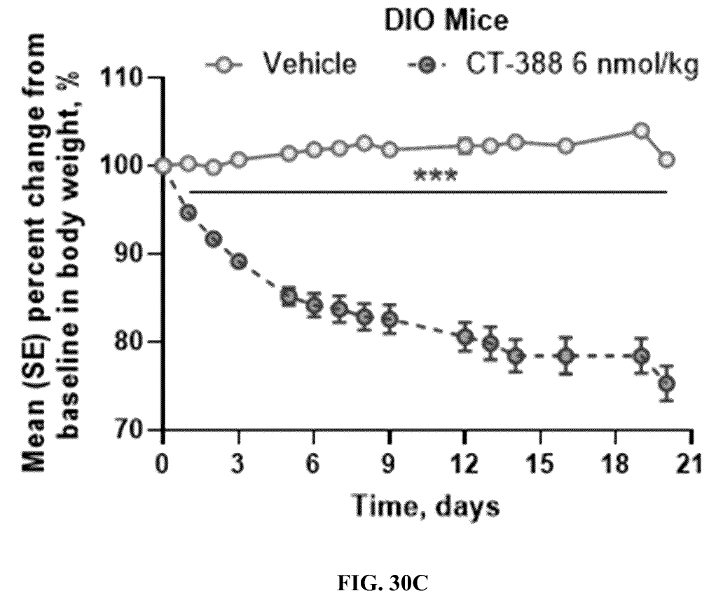

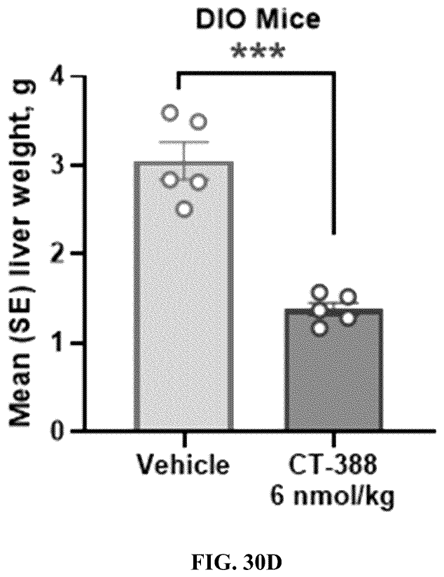

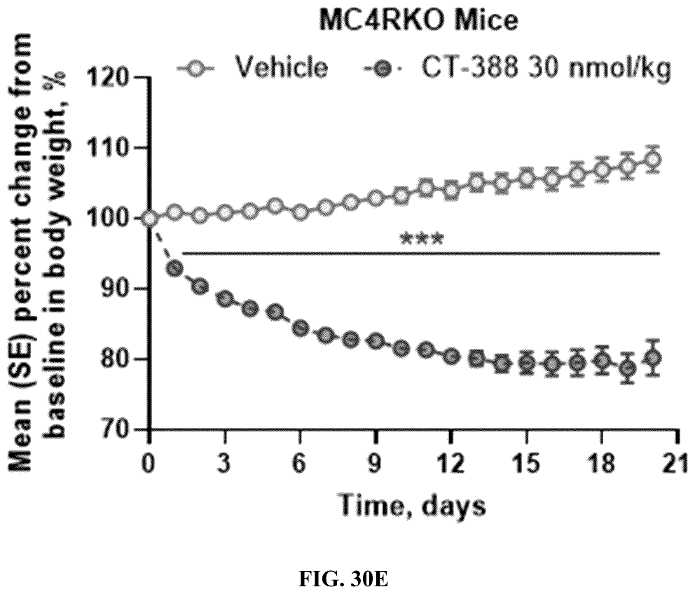

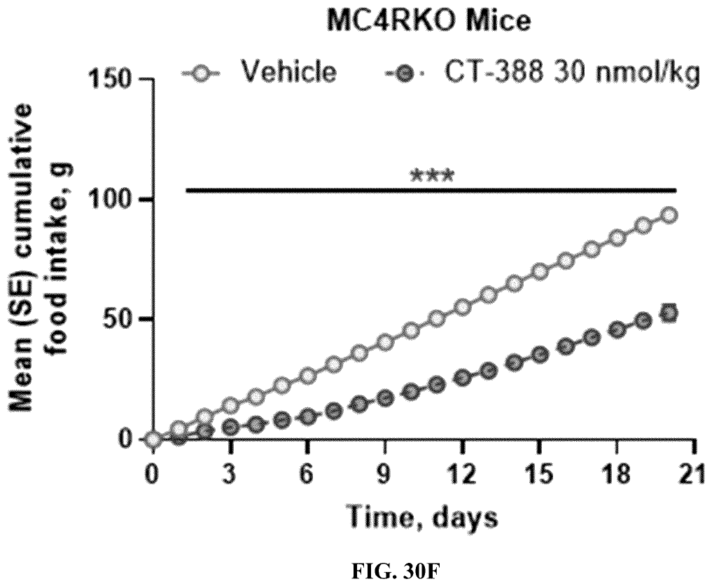

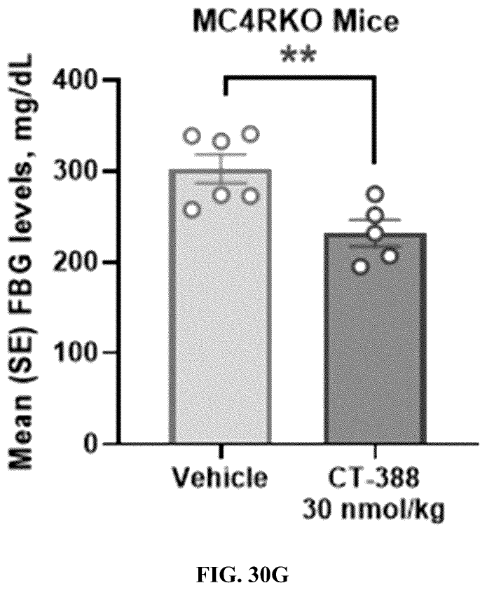

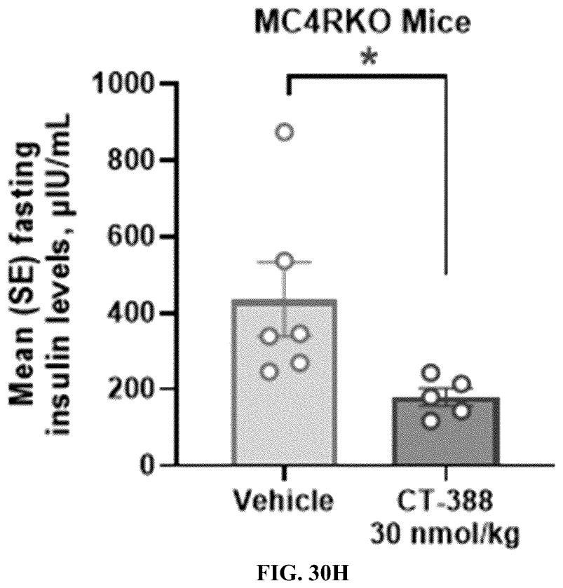

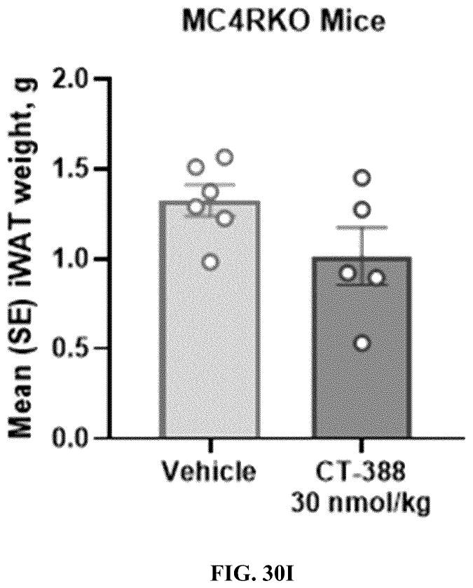

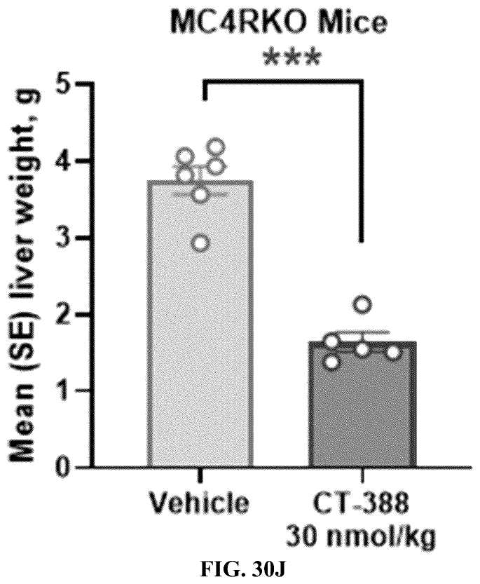

The impact of CT-388 on body weight and food consumption in both acute and sub-chronic settings as well as in normal mice and mouse models of obesity (both diet-induced and genetically induced [MC4RKO] obesity) was also studied. Following a single dose (6 nmol/kg), CT-388 was found to lower body weight in lean mice by approximately 10% compared with vehicle within 24 hours of administration (P<0.001); body weight was observed to return toward baseline levels by 96 hours post-treatment (FIG. 30A). A single dose of CT-388 6 nmol/kg also reduced food intake in lean mice between 24 and 96 hours post-administration compared with vehicle controls (P<0.001 at 24 and 48 hours post-administration, P<0.05 at 96 hours post-administration; FIG. 30B). Body weight and food intake were also found to be significantly decreased in DIO mice and MC4RKO mice (diet-induced and genetic-induced models of obesity, respectively) treated with CT-388 sub-chronically for 21 days compared with vehicle control. After 21 days of dosing in DIO mice, CT-388 at 6 nmol/kg was found to reduce body weight by approximately 25% compared with vehicle (P<0.001; FIG. 30C); the reduced body weight in DIO mice was also associated with a significant reduction in liver weight (FIG. 30D). In MC4RKO mice after 21 days of treatment, CT-388 at 30 nmol/kg was found to reduce body weight from baseline by approximately 20% compared with an increase from baseline of approximately 8% with vehicle (P<0.001; FIG. 30E) and the accumulated food intake was found to decrease by approximately 41 g (43.5%) compared with vehicle (P<0.001; FIG. 30F). CT-388 treatment at 30 nmol/kg was also found to reduce 12-hour fasting glucose levels in MC4RKO mice compared with vehicle (P<0.01; FIG. 30G). CT-388 treatment also reduced fasting insulin levels (P<0.05; FIG. 3011), inguinal white adipose tissue weight (FIG. 30I), and liver weight (FIG. 30J) in MC4RKO mice compared with vehicle controls.

### Chronic Effects of CT-388 on Body Weight and MASH in GAN-DIO Mice

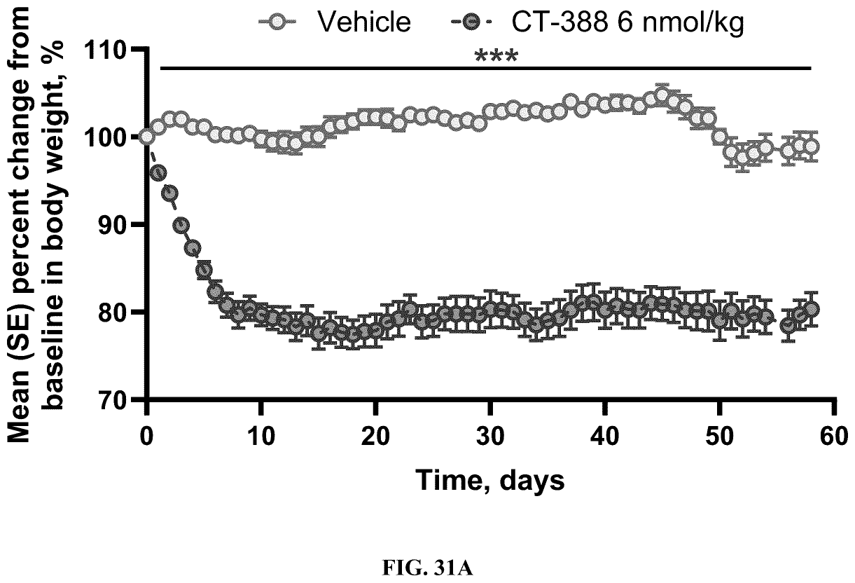

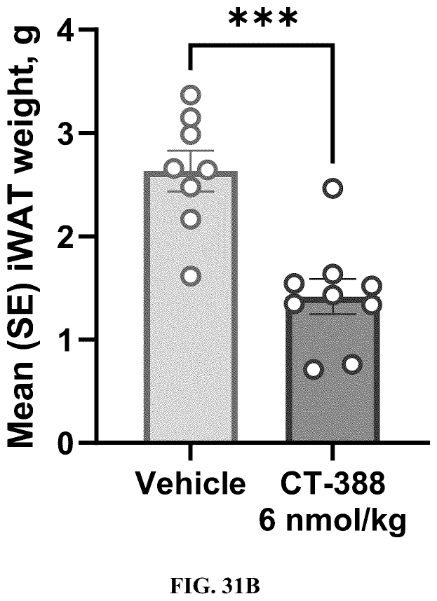

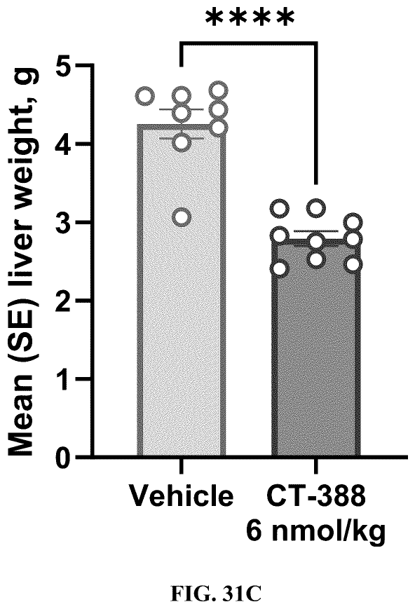

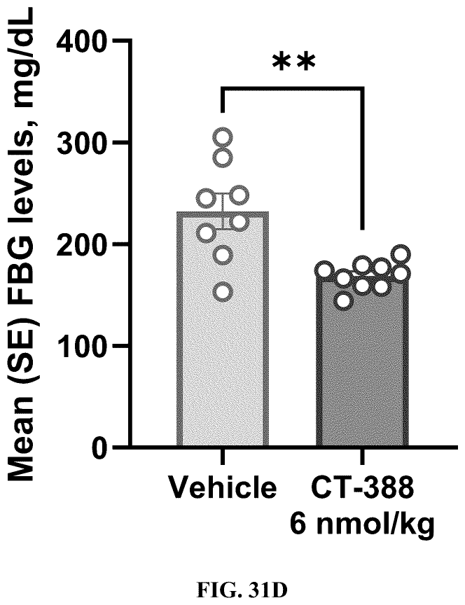

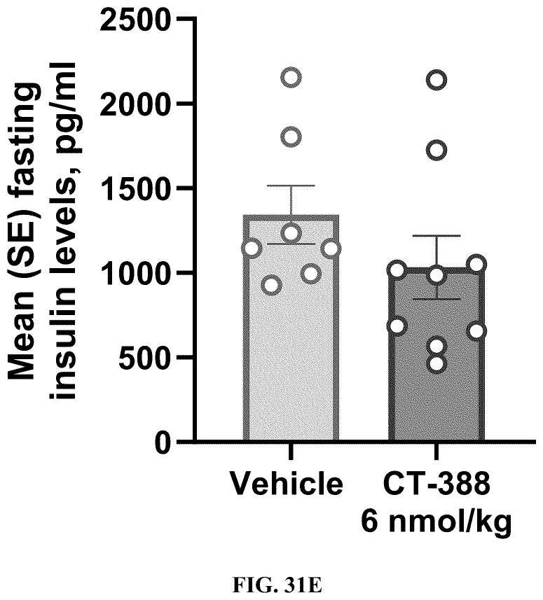

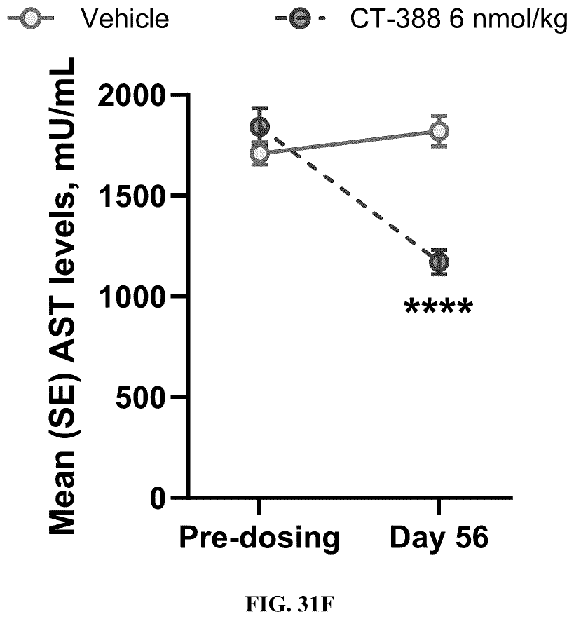

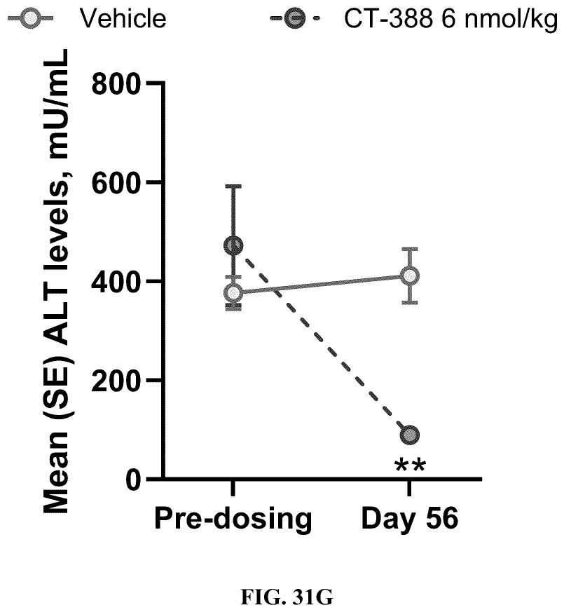

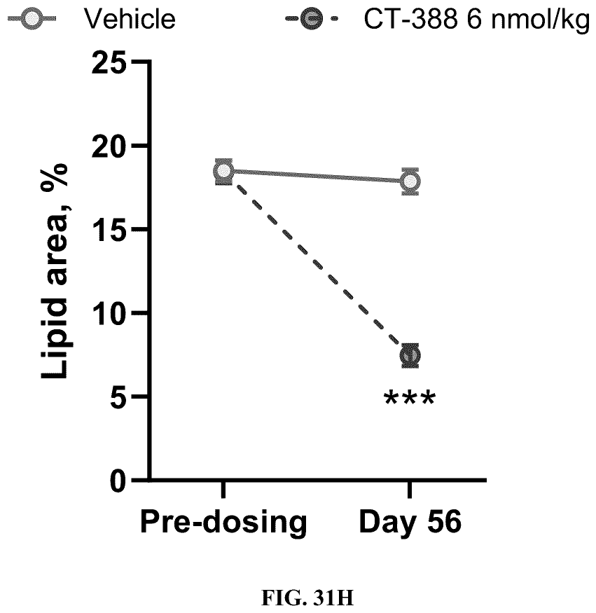

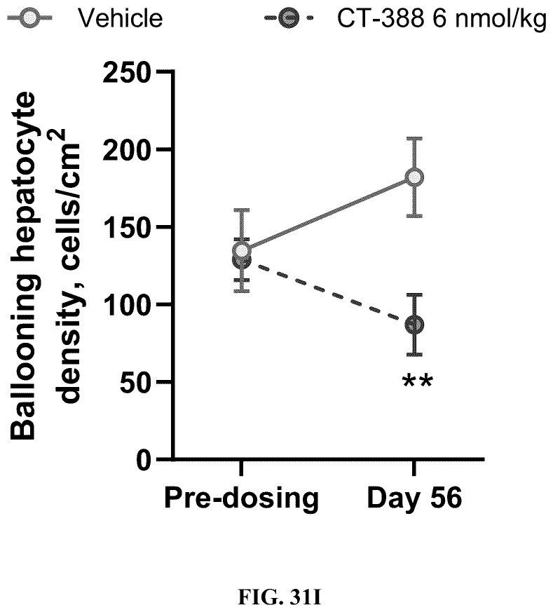

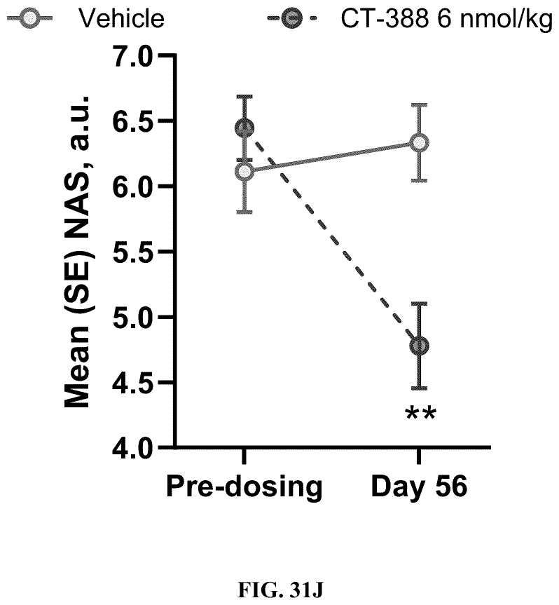

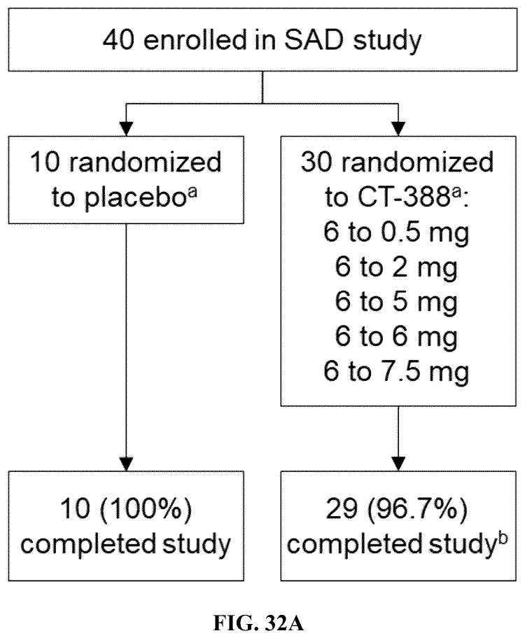

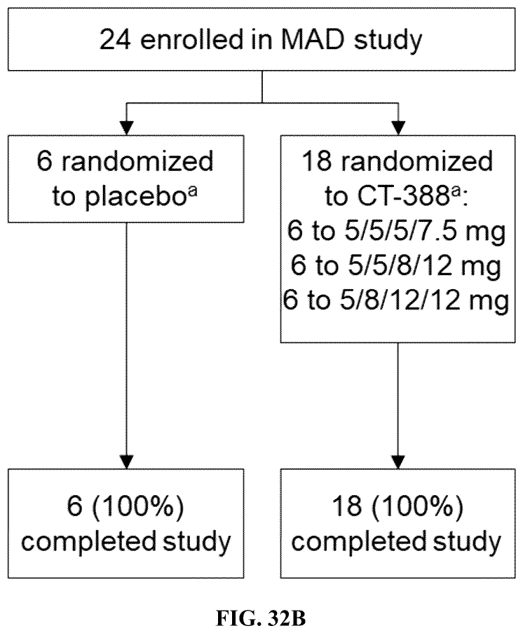

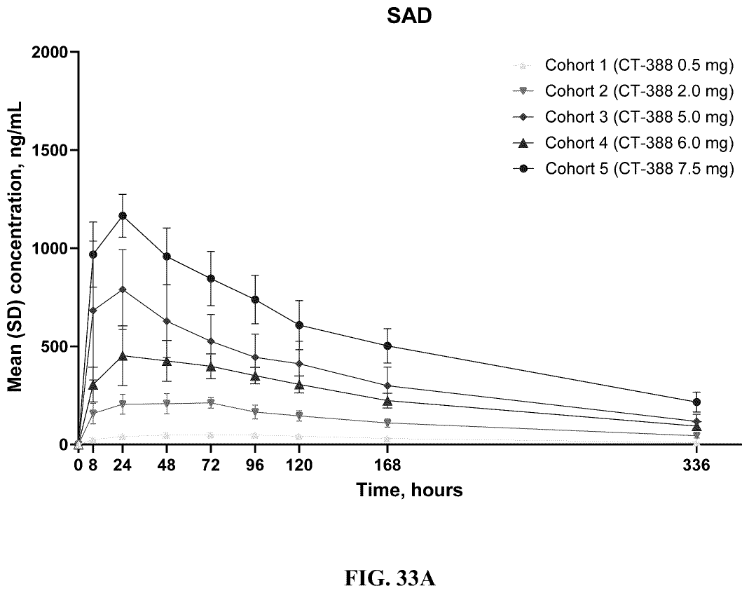

In GAN-DIO mice (a model of obesity associated with similar liver morphological characteristics as seen in patients with metabolic dysfunction-associated steatohepatitis (MASH)), an 8-week treatment with 6 nmol/kg of CT-388 was found to robustly and durably reduce body weight; after 8 weeks, mean body weight was found to be reduced by approximately 20% with CT-388 vs 1% with vehicle (P<0.001; FIG. 31A). Reductions in body weight after CT-388 treatment were also associated with significantly reduced inguinal white adipose tissue and liver weights (both P<0.001 vs vehicle; FIGS. 31B-C). Fasting blood glucose levels were significantly decreased in GAN-DIO mice treated with CT-388 as compared to vehicle, while fasting insulin levels were not significantly different between the two treatment groups (FIGS. 31D and 31E). Circulating liver enzyme measurements also show that CT-388 treatment greatly reduced alanine aminotransferase (ALT) and aspartate aminotransferase (AST) levels (FIGS. 31F-G). More importantly, histomorphometric analysis of the liver, a key component of MASH assessment, indicated that CT-388 substantially reduced lipid accumulation and hepatocyte ballooning (FIGS. 32H-I). As such, CT-388 significantly reduced the nonalcoholic fatty liver disease activity score (FIG. 31J).

### Phase 1 SAD and MAD Clinical Studies

Participant demographics and baseline characteristics: CT-388 was advanced into clinical testing in a first-in-human, phase 1 study in participants with overweight or obesity. Each SAD and MAD cohort included six actively treated participants and two placebo-treated participants (FIGS. 32A-B).

In the SAD portion of the study (cohorts 1-5), a total of 40 participants with overweight or obesity enrolled, with 30 participants exposed to a single dose of CT-388. Placebo-treated participants from cohorts 1-5 were pooled together to form the placebo group (n=10). There was some variability in baseline demographics across the SAD CT-388-dose groups and placebo group; mean (SD) age ranged from 29.7 (11.0) to 40.8 (11.6) years, mean (SD) BMI ranged from 28.3 (3.4) to 33.9 (3.6) kg/m², and the percentage of female participants ranged from 16.7% to 83.3% (Tables 26 and 27). Most participants were White. All participants received one dose of CT-388. One participant who received a single dose of 6 mg of CT-388 discontinued the study (due to withdrawal by the participant).

TABLE 26
Baseline Demographics and Characteristics of Patients in SAD Study

| | Pooled | | | | | | | | | | | |
|---|---|---|---|---|---|---|---|---|---|---|---|---|
| | Placebo | | CT-388 | | CT-388 | | CT-388 | | CT-388 | | CT-388 | |
| | Cohorts | | Cohort 1 | | Cohort 2 | | Cohort 3 | | Cohort 4 | | Cohort 5 | |
| | 1-5 | | (0.5 mg) | | (2.0 mg) | | (5.0 mg) | | (6.0 mg) | | (7.5 mg) | |
| | (n = 10) | | (n = 6) | | (n = 6) | | (n = 6) | | (n = 6) | | (n = 6) | |
| Age, years, | 36.6 | (13.2) | 35.5 | (12.5) | 34.8 | (14.6) | 40.8 | (11.6) | 37.3 | (10.1) | 29.7 | (11.0) |
| mean (SD) | | | | | | | | | | | | |
| Female, n (%) | 6 | (60.0) | 5 | (83.3) | 5 | (83.3) | 3 | (50.0) | 1 | (16.7) | 4 | (66.7) |
| Race, n (%) | 0 | | 0 | | 0 | | 0 | | 0 | | 0 | |
| White | 8 | (80.0) | 4 | (66.7) | 6 | (100) | 5 | (83.3) | 4 | (66.7) | 5 | (83.3) |
| Asian | 1 | (10.0) | 0 | | 0 | | 1 | (16.7) | 2 | (33.3) | 1 | (16.7) |
| Other | 1 | (10.0) | 2 | (33.3) | 0 | | 0 | | 0 | | 0 | |
| Ethnicity, | 0 | | 0 | | 0 | | 0 | | 0 | | 0 | |
| Hispanic or | | | | | | | | | | | | |
| Latino, | | | | | | | | | | | | |
| n (%) | | | | | | | | | | | | |
| Weight, kg, | 95.6 | (16.9) | 81.5 | (6.2) | 96.6 | (20.4) | 94.9 | (25.5) | 109.4 | (24.6) | 79.0 | (12.2) |
| mean (SD) | | | | | | | | | | | | |
| BMI, kg/m², | 32.2 | (2.9) | 30.2 | (2.3) | 33.9 | (3.6) | 31.4 | (4.2) | 33.4 | (4.8) | 28.3 | (3.4) |
| mean (SD) | | | | | | | | | | | | |
| Fasting | 94.6 | (77.5, 108.1) | 91.0 | (86.5, 95.5) | 97.3 | (91.9, 108.1) | 92.8 | (84.7, 102.7) | 95.5 | (90.1, 100.9) | 90.1 | (84.7, 97.3) |
| glucose, | | | | | | | | | | | | |
| mg/dL, | | | | | | | | | | | | |
| median | | | | | | | | | | | | |
| (min, max) | | | | | | | | | | | | |
| HOMA-IR, | 3.4 | (1.7, 7.2) | 1.8 | (0.8, 4.6) | 4.1 | (2.2, 6.1) | 3.5 | (1.1, 8.4) | 3.1 | (2.5, 6.3) | 2.8 | (1.5, 4.3) |

median
(min, max)

BMI, body mass index; HbA1c, glycated hemoglobin; HOMA-IR, Homeostatic Model Assessment for Insulin Resistance; MAD, multiple ascending dose; max, maximum; min, minimum; SAD, single ascending dose.

TABLE 27
Baseline Demographics and Characteristics of Patients in MAD Study

| | Pooled | | CT-388 | | CT-388 | | CT-388 | |
|---|---|---|---|---|---|---|---|---|
| | Placebo | | Cohort 6 | | Cohort 7 | | Cohort 8 | |
| | Cohorts 6-8 | | (5/5/5/7.5 mg) | | (5/5/8/12 mg) | | (5/8/12/12 mg) | |
| | (n = 6) | | (n = 6) | | (n = 6) | | (n = 6) | |
| Age, years, mean | 41.5 | (10.7) | 34.0 | (10.9) | 34.3 | (14.0) | 26.3 | (7.9) |
| (SD) | | | | | | | | |
| Female, n (%) | 4 | (66.7) | 5 | (83.3) | 1 | (16.7) | 2 | (33.3) |
| Race, Other, n | 6 | (100) | 6 | (100) | 6 | (100) | 6 | (100) |
| (%) | | | | | | | | |
| Ethnicity, | 6 | (100) | 6 | (100) | 6 | (100) | 6 | (100) |
| Hispanic or | | | | | | | | |
| Latino, n (%) | | | | | | | | |
| Weight, kg, | 90.1 | (14.1) | 81.8 | (16.7) | 107.2 | (19.3) | 97.1 | (18.9) |
| mean (SD) | | | | | | | | |
| BMI, kg/m², | 34.0 | (1.1) | 31.3 | (3.8) | 35.6 | (2.8) | 34.8 | (4.0) |
| mean (SD) | | | | | | | | |
| Fasting glucose, | 98.0 | (86.0, 115.0) | 91.5 | (90.0, 104.0) | 96.5 | (88.0, 100.0) | 88.5 | (82.0, 97.0) |
| mg/dL, median | | | | | | | | |
| (min, max) | | | | | | | | |
| HOMA-IR, | 3.8 | (2.0, 7.5) | 2.2 | (1.5, 3.5) | 5.0 | (2.5, 8.8) | 3.9 | (1.3, 7.6) |
| median | | | | | | | | |
| (min, max) | | | | | | | | |
| HbA1c (%) | 5.5 | (4.9, 5.8) | 5.2 | (4.9, 5.6) | 5.3 | (4.7, 5.8) | 5.4 | (5.1, 5.5) |

median
(min, max)

The MAD portion of the study (cohorts 6-8) included 24 participants with overweight or obesity who were otherwise healthy (18 who received CT-388 and six in the pooled placebo group). Variability in baseline BMI and body weight between cohorts reflected the inclusion criteria of participants with overweight or obesity in cohort 6, as compared to the inclusion of only participants with obesity in cohorts 7 and 8. Mean (SD) age also varied across cohorts, ranging from 26.3 (7.9) to 41.5 (10.7) years. All participants in the MAD portion of the study were of Hispanic or Latino ethnicity and reported a race of “Other.” The study was conducted in Mexico. All participants completed the MAD portion of the study, and their study treatment was performed according to the prespecified schedule without any dose modifications such as withholding a dose or down-titrations.

Safety: In the SAD portion of the study, most of the participants experienced at least one TEAE in the placebo group and each of the CT-388 dose groups; the majority of TEAEs were mild or moderate in severity (Table 28).

TABLE 28
Safety Summary from the Phase 1 SAD Portion of the Study

| | Pooled | | CT-388 | | CT-388 | | CT-388 | | CT-388 | | CT-388 | |
|---|---|---|---|---|---|---|---|---|---|---|---|---|
| | Placebo | | Cohort 1 | | Cohort 2 | | Cohort 3 | | Cohort 4 | | Cohort 5 | |
| | Cohorts 1-5 | | (0.5 mg) | | (2.0 mg) | | (5.0 mg) | | (6.0 mg) | | (7.5 mg) | |
| TEAEs, n (%) | (n = 10) | | (n = 6) | | (n = 6) | | (n = 6) | | (n = 6) | | (n = 6) | |
| Participants with | 8 | (80.0) | 6 | (100) | 6 | (100) | 5 | (83.3) | 5 | (83.3) | 6 | (100) |

| any TEAEs | | | | | | | | | | | |
|---|---|---|---|---|---|---|---|---|---|---|---|
| Serious TEAEs | 0 | | 0 | 0 | | 0 | | 1 | (16.7)^{a} | 1 | (16.7)^{b} |
| Participants who | 0 | | 0 | 0 | | 0 | | 1 | (16.7)^{c} | 0 | |
| discontinued due to | | | | | | | | | | | |
| TEAEs | | | | | | | | | | | |
| Fatal TEAEs | 0 | | 0 | 0 | | 0 | | 0 | | 0 | |
| Drug-related | 4 | (40.0) | 0 | 4 | (66.7) | 4 | (66.7) | 3 | (50.0) | 6 | (100) |

TEAEs^{d}

Severity

| | | | | | | | | | | | | |
|---|---|---|---|---|---|---|---|---|---|---|---|---|
| Mild | 8 | (80.0) | 6 | (100) | 6 | (100) | 5 | (83.3) | 2 | (33.3) | 1 | (16.7) |

| Moderate | 0 | | 0 | 0 | | 0 | | 2 | (33.3) | 4 | (66.7) |
|---|---|---|---|---|---|---|---|---|---|---|---|
| Severe | 0 | | 0 | 0 | | 0 | | 1 | (16.7)^{a} | 1 | (16.7)^{e} |
| GI-related TEAEs | 4 | (40.0) | 0 | 2 | (33.3) | 2 | (33.3) | 2 | (33.3) | 6 | (100) |

| | | | | | | | | |
|---|---|---|---|---|---|---|---|---|
| Abdominal | 0 | 0 | 0 | 1 | (16.7) | 0 | 2 | (33.3) |

| distention | | | | | | | | | | |
|---|---|---|---|---|---|---|---|---|---|---|
| Abdominal pain | 1 | (10.0) | 0 | 1 | (16.7) | 0 | 1 | (16.7) | 1 | (16.7) |
| Abdominal pain, | 0 | | 0 | 0 | | 0 | 0 | | 1 | (16.7) |
| upper | | | | | | | | | | |
| Change of bowel | 0 | | 0 | 0 | | 0 | 0 | | 1 | (16.7) |
| habit | | | | | | | | | | |
| Constipation | 2 | (20.0) | 0 | 1 | (16.7) | 0 | 0 | | 0 | |
| Diarrhea | 1 | (10.0) | 0 | 1 | (16.7) | 0 | 1 | (16.7) | 3 | (50.0) |

| | | | | | | | |
|---|---|---|---|---|---|---|---|
| Dyspepsia | 0 | 0 | 0 | 1 | (16.7) | 0 | 0 |

| Gastroesophageal | 0 | | 0 | 0 | 0 | 0 | | 3 | (50.0) |
|---|---|---|---|---|---|---|---|---|---|
| reflux disease | | | | | | | | | |
| Mallory-Weiss | 0 | | 0 | 0 | 0 | 0 | | 1 | (16.7) |
| syndrome | | | | | | | | | |
| Nausea | 1 | (10.0) | 0 | 0 | 0 | 1 | (16.7) | 5 | (83.3) |

| | | | | | | | | | | |
|---|---|---|---|---|---|---|---|---|---|---|
| Reduced appetite | 0 | 0 | 2 | (33.3) | 1 | (16.7) | 2 | (33.3) | 3 | (50.0) |

| | | | | | | | | | |
|---|---|---|---|---|---|---|---|---|---|
| Vomiting | 0 | 0 | 0 | 2 | (33.3) | 2 | (33.3) | 3 | (50.0) |

GI, gastrointestinal; SAD, single ascending dose; TEAE, treatment-emergent adverse event.
^{a}Severe, spontaneous retinal detachment during the follow-up period; occurred seven days after dosing and was considered unrelated to study drug.
^{b}Severe nausea; considered probably related to study drug.
^{c}Moderate intractable vomiting; considered probably related to study drug.
^{d}Relationship is categorized as “possible” or “probable.”
^{e}One participant experienced moderate vomiting, mild diarrhea, and mild abdominal pain (all of which were considered possibly related to study drug) and discontinued the study 1 day after dosing.

There were two serious TEAEs in the SAD portion: one participant who received 6.0 mg of CT-388 experienced a severe spontaneous retinal detachment during the follow-up period (seven days after dosing; considered unrelated to study drug) and one participant who received 7.5 mg of CT-388 experienced moderate vomiting and severe nausea (considered probably related to study drug). One participant in the SAD portion of the study who received 6 mg of CT-388 discontinued the study one day after dosing (withdrawal by participant); this participant experienced moderate vomiting, mild diarrhea, and mild abdominal pain, all of which were considered possibly related to study drug. There were no deaths in the SAD portion of the study. The most common TEAEs were decreased appetite, nausea, vomiting, and headache. Overall, no dose-dependent differences in TEAEs were observed. The highest rate of drug-related TEAEs was observed in participants who received 7.5 mg of CT-388; this dose group had a lower mean baseline BMI than the 5-mg or 6-mg group receiving CT-388, suggesting that baseline body weight may impact the CT-388 tolerability profile (potentially by affecting drug exposure). Gastrointestinal (GI)-related TEAEs were experienced by 40% of participants in both the placebo group and the CT-388 groups combined in the SAD portion of the study. The number and severity of GI-related TEAEs were generally low in all SAD treatment groups. Similar tolerability was observed in participants receiving 2 mg or 5 mg of CT-388 treatment. No participants experienced moderate or severe TEAEs, and 33% of participants in both groups experienced GI-related events. There were no clinically significant changes in laboratory values (ALT, AST, total bilirubin, gamma-glutamyl transferase, alkaline phosphatase, serum creatinine, or blood urea nitrogen levels) in any SAD treatment group, except for one participant in the 7.5 mg-dose group who experienced an isolated and transient increase in ALT and AST, peaking at more than three-fold the upper limit of normal 1 day post-dosing and returning spontaneously to baseline by 6 days post-dosing without any interventions. No SAD participants experienced TEAEs related to laboratory values and no notable changes in vital sign measurements, ECG parameters, or physical examination findings were reported.

In the MAD portion of the study, all TEAEs were mild to moderate in severity; there were no serious or severe TEAEs, discontinuations due to TEAEs, or deaths (Table 29).

TABLE 29
Safety Summary from the Phase 1 MAD Portion of the Study

| | Pooled | | CT-388 | | CT-388 | | CT-388 | |
|---|---|---|---|---|---|---|---|---|
| | Placebo | | Cohort 6 | | Cohort 7 | | Cohort 8 | |
| | Cohorts 6-8 | | (5/5/5/7.5 mg) | | (5/5/8/12 mg) | | (5/8/12/12 mg) | |
| TEAEs, n (%) | (n = 6) | | (n = 6) | | (n = 6) | | (n = 6) | |
| Participants | 5 | (83.3) | 6 | (100) | 6 | (100) | 6 | (100) |
| with any | | | | | | | | |
| TEAEs | | | | | | | | |
| Serious TEAEs | 0 | | 0 | | 0 | | 0 | |
| Participants | 0 | | 0 | | 0 | | 0 | |
| who | | | | | | | | |
| discontinued | | | | | | | | |
| due to TEAEs | | | | | | | | |
| Fatal TEAEs | 0 | | 0 | | 0 | | 0 | |
| Drug-related | 4 | (66.7) | 6 | (100) | 6 | (100) | 6 | (100) |

TEAEs^{a}

Severity

| Mild | 1 | (16.7) | 0 | | 5 | (83.3) | 3 | (50.0) |
|---|---|---|---|---|---|---|---|---|
| Moderate | 4 | (66.7) | 6 | (100) | 1 | (16.7) | 3 | (50.0) |
| Severe | 0 | | 0 | | 0 | | 0 | |
| GI-related | 3 | (50.0) | 6 | (100) | 4 | (66.7) | 5 | (83.3) |
| TEAEs | | | | | | | | |
| Abdominal | 1 | (16.7) | 3 | (50.0) | 0 | | 0 | |
| distention | | | | | | | | |
| Constipation | 0 | | 3 | (50.0) | 0 | | 1 | (16.7) |
| Diarrhea | 3 | (50.0) | 2 | (33.3) | 2 | (33.3) | 3 | (50.0) |
| Dyspepsia | 1 | (16.7) | 1 | (16.7) | 0 | | 0 | |
| Eructation | 0 | | 0 | | 2 | (33.3) | 1 | (16.7) |
| Gastroesophageal | 0 | | 0 | | 0 | | 2 | (33.3) |
| reflux disease | | | | | | | | |
| Nausea | 1 | (16.7) | 6 | (100) | 1 | (16.7) | 3 | (50.0) |

| Reduced | 0 | 6 | (100) | 6 | (100) | 6 | (100) |
|---|---|---|---|---|---|---|---|
| appetite | | | | | | | |
| Vomiting | 0 | 5 | (83.3) | 2 | (33.3) | 2 | (33.3) |

GI, gastrointestinal; MAD, multiple ascending dose; TEAE, treatment-emergent adverse event.
^{a}Relationship is categorized as “possible” or “probable.”

Most (83.3%) participants who received CT-388 in the MAD portion of the study reported one or more GI-related TEAL; the most common TEALs were decreased appetite, nausea, vomiting, and diarrhea. Decreased appetite was the most common TEAL overall, consistent with the mechanism of action of CT-388, and was experienced by 1000 of participants who received CT-388 treatment compared with no participants in the placebo group. GI-related TEALs were common in all CT-388 dosing groups, but they were primarily mild or moderate in severity; GI-related events were generally transient with no temporal patterns observed around dose up-titration periods and no association between the dose and event onset. The frequency and severity of GI-related TEAEs tended to be lower in participants in cohorts 7 and 8 treated with CT-388 than in participants in cohort 6, despite the faster up-titration dosing scheme and higher target dose (potentially due to differences in baseline body weight per the eligibility criteria).

In the MAD portion of the study, there were no clinically significant changes in main biochemistry parameters, including biomarkers of liver function (ALT, AST, total bilirubin, gamma-glutamyl transferase, alkaline phosphatase levels) or renal function (creatinine levels; Table 30).

TABLE 30
Biochemistry Safety Parameters During the MAD Portion of the Study

| | | | | |
|---|---|---|---|---|
| | Pooled | CT-388 | CT-388 | CT-388 |
| | Placebo | Cohort 6 | Cohort 7 | Cohort 8 |
| Parameter, | Cohorts 6-8 | (5/5/5/7.5 mg) | (5/5/8/12 mg) | (5/8/12/12 mg) |
| mean (SD) | (n = 6) | (n = 6) | (n = 6) | (n = 6) |

Alanine aminotransferase, IU/L

| | | | | | | | | |
|---|---|---|---|---|---|---|---|---|
| Baseline | 22.7 | (9.35) | 14.3 | (5.72) | 28.0 | (14.46) | 45.5 | (31.08) |
| Change from | −7.0 | (13.43) | 1.3 | (8.45) | 2.7 | (10.98) | −15.3 | (30.53) |
| baseline at day 29 | | | | | | | | |

Aspartate aminotransferase, IU/L

| | | | | | | | | |
|---|---|---|---|---|---|---|---|---|
| Baseline | 21.7 | (3.88) | 14.3 | (3.01) | 18.8 | (3.97) | 29.0 | (13.71) |
| Change from | −4.2 | (6.01) | 0.7 | (2.42) | 3.5 | (5.13) | −7.0 | (8.88) |
| baseline at day 29 | | | | | | | | |

Bilirubin total, μmol/L

| | | | | | | | | |
|---|---|---|---|---|---|---|---|---|
| Baseline | 8.3 | (3.32) | 6.0 | (2.10) | 6.8 | (2.16) | 6.6 | (2.74) |
| Change from | −3.1 | (3.32) | 1.7 | (2.16) | −0.6 | (2.58) | 0.0 | (1.08) |
| baseline at day 29 | | | | | | | | |

Creatinine, μmol/L

| | | | | | | | | |
|---|---|---|---|---|---|---|---|---|
| Baseline | 64.8 | (10.71) | 54.5 | (6.65) | 67.8 | (7.22) | 61.9 | (15.81) |
| Change from | −11.5 | (16.54) | 7.4 | (15.23) | 1.6 | (11.35) | −1.5 | (6.65) |
| baseline at day 29 | | | | | | | | |

Creatine kinase, IU/L

| | | | | | | | | |
|---|---|---|---|---|---|---|---|---|
| Baseline | 67.8 | (21.04) | 47.3 | (10.76) | 95.8 | (50.23) | 205.3 | (262.20) |
| Change from | −4.5 | (35.76) | 16.5 | (27.40) | 39.0 | (129.03) | −68.0 | (215.84) |
| baseline at day 29 | | | | | | | | |

Uric acid, mmol/L

| | | | | | | | | |
|---|---|---|---|---|---|---|---|---|
| Baseline | 0.3 | (0.06) | 0.3 | (0.05) | 0.4 | (0.08) | 0.3 | (0.11) |
| Change from | −0.0 | (0.05) | 0.0 | (0.04) | −0.02 | (0.08) | 0.0 | (0.05) |

baseline at day 29

IU, international units; MAD, multiple ascending dose.

Five participants treated with CT-388 experienced mild TEAEs related to laboratory values (blood creatinine phosphokinase levels increased, n=2; ALT levels increased, n=1; lactate dehydrogenase levels increased, n=1; activated partial thromboplastin time prolonged, n=1); none of these TEAEs were considered related to study drug. There were no hypersensitivity, hyposensitivity, or injection site reactions. There were no significant changes in hematology parameters.

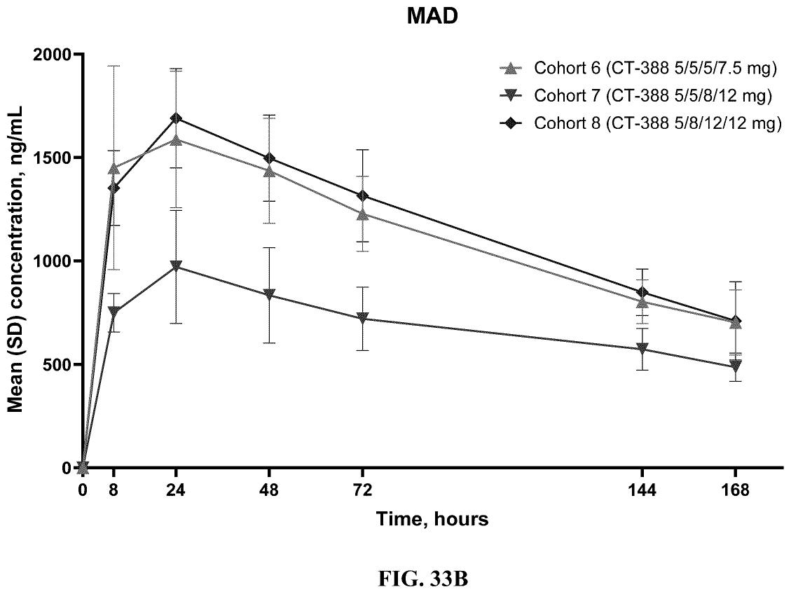

Pharmacokinetics: In the SAD portion of the study, Cₘₐₓ after a single dose ranged from 52.4 to 1165.0 ng/mL across CT-388 doses (Table 31, FIG. 33A). Median tₘₐₓ ranged from 71.9 hours post-dose with 0.5 mg of CT-388 to 24.0-24.1 hours post-dose with 5.0 mg, 6.0 mg, and 7.5 mg of CT-388, demonstrating prolonged absorption from the injection site. Mean t_{1/2} was consistent across the examined dose ranges in the SAD portion of the study (122.4-135.8 hours). Clearance at steady state and apparent volume of distribution increased proportionally to baseline body weight. A dose-proportional increase in Cₘₐₓ and AUC was observed, except with 6.0 mg of CT-388; however, a power model analysis showed no significant departure from dose proportionality.

TABLE 31
PK Parameters after a Single Dose of CT-388

| | tₘₐₓ | C^{max} | AUC_{0-inf} | t_{1/2} | CL/F | Vz/F | MRTₗₐₛₜ |
|---|---|---|---|---|---|---|---|
| Cohort | (h)^{a} | (ng/mL) | (h*ng/mL) | (h) | (mL/h) | (L) | (h) |
| 1 | 71.9 | 52.4 | 13064.7 | 122.4 | 38.5 | 6.3 | 140.2 |
| (0.5 mg) | (48.1, 95.6) | (10.0) | (1012.7) | (21.1) | (3.0) | (0.6) | (6.7) |
| 2 | 36.0 | 216.0 | 46822.4 | 128.2 | 45.1 | 7.9 | 128.3 |
| (2.0 mg) | (24.0, 48.1) | (51.4) | (10539.9) | (19.0) | (13.6) | (1.7) | (2.9) |
| 3 | 24.0 | 831.0 | 137686.8 | 123.2 | 39.2 | 6.9 | 119.5 |
| (5.0 mg) | (8.1, 24.0) | (262.6) | (41137.5) | (11.0) | (12.0) | (2.0) | (2.4) |
| 4 | 24.1 | 521.2 | 96429.3 | 132.8 | 62.3 | 11.4 | 129.0 |
| (6.0 mg) | (24.0, 71.9) | (61.3) | (5029.9) | (16.8) | (3.2) | (1.1) | (8.6) |
| 5 | 24.0 | 1165.0 | 221348.6 | 135.8 | 34.7 | 6.6 | 124.4 |
| (7.5 mg) | (8.0, 24.0) | (109.7) | (36707.8) | (14.6) | (6.4) | (1.3) | (5.2) |

AUC_{0-inf}, area under the concentration-time curve from time zero to infinity; CL/F, oral clearance; Cₘₐₓ, maximum observed concentration; h, hour; MRTₗₐₛₜ, mean residence time from the time of dosing to the time of the last measurable concentration; t_{1/2}, elimination half-life; tₘₐₓ, time to maximum concentration; Vz/F, volume of distribution.
All values are mean (SD) unless otherwise specified.
^{a}Median (minimum, maximum).

In the MAD portion of the study, plasma concentrations after the second weekly CT-388 dose (day 8) followed a similar time course as single doses from the SAD portion (FIG. 33B). Cₘₐₓ ranged from 984.0-1690 ng/mL on day 8, 1420.0-2690.0 ng/mL on day 15, and 1985.0-2891.7 ng/mL on day 22 (Table 32). Dose-proportional increases in Cₘₐₓ and AUC were observed across dose titrations within each cohort; however, Cₘₐₓ and AUC were lower in cohorts 7 and 8 (participants with obesity) than those in cohort 6 (participants with overweight). For example, the mean Cₘₐₓ on day 8 was approximately 40% lower in cohort 7 than the mean Cₘₐₓ in cohort 6, despite both cohorts receiving 5.0 mg of CT-388, suggesting differences in PK profiles for participants with overweight vs obesity. Median Tmax was approximately 24 hours and comparable across dosing regimens on days 8, 15, and 22. The mean t_{1/2} ranged from 110.4 to 197.4 hours, consistent with the t_{1/2} values observed in the SAD arm of the study, supporting once-weekly dosing. A correlation between body weight and clearance at steady state, and between body weight and apparent volume of distribution, was observed in the MAD portion of the study; these are similar to findings in the SAD portion of the study.

TABLE 32
PK Parameters in the Phase 1 MAD Portion of the Study

| | | | | | |
|---|---|---|---|---|---|
| tₘₐₓ | Cₘₐₓ | AUC_{0-inf} | t_{1/2} | CL/F | Vz/F |
| (h)^{a} | (ng/mL) | (h*ng/mL) | (h) | (mL/h) | (L) |

Cohort 6 (5/5/5/7.5 mg)

| | | | | | | |
|---|---|---|---|---|---|---|
| Day 8 | 16.0 | 1680.0 | NA | 115.0 | 27.0 | 4.5 |
| | (8.0, 24.0) | (362.8) | | (19.3) | (5.3) | (1.2) |
| Day 15 | 24.0 | 1765.0 | NA | 125.3 | 37.8 | 4.7 |
| | (8.0, 24.1) | (371.7) | | (28.6) | (8.2) | (2.2) |
| Day 22 | 24.0 | 2288.3 | 493144.1 | 151.5 | 28.4 | 6.2 |
| | (8.0, 48.0) | (362.4) | (106611.1) | (17.1) | (4.8) | (1.0) |

Cohort 7 (5/5/8/12 mg)

| | | | | | | |
|---|---|---|---|---|---|---|
| Day 8 | 24.0 | 984.0 | NA | 197.4 | 44.0 | 12.9 |
| | (24.0, 48.0) | (261.6) | | (72.8) | (8.4) | (6.4) |
| Day 15 | 24.0 | 1420.0 | NA | 125.9 | 49.2 | 8.8 |
| | (24.0, 48.0) | (374.0) | | (9.2) | (11.0) | (2.4) |
| Day 22 | 24.0 | 1985.0 | 399911.4 | 130.3 | 52.8 | 9.9 |
| | (24.0, 48.0) | (476.7) | (101134.7) | (10.2) | (11.2) | (2.4) |

Cohort 8 (5/8/12/12 mg)

| | | | | | | |
|---|---|---|---|---|---|---|
| Day 8 | 24.0 | 1690.0 | NA | 118.3 | 40.9 | 7.0 |
| | (24.0, 24.0) | (240.4) | | (36.9) | (5.5) | (2.4) |
| Day 15 | 24.0 | 2690.0 | NA | 110.4 | 37.8 | 6.0 |
| | (36.0, 48.0) | (482.9) | | (36.2) | (8.2) | (2.4) |
| Day 22 | 24.0 | 2891.7 | 666796.9 | 131.3 | 33.4 | 6.2 |
| | (8.1, 72.0) | (671.5) | (185266.0) | (20.3) | (7.4) | (0.7) |

AUC_{0-inf}, area under the concentration-time curve from time zero to infinity; CL/F, oral clearance; Cₘₐₓ, maximum observed concentration; h, hour; NA, not assessed; t_{1/2}, elimination half-life; tₘₐₓ, time to maximum concentration; Vz/F, volume of distribution.
All values are mean (SD) unless otherwise specified.
^{a}Median (minimum, maximum).

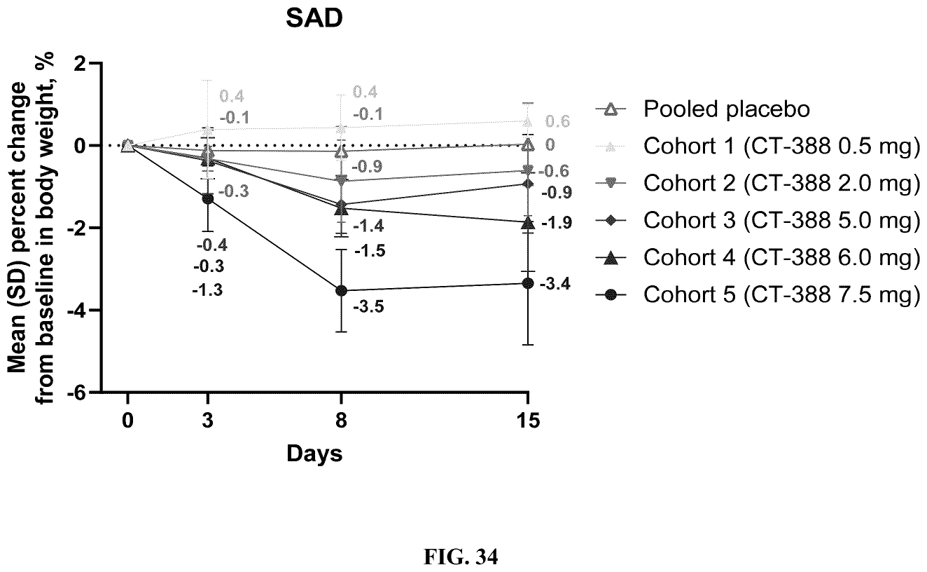

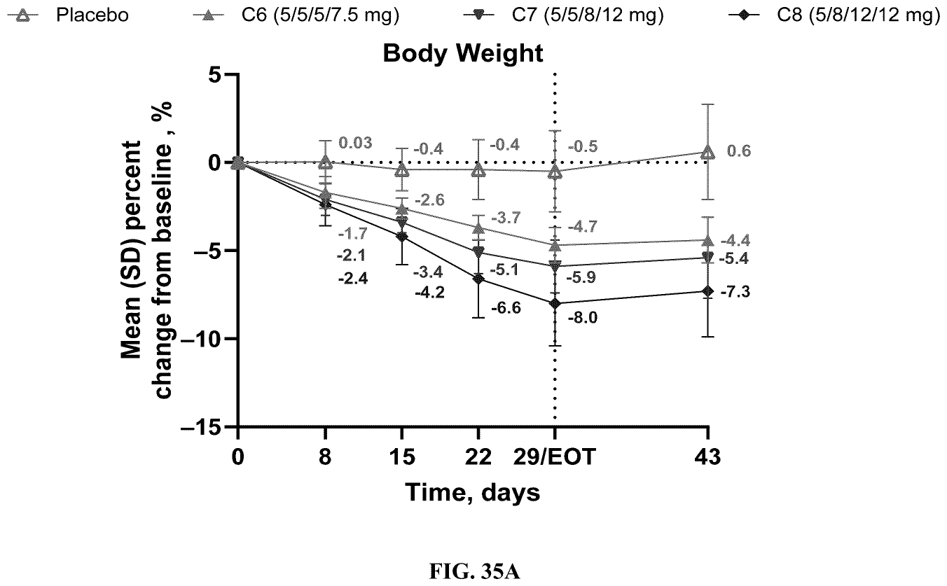

Pharmacodynamic effects of CT-388 on body weight: CT-388 treatment resulted in substantial, dose- and time-dependent decreases in body weight at most dose levels tested. In the SAD portion of the study, a single dose of CT-388 at doses ≥2 mg reduced body weight, with doses ≥5 mg reaching clinically significant weight loss vs placebo (FIG. 34); at day 8, the placebo-adjusted least squares mean (9500 CI) percent change from baseline in body weight was 0.8% (−0.1, 1.7) with 0.5 mg of CT-388, −0.8% (−1.6, 0.1) with 2 mg of CT-388, −1.3% (−2.2, −0.4) with 5 mg of CT-388, −1.6% (−2.5, −0.7) with 6 mg of CT-388, and −3.1% (−4.0, −2.2) with 7.5 mg of CT-388. For short-term duration of treatment (1 dose), 1.0% decrease within 1 week is clinically significant. In general, for longer treatment durations, ≥5% body weight reduction is considered to be clinically meaningful, and ≥10% is considered to represent a disease-modifying effect.

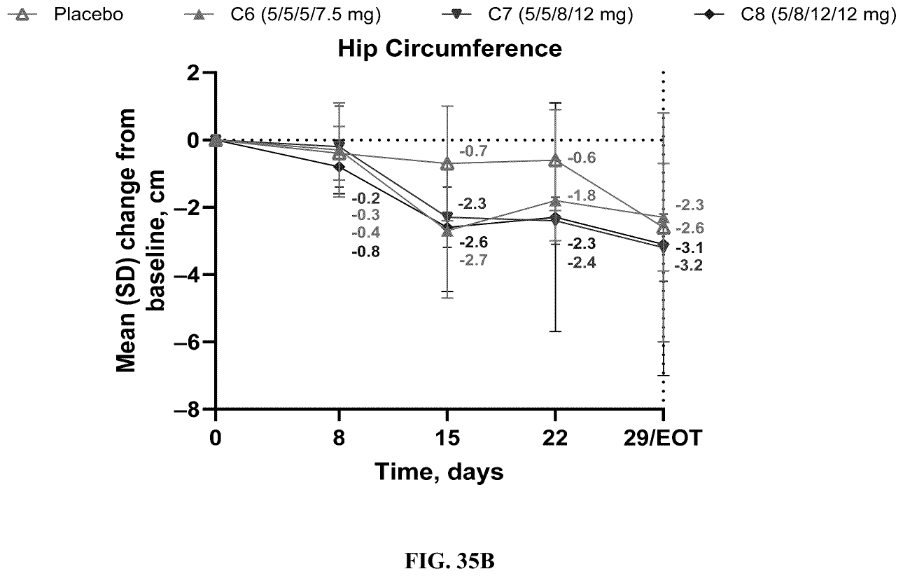

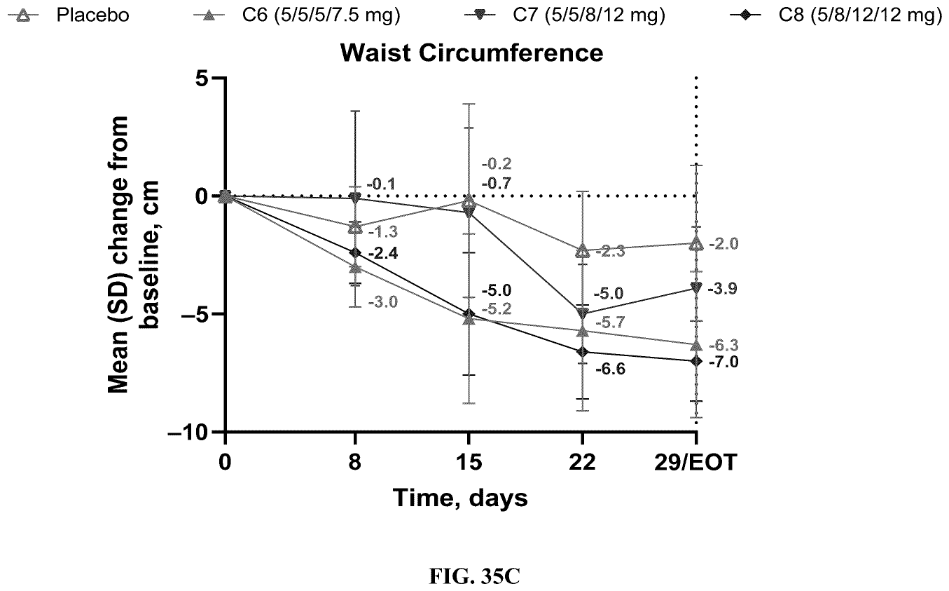

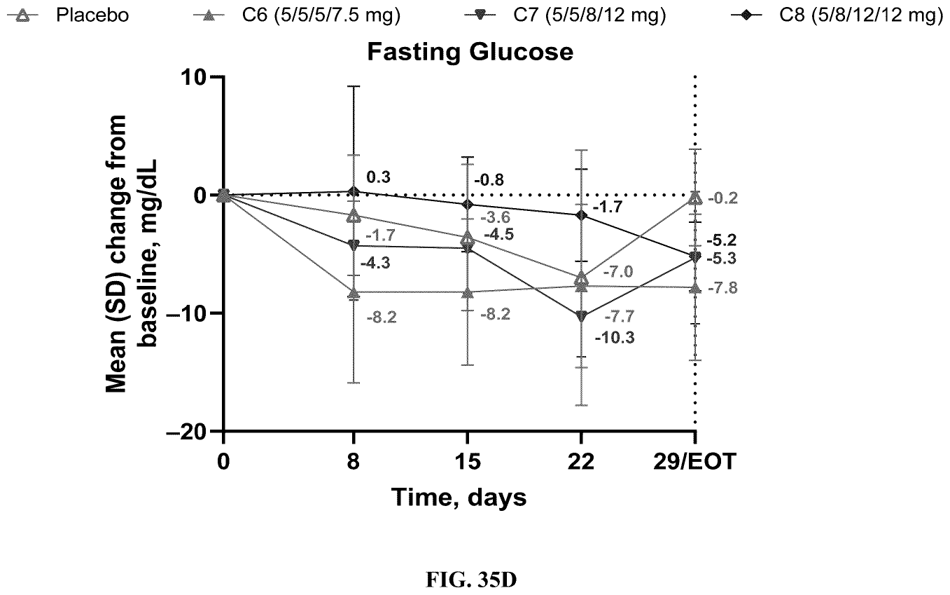

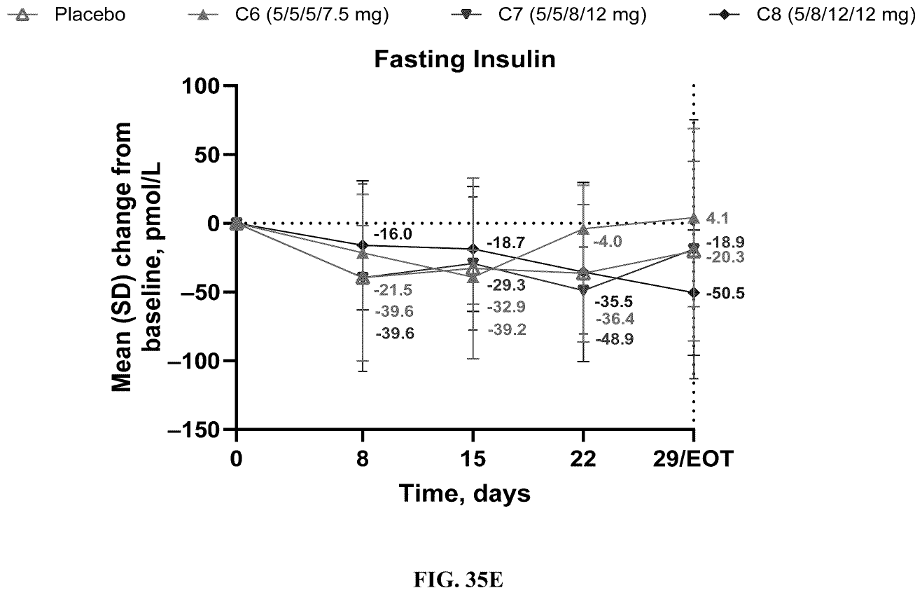

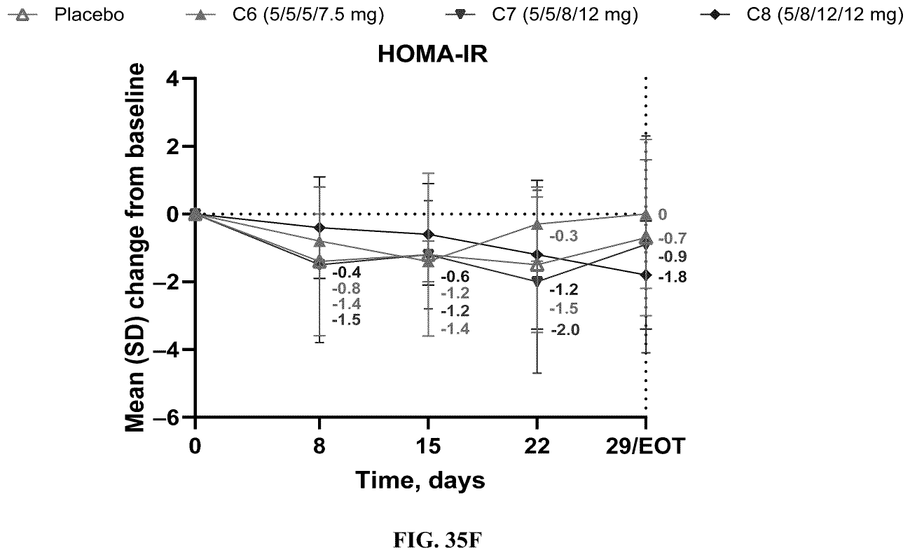

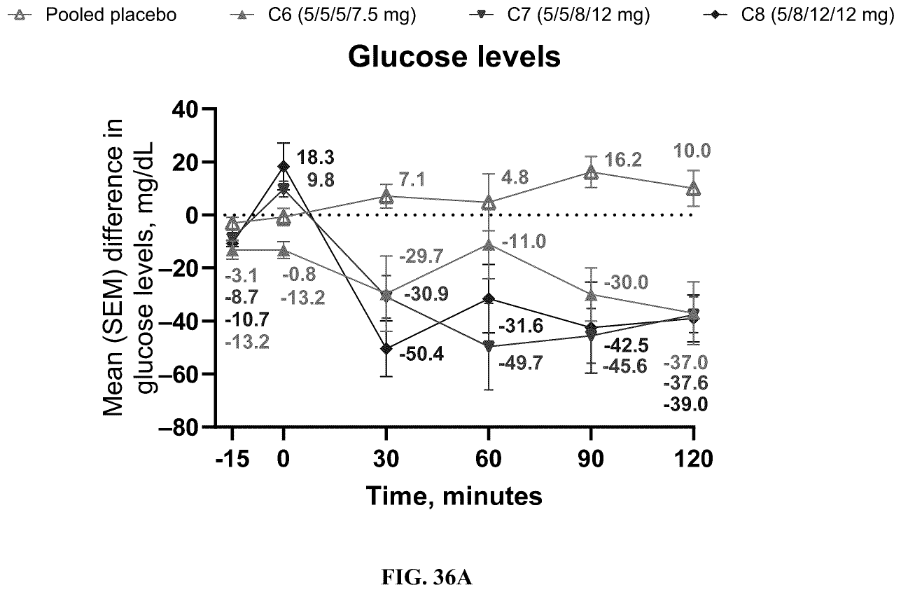

In the MAD portion of the study, CT-388 treatment yielded clinically meaningful dose-dependent weight loss compared to placebo after four weeks of treatment. Clinically significant weight loss was observed after the first dose of CT-388 and progressed with additional doses. There were no weight loss plateaus, and weight loss was maintained for up to 2 weeks beyond the cessation of treatment. Mean percent change from baseline in body weight is presented in FIG. 35A; the placebo-adjusted least squares mean (95% CI) percent change from baseline in body weight at day 29 among participants treated with CT-388 was −4.0% (−5.9, −2.0) in cohort 6, −5.9% (−7.9, −3.8) in cohort 7, and −7.6% (−9.6, −5.7) in cohort 8. Clinically meaningful decreases in hip and waist circumference were also observed after CT-388 treatment (FIGS. 35B-C).

Pharmacodynamic effects of CT-388 on glucose homeostasis: In the MAD portion of the study, reductions in fasting glucose levels at day 29 were observed in all three CT-388 treatment groups but not the pooled placebo group; mean change from baseline ranged from −5.2 to −7.8 mg/dL across CT-388 treatment groups compared to −0.2 mg/dL with placebo (FIG. 35D). Notably, improvements were greater and occurred more quickly among participants treated with CT-388 with higher baseline fasting glucose levels (i.e., cohort 7) than those with lower baseline levels (i.e., cohort 8). Changes in fasting insulin levels and HOMA-IR varied between treatment groups (FIGS. 35E-F).

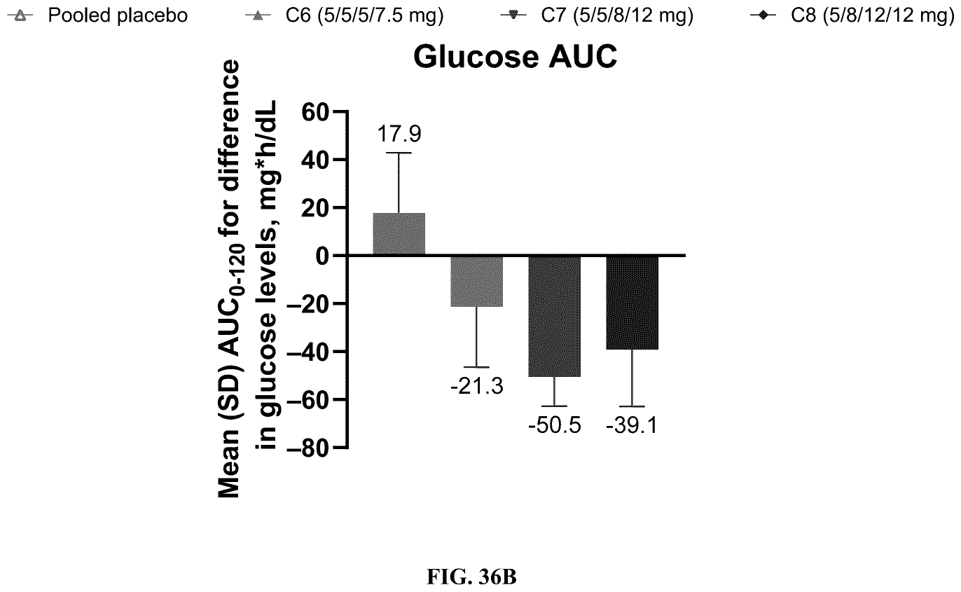

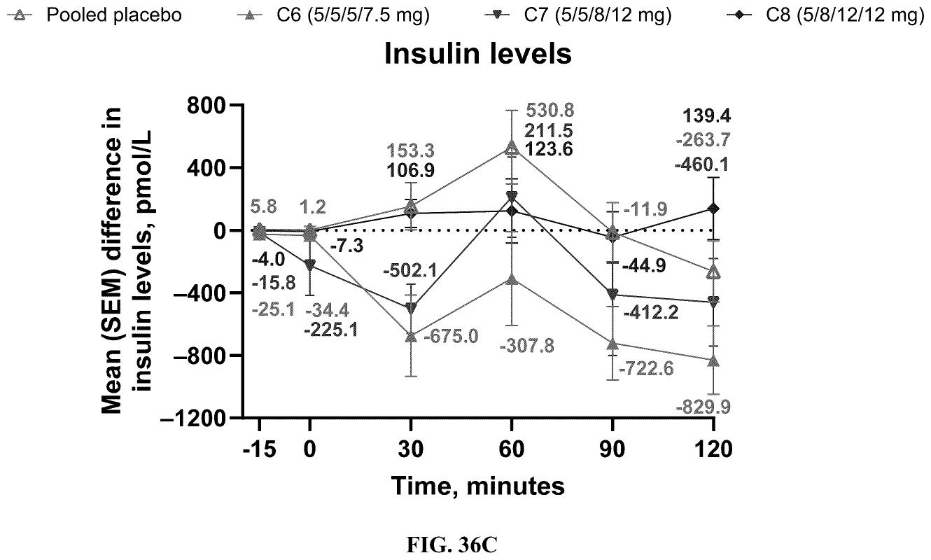

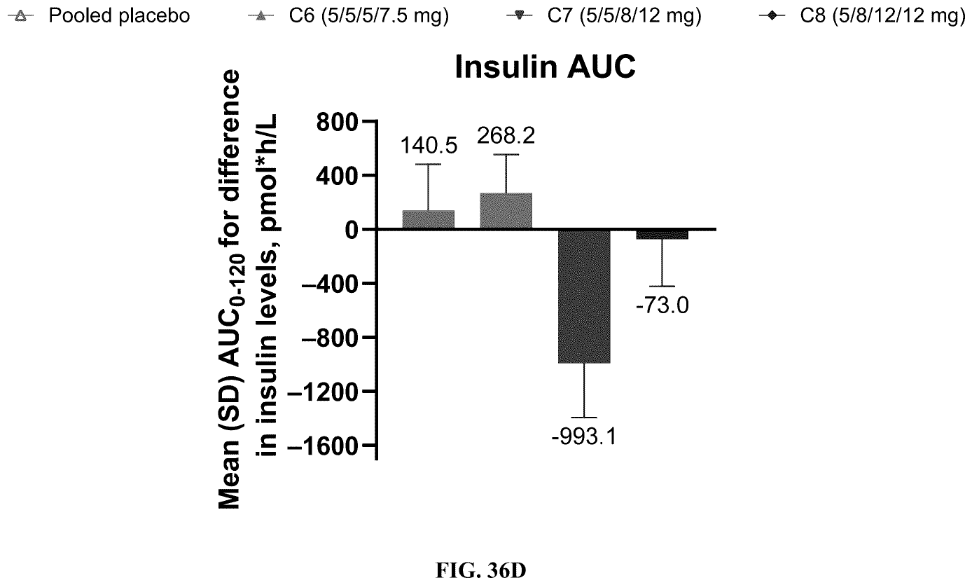

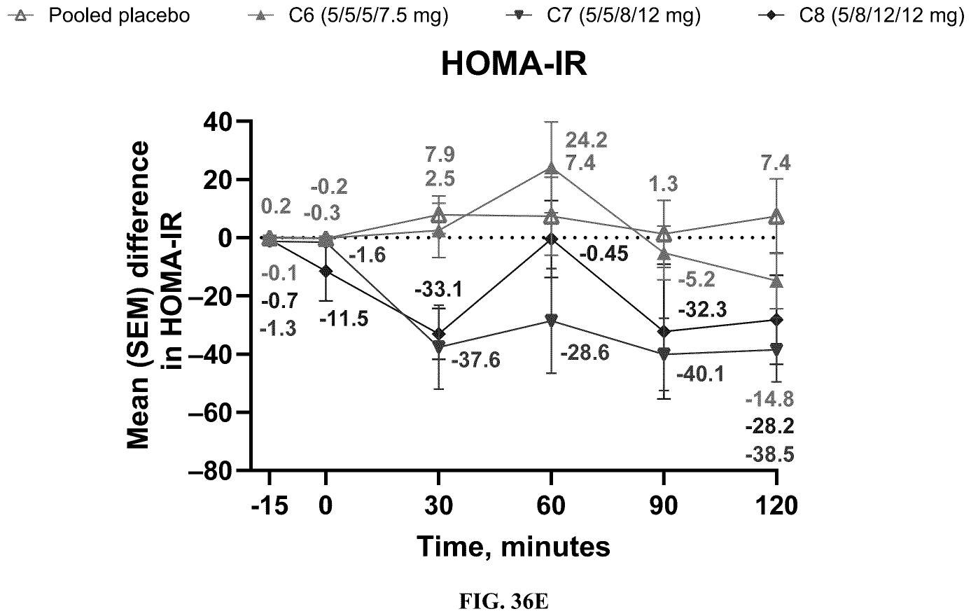

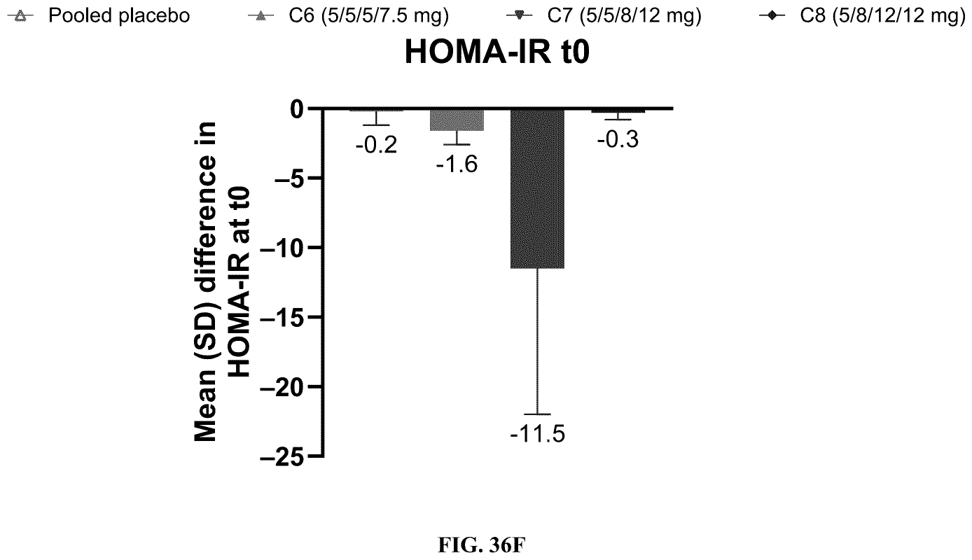

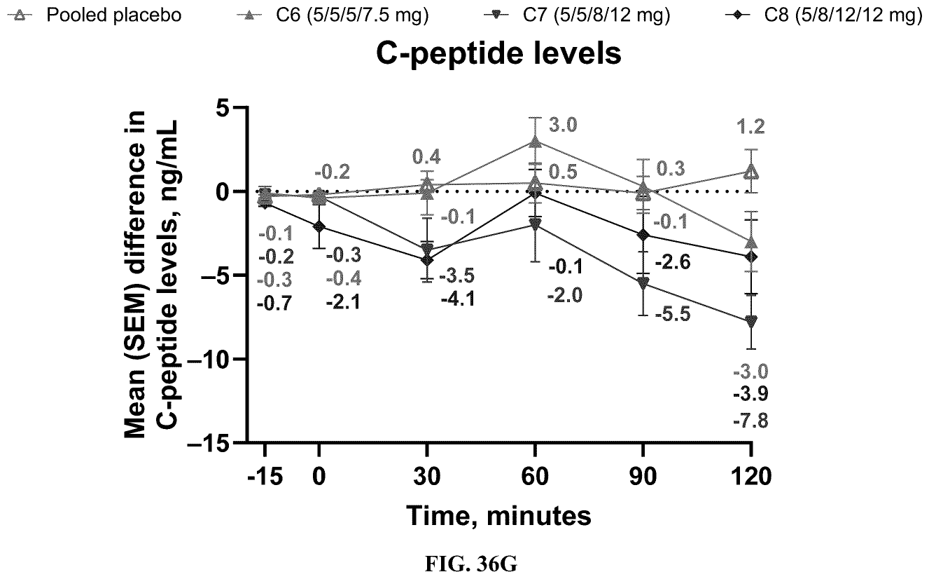

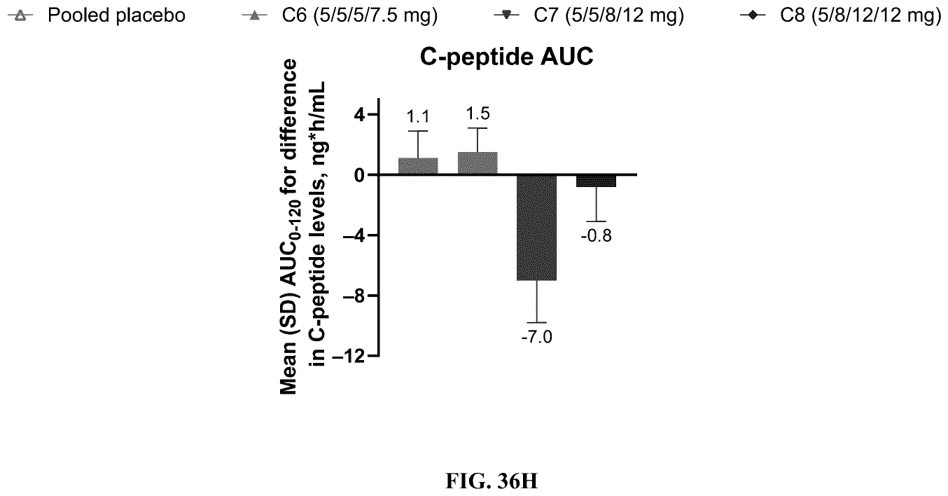

Results from an OGTT showed generally greater improvements from baseline to day 23 in glucose tolerance in all CT-388 treatment arms vs placebo, especially in participants with obesity who had elevated fasting glucose levels and HOMA-IR at baseline (i.e., cohort 7). After 23 days of CT-388 treatment, mean glucose levels during an OGTT were substantially lower vs baseline in all CT-388-dose groups (mean difference between day −1 and day 23: CT-388 dose groups, −37.0 to −39.0; placebo group, 10.0; FIG. 36A); the decrease in AUC_{0-120 min} glucose from baseline showed a similar pattern (FIG. 36B). Decreased insulin, HOMA-IR, and C-peptide were also observed during OGTT among CT-388-treated participants vs placebo (FIGS. 36C-H), indicating improved insulin sensitivity. Similar trends were observed for glucagon levels (data not shown).

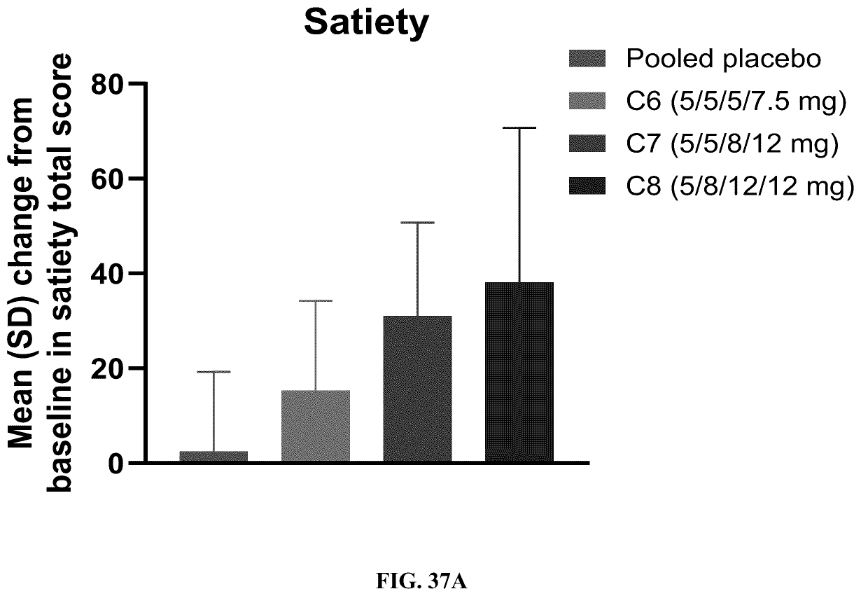

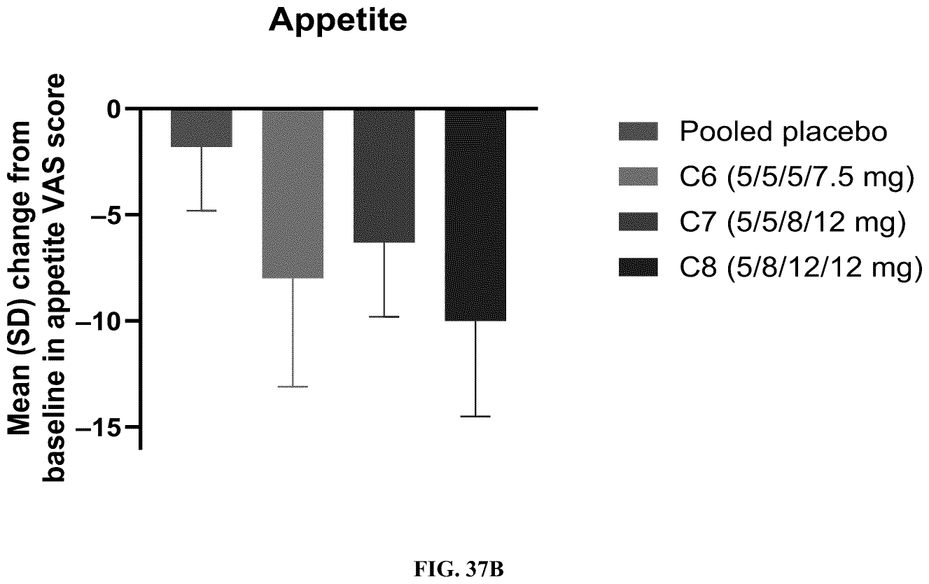

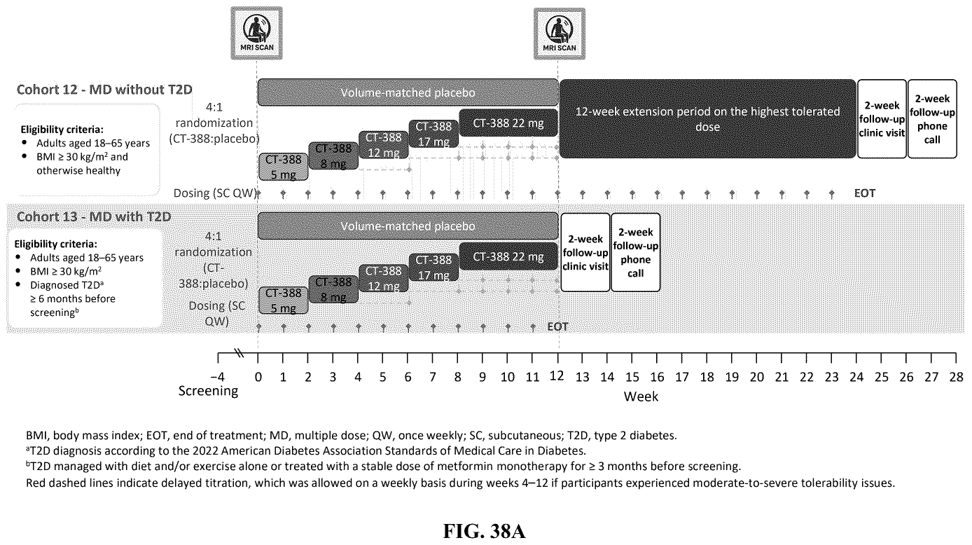

Pharmacodynamic effects of CT-388 on satiety and appetite: In the MAD portion of the study, dose-dependent improvements in satiety and appetite were observed with CT-388 treatment vs placebo. Satiety total scores were substantially increased (FIG. 37A) and the visual analog scale scores for appetite were decreased (FIG. 37B) at day 29 among participants who received CT-388 compared with placebo.

### C. Discussion

CT-388, a unimolecular dual GLP-1/GIP receptor agonist, was identified through chemotype evolution. CT-388 was found to potently activate both the GLP-1 and GIP receptors, with potency skewed toward the GLP-1 receptor. In contrast, receptor activation with tirzepatide has been shown to be skewed toward the GIP receptor (Coskun et al., Mol Metab. (2018) 18:3-14). Furthermore, CT-388 was also demonstrated to have complete or substantial signaling bias in engaging cAMP signaling over β-arrestin recruitment and internalization at both the GLP-1 and GIP receptors. While tirzepatide shows cAMP signaling bias on the GLP-1 receptor, it has been shown to be unbiased on the GIP receptor and to drive GIP receptor internalization (Willard et al., JCI Insight. (2020) 5:e140532). Bias for cAMP signaling with GLP-1 receptor agonists has been observed to prolong the PD effects and may improve efficacy (Guo et al., Mol Metab. (2023) 75:101762; Hinds et al., Diabetes Obes Metab. (2024) 26:65-77; Lucey et al., Mol Metab. (2020) 37:100991; Jones et al., Nat Commun. (2018) 9:1602; Pickford et al., Br J Pharmacol. (2020) 177:3905-23; Rodriguez et al., Cell Rep Med. (2025) 6:102156). That being said, the mechanism by which signaling bias influences prolonged effects or improved efficacy remains unclear.

CT-388 was pharmacologically characterized first by testing its effects on GSIS given that both native GLP-1 and GIP stimulate GSIS physiologically. Using primary human beta cells (EndoC-PH5), CT-388 was shown to directly and dose-dependently enhanced GSIS. This GSIS enhancement was confirmed in vivo in cynomolgus monkeys, where plasma insulin excursion was observed to be dose-dependently increased by single-dose CT-388 treatment after an intravenous bolus glucose challenge. Correspondingly, lower plasma glucose concentrations were observed as insulin enhanced the elimination of glucose from the circulation. The effects of CT-388 on plasma glucose were further demonstrated in mice, with both acute (ipGTT in lean mice) and chronic (MC4RKO and GAN-DIO mice) administration; these observations were also translated into humans. Significant improvements in glucose excursion were observed during the OGTT in all MAD cohorts treated with CT-388 for 23 days. Unlike findings on the ivGTT in monkeys, insulin or C-peptide concentrations were not found to increase, but rather were generally found to decrease with CT-388 treatment. One potential explanation for this observation is that CT-388, like native incretins or GLP-1 receptor agonists (van Can et al., Int J Obes (Lond). (2014) 38:784-793), was found to effectively reduce circulating glucose; therefore, increased insulin levels were not observed. HOMA-IR was reduced among cohorts treated with CT-388 during the OGTT, suggesting improved insulin sensitivity which may also contribute to the observed effective glucose regulation. The role of CT-388 in glucose homeostasis was further confirmed by improvements in fasting glucose levels at day 29 among participants treated with CT-388 who had higher baseline fasting glucose levels (i.e., cohort 7).

Consistent with known effects of incretins, CT-388 significantly reduced food intake in both lean and obese mice. Although food intake was not evaluated in the clinical trial, satiety and appetite assessments were conducted using satiety total scores and visual analog scale scores for appetite during the MAD portion of the study. On day 29, satiety was dose-dependently and substantially increased with CT-388 treatment, and appetite was suppressed in all CT-388 cohorts.

In association with reduced food intake, approximately 20-25% body weight reduction was consistently observed with a modest dose of CT-388 in multiple mouse models of obesity, demonstrating robust and durable efficacy of CT-388 in obesity with different etiology (diet induced or genetic mutation). Extending these preclinical observations, a dose-dependent reduction in body weight was observed with CT-388 during the SAD trial in overweight or obese participants. Single-dose treatments of 7.5 mg of CT-388 led to a 3.1% placebo-adjusted least squares mean decrease in body weight in just eight days. During the MAD portion of the study, progressive body weight loss was observed in all CT-388 cohorts. In cohort 8 (BMI, 34.8±4.0 kg/m²), participants treated with CT-388 at the 5/8/12/12 mg titration scheme had a 7.6% (7.3 kg) placebo-adjusted least squares mean decrease in body weight at day 29.

These data suggest that CT-388 treatment could lead to weight loss and improvements in weight-related comorbidities. The effects of CT-388 on a well-established MASH model (GAN-DIO mice) were evaluated. In this model, CT-388 significantly improved hepatic steatosis and reduced hepatocyte ballooning degeneration. Additionally, CT-388 significantly improved liver enzymes as demonstrated by substantial reductions in ALT and AST. The PK profiles observed in the SAD and MAD cohorts generally showed a dose-proportional increase in Cₘₐₓ and AUC in each cohort, and t_{1/2} was supportive of once-weekly dosing with CT-388.

The MAD portion of the study was designed to evaluate the safety profile of efficacious starting doses with rapid up-titrations. CT-388 was generally well tolerated in participants with overweight and obesity, and no unexpected safety findings were reported. Although most participants experienced GI-related adverse events, the majority were mild to moderate in severity and there were no SAEs or clinically meaningful changes in vital signs or laboratory values. In the SAD portion of the study, one participant who received 6.0 mg of CT-388 discontinued due to vomiting, mild diarrhea, and mild abdominal pain, considered possibly related to study drug; in the MAD portion of the study, no participants discontinued due to TEAEs. While severity of TEAEs and the incidence of GI-related TEAEs varied across cohorts, these differences may be attributed to baseline body weight; per the eligibility criteria mean body weight and BMI at baseline were lower in cohort 6 than in cohorts 7 and 8. These findings are consistent with other studies reporting an inverse relationship between baseline BMI and the incidence of GI-related TEAEs (Verma et al., J Can Assoc Gastroenterol. (2025) 8(Supplement 1):i31-i32). Lower starting doses may also be warranted for people with overweight; follow-up studies evaluating lower starting doses (e.g., 2 mg with at least four weeks for up-titration) in participants with overweight and obesity are planned to improve the tolerability profile in both populations.

The results herein show strong translatability across species, and the high degree of consistent clinical responses in participants who received CT-388 compared with placebo demonstrate the robust pharmacodynamic activity of the CT-388. Despite the SAD and MAD studies being conducted in different geographies and ethnicities, a single 5 mg dose of CT-388 resulted in a plasma exposure range and yielded a similar degree of weight loss across both SAD and MAD studies, indicating the drug effects are likely to be minimally affected by geographic, racial, or ethnic differences.

In conclusion, CT-388, a cAMP-biased dual GLP-1/GIP receptor agonist, was demonstrated to effectively regulate glucose homeostasis and reduce body weight in multiple models of obesity and different preclinical species, including rodents and non-human primates. These preclinical observations translated into humans, as demonstrated in the phase 1 clinical trial in participants with overweight or obesity, with up to an approximately 8% reduction in body weight after four weeks of CT-388 treatment. Furthermore, the phase 1 results showed a favorable safety/tolerability profile, as well as PK consistency with once-weekly dosing. Together, these data support the continued clinical development of CT-388 for chronic weight management, glycemic control, as well as in other obesity-related comorbidities in adults with overweight or obesity.

### Example 8: Effect of CT-388 on Liver-Related Parameters in Participants with Obesity With and Without T2D

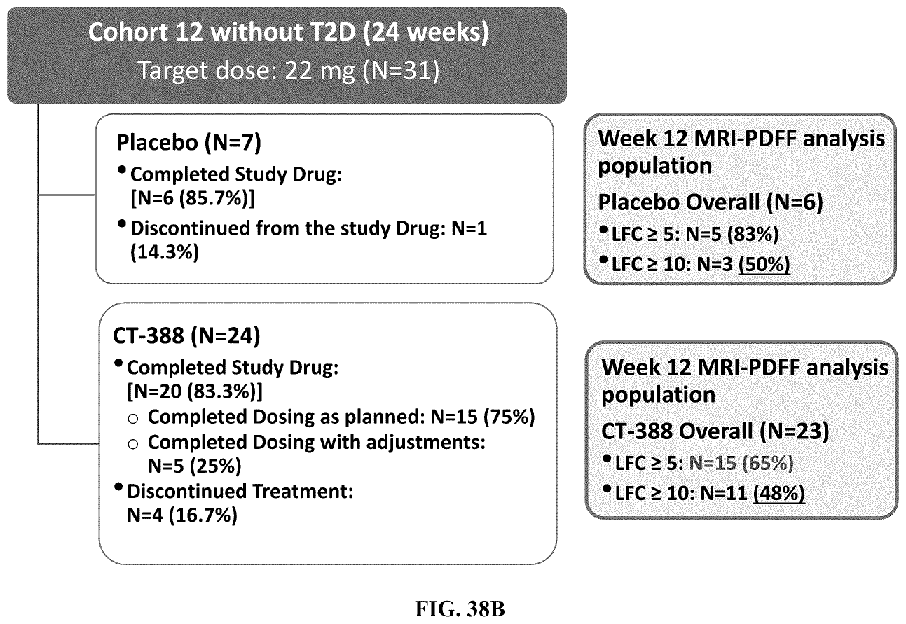

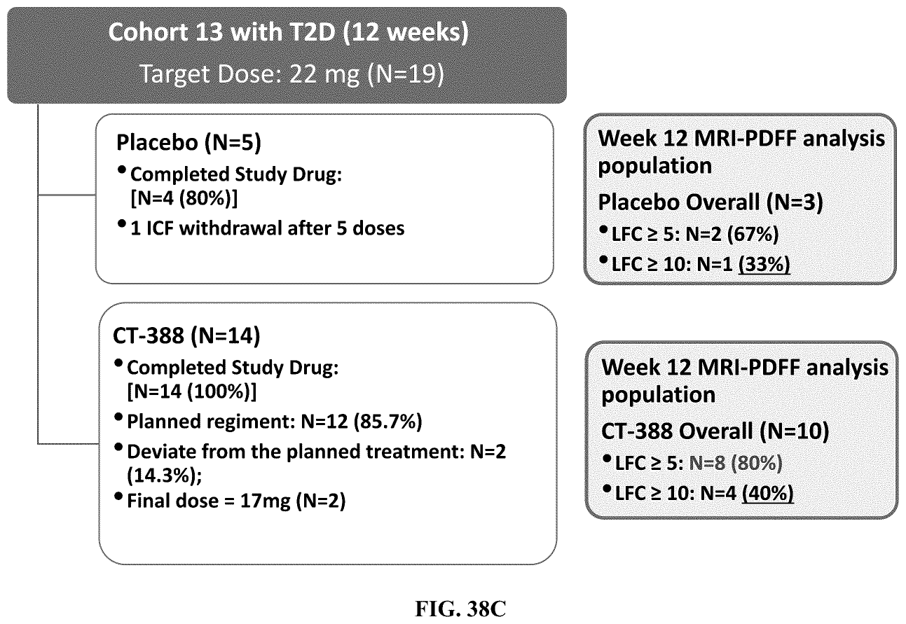

This Example describes analyses performed using magnetic resonance imaging protein density fat fraction (PDFF) to evaluate the change in liver fat content (LFC) in Cohort 12 and Cohort 13 after 12 weeks of treatment with CT-388 up to a target dose of 22 mg or placebo. A 4:1 randomization ratio was used (4 participants treated with 22 mg of CT-388 (via up-titration) per participant treated with placebo) (FIG. 38A). Percentage changes in MRI-PDFF from baseline and changes in cT1 from baseline were analyzed using a mixed model for repeated measures.

Cohort 12 included 31 otherwise healthy adults with obesity (BMI≥30.0 kg/m²). MRI was performed at baseline (BL) and weeks 12 and 24 to evaluate liver fat content (LFC) via MRI-PDFF and inflammation via cT1. 29 patients with complete MRI data (i.e., MRI measures taken at both timepoints; 23 of which were treated with CT-388 and six of which were treated with placebo) were analyzed for percent change in liver fat content (LFC) and absolute reduction in cT1 from baseline using a mixed model for repeated measures. FIG. 38B shows the subject dispositions for the Cohort 12 MRI-PDFF analysis population. 75% of participants were dosed per planned path and completed treatment on 22 mg. 25% had dose down-titration and completed treatment on 12 mg. There were no participants who completed treatment on 17 mg.

Cohort 13 included 19 otherwise healthy adults with obesity who were diagnosed with T2D≥6 months before screening (BMI≥30.0 kg/m²). T2D was diagnosed according to the 2022 American Diabetes Association Standards of Medical Care in Diabetes, and was managed with diet and/or exercise alone or treated with a stable dose of metformin monotherapy for ≥3 months before screening (American Diabetes Association (ADA), Diabetes Care (2025) 48:5207-38). MRI was performed at baseline (BL) and week 12 to evaluate LFC via MRI-PDFF and inflammation via cT1. 19 patients with complete MRI data (i.e., MRI measures taken at both timepoints; 14 of which were treated with CT-388 and five of which were treated with placebo) were analyzed for percent change in liver fat content (LFC) and absolute reduction in cT1 from baseline using a mixed model for repeated measures. FIG. 38C shows the subject dispositions for the Cohort 13 MRI-PDFF analysis population. 85.7% of participants were dosed per planned path and completed treatment on 22 mg. 14.3% had dose down-titration and completed treatment on 17 mg.

The demographic and baseline characteristics of the MRI analysis population in the CT-388-101 MD (Cohorts 12 and 13) are summarized in Table 33. Participants with T2D were generally slightly older, with lower body weight and BMI at baseline than those without T2D. Overall, 71% (30/42) of participants had baseline LFC≥5% (indicative of liver steatosis) and 45% (19/42) had baseline LFC≥10% (indicative of severe liver steatosis).

Participants treated with CT-388 in Cohort 12 (otherwise healthy adults with obesity) were mostly females (65%), with a median age of29 yrs [range: 21-51], BMI of39 kg/m² [range: 30-52], baseline MRI-PDFF Liver Fat Content (LFC) of 8.5% [range: 1.7-30.7%] and M of 851 ms [756-985]. Placebo participants were males (100%), with a median age of29 years [range: 21-50], BMI of236 kg/m² [range: 31-40], MRI-PDFF LFC 9(5% [range: 3.4-13.3%] and cT1 of804 ms [range: 725-875].

Participants treated with CT-388 in Cohort 13 (otherwise healthy adults with obesity who were diagnosed with T2D≥6 months before screening) were 508 females, with a median age of47 years [range: 30-61], BMI of33 kg/m² [range: 30-90], baseline MRI-PDFF Liver Fat Content (LFC) of 9.5% [range: 2.3-20.5%] and cT1 of 834 ms [range: 718-942]. Placebo participants were females (100%), with a median age of 43 years [range: 35-49], BMI of 38 kg/m² [range: 31-39], MIRI-PDFF LFC 8.0% [range: 4.9-15.5%] and cT of 853 ms [range: 848-898].

TABLE 33
Demographic and Baseline Characteristics, Week 12 Liver MRI Analysis Population

| | | | Cohort 12 | | | | Cohort 13 | |
|---|---|---|---|---|---|---|---|---|
| | Cohort 12 | | Planned | | Cohort 13 | | Planned | |
| | Pooled | | Target CT-388 | | Pooled | | Target CT -388 | |
| | Placebo | | Dose (22 mg)^{b} | | Placebo | | Dose (22 mg)^{b} | |
| | (N = 6) | | (N = 23) | | (N = 3) | | (N = 10) | |
| Age (years), Median | 29 | (21-50) | 29 | (21-51) | 43 | (35-49) | 47 | (30-61) |
| (range) | | | | | | | | |
| Sex, females n (%) | 0 | (0) | 15 | (65) | 3 | (100) | 5 | (50) |
| Hispanic or Latino, n (%) | 6 | (100) | 23 | (100) | 3 | (100) | 10 | (100) |
| Body Weight (kg), Median | 100 | (87-115) | 106 | (77-143) | 96 | (78-96) | 87 | (74-111) |
| (range) | | | | | | | | |
| BMI (kg/m²), Median | 36 | (31-40) | 39 | (30-52) | 38 | (31-39) | 33 | (30-39) |
| (range) | | | | | | | | |
| HbA1c (%), Median | 5.5 | (5.0-5.9) | 5.4 | (4.6-5.9) | 10.0 | (8.8-10.0) | 8.15 | (7.0-10.1) |
| (range) | | | | | | | | |
| LFC (%), Median (range)^{a} | 9.5 | (3.4-13.3) | 8.5 | (1.7-30.7) | 8.0 | (4.9-15.5) | 9.5 | (2.3-20.5) |
| LFC ≥5% at baseline | n = 5 | | n = 15 | | n = 2 | | n = 8 | |
| LFC (%), Median (range) | 10.5 (5.6-13.3) | | 10.8 (5.0-30.7) | | 11.8 (8.0-15.5) | | 10.5 (6.6-20.5) | |
| LFC ≥10% at baseline | n = 3 | | n = 11 | | n = 1 | | n = 4 | |
| LFC (%), Median (range) | 12.0 (10.5-13.3) | | 14.2 (10.0-30.7) | | 15.5 (N/A) | | 14.7 (11.2-20.5) | |
| cT1, (ms), Median (range)^{b} | 804 | (725-875) | 851 | (756-985) | 853 | (848-898) | 834 | (718-942) |
| ALT, (U/L), Median | 31 | (23-58) | 24 | (8-59) | 36 | (18-105) | 21 | (14-79) |
| (range)^{c} | | | | | | | | |
| AST, (U/L), Median | 25 | (19-36) | 22 | (14-40) | 24 | (15-68) | 18 | (13-41) |
| (range)^{d} | | | | | | | | |
| ProC3, (ng/mL), Median | 48 | (28-97) | 52 | (27-88) | 36 | (27-37) | 40 | (21-68) |

(range)^{e}

ALT = alanine aminotransferase, AST = aspartate aminotransferase, BMI = body mass index, cT1 = iron corrected T1, LFC = liver fat content, MASH = metabolic dysfunction-associated steatohepatitis, MASLD = metabolic dysfunction-associated steatotic liver disease, MRI = magnetic resonance imaging, PDFF = proton density fat fraction, ProC3 = N-terminal type III collagen propeptide.
Baseline is defined as the last observation prior to the first dose of the study drug.
^{a}LFC was measured using MDF-PDFF, which accurately quantifies fat content within liver tissue (Tang et al., Radiology. (2015) 274: 416-25). A healthy LFC is <5%; LFC ≥5% indicates MASLD; and LFC ≥10% may indicate severe liver steatosis (Andersson et al., Clin Gastroenterol Hepatol. (2022) 20: 2451-61).
^{b}cT1 is a marker of liver fibrosis and inflammation (Andersson et al., Clin Gastroenterol Hepatol. (2022) 20: 2451-61; Alkhouri et al., J Hepatol. (2025) 82: 438-45). A healthy cT1 reference range is ~600-800 ms; cT1 ≥800 ms is predictive of MASH; and cT1 ≥875 ms indicates advanced MASH (Andersson et al., Clin Gastroenterol Hepatol. (2022) 20: 2451-61; Mojtahed et al., Abdom Radiol (NY). (2019) 44: 72-84).
^{c}Healthy range for ALT is: ~29-33 U/L (males), ~19-25 U/L (females) (Kwo et al., Am J Gastroenterol. (2017) 112: 18-35).
^{d}Healthy upper reference limit for AST is ~18 U/L (Erhardtsen et al., JHEP Rep. (2021) 3: 100317).
^{e}Healthy reference range for ProC3 is 6.1-14.7 ng/mL (Erhardtsen et al., JHEP Rep. (2021) 3: 100317).

### Liver-Related Imaging Parameters

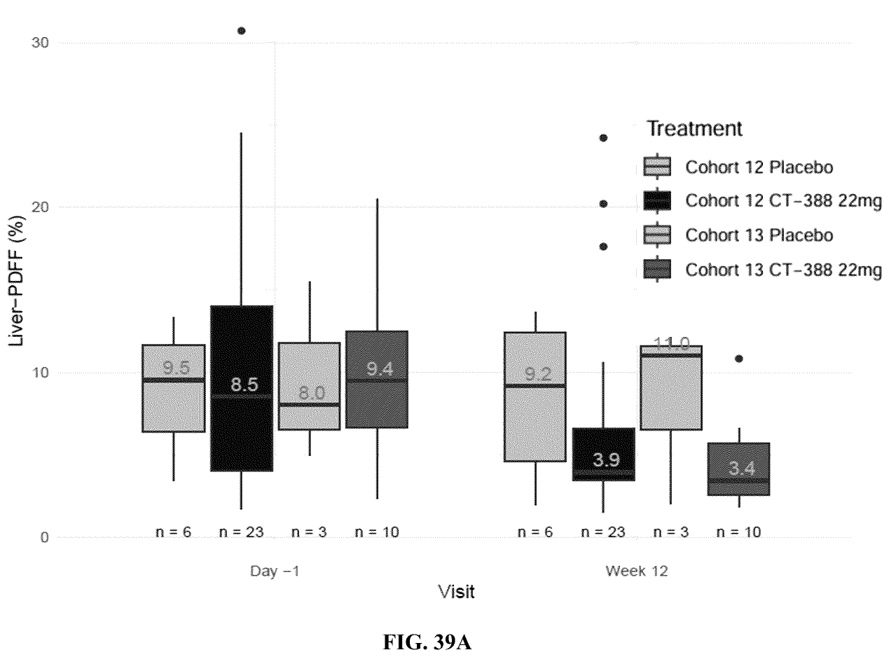

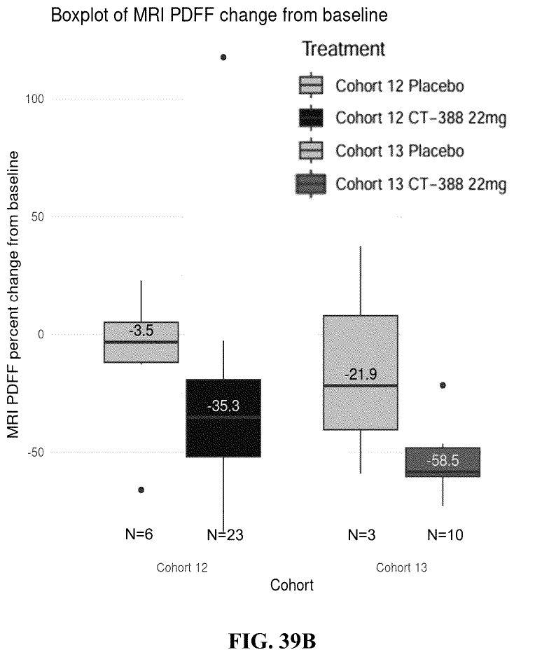

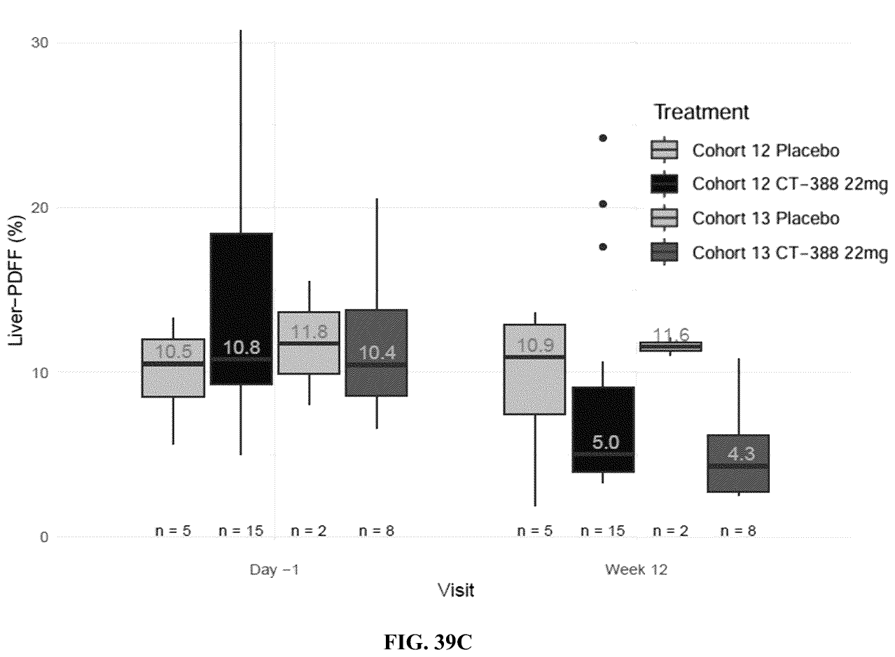

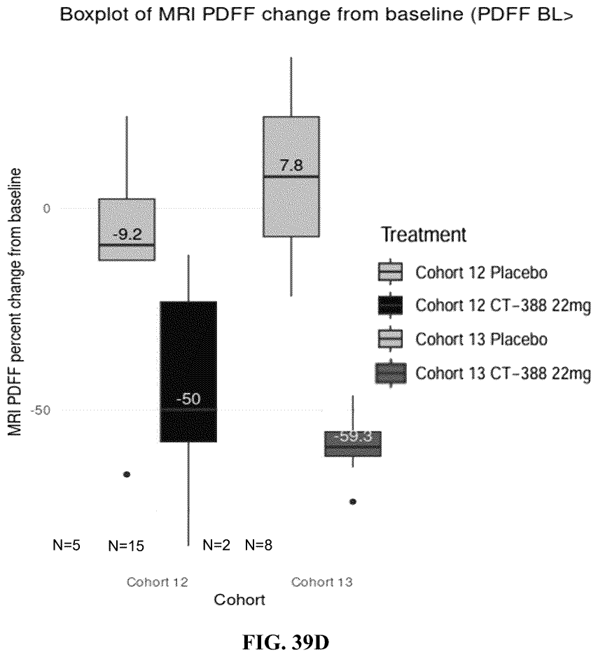

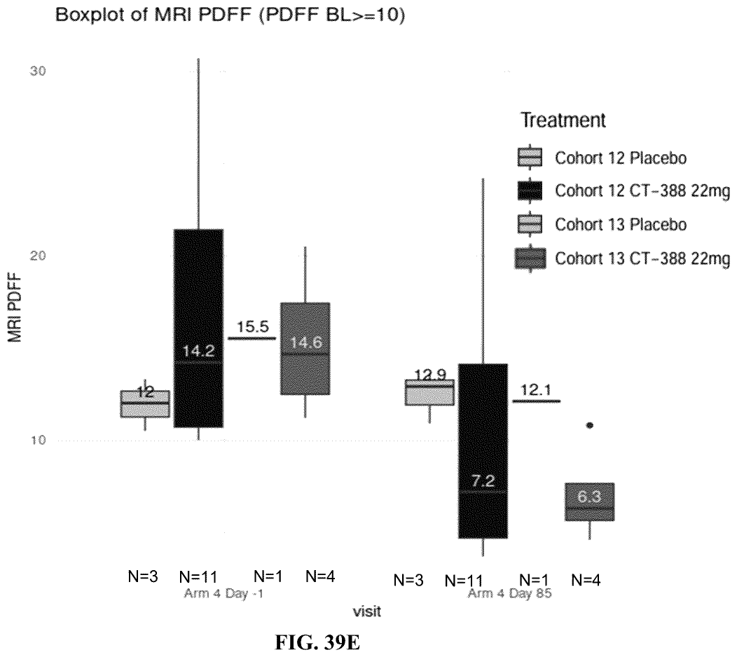

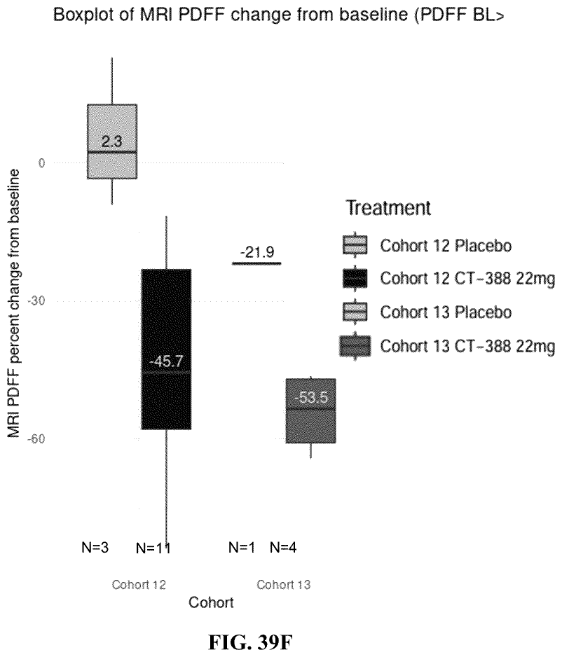

CT-388 resulted in significant reduction in liver steatosis in participants with obesity. After 12 weeks, in Cohort 12, the median reduction in liver fat content was 35.3% (range: −66.1, 22.9), whereas in Cohort 13, the median reduction in liver fat content was 58.5% (range: −72.8, 21.7) (FIGS. 39A and 39B; Table 34).

TABLE 34
Analysis of Percent Change from Baseline in Median
Liver PDFF in Cohort 12 and 13 Participants

| | | Cohort 12 | | Cohort 13 |
|---|---|---|---|---|
| | Cohort 12 | Planned | Cohort 13 | Planned |
| | Pooled | Target CT-388 | Pooled | Target CT -388 |
| | Placebo | Dose (22 mg) | Placebo | Dose (22 mg) |
| | (N = 6) | (N = 23) | (N = 3) | (N = 10) |
| LFC (%), Median (range) | −3.5 | −35.3 | −21.9 | −58.5 |
| | (−66.1, 22.9) | (−83.8, 117.6) | (−59.2, 37.5) | (−72.8, 21.7) |

The effect of CT-388 on liver MRI-PDFF in participants with a baseline MIRI-PDFF of ≥5% was tested. Robust reductions in LFC were seen in all CT-388-treated participants (with and without T2D3), with a greater reduction in LFC seen in participants with MASLD (LFC≥5%) at baseline (Cohort 13). In participants with liver steatosis (LFC≥5%) at baseline, LFC was reduced by 59.3% in participants with T2D (Cohort 13) and by 50% in participants without T2D (Cohort 12) after 12 weeks of CT-388 treatment (FIGS. 39C and 39D; Table 35). In participants with significant liver steatosis (baseline LFC≥10%), similar results were observed with CT-388.

The effect of CT-388 on liver MRI-PDFF in participants with a baseline MRI-PDFF of ≥10% was also tested. In participants with liver steatosis ≥10%, a consistent trend of reduction in liver steatosis was observed in T2D (Cohort 13) and non-T2D (Cohort 12) participants with obesity. Median MRI-PDFF was 7.2% and 6.3% at week 12, compared to 14.2% and 14.6% at baseline, for non-T2D and T2D participants, respectively (FIG. 39E). Reduction was noted after 12 weeks of treatment, wherein the median reduction was 45.7% and 53.5% for non-T2D and T2D participants, respectively (FIG. 39F).

TABLE 35
Analysis of Percent Change from Baseline in Median Liver PDFF
(LFC ≥5% at Baseline) in Cohort 12 and 13 Participants

| | | Cohort 12 | | Cohort 13 |
|---|---|---|---|---|
| | Cohort 12 | Planned | Cohort 13 | Planned |
| | Pooled | Target CT-388 | Pooled | Target CT -388 |
| | Placebo | Dose (22 mg) | Placebo | Dose (22 mg) |
| | (N = 5) | (N = 15) | (N = 2) | (N = 8) |
| LFC ≥5% at baseline | −9.2 | −50.0 | 7.8 | −59.3 |
| LFC (%), Median (range) | (−66.1, 22.9) | (−83.8, 11.6) | (−21.9, 37.5) | (−72.8, −44.6) |

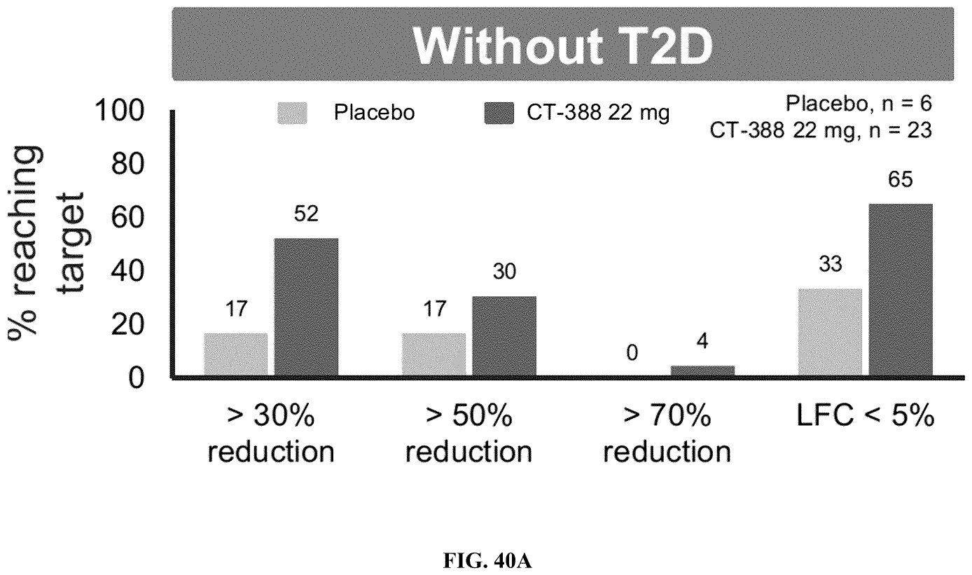

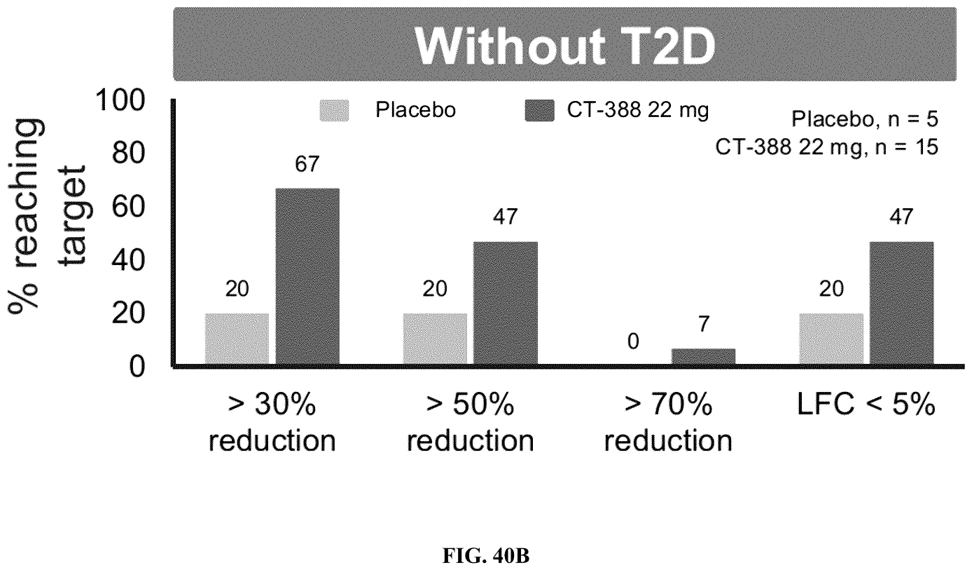

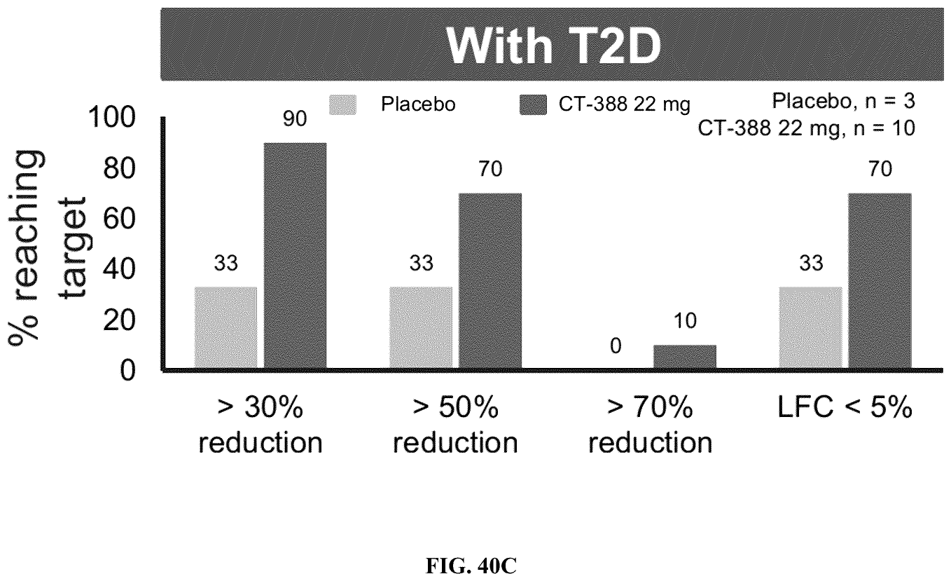

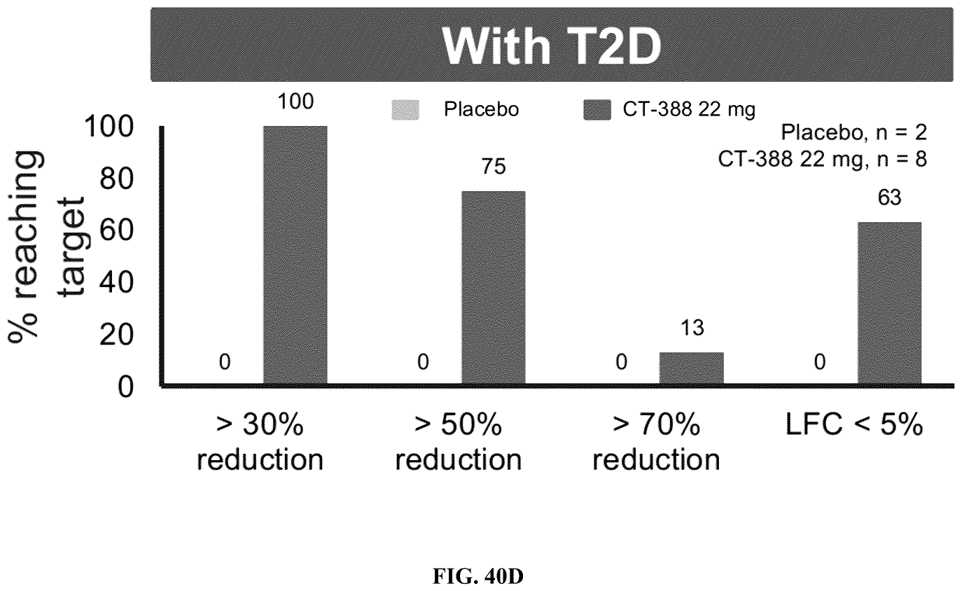

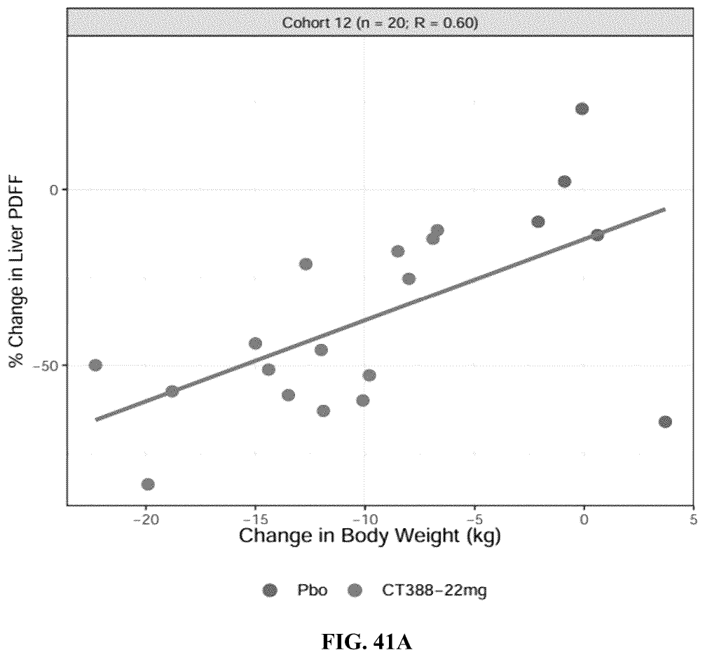

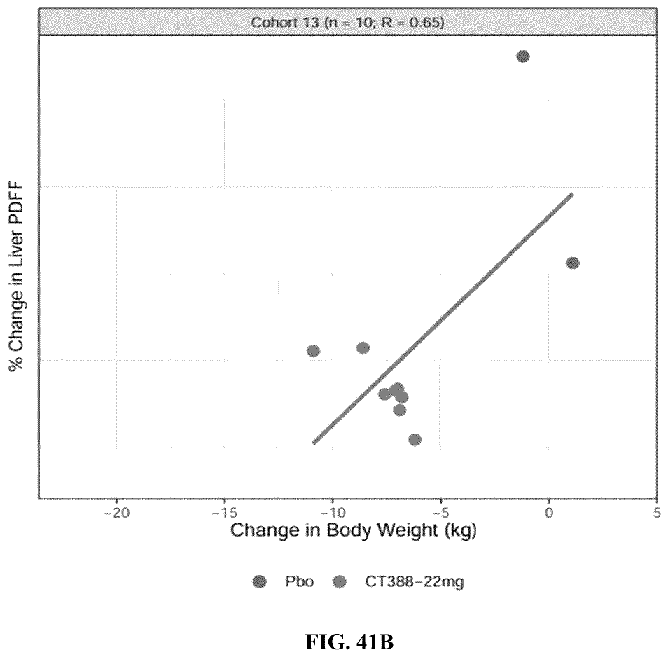

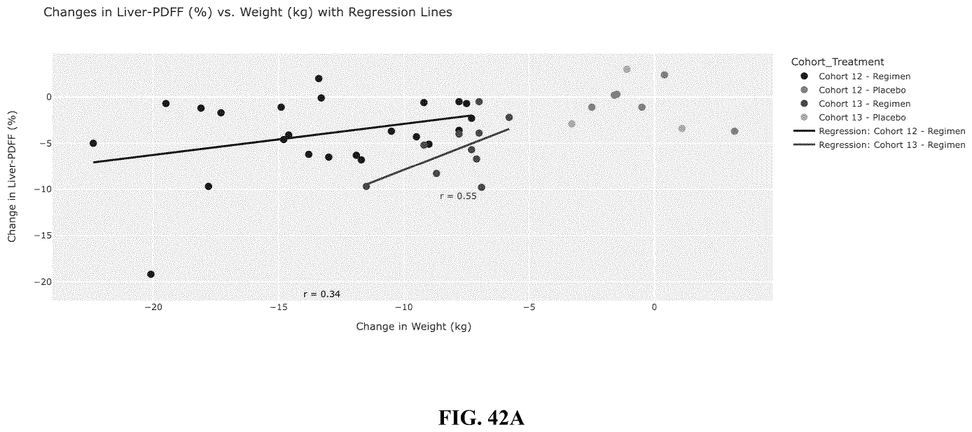

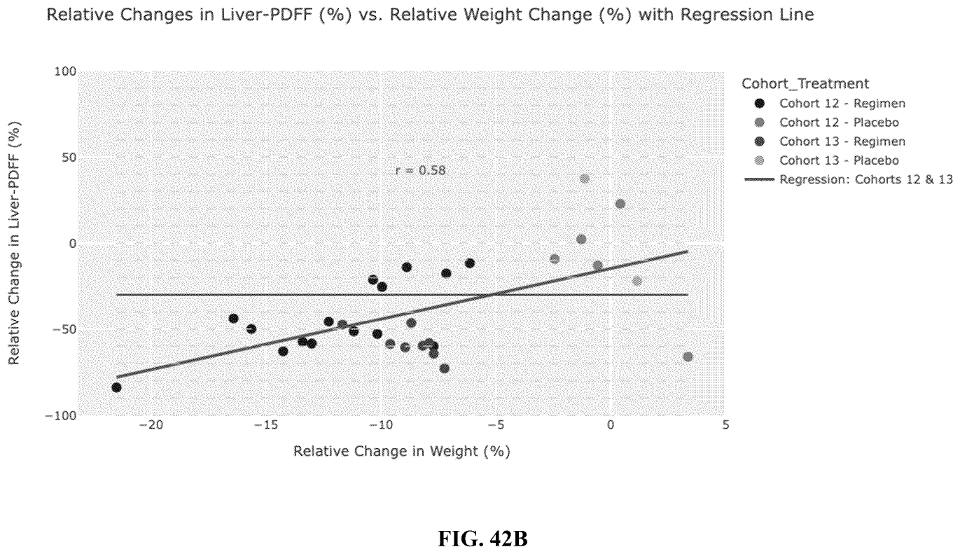

Participants receiving CT-388 exhibited reductions in LFC. Specifically, 78% (18/23) of participants receiving CT-388 (52% with and 90% without T2D) achieved an LFC reduction ≥30%, and 53% (12/23; 65% without and 70% with T2D) of participants achieved normal (<5%) LFC levels after 12 weeks of treatment (FIGS. 40A-40D). Furthermore, there was a positive correlation between reduction in body weight and LFC from baseline to week 12 for participants receiving CT-388 (FIGS. 41A and 41B; FIGS. 42A and 42B).

Liver inflammation (cT1≥800 ms) was elevated in both Cohorts 12 and 13 (833 ms and 851 ms for participants with and without T2D—Cohorts 12 and 13, respectively) (Table 37). In participants with liver steatosis ≥5% at baseline, 56.5% showed a reduction ≥40 ms (beyond the noise level). Among these participants, 17.3% showed a decrease ≥80 ms, which is clinically significant. Liver blood biomarkers at week 12 showed a trend towards improvement that was in line with imaging biomarkers (Table 36). No liver-related adverse events were reported with CT-388 treatment.

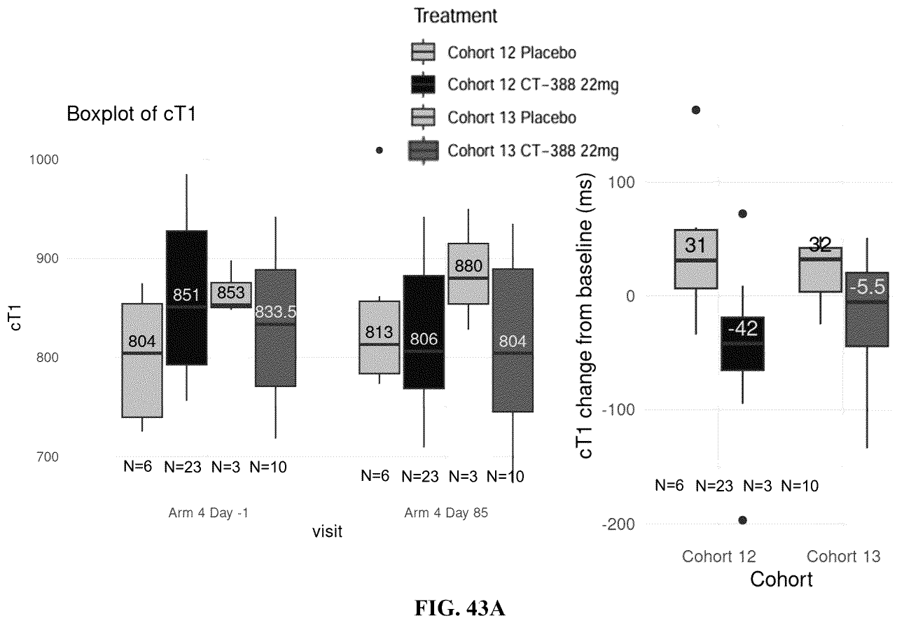

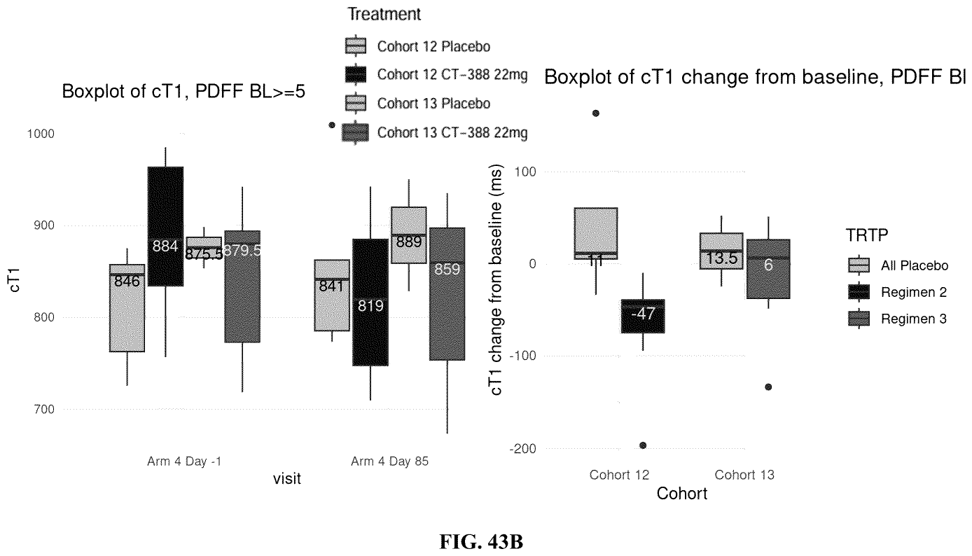

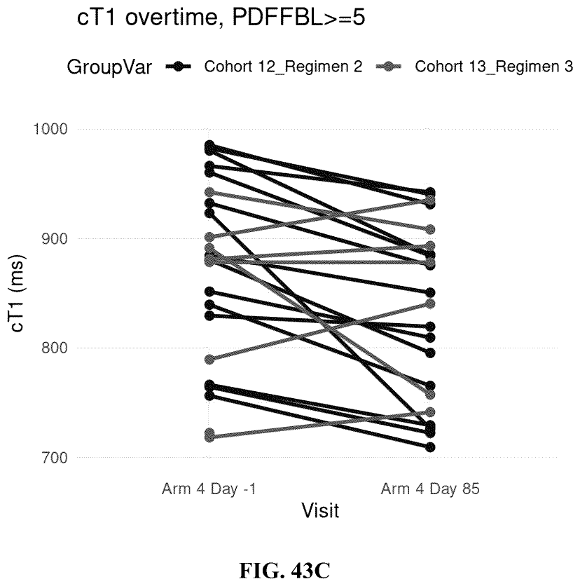

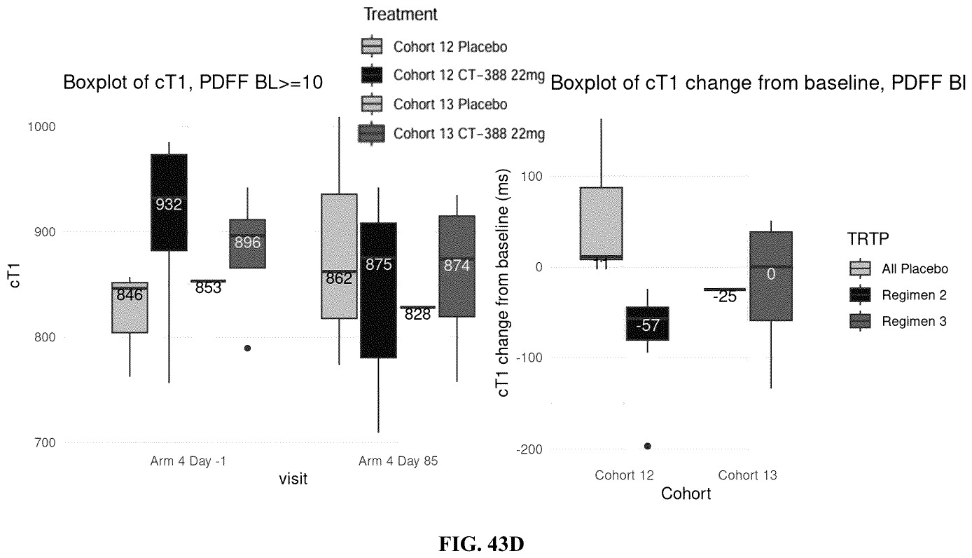

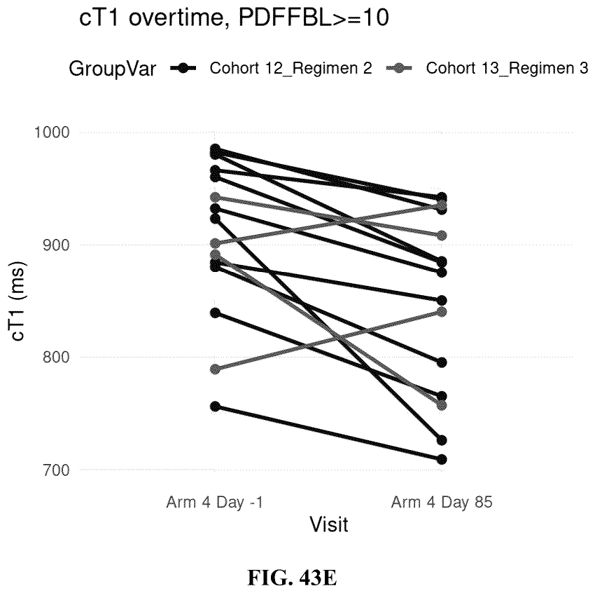

The effect of CT-388 on cT1 was also assessed in the MRI-PDFF analysis population. After 12 weeks of treatment, in patients with obesity, without T2DM (Cohort 12), C improved, while in patients with obesity, with T2DM (Cohort 13), 3 remained similar to baseline values (FIG. 43A). The same trend was observed in patients with liver steatosis ≥500 at baseline (FIGS. 43B and 43C) and ≥1000 at baseline (FIGS. 43D and 43E).

TABLE 36
Imaging and Blood Biomarkers at Baseline and Week 12 in the Overall
MRI Analysis Population and Participants with LFC >5% at Baseline

| All Participants | | | | Participants with Baseline LFC ≥5% | | | |
|---|---|---|---|---|---|---|---|
| Cohort 12 | Cohort 12 | C13 | C13 | C12 | C12 | C13 | C13 |
| Placebo | CT-388 | Placebo | CT-388 | Placebo | CT-388 | Placebo | CT-388 |
| N = 6 | N = 23 | N = 3 | N = 10 | N = 5 | N = 15 | N = 2 | N = 8 |

Imaging biomarkers

| | | | | | | | | | |
|---|---|---|---|---|---|---|---|---|---|
| | Baseline (U/L), | 799.5 | 861.4 | 866.3 | 826.9 | — | — | — | — |
| | mean (SD) | (67.4) | (79.1) | (27.5) | (80.6) | | | | |
| cT1 | Week 12 (U/L), | 42.7 | −44.0 | 19.7 | −15.6 | — | — | — | — |
| | mean (SD) | (68.0) | (49.3) | (40.0) | (53.7) | | | | |
| | Mean change | 31 | −42 | 32 | −5.5 | — | — | — | — |
| | from | | | | | | | | |
| | baseline, ms | | | | | | | | |

Blood biomarkers

| | | | | | | | | | |
|---|---|---|---|---|---|---|---|---|---|
| ALT | Baseline (U/L), | 36.8 | 26.6 | 53.0 | 27.4 | 39.4 | 31.20 | 70.5 | 30.5 |
| | mean (SD) | (15.9) | (13.2) | (45.9) | (19.8) | (16.3) | (13.8) | (48.8) | (21.2) |
| | Week 12 (U/L), | 32.8 | 26.4 | 41.7 | 17.0 | 36.2 | 27.3 | 58.0 | 17.9 |
| | mean (SD) | (10.2) | (18.3) | (49.0) | (5.6) | (6.8) | (14.1) | (56.6) | (5.7) |
| | Mean change | −5.1 | +3.5 | −35.6 | −25.4 | +0.6 | −9.3 | −28.3 | −29.3 |
| | from | | | | | | | | |
| | baseline, % | | | | | | | | |
| AST | Baseline (U/L), | 26.7 | 21.3 | 35.7 | 21.3 | 28.00 | 22.9 | 46.0 | 22.9 |
| | mean (SD) | (7.6) | (5.9) | (28.4) | (10.6) | (7.7) | (6.1) | (31.1) | (11.4) |
| | Week 12 (U/L), | 23.2 | 20.1 | 28.0 | 17.4 | 24.20 | 21.9 | 36.50 | 17.9 |
| | mean (SD) | (4.5) | (6.6) | (26.9) | (3.6) | (4.2) | (7.0) | (31.8) | (3.8) |
| | Mean change | −9.3 | −4.1 | −27.2 | 8.5 | −9.2 | −2.8 | −27.45 | −11.8 |
| | from | | | | | | | | |
| | baseline, % | | | | | | | | |
| ProC3 | Baseline (U/L), | 52.5 | 50.6 | 33.4 | 42.3 | 53.4 | 52.0 | 31.6 | 44.4 |
| | mean (SD) | (23.7) | (13.9) | (5.3) | (15.8) | (26.4) | (14.1) | (6.0) | (17.1) |
| | Week 12 (U/L), | 55.2 | 36.3 | 38.8 | 35.2 | 56.3 | 37.2 | 35.6 | 35.7 |
| | mean (SD) | (15.1) | (9.2) | (13.1) | (11.8) | (16.6) | (10.3) | (16.6) | (13.3) |
| | Mean change | +13.5 | −23.7 | +13.9 | −13.2 | +15.6 | −24.1 | 9.6 | −16.5 |
| | from | | | | | | | | |
| | baseline, % | | | | | | | | |

ALT = alanine aminotransferase, AST = aspartate aminotransferase, cT1 = iron corrected T1, LFC = liver fat content, MRI = magnetic resonance imaging, PDFF = proton density fat fraction, ProC3 = N-terminal type III collagen propeptide.

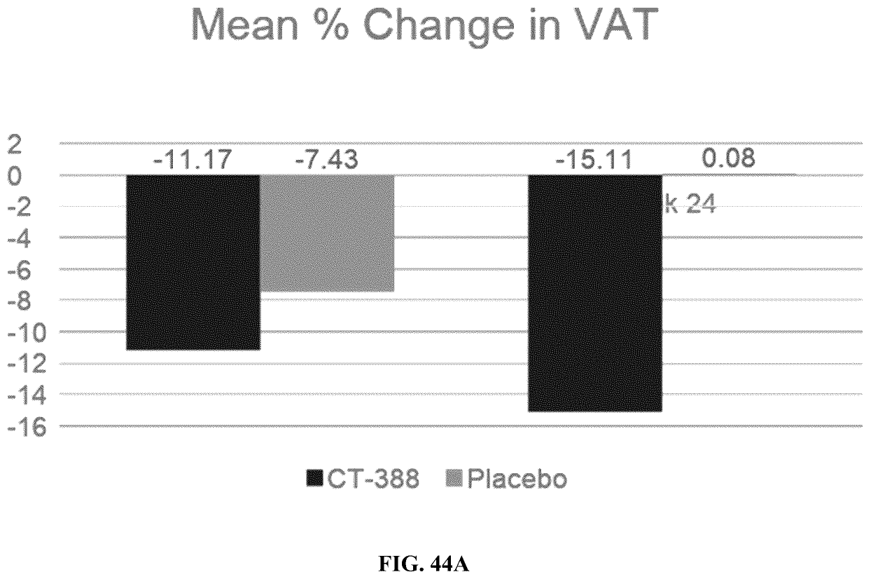

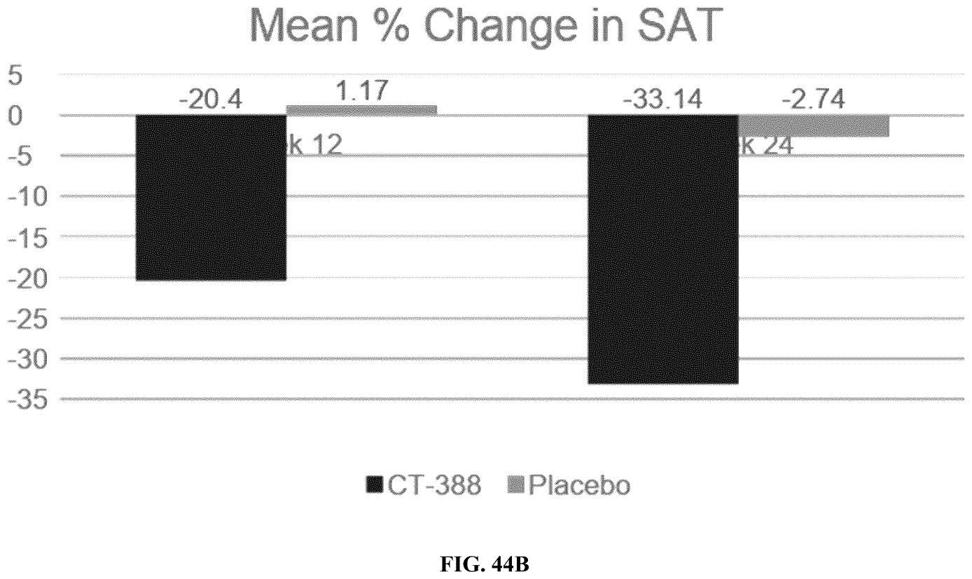

VAT and SAT were measured in patients of Cohort 12 at 12 and 24 weeks. The mean change in VAT at week 12 was −11.17% in participants treated with CT-388 compared to −7.43% in placebo participants. At week 24, the median change in VAT was −15.11% in CT-388 participants compared to −0.08 in placebo participants (FIG. 44A and Table 37). The mean change in SAT at week 12 was 424.43 in CT-388 participants, and +1.17 in placebo participants. At week 24, the median change in SAT was −33.14% in CT-388 participants, compared to −2.74% in placebo participants (FIG. 44B and Table 38).

TABLE 37
Observed Values of VAT

| | Baseline | Week 12 | Week 24 |
|---|---|---|---|
| CT-388 | N = 24 | 23 | 20 |
| Mean (SD) | 169.60 (68.2) | 144.26 (54.2) | 137.3 (47.8) |
| Placebo | N = 6 | N = 6 | N = 6 |
| Mean (SD) | 214.43 (40.3) | 197.8 (46.2) | 213.68 (39.0) |

TABLE 38
Observed Values of SAT

| | Baseline | Week 12 | Week 24 |
|---|---|---|---|
| CT-388 | N = 24 | 19 | 17 |
| Mean (SD) | 450.8 (80.5) | 356.52 (97.2) | 311.66 (125.6) |
| Placebo | N = 6 | N = 6 | N = 6 |
| Mean (SD) | 372.9 (80.5) | 377.85 (270.8) | 361.77 (74.5) |

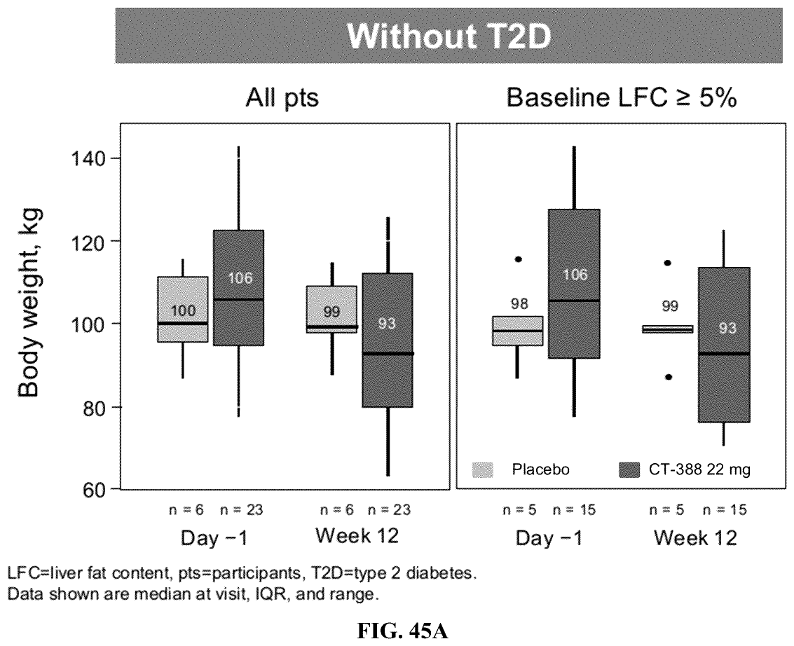

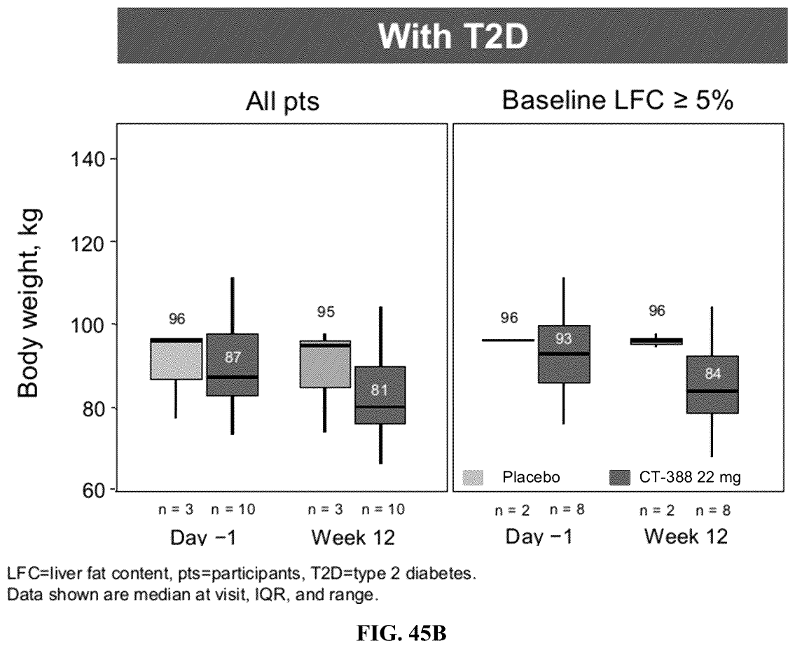

Additionally, clinically meaningful body weight loss was observed in patients with and without T2D after 12 weeks of treatment with CT-388 (FIGS. 45A and 45B). A trend of positive correlation between improvement in LFC and body weight loss was observed, with R=0.60 for pts without T2D and R=0.65 for pts with T2D.

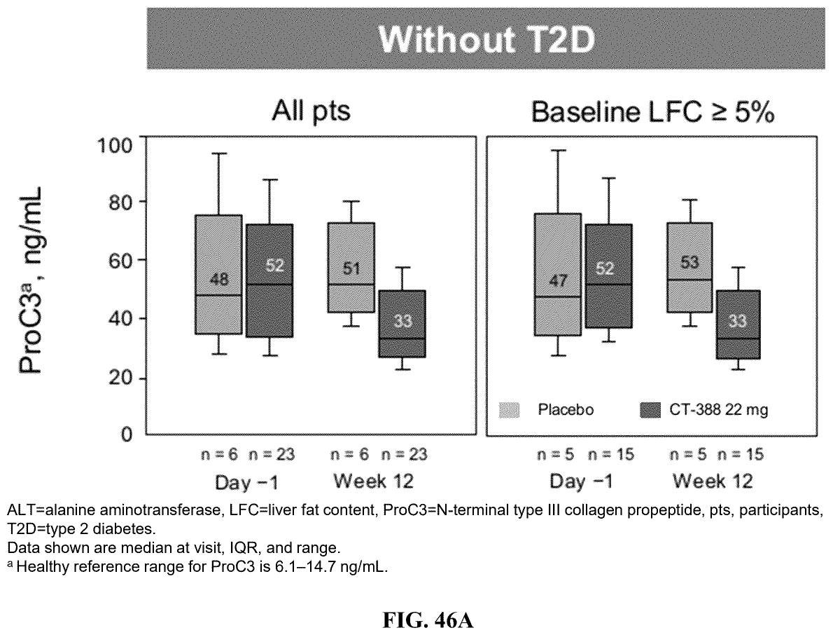

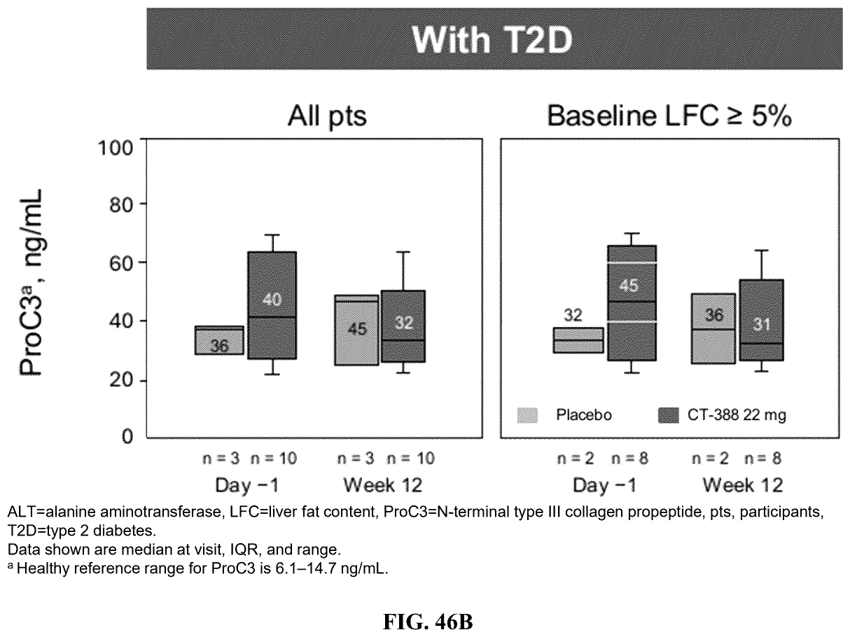

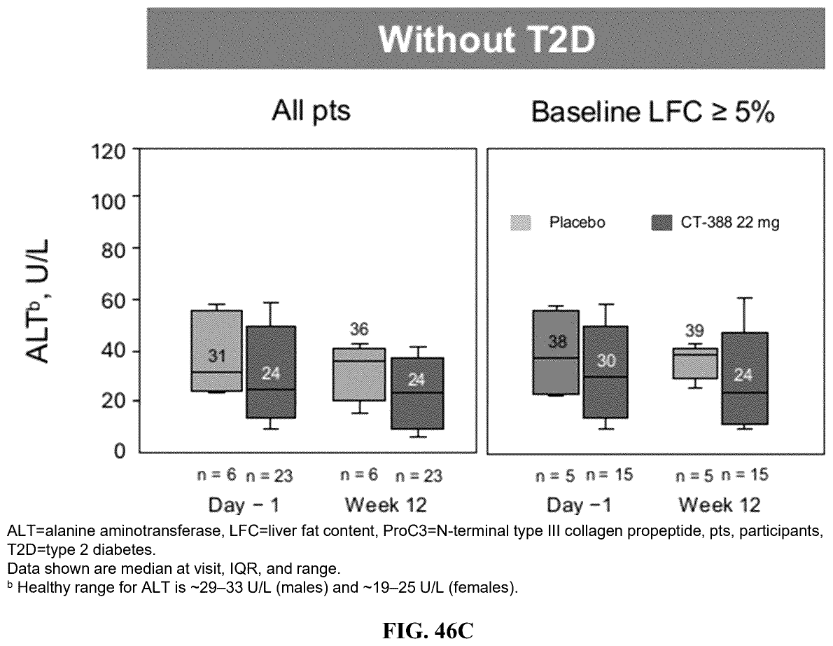

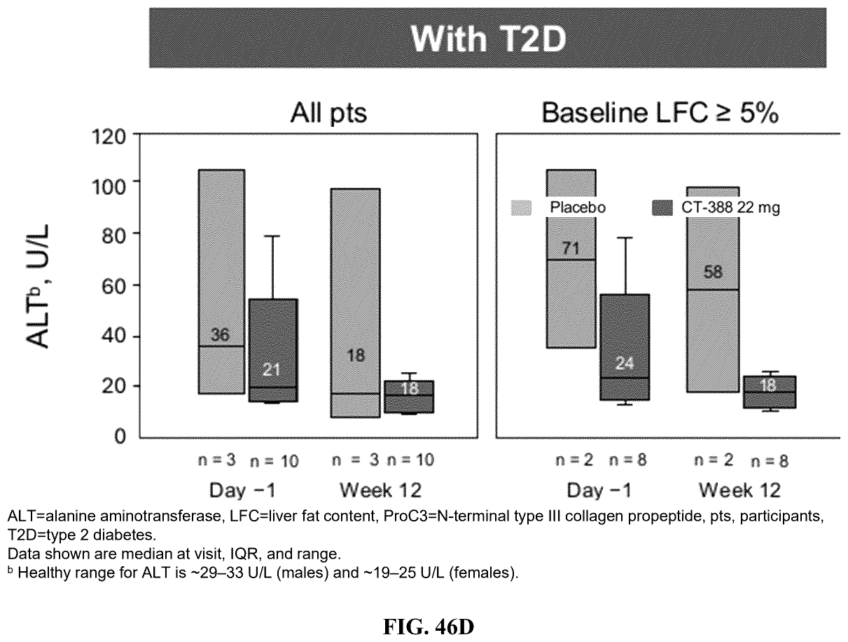

Furthermore, biomarkers analyzed at week 12 demonstrated a trend towards improvement. In participants with LFC≥5% at baseline, 56.5% had a clinically meaningful reduction in cT1 of ≥40 ms. N-terminal type III collagen propeptide was elevated at baseline and demonstrated reductions after 12 weeks of treatment with CT-388. ProC3 and ALT levels were also measured (FIGS. 46A-46D) (see, e.g., Erhardtsen et al., JHEP Rep. (2021) 3:100317 and Kwo et al., Am J Gastroenterol. (2017) 112:18-35).

Overall, in this Phase 1 trial, CT-388 treatment was associated with clinically meaningful reductions in LFC in participants with obesity, with or without T2D. Treatment with CT-388 at 22 mg for 12 weeks led to a robust decrease in LFC. This was evaluated via MRI-PDFF, with >50% of participants returning to normal LFC levels within 12 weeks. The CT-388 treatments were also associated with improvements in cT1 values, ALT, and ProC3 levels, thus suggesting a benefit in liver health. Together with weight loss and glycemic control improvements for CT-388 treatments, this suggests enhancements in metabolic health and a beneficial effect on liver steatosis in participants with obesity, regardless of whether they have T2D.

### Example 9: Efficacy and Safety of CT-388, a Signal-Biased, Dual GLP-1/GIP Receptor Agonist, in Adults With Obesity, With and Without T2D: A Randomized, Double-blind, Placebo-controlled, Phase 1 Trial

### Introduction

Obesity is a complex, chronic disease and a key driver of other cardiovascular, renal and metabolic diseases, including type 2 diabetes (T2D) (Ndumele et al, Circulation (2023) 148(20):1606-1635).

More than 16% of the global adult population is living with obesity (World Health Organization, “Overweight and Obesity” (2024)) and over half are expected to be living with obesity/overweight by 2050 (Collaborators GBDAB, Lancet (2025) 405(10481):813-838; Rubino et al., Lancet Diabetes Endocrinol (2025) 13(3):221-262; Yuen, Gastroenterol Clin North Am. (2023) 52(2):363-380). Guidelines recommend a comprehensive management approach for obesity involving lifestyle modification, medications and surgery (Garvey et al., Endocr Pract. (2016) 22(Suppl 3):1-203; Wharton et al., CMAJ. (2020) 192(31):E875-E891.

However despite a range of pharmacological options including glucagon-like peptide-1 (GLP-1) and GLP-1/glucose-dependent insulinotropic polypeptide (GIP) receptor agonists (Collins et al., StatPearls (2025) there remains a considerable unmet treatment need including variable weight loss, lack of weight loss maintenance and tolerability issues (Grandl G, et al., Lancet Reg Health Eur. (2024) 47:101100; Reiss A B et al., Biomolecules (2025) 15(3); Xie et al., Diabetes Metab Syndr Obes (2025) 18:2837-2849.

CT-388 is a unimolecular GLP-1/GIP receptor agonist that activates cAMP signaling with minimal β-arrestin recruitment at both receptors, leading to decreased internalization of both the GLP-1 and GIP receptors in cell-based assays. Preclinical studies showed that CT-388 has promising effects in improving glycemic control, reducing bodyweight, suppressing appetite and improving metabolic dysfunction-associated steatohepatitis pathology.

The first-in-human CT-388-101 phase 1 trial investigated the safety, tolerability, pharmacokinetics and pharmacodynamics of CT-388 in adults with obesity/overweight, with or without T2D. The trial consisted of several portions; the single ascending dose (SAD cohorts 1-5) and multiple ascending dose (MAD cohorts 6-8) portions of the trial in adults with obesity/overweight have been reported previously. Findings from these portions of the trial showed that CT-388 was generally well tolerated, improved glycemic parameters and led to weight loss over 4 weeks of treatment, and pharmacokinetics support once weekly (QW) dosing. Here we focus on the phase 1b multiple dose (MD) QW portion of the CT-388-101 trial in adults with obesity, with and without T2D.

### Materials and Methods

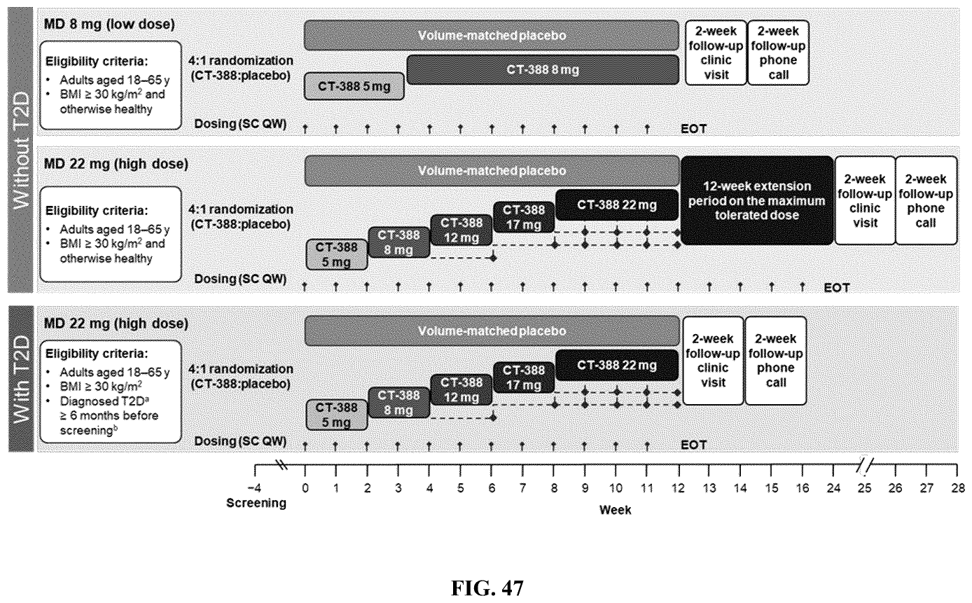

Trial Design, Participants and Treatment: Following the previously reported SAD and MAD portions of the double-blind, placebo-controlled, randomized CT-388-101 phase 1 trial (NCT04838405), the MD QW portion of the trial was conducted at a single trial site in Mexico and included three cohorts of adults. Two of the cohorts included participants with obesity without T2D (non-T2D cohorts) and one cohort included participants with obesity and T2D (T2D cohort) (FIG. 47). Inclusion criteria were age 18-65 years old and a BMI of ≥30 kg/m². For non-T2D cohorts, participants with diabetes were excluded but participants with prediabetes were allowed (if their condition was controlled and maintained using diet and exercise alone). For the T2D cohort, participants were required to have T2D diagnosed ≥6 months before screening and be managed with diet and/or exercise alone or treated with a stable dose of metformin monotherapy for ≥3 months before screening. Diabetes and prediabetes defined according to the 2022 American Diabetes Association Standards of Medical Care in Diabetes (American Diabetes Association Professional Practice, Diabetes Care. (2022; 45(Suppl 1):S17-S38).

Participants received CT-388 or volume-matched placebo QW by subcutaneous injection into the abdomen. The low-dose non-T2D cohort received 5 mg for the first three weeks, titrated to 8 mg for the following nine weeks (low-dose cohort). The high-dose cohorts received up-titration every 2 weeks for 8 weeks, starting with 5 mg before progressing to 8 mg, 12 mg and 17 mg, and then 22 mg for the following 4 weeks (12 weeks of treatment: high-dose cohorts). The high-dose non-T2D cohort then maintained the maximum tolerated dose an additional 12 weeks (the maintenance period).

This trial was conducted in compliance with the Declaration of Helsinki (World Medical Association, “WMA Declaration of Helsinki-Ethical Principles for Medical Research Involving Human Participants” (2024)), Council for International Organizations of Medical Sciences International Ethical Guidelines (Council for International Organizations of Medical Sciences, “International Ethical Guidelines for Health-related Research Involving Humans.” (2016)), and International Council for Harmonisation Good Clinical Practice Guidelines (Harmonisation ICf, “Guideline for Good Clinical Practice E6(R3)” (2025) and all relevant trial materials received approval from Independent Ethics Committee and/or Institutional Review Board before the study was initiated. All participants were required to provide written informed consent before initiation of trial activities.

Assessments: The primary objectives of the trial were safety and tolerability. Safety assessments included monitoring of treatment-emergent adverse events (TEAEs), serious adverse events (SAEs), TEAEs leading to study drug discontinuation, safety laboratory parameters, vital signs and electrocardiogram (ECG) parameters. TEAEs were defined as adverse events that began on or after the time of first study drug administration and were coded using the Medical Dictionary for Regulatory Activities (version 25.1) system organ class and preferred terms (MedDRA, “Introductory Guide MedDRA Version 25.1 (2022)). Secondary objectives included evaluating the pharmacodynamic effect of CT-388 on body weight and glucose homeostasis in the fasted state and in the oral glucose tolerance test (OGTT) setting. Changes from baseline in body weight, waist circumference, glycated hemoglobin (HbA1c), fasting glucose and fasting insulin levels were assessed on days 1 (pre-dose), 43 (Week 6) and 85 (Week 12) in all cohorts, and additionally on Day 169 (Week 24) in the high-dose non-T2D cohort. Glucose and insulin responses were assessed after an oral glucose tolerance test (OGTT) performed on Day −1 (one day before the first CT-388 dose), Day 44 (Week 6) and Day 79 (Week 12) in all cohorts and on Day 163 (Week 24) in the high-dose non-T2D cohort. Following an overnight fast, participants drank a solution containing 75 g glucose for the OGTT and blood samples were taken pre-drink at t=−0.25 hours and t=0, and post-drink at t=0.5, 1, 1.5 and 2 hours. Matsuda Index was calculated using 10,000/square root of (fasting glucose×fasting insulin)×(mean OGTT glucose concentration×mean OGTT insulin concentration) (Matsuda et al., Diabetes Care. (1999) 22(9):1462-1470). Additionally, 7-point self-monitoring of blood glucose (SMBG) was assessed on days −2, 44 (Week 6) and 79 (Week 11) in the T2D cohort.

Exploratory objectives included magnetic resonance imaging (MRI)-derived proton density fat fraction (PDFF) and adiposity, and biomarkers (iron-corrected T1 [cT1], alanine aminotransferase [ALT], N-terminal type III collagen propeptide [Pro-C3]). Primary and secondary objectives were assessed over 12 weeks for all cohorts and additionally up to 24 weeks for the high-dose non-T2D cohort. MRI was carried out at screening, Day 85 (Week 12 in all cohorts) and Day 169 (Week 24 in the high-dose non-T2D cohort) and PDFF was assessed as a measure of liver fat content (LFC), in addition to a body composition (lean and fat mass) analysis (skeletal muscle, visceral fat and subcutaneous fat at the L3 vertebral body level).

Statistical Methods: Data were summarized using descriptive statistics by treatment group (placebo groups were pooled for low- and high-dose non-T2D cohorts).

### Results

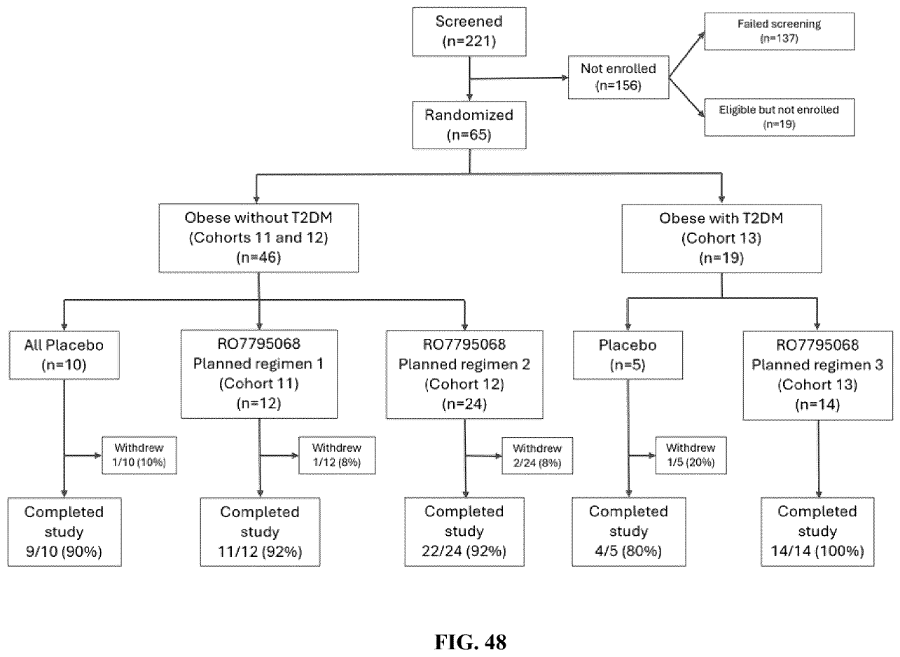

A total of 65 participants were randomized (n=46 without T2D and n=19 with T2D). Most participants completed treatment as planned (FIG. 48). One participant in each of the non-T2D and T2D placebo groups withdrew due to participant decision and consent withdrawal. In the non-T2D CT-388 group, one participant receiving 8 mg withdrew due to a positive pregnancy test, and two participants receiving 22 mg withdrew; one due to positive pregnancy test and one due to consent withdrawal. Of those completing the trial in the non-T2D groups, 100% on low-dose CT-388 completed on 8 mg while 63% (15/24) on high-dose CT-388 completed on 22 mg with the remainder (9/24) completing on 12 mg due to dose modifications. All participants (14/14) in the T2D high-dose CT-388 group completed the trial and most (12/14) completed on 22 mg, while the other two participants had dose modifications and completed on 17 mg.

All participants were Hispanic or Latinx (Table 39). Overall participants with T2D were older and had a lower body weight at baseline than those without T2D. In the T2D cohort, there was a longer duration of T2D in the placebo group than in the CT-388 group (mean 10.9 vs 5.9 years) with an accordingly higher fasting glucose (mean 205 vs 165 mg/dL) and HbA1c (mean 9.4 vs 5.2%). All participants with T2D were on metformin at baseline and one participant in the T2D placebo group had their metformin dose increased during the trial.

TABLE 39
Baseline Demographics and Characteristics

| | Non-T2D | | | | | | T2D | | | |
|---|---|---|---|---|---|---|---|---|---|---|
| | | | CT-388 | | CT-388 | | | | CT-388 | |
| | Placebo^{a} | | 8 mg | | 22 mg | | Placebo | | 22 mg | |
| | n = 10 | | n = 12 | | n = 24 | | n = 5 | | n = 14 | |
| Age, years | 37.3 | (12.7) | 37.4 | (9.8) | 32.3 | (9.1) | 41.8 | (13.7) | 46.7 | (9.3) |
| Sex, female, n (%) | 3 | (30) | 10 | (83) | 15 | (63) | 4 | (80) | 7 | (50) |
| Hispanic or Latinx, n (%) | 10 | (100) | 12 | (100) | 24 | (100) | 5 | (100) | 14 | (100) |
| Body weight, kg | 98.2 | (13.3) | 96.7 | (18.2) | 107.9 | (19.0) | 95.6 | (11.0) | 94.8 | (13.6) |
| BMI, kg/m² | 34.6 | (3.0) | 37.5 | (5.9) | 38.6 | (5.6) | 36.5 | (3.4) | 35.1 | (4.7) |
| HbA1c, % | 5.5 | (0.3) | 5.3 | (0.3) | 5.4 | (0.3) | 9.4 | (0.6) | 8.7 | (1.1) |
| Glycemic status, n (%)^{b} | | | | | | | | | | |
| Normoglycemia | 5 | (50) | 4 | (33.3) | 15 | (62.5) | 0 | | 0 | |
| Prediabetes | 5 | (50) | 7 | (58.3) | 7 | (29.2) | 0 | | 0 | |
| Diabetes | 0 | | 1 | (8.3) | 2 | (8.3) | 5 | (100) | 14 | (100) |
| T2D duration, years | N/A | | N/A | | N/A | | 10.9 | (9.0) | 5.9 | (5.3) |
| Metformin use, n (%) | 0 | | 0 | | 0 | | 5 | (100) | 14 | (100) |
| Metformin dose, mg | N/A | | N/A | | N/A | | 1760.0 | (615.8) | 1303.6 | (693.2) |
| Fasting glucose, mg/dL | 89.6 | (7.6) | 89.3 | (8.1) | 91.1 | (7.2) | 204.6 | (46.8) | 164.9 | (34.7) |
| Fasting insulin, μIU/mL | 20.5 | (7.4) | 17.3 | (7.4) | 18.7 | (7.7) | 23.9 | (15.3) | 16.0 | (7.5) |
| Glucose AUC, h × mg/dL | 272.1 | (48.4) | 294.4 | (46.5) | 265.8 | (46.9) | 601.9 | (118.7) | 495.1 | (99.5) |
| Insulin AUC, h × μIU/mL | 250.8 | (110.0) | 206.0 | (88.6) | 201.8 | (88.6) | 73.2 | (57.2) | 71.3 | (38.7) |
| C-peptide AUC, h × ng/mL | 20.8 | (6.1) | 18.7 | (4.0) | 18.3 | (4.4) | 9.8 | (4.9) | 10.0 | (3.1) |
| Matsuda Index | 2.2 | (0.70) | 2.9 | (1.7) | 3.4 | (2.2) | 2.3 | (1.3) | 3.1 | (1.6) |

Data are mean (SD) unless otherwise stated.
^{a}Placebo groups for low- and high-dose non-T2D cohorts are pooled for this analysis.
^{b}Post hoc derivation of glycemic status using fasting glucose, glucose values at 0 and 2 hours post OGTT, or HbA1c per 2022 American Diabetes Association Standards of Medical Care in Diabetes.
AUC = area under the curve, BMI = body mass index, HbA1c = glycated hemoglobin, N/A = not applicable, OGTT = oral glucose tolerance test, SD = standard deviation, T2D = type 2 diabetes.

Primary endpoint: All 36 participants who received CT-388 and 90% (9/10) of participants who received placebo experienced at least one TEAE up to Week 12 (Table 40). One participant in the non-T2D) CT-388 8 mg groups experienced an SAE of complete abortion unrelated to study drug, which led to discontinuation of trial treatment and withdrawal from the trial. No other participants discontinued due to a TEAE up to Week 12 and there were no deaths. Decreased appetite and other GI-related events were the most frequent TEAEs and were mostly mild or moderate in severity.

TABLE 40
Treatment-Emergent Adverse Events Over 12 Weeks

| | Non-T2D | | | | | | T2D | | | |
|---|---|---|---|---|---|---|---|---|---|---|
| | Placebo^{a} | | CT-388 8 mg | | CT-388 22 mg | | Placebo | | CT-388 22 mg | |
| | n = 10 | | n = 12 | | n = 24 | | n = 5 | | n = 14 | |
| ≥1 TEAE | 9 | (90) | 12 | (100) | 24 | (100) | 5 | (100.0) | 14 | (100.0) |
| SAE | 0 | | 1 | (8.3)^{b} | 0 | | 0 | | 0 | |
| Death | 0 | | 0 | | 0 | | 0 | | 0 | |
| ≥1 treatment-related TEAE | 7 | (70) | 12 | (100) | 24 | (100) | 4 | (80.0) | 14 | (100.0) |
| Mild | 3 | (30) | 4 | (33.3) | 11 | (45.8) | 4 | (80.0) | 5 | (35.7) |
| Moderate | 5 | (50) | 7 | (58.3) | 12 | (50.0) | 1 | (20.0) | 9 | (64.3) |
| Severe | 1 | (10) | 0 | | 1 | (4.2)^{d} | 0 | | 0 | |
| Life-threatening | 0 | | 1 | (8.3)^{b} | 0 | | 0 | | 0 | |
| TEAE leading to treatment | 0 | | 1 | (8.3)^{b} | 1 | (4.2)^{e} | 0 | | 0 | |
| discontinuation | | | | | | | | | | |
| TEAE leading to dose | 0 | | 0 | | 1 | (3.2)^{f} | 0 | | 1 | (7.1)^{g} |
| interruption | | | | | | | | | | |
| TEAE leading to dose reduction | 0 | | 0 | | 7 | (29.2) | 0 | | 1 | (7.1)^{g} |
| Most common GI TEAEs | | | | | | | | | | |
| Nausea | 2 | (20.0) | 5 | (41.7) | 20 | (83.3) | 3 | (60.0) | 10 | (71.4) |
| Mild | 2 | (20.0) | 4 | (33.3) | 14 | (58.3) | 2 | (40.0) | 8 | (57.1) |
| Moderate | 0 | | 1 | (8.3) | 6 | (25.0) | 1 | (20.0) | 2 | (14.3) |
| Severe | 0 | | 0 | | 0 | | 0 | | 0 | |
| Vomiting | 1 | (10.0) | 3 | (25.0) | 18 | (75.0) | 0 | | 9 | (64.3) |
| Mild | 1 | (10.0) | 2 | (16.7) | 9 | (37.5) | 0 | | 8 | (57.1) |
| Moderate | 0 | | 1 | (8.3) | 9 | (37.5) | 0 | | 1 | (7.1) |
| Severe | 0 | | 0 | | 0 | | 0 | | 0 | |
| Constipation | 0 | | 9 | (75.0) | 14 | (58.3) | 1 | (20.0) | 7 | (50.0) |
| Mild | 0 | | 9 | (75.0) | 13 | (54.2) | 1 | (20.0) | 7 | (50.0) |
| Moderate | 0 | | 0 | | 1 | (4.2) | 0 | | 0 | |
| Severe | 0 | | 0 | | 0 | | 0 | | 0 | |
| Diarrhea | 2 | (20.0) | 6 | (50.0) | 13 | (54.2) | 3 | (60.0) | 7 | (50.0) |
| Mild | 2 | (20.0) | 6 | (50.0) | 11 | (45.8) | 3 | (60.0) | 5 | (35.7) |
| Moderate | 0 | | 0 | | 2 | (8.3) | 0 | | 2 | (14.3) |
| Severe | 0 | | 0 | | 0 | | 0 | | 0 | |
| Most common other TEAEs | | | | | | | | | | |
| Decreased appetite | 4 | (40.0) | 11 | (91.7) | 22 | (91.7) | 4 | (80.0) | 14 | (100.0) |
| Dyspepsia | 0 | | 1 | (8.3) | 14 | (58.3) | 3 | (60.0) | 9 | (64.3) |
| Eructation | 2 | (20.0) | 7 | (58.3) | 13 | (54.2) | 2 | (40.0) | 8 | (57.1) |
| Feces soft | 1 | (10.0) | 5 | (41.7) | 12 | (50.0) | 2 | (40.0) | 7 | (50.0) |

| | | | | | | | | |
|---|---|---|---|---|---|---|---|---|
| Early satiety | 0 | 0 | 11 | (45.8) | 2 | (40.0) | 9 | (64.3) |

| Abdominal distension | 1 | (10.0) | 3 | (25.0) | 5 | (20.8) | 0 | 3 | (21.4) |
|---|---|---|---|---|---|---|---|---|---|
| Abdominal pain | 0 | | 0 | | 5 | (20.8) | 0 | 3 | (21.4) |
| Dizziness | 1 | (10) | 1 | (8.3) | 4 | (16.7) | 0 | 3 | (21.4) |

| | | | | | | |
|---|---|---|---|---|---|---|
| Somnolence | 0 | 0 | 0 | 0 | 3 | (21.4) |

^{a}Placebo groups for low- and high-dose non-T2D cohorts are pooled for this analysis.
^{b}SAE of spontaneous abortion; unrelated to study drug, outcome of an unplanned pregnancy managed with postcoital contraception and progesterone. The study drug was discontinued immediately after the positive pregnancy test.
^{c}Severe, non-serious TEAE of creatine kinase elevation during the follow-up period.
^{d}Severe gastroenteritis (unrelated to study drug; n = 1).
^{e}Pregnancy (unrelated to study drug) occurred due to non-compliance of study-required contraception (outcome: delivery of a healthy newborn).
^{f}Food poisoning unrelated to study drug was recorded as “dose interruption due to AE.”
^{g}Drug interrupted and then dose subsequently reduced in the same participant: dose interrupted for multiple mild and moderate gastrointestinal-related AEs (resolved, possible or unrelated to CT-388), resumed up-titration following interruption; dose subsequently reduced for TEAE of mild palpitations (resolved while on drug, potentially related to CT-388, medical history of arterial hypertension), completed treatment on 17 mg.
GI = gastrointestinal, SAE = serious adverse event, TEAE = treatment-emergent adverse event, T2D = type 2 diabetes.

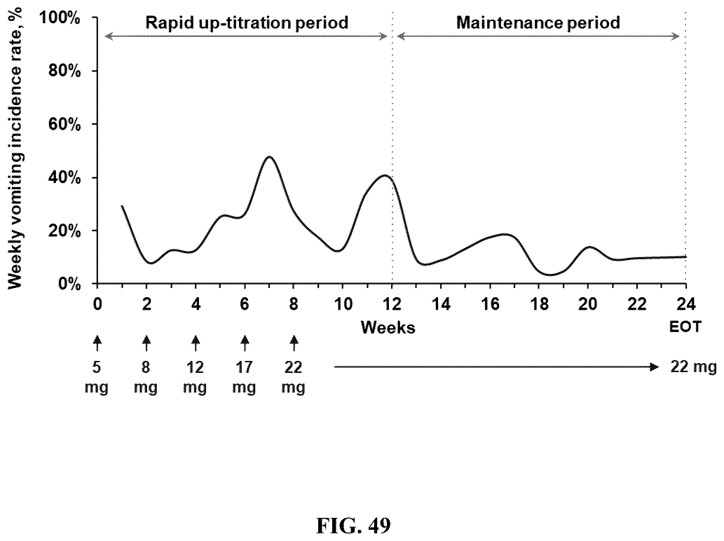

In the non-T2D CT-388 22 mg group, GI-related TEAEs decreased during the maintenance period (Week 12 to 24) despite two-thirds of participants continuing the highest dose (Table 41). One participant experienced anon-serious TEA of food poisoning that led to discontinuation of trial treatment at approximately Week 22 but not withdrawal from the trial. Vomiting occurred after the first dose (starting dose 5 mg), increased during the rapid up-titration period (Day 1 to Week 12), and decreased in frequency and severity during the maintenance period (FIG. 49).

TABLE 41
Treatment-emergent Adverse Events Over 12 and
24 Weeks in the High-dose Non-T2D Cohort

| | Day 1 to Week 12 | | | | Week 12 to Week 24 | | | |
|---|---|---|---|---|---|---|---|---|
| | | | CT-388 | | | | CT-388 | |
| | Placebo | | 22 mg | | Placebo | | 22 mg | |
| | n = 7 | | n = 24 | | n = 7 | | n = 24 | |
| ≥1 TEAE | 6 | (85.7) | 24 | (100) | 6 | (85.7) | 23 | (95.8) |
| SAE | 0 | | 0 | | 0 | | 2 | (8.3) |
| Death | 0 | | 0 | | 0 | | 0 | |
| ≥1 treatment-related TEAE | 6 | (85.7) | 24 | (100) | 4 | (57.1) | 20 | (83.3) |
| Mild | 3 | (30) | 11 | (45.8) | 4 | (57.1) | 2 | (28.6) |
| Moderate | 5 | (50) | 12 | (50.0) | 2 | (28.6) | 4 | (57.1) |
| Severe | 1 | (10)^{a} | 1 | (4.2)^{b} | 0 | | 0 | |
| TEAE leading to treatment | 0 | | 0 | | 0 | | 1 | (4.2) |
| discontinuation | | | | | | | | |
| TEAE leading to dose interruption | 0 | | 1 | (4.2)^{c} | 0 | | 1 | (4.2) |
| TEAE leading to dose reduction | 0 | | 7 | (29.2) | 0 | | 3 | (12.5) |
| Most common GI TEAEs | | | | | | | | |
| Nausea | 2 | (20.0) | 20 | (83.3) | 0 | | 13 | (54.2) |
| Mild | 2 | (20.0) | 14 | (58.3) | 0 | | 13 | (54.2) |
| Moderate | 0 | | 6 | (25.0) | 0 | | 0 | |
| Severe | 0 | | 0 | | 0 | | 0 | |
| Vomiting | 1 | (10.0) | 18 | (75.0) | 0 | | 8 | (33.3) |
| Mild | 1 | (10.0) | 9 | (37.5) | 0 | | 2 | (20.8) |
| Moderate | 0 | | 9 | (37.5) | 0 | | 3 | (12.5) |
| Severe | 0 | | 0 | | 0 | | 0 | |
| Constipation | 0 | | 14 | (58.3) | 0 | | 12 | (50.0) |
| Mild | 0 | | 13 | (54.2) | 0 | | 12 | (50.0) |
| Moderate | 0 | | 1 | (4.2) | 0 | | 11 | (45.8) |
| Severe | 0 | | 0 | | 0 | | 1 | (4.2) |
| Diarrhea | 2 | (20.0) | 13 | (54.2) | 0 | | 12 | (50.0) |
| Mild | 2 | (20.0) | 11 | (45.8) | 0 | | 10 | (41.7) |
| Moderate | 0 | | 2 | (8.3) | 0 | | 2 | (8.3) |
| Severe | 0 | | 0 | | 0 | | 0 | |
| Most common other TEAEs | | | | | | | | |
| Decreased appetite | 4 | (40.0) | 22 | (91.7) | 0 | | 1 | (4.2) |
| Dyspepsia | 0 | | 14 | (58.3) | 0 | | 9 | (37.5) |
| Eructation | 2 | (20.0) | 13 | (54.2) | 1 | (14.3) | 9 | (37.5) |
| Feces soft | 1 | (10.0) | 12 | (50.0) | 1 | (14.3) | 8 | (33.3) |

| Early satiety | 0 | | 11 | (45.8) | 0 | 0 | |
|---|---|---|---|---|---|---|---|
| Abdominal distension | 1 | (10.0) | 5 | (20.8) | 0 | 0 | |
| Abdominal pain | 0 | | 5 | (20.8) | 0 | 1 | (4.2) |
| Dizziness | 1 | (10) | 4 | (16.7) | 0 | 2 | (8.3) |

^{a}Severe, non-serious TEAE of creatine kinase elevation during the follow-up period.
^{b}Severe gastroenteritis (unrelated to study drug; n = 1).
^{c}Food poisoning unrelated to study drug was recorded as “dose interruption due to AE.”
GI = gastrointestinal, SAE = serious adverse event, T2D = type 2 diabetes, TEAE = treatmentemergent adverse event.

No level 2 or 3 hypoglycemia (as defined by ADA; American Diabetes Association Professional Practice, Diabetes Care. (2025); 48(1 Suppl 1): S128-S145)) was reported. There were transient increases in pulse rate with CT-388 compared with placebo and a trend for decrease in systolic blood pressure with CT-388 22 mg compared with placebo in non-T2D and T2D3 cohorts. No clinically significant changes in safety laboratory values were reported.

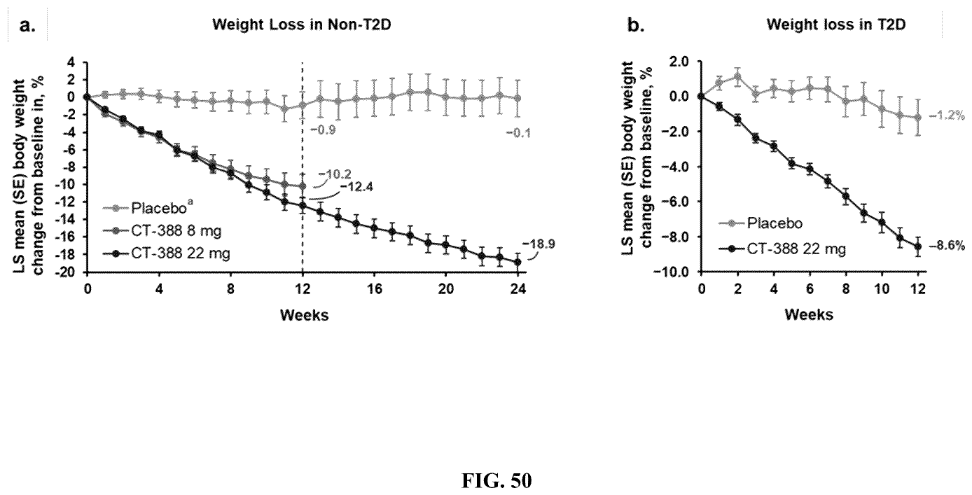

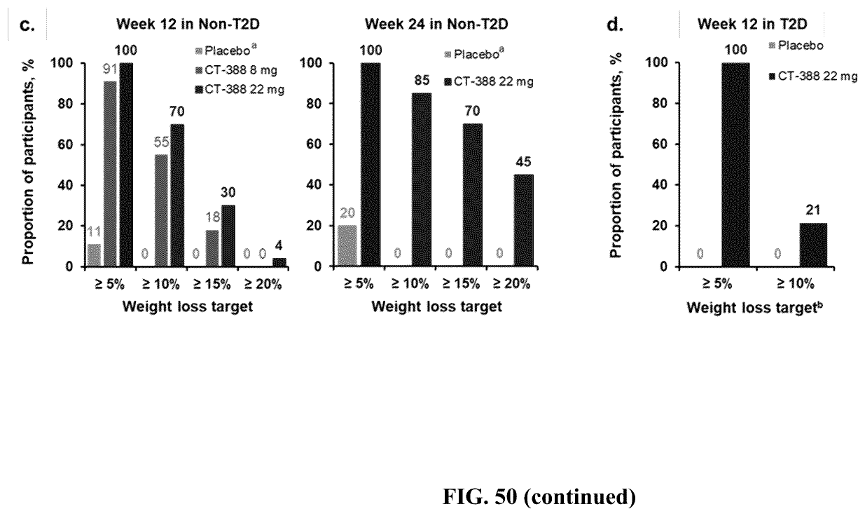

Secondary endpoints: In both non-T2D and T2D) cohorts, weight loss was observed early as Week 1 and body weight progressively decreased over the treatment period without plateauing (FIG. 50). Placebo-adjusted least squares (LS) mean (9500 confidence interval [CI]) percent change from baseline in body weight at Week 12 was −9.300 (−13.4, −5.2) for CT-388 8 mg and −11.4% (−15.1, −7.9) for CT-388 22 mg in the non-T2D cohorts (panel (a)), and −7.4% (−9.8, −4.9) for the CT-388 22 mg T2D3 cohort (panel (b)). This equated to a mean (9500 CI) weight loss of −9.5 (−14.3, 2.9) kg and −13.1 (−22.3, −6.7) kg for CT-388 8 mg and 22 mg compared with −0.1 (−8.9, 3.7) kg for placebo in non-T2D cohorts, and −7.1 (−13.2, −6.2) kg for the CT-388 22 mg compared with −0.7 (−3.4, 1.1) for placebo in the T2D cohort. At Week 12, 91% (10/11), 55% (6/11), 18% (2/11) and 0% of participants receiving CT-388 8 mg and 100% (23/23), 70% (16/23), 30% (7/23) and 4% (1/23) of participants receiving CT-388 22 mg achieved ≥5%, ≥10%, ≥15% and ≥20% weight loss in the non-T2D groups, respectively (panel (c)). In the T2D cohort, 100% (14/14) and 21.49 (3/14) of participants receiving CT-388 22 mg achieved ≥5% and ≥10% weight loss at Week 12, respectively (none achieved ≥15% weight loss) (panel (d)). At week 24, in the non-T2D cohort, placebo-adjusted LS mean (950 CI) percent change from baseline in body weight was −18.8 (−23.6, −14.0) for CT-388 22 mg (panel (a)), with 100% (20/20), 85% (17/20), 70% (14/20) and 45% (9/20) of participants achieving ≥5%, ≥10%, ≥15% and ≥20% weight loss, respectively (panel (c)).

Clinically meaningful reductions in waist circumference were observed in all CT-388 groups compared with placebo. Placebo-adjusted LS mean difference in the change from baseline in waist circumference ranged between −6.7 and −7.8 cm across the CT-388 groups at Week 12, and at Week 24 it was −13.4 cm in the CT-388 22 mg non-T2D group (Table 42).

TABLE 42
Waist Circumference at Baseline and Change From Baseline at Week 12 and Week 24

| | Non-T2D | | | T2D | |
|---|---|---|---|---|---|
| | | CT-388 | CT-388 | | CT-388 |
| | Placebo^{a} | 8 mg | 22 mg | Placebo | 22 mg |
| Baseline, n | 9 | 12 | 24 | 5 | 14 |
| Median | 112.3 (106.0, 121.2) | 109.2 (94.5, 137.9) | 114.5 (97.5, 147.7) | 116.2 (96.0, 119.0) | 109.8 (98.0, 124.0) |
| (min, max), cm | | | | | |
| CFB at Week 12, n | 9 | 11 | 23 | 4 | 14 |
| LS mean estimate | −2.0 | −9.8 | −9.3 | −1.2 | −7.8 |
| Difference in LS mean | | −7.8 (−11.7, −3.9) | −7.3 (−10.7, −3.8) | | −6.7 (−9.4, −3.9) |
| from placebo (95% CI) | | | | | |
| CFB at Week 24, n | 5 | NA | 20 | NA | NA |
| LS mean estimate | −2.6 | | −15.9 | | |
| Difference in LS mean | | | −13.4 (−17.8, −9.0) | | |
| from placebo (95% CI) | | | | | |

^{a}Placebo groups for low- and high-dose non-T2D cohorts are pooled for this analysis.
CI = confidence interval, CFB = change from baseline; LS = least squares, max = maximum, min = minimum, NA = not applicable, SE = standard error, T2D = type 2 diabetes.

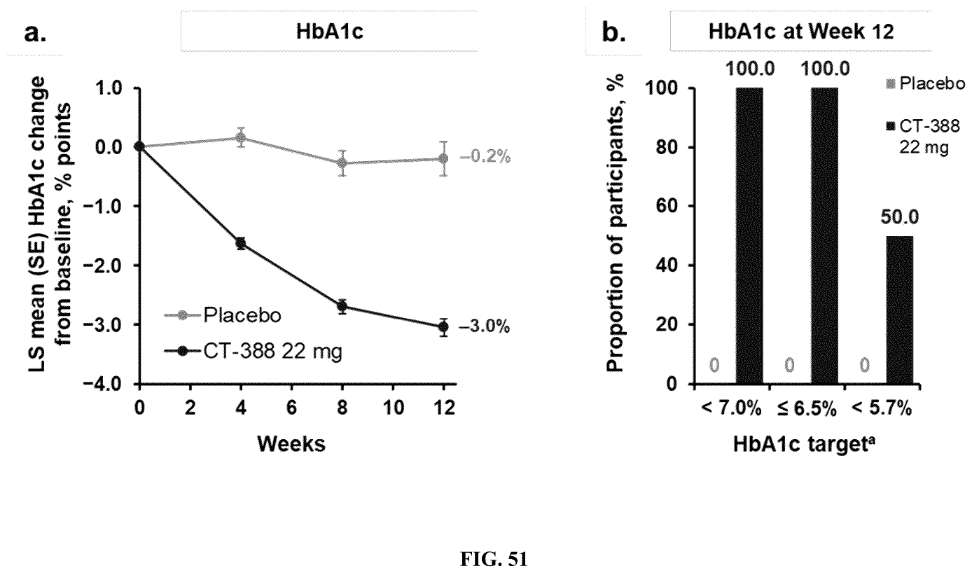

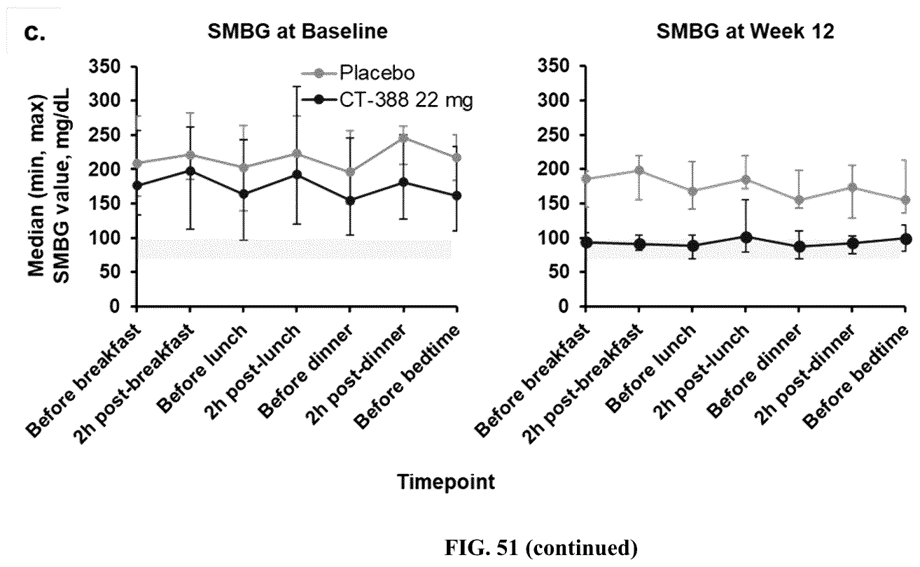

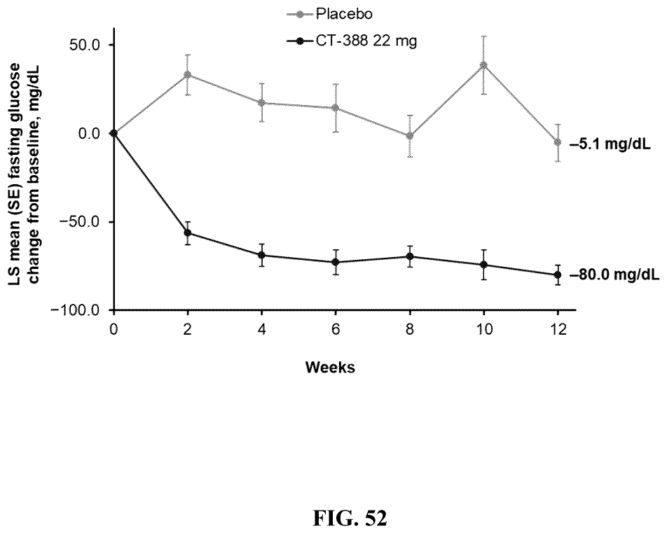

In the T2D3 cohort, an HbA1c reduction with CT-388 22 mg was observed at Week 4 and HbA1c continued to decrease over time resulting in a placebo-adjusted LS mean change from baseline of −2.8%-points (95% CI: −3.6, −2.1%) at 12 weeks (FIG. 51, panel (a)). All (14/14) participants receiving CT-388 met the target HbA1c level of ≤6.5% and half (7/14) reached normoglycemia (<5.7%) at 12 weeks (FIG. 51, panel (b)). Fasting glucose reduction with CT-388 22 mg was observed as early as Week 2 and was sustained up to Week 12 resulting a placebo-adjusted LS mean change from baseline of −74.9 mg/dL (95% CI: −100.8, −49.0) (FIG. 52). Additionally, CT-388 22 mg led to clinically meaningful improvements in 7-point SMBG at Week 12 at all time-points (FIG. 51, panel (c)).

TABLE 43
Glycemic Parameters Change From Baseline in the Non-T2D Cohort

| | Week 12 | | | | | Week 24 | | |
|---|---|---|---|---|---|---|---|---|
| | Placebo^{a} | CT-388 8 mg | | CT-388 22 mg | | Placebo | CT-388 22 mg | |
| | n = 9 | n = 11 | | n = 23 | | n = 5 | n = 20 | |
| HbA1c | | | | | | | | |
| LS mean (SE), | −0.06 (0.06) | −0.38 | (0.06) | −0.44 | (0.04) | 0.02 (0.08) | −0.43 | (0.04) |
| %-point | | | | | | | | |
| Placebo-adjusted | | −0.33 | (−0.50, −0.15) | −0.39 | (−0.54, −0.24) | | −0.44 | (−0.63, −0.26) |
| LS mean (95% | | | | | | | | |
| CI) | | | | | | | | |
| Fasting glucose | | | | | | | | |
| LS mean (SE), | 5.0 (1.6) | −2.8 | (1.4) | −4.3 | (1.0) | 2.6 (2.0) | −7.7 | (1.0) |
| mg/dL | | | | | | | | |
| Placebo-adjusted | | −7.8 | (−12.1, −3.5) | −9.2 | (−13.0, −5.5) | | −10.3 | (−15.0, −5.6) |
| LS mean (95% | | | | | | | | |
| CI) | | | | | | | | |
| Fasting insulin | | | | | | | | |
| LS mean (SE), | 0.3 (3.2) | −2.5 | (2.9) | −4.2 | (2.0) | −1.4 (3.9) | −5.5 | (1.9) |
| μIU/mL | | | | | | | | |
| Placebo-adjusted | | −2.8 | (−11.5, 5.8) | −4.5 | (−12.1, 3.1) | | −4.1 | (−13.2, 4.9) |
| LS mean (95% | | | | | | | | |
| CI) | | | | | | | | |

^{a}Placebo groups for low- and high-dose non-T2D cohorts are pooled for this analysis.
CI = confidence interval, HbA1c = glycated hemoglobin, LS = least squares, SE = standard error, T2D = type 2 diabetes

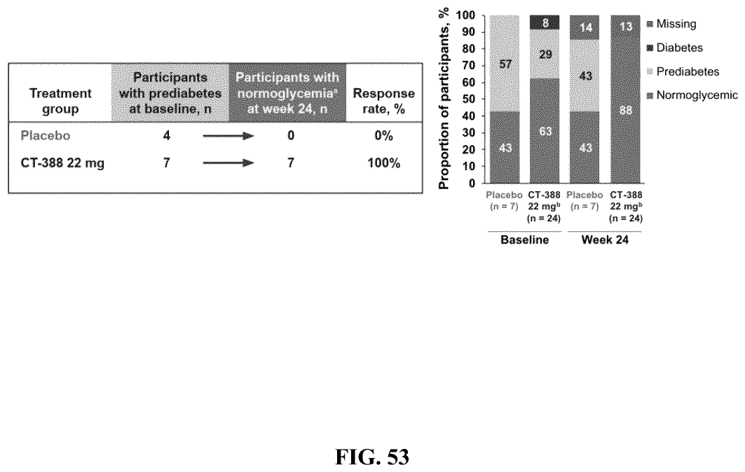

In the non-T2D cohort, HbA1c, fasting glucose and insulin were all reduced from baseline at Week 12 with CT-388 8 mg and 22 mg, and at Week 24 with CT-388 22 mg (Table 43). At Week 24, all participants in the non-T2D cohort who completed CT-388 22 mg treatment had a normal glycemic status, including those who were determined to have diabetes or prediabetes at baseline by post hoc fasting glucose, 0 and 2 hours post-OGTT, or HbA1c (FIG. 53).

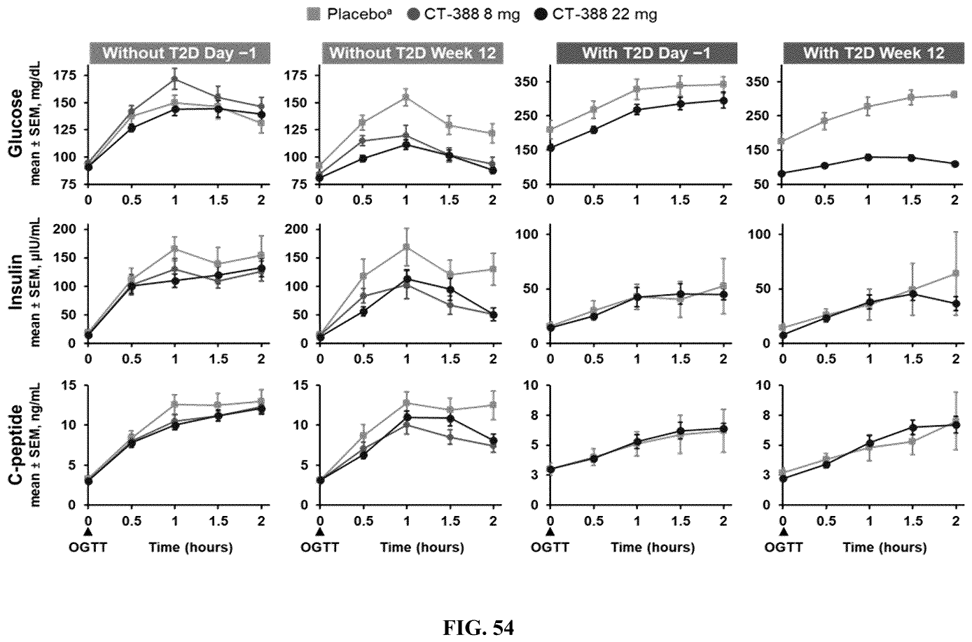

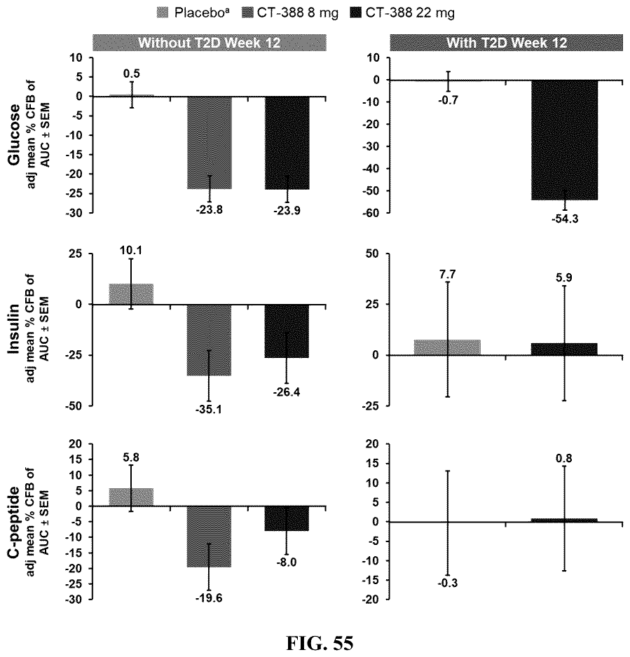

Glucose tolerance following OGTT was improved in all CT-388 groups compared with placebo at Week 12 (FIGS. 54 and 55). At Day −1, OGTT glucose excursions were similar between placebo and CT-388 in all three cohorts. At Week 12, difference in adjusted means for percent change from baseline (% CFB) for glucose area under the curve (AUC) (95% CI) was −24.3 (−33.6, −14.9) and −24.4 (−32.5, −16.3) with CT-388 8 mg and 22 mg, respectively, compared with placebo in participants without T2D, and −53.6 (−64.6, −42.6) with CT-388 22 mg compared with placebo in participants with T2D.

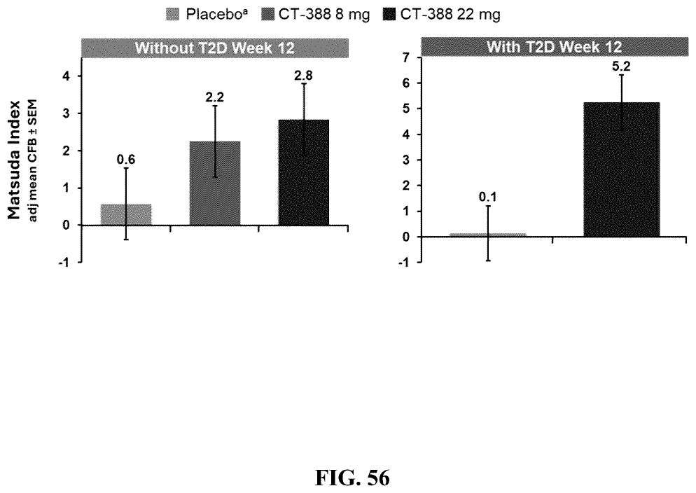

Insulin excursions following OGTT were reduced with CT-388 compared with placebo in participants without T2D at Week 12 (FIGS. 54 and 55). In participants with T2D, there was little difference in insulin excursions between CT-388 and placebo at Week 12, despite the marked reduction in glucose excursions. Similar results were observed for C-peptide. At Week 12, difference in adjusted means for % CFB for insulin AUC (95% CI) was −45.2 (−79.1, −11.3) and −36.4 (−66.6, −6.3) with CT-388 8 mg and 22 mg, respectively, compared with placebo in participants without T2D, and −1.8 (−69.8, 66.3) with CT-388 22 mg compared with placebo in participants with T2D. Improvements from baseline in insulin sensitivity, assessed using the Matsuda Index, were observed in all CT-388 groups by Week 12 (FIG. 56).

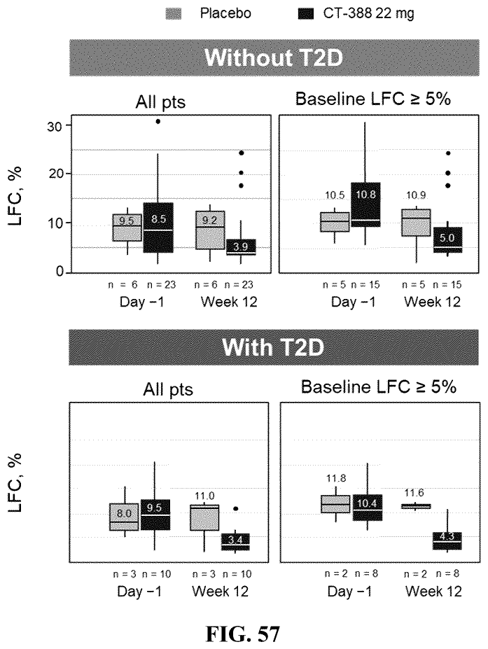

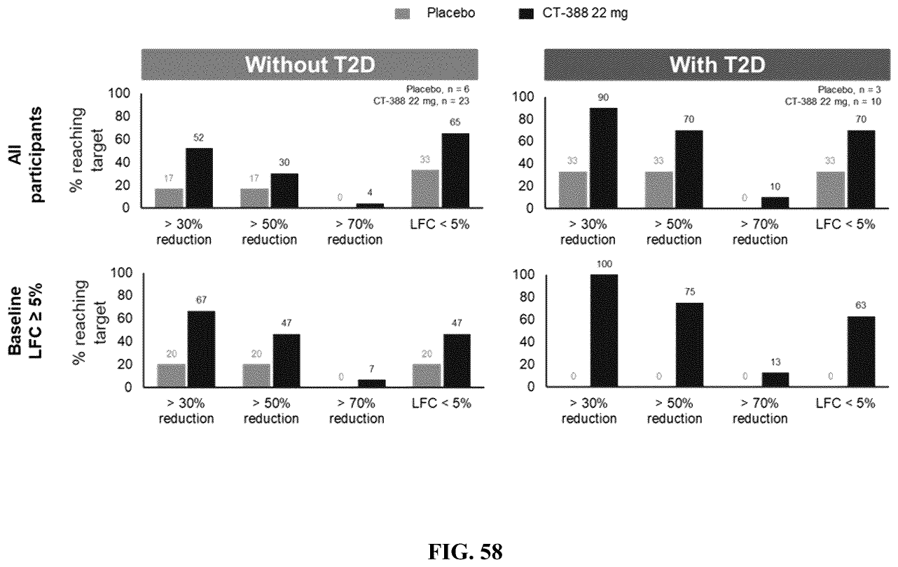

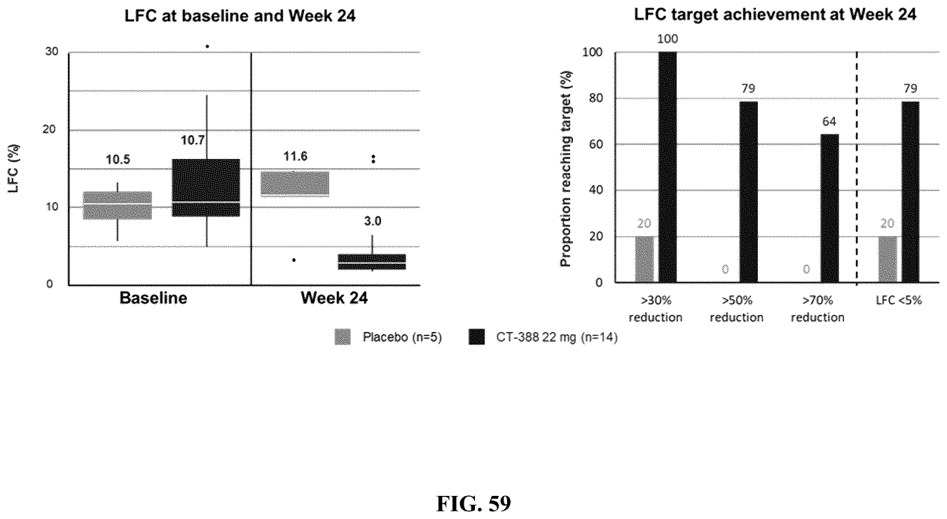

Exploratory endpoints: Overall, 71% (30/42) of participants in the high-dose cohorts had an incidental finding of baseline LFC≥5% (indicative of metabolic dysfunction-associated steatotic liver disease [MASLD](Andersson et al., Clin Gastroenterol Hepatol. (2025)) and 45% (19/42) had baseline LFC≥10% (indicative of severe MASLD). Reductions in the percentage of LFC were observed in non-T2D and T2D cohorts after 12 weeks of treatment with CT-388, with the reduction more prominent in participants with LFC≥5% than participants overall at baseline (FIG. 57). After 12 weeks of treatment, 78% (18/23) of participants receiving CT-388 22 mg (52% with and 90% without T2D) achieved an LFC reduction ≥30% and 53% (12/23; 65% without and 70% with T2D) of participants receiving CT-388 22 mg achieved normal (<5%) LFC levels (FIG. 58). After 24 weeks of treatment, 100% of participants receiving CT-388 22 mg in the non-T2D cohort achieved an LFC reduction ≥30% (FIG. 59).

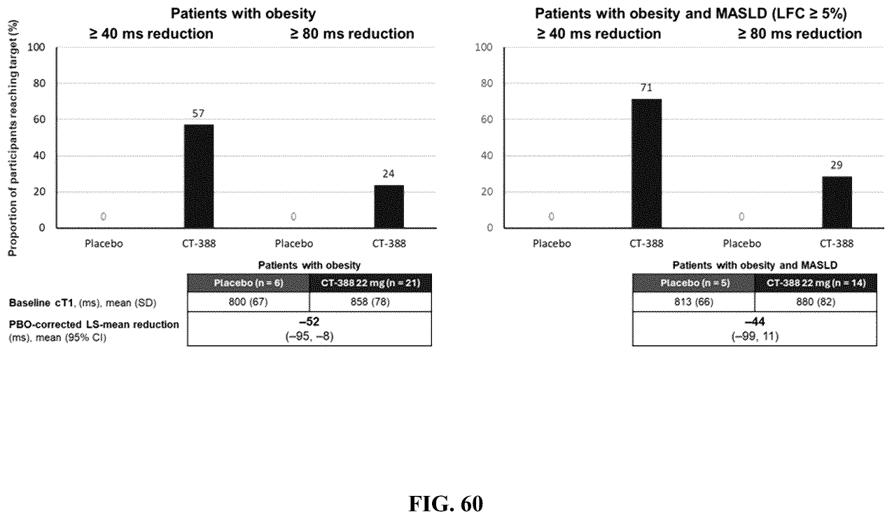

In the high-dose non-T2D cohort, iron-corrected T1 (cT1, the imaging biomarker of fibro-inflammation) was elevated at baseline. Reductions in cT1 of ≥40 ms and ≥80 ms were observed in 57% and 24% of participants overall, respectively, and 71% and 29% of participants with LFC≥5% at baseline, respectively (FIG. 60). ALT was normal at baseline whereas Pro-C3 was elevated at baseline, but both showed a trend towards improvement after 24 weeks of treatment with CT-388 (Table 44).

TABLE 44
Blood Biomarkers by Week 24 in Participants with
Obesity ± MASLD (LFC ≥5%) at Baseline

| | | | Patients with obesity and | |
|---|---|---|---|---|
| | Patients with obesity | | MASLD (LFC ≥5%) | |
| | Placebo | CT-388 22 mg | Placebo | CT-388 22 mg |
| | (n = 6) | (n = 21) | (n = 5) | (n = 14) |
| ALT | | | | |
| Baseline (U/L), mean (SD) | 37 (16) | 27 (13) | 39 (16) | 31 (14) |
| Week 24 (U/L), mean (SD) | 42 (21) | 17 (10) | 46 (21) | 19 (11) |
| Mean change from baseline, % | +13% | −32% | +16% | −37% |
| ProC3 | | | | |
| Baseline (ng/mL), mean (SD) | 52 (24) | 51 (14) | 53 (26) | 53 (14) |
| Week 24 (ng/mL), mean (SD) | 48 (14) | 32 (8) | 49 (16) | 34 (8) |
| Mean change from baseline, % | −2% | −34% | −2% | −33% |

ALT = alanine aminotransferase, LFC = liver fat content, MASLD = metabolic dysfunction-associated steatotic liver disease, ProC3 = N-terminal type III collagen propeptide, SD = standard deviation.

### Discussion

In adults with obesity, with or without T2D, CT-388 was generally well tolerated despite a rapid and steep up-titration. No unexpected safety/tolerability findings were reported; mild or moderate GI events were the most frequent TEAEs and there were no treatment-related TEAE leading to discontinuations. Clinically meaningful weight loss and robust improvements in glucose homeostasis were observed in both the fasting state and OGTT setting with CT-388 compared with placebo over 12 weeks in participants with and without T2D. Further weight loss was observed with CT-388 22 mg compared with placebo over 12-24 weeks in participants without T2D. Additionally, an exploratory analysis showed that CT-388 reduced LFC in adults with obesity and MASLD (liver fat content ≥5% at baseline) with or without T2D compared with placebo, suggesting CT-388 may have potential for the treatment of MASLD.

The frequency of GI-related TEAEs was consistent with the rapid up-titration and early stage of clinical development and is in line with findings with other incretin-based therapies (Veniant et al., Nat Metab. (2024) 6(2):290-303; Coskun et al., Mol Metab. (2018) 18:3-14). Rates of GI-related TEAEs with incretin-based therapies typically improve with slower titrations over time (Jastreboff et al., N Engl J Med. 2022; 387(3):205-216.

The T2D cohort achieved approximately one-third less weight loss at 12 weeks (7.4%) compared with the non-T2D cohort (11.4%). Improvements in glucose homeostasis were greatest in participants with T2D, with all participants achieving an HbA1c target of ≤6.5%, clinically meaningful improvements in 7-point SMBG, and a reduction in fasting glucose. There was a reduction in postprandial glucose and insulin in participants with obesity without T2D, and a marked reduction in postprandial glucose in participants with obesity and T2D. The limited difference in insulin excursions with CT-388 compared with placebo in participants with T2D at Week 12 may be because the 12-week duration was insufficient to produce a meaningful effect on β-cell function.

The analysis of LFC showed signals of robust reductions in participants with obesity with or without T2D over 12 weeks. Improvements in cT1 values, ALT levels and ProC3 over 24 weeks further suggest a benefit to liver health in participants with obesity without T2D. Indeed, reductions of ≥40 ms in cT1 are considered to be clinically meaningful (Andersson et al., Clin Gastroenterol Hepatol (2025); Bachtiar et al., PLoS One (2019) 14(4):e0214921; Beyer et al., J Magn Reson Imaging. 2025; 61(4):1947-1955) and reductions of ≥80 ms may predict resolution of steatohepatitis (Alkhouri et al., J Hepatol (2025) 82(3):438-445). ProC3 values over 20 ng/mL are predictive of fibrosis progression and a worse metabolic state (Nielsen M J et al., J Hepatol. (2021) 75(6):1292-1300), and there was a trend towards reduction in ProC3 from baseline to Week 24 with CT-388. These results indicate potential for CT-388 in the treatment of MASLD.

In conclusion, CT-388 was well tolerated, and led to clinically meaningful weight loss and robust improvements in glucose homeostasis over 12 weeks compared with placebo in participants with obesity, with or without T2D.

1. A method of treating metabolic dysfunction-associated steatohepatitis (MASH), metabolic dysfunction-associated steatotic liver disease (MASLD), or metabolic dysfunction-associated fatty liver disease (MAFLD) in an adult patient in need thereof, comprising administering by subcutaneous injection to the patient a compound of Formula I, or a pharmaceutically acceptable salt or ester thereof: 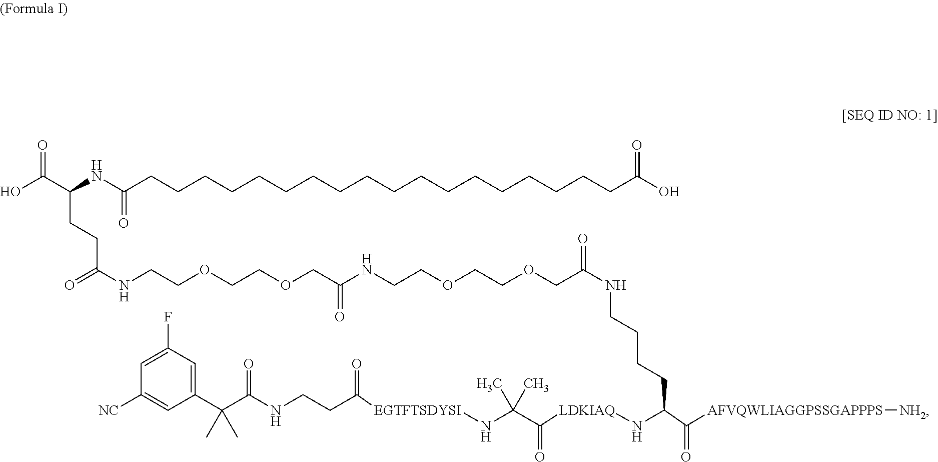
wherein the compound is administered at a body weight-independent dose of about 2.0 mg to about 25 mg.

2. The method of claim 1, wherein the method is for treating MASLD or MAFLD, optionally wherein the patient has a magnetic resonance imaging-proton density fat fraction (MRI-PDFF) of ≥5%.

3. The method of claim 1, wherein the method is for treating MASH, optionally wherein the patient has an MRI-PDFF of ≥10%.

4. A method of
decreasing liver fat as compared to baseline, optionally by 30% or more,
decreasing liver fat content to <5%,
decreasing cT1 as compared to baseline, optionally by 40 ms or more,
decreasing Pro-C3 as compared to baseline, optionally by 15 ng/mL or more,
decreasing ALT as compared to baseline, optionally by 10% or more,
decreasing AST as compared to baseline, optionally by 5% or more, or
preventing progression of liver fibrosis, optionally improving liver fibrosis, optionally by at least one stage,
in an adult patient in need thereof, comprising administering by subcutaneous injection to the patient a compound of Formula I, or a pharmaceutically acceptable salt or ester thereof: 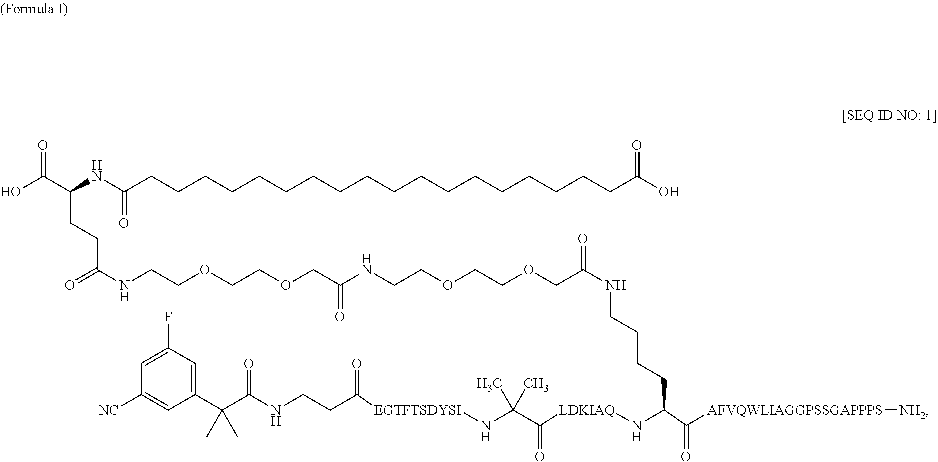
wherein the compound is administered at a body weight-independent dose of about 2.0 mg to about 25 mg.

5. The method of claim 1, wherein the patient is overweight or obese.

6. The method of claim 1, wherein the patient has a BMI of ≥25 kg/m², ≥27 kg/m², ≥30 kg/m², ≥35 kg/m², or ≥40 kg/m².

7. The method of claim 1, wherein the patient is obese, optionally wherein the patient has a BMI of 30.0 kg/m².

8. The method of claim 1, wherein the patient has liver fibrosis.

9. The method of claim 8, wherein the liver fibrosis is mild, moderate, or advanced.

10. The method of claim 8, wherein the patient has stage F1, F2, or F3 fibrosis.

11. The method of claim 1, wherein the patient receives an additional therapy, optionally wherein the additional therapy is diet therapy or exercise therapy.

12. The method of claim 1, wherein the patient does not have type 2 diabetes mellitus, or is prediabetic.

13. The method of claim 1, wherein the patient has type 2 diabetes mellitus.

14. The method of claim 1, wherein the patient has one or more weight-related comorbidities, optionally prediabetes, hypertension, dyslipidemia, obstructive sleep apnea, or previously diagnosed cardiovascular disease.

15. The method of claim 1, wherein the administering step is repeated once every week, once every two weeks, once every four weeks, or once every month.

16. The method of claim 1, wherein the dose is about 5.0, about 8.0, about 12.0, about 17.0, or about 22.0 mg, optionally wherein the dose is up-titrated over a period of 55-80, optionally 57-78 or 57, days.

17. The method of claim 16, wherein the starting dose for the up-titration is about 5 mg.

18. The method of claim 16, wherein the maximum dose for the up-titration is about 22 mg.

19. An article of manufacture or kit for use in the method of claim 1, wherein the article of manufacture or kit comprises one or more units of said dose.

20. The article of manufacture or kit of claim 19, wherein the article of manufacture or kit comprises a syringe or an injector, optionally a single-use syringe or injector.
